(19) United States
(12) Patent Application Publication
(10) Pub. No.: US 2023/0002387 A1
(43) Pub. Date: Jan. 5, 2023

(54) 2-AMINO-S6-SUBSTITUTED THIOPURINE COMPOUNDS AS INHIBITORS OF THE ENPP1 PROTEIN

(71) Applicants: ATEN PORUS LIFESCIENCES PVT. LTD., Bommanahalli Bangalore (IN); AVAMMUNE THERAPEUTICS INC, Levittown, PA (US)

(72) Inventors: Aditya KULKARNI, Bommanahalli Bangalore (IN); Sandeep GOYAL, Bommanahalli Bangalore (IN); Princy KHURANA, Bommanahalli Bangalore (IN); Ketul PATEL, Bangalore (IN); Rajath CYRIAC, Bommanahalli Bangalore (IN); Bala ANOOP SIRISH KATARU, Bommanahalli Bangalore (IN); Mukesh GANGAR, Bommanahalli Bangalore (IN); Apurba MUKHERJEE, Bommanahalli Bangalore (IN)

(73) Assignees: ATEN PORUS LIFESCIENCES PVT. LTD., Bommanahalli Bangalore (IN); AVAMMUNE THERAPEUTICS INC, Levittown, PA (US)

(21) Appl. No.: 17/760,828

(22) PCT Filed: Sep. 15, 2020
(86) PCT No.: PCT/IB2020/058558

§ 371 Date: Mar. 16, 2022

(30) Foreign Application Priority Data
Sep. 16, 2019 (IN) 201941037291
Apr. 24, 2020 (IN) 202041017699

(51) Int. Cl. C07D 473/24

(52) U.S. Cl. CPC C07D 473/24

(57) ABSTRACT

Compounds, pharmaceutical compositions, and methods are provided herein that may be used to treat cancer, infectious disease, and other conditions associated with ectonucleotide pyrophosphatase pyrophosphatase-phosphodiesterase (ENPP1) dysfunction.

### CROSS-REFERENCE TO RELATED APPLICATIONS

This application claims the benefit of and priority to Indian Provisional Application No. 201941037291 filed Sep. 16, 2019 and Indian Provisional Application No. 202041017699 filed Apr. 24, 2020, each of which is hereby incorporated by reference in its entirety for all purposes.

### BACKGROUND

Ectonucleotide Pyrophophatase/Phosphodiesterase (ENPP) family members include seven isoforms, ENPP1-7, which are type II transmembrane glycoproteins or ectoenzymes. One isoform, ENPP1(Plasma cell membrane glycoprotein-1, PC-1), has been implicated in a number of physiological processes, such as development, formation and trafficking, as well as in pathophysiological conditions. Aberrant ENPP1 expression has been detected in breast cancers relative to normal mammary epithelium, and there is evidence of its potential in the development of bone metastasis (occurs in approximately 80% cases), Hodgkin's lymphoma, hepatocellular carcinoma, follicular lymphoma, glioblastoma and in other malignant tumor tissues. In addition, mutations in ENPP1 have been associated with several disorders including infantile arterial calcification (generalized arterial calcification of infancy or GACI), ossification of the posterior longitudinal ligament of the spine and insulin signaling and resistance. ENPP1 expression is high in bone and cartilage and is implicated in lung and kidney fibrosis. A correlation was also found between expression of ENPP1 and the grading of astrocytic tumors. Another study reported that ENPP1 was required to maintain the undifferentiated and proliferative state of glioblastoma stem-like cells. Therefore, ENPP1 appears to bea viable target for the development of novel anticancer, cardiovascular, diabetes, obesity and anti-fibrotic therapeutics. Furthermore, ENPP1 activity has also been implicated in diseases caused by bacteria and/or viruses, and therefore modulators of ENPP1 may be useful in treating bacterial and/or viral diseases and conditions.

### BRIEF SUMMARY

Described herein are various embodiments directed to compounds, compositions, and methods useful for treating diseases and conditions associated with ENPP1 dysfunction. In some embodiments, the compounds disclosed herein are inhibitors of ENPP1.

In some embodiments, the present disclosure provides a compound of Formula (X) or a pharmaceutically acceptable salt, hydrate, or tautomer thereof:

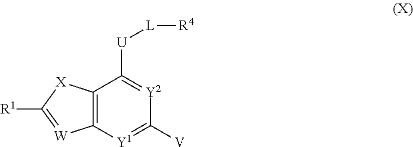

wherein:

L is a linker selected from alkylene, alkenylene, alkylene-S—, alkylene-O—, optionally substituted -alkylene-(NR⁵)—, optionally substituted

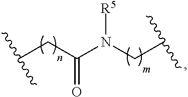

optionally substituted

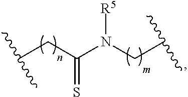

optionally substituted

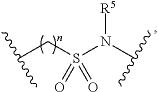

optionally substituted

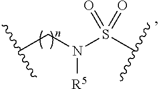

optionally substituted

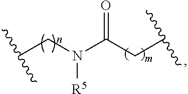

optionally substituted

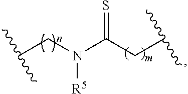

optionally substituted

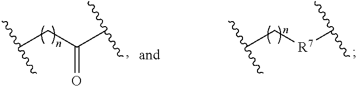

U is S or NH;

V is OH, NR²N³ or V and Y¹ taken together with the atoms to which they are attached form an optionally substituted phenyl or pyridinyl ring;

W is CH or N;

X is O, S, NR⁶, —CH═CH—, or —CH═N—;

Y¹ and Y² are each independently CH or N;

R¹ is H, OH, O-alkyl, alkyl or carbocyclyl;

R² and R³ are each independently H, alkyl, alkylenearyl, or —C(O)alkyl;

R⁴ is carbocyclyl, heterocyclyl, aryl, or heteroaryl, each of which is optionally substituted;

R⁵ is H, alkyl, —C(O)alkyl, carbocyclyl, alkylenecarbocyclyl, or alkylenearyl;

R⁶ is H, alkyl, carbocyclyl, alkylenecarbocyclyl, alkylenearyl, —C(O)alkyl, or —C(O)Oalkylenearyl;

R⁷ is carbocyclyl, heterocyclyl, or heteroaryl;

m is 0, 1, or 2; and

n is 1, 2, or 3.

In further embodiments, the present disclosure provides a compound of Formula (Y) or a pharmaceutically acceptable salt, hydrate, or tautomer thereof:

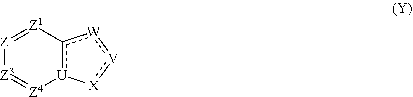

wherein:

U is C or N;
wherein
when U is C, Y is

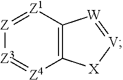

or
when U is N, Y is

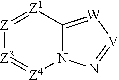

V is N or CR¹⁰;

W is CH or N;

X is S, O, N-L-R¹¹, or NR¹²;

L is selected from alkylene, alkenylene, optionally substituted -alkylene-(NR¹²)—, optionally substituted

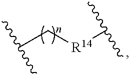

optionally substituted

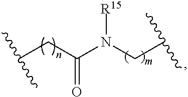

optionally substituted

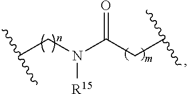

optionally substituted

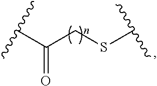

optionally substituted

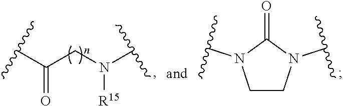

R¹⁰ is H, alkyl, —O-alkyl, —S-alkyl, carbocyclyl, alkylenecarbocyclyl, —O-L-R¹¹, —S-L-R¹¹, —N(R¹²)-L-R¹¹, -L-R¹¹;

R¹¹ is alkyl, carbocyclyl, heterocyclyl, aryl, or heteroaryl, each of which is optionally substituted;

R¹² is each independently H, alkyl, alkylenecarbocyclyl, or carbocyclyl, wherein two R¹² groups taken together with the carbon atom to which they are attached can form a heterocyclyl;

R¹⁴ is carbocyclyl, heterocyclyl, or heteroaryl;

R¹⁵ is H, alkyl, carbocyclyl, alkylenecarbocyclyl, or alkylenearyl;
wherein:
   when X is N-L-R¹¹, V is N or CR¹⁰, wherein R¹⁰ is H, alkyl, —O-alkyl, —S-alkyl, carbocyclyl, or alkylenecarbocyclyl;
   when X is S, O, NR¹²; V is CR¹⁰, wherein R¹⁰ is —O-L-R¹¹, —S-L-R¹¹, —N(R¹²)-L-R¹¹, or -L-R¹¹; or
   when U is N, V is CR¹⁰, wherein R¹⁰ is —O-L-R¹¹, —S-L-R¹¹, —N(R¹²)-L-R¹¹, or -L-R¹¹;

Z¹, Z², Z³, and Z⁴ are each independently CR¹³ or N;

R¹³ is H, halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN, wherein two R¹³ taken together with the atoms to which they are attached can form carbocyclyl, heterocyclyl, or heteroaryl, each of which is optionally substituted;

m is 0, 1, or 2; and

n is 1, 2, or 3.

In still further embodiments, the present disclosure provides a compound of Formula (ZZ) or a pharmaceutically acceptable salt, hydrate, or tautomer thereof:

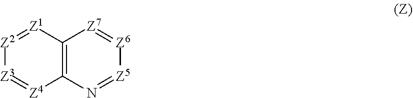

wherein:

Z¹, Z², Z³, Z⁴, Z⁵, Z⁶, and Z⁷ are each independently N or CR²², provided that
(a) one of Z¹, Z², Z³, Z⁴, Z⁵, Z⁶, or Z⁷ is -L-R¹⁸—;
(b) no more than two of Z¹, Z², Z³, or Z⁴ are N; and
(c) one of Z⁶ or Z⁷ is N;

wherein:

L is a linker selected from —N(R¹⁹)—, -alkylene-(NR¹⁹)—

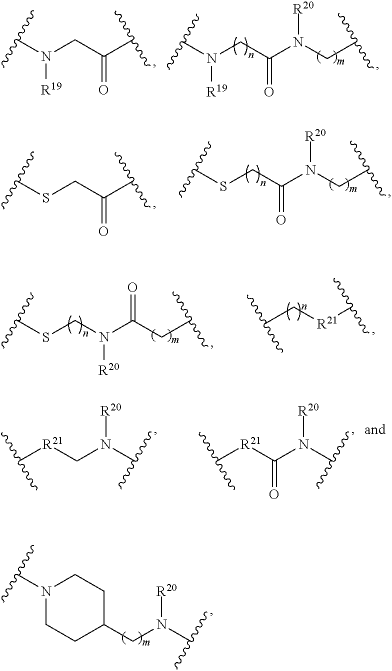

and each of which is optionally substituted;

R¹⁸ is alkyl, carbocyclyl, heterocyclyl, aryl, or heteroaryl, each of which is optionally substituted;

R¹⁹ is H, alkyl, carbocyclyl, alkylenecarbocyclyl, or alkylenearyl;

R²⁰ is H, alkyl, alkylenecarbocyclyl, alkylenearyl;

R²¹ is carbocyclyl, heterocyclyl, or heteroaryl;

R²² is each independently halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN;

m is 0, 1, or 2; and

n is 1, 2, or 3.

### BRIEF DESCRIPTION OF THE FIGURES

The skilled artisan will understand that the drawings primarily are for illustrative purposes and are not intended to limit the scope of the inventive subject matter described herein.

FIG. 1 shows the role of ENPP1 inhibitors in helping regulate the cGAS-c-GAMP-STING pathway, which is an innate immune pathway activated during infection or by a patho-physiological condition (e.g., cancer, autoimmune disorder, etc.).

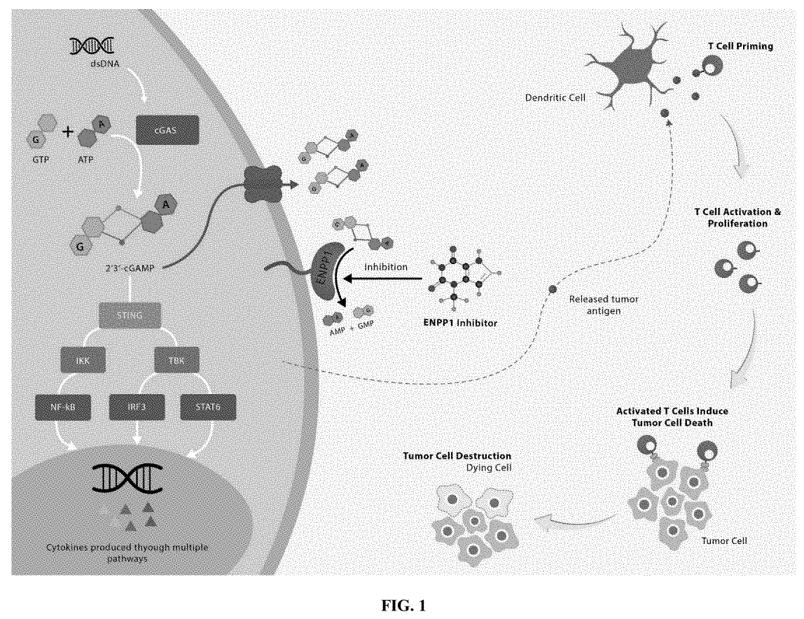

FIG. 2 provides a graph of tumor growth kinetics in an LLC1 syngeneic tumor model upon treatment with Compound 155 dosed intravenously (IV) alone or in combination with an anti-PD-1 antibody.

FIG. 3 provides a graph of tumor growth kinetics in an LLC1 syngeneic tumor model upon treatment with Compound 155 dosed orally (PO) alone or in combination with an anti-PD-1 antibody.

FIG. 4 provides a graph comparing IV and PO dosing of Compound 155 on tumor growth kinetics in an LLC1 syngeneic tumor model when provided alone or in combination with an anti-PD-1 antibody.

FIG. 5 provides a graph of tumor growth kinetics in an LLC1 syngeneic tumor model upon treatment with Compound 173 dosed intravenously (IV) alone or in combination with an anti-PD-1 antibody.

FIG. 6 provides a graph of tumor growth kinetics in an LLC1 syngeneic tumor model upon treatment with Compound 173 dosed orally (PO) alone or in combination with an anti-PD-1 antibody.

FIG. 7 provides a graph comparing IV and PO dosing of Compound 173 on tumor growth kinetics in an LLC1 syngeneic tumor model when provided alone or in combination with an anti-PD-1 antibody.

FIG. 8 provides a graph of tumor growth kinetics in an LLC1 syngeneic tumor model upon treatment with Compound 174 dosed intravenously (IV) alone or in combination with an anti-PD-1 antibody.

FIG. 9 provides a graph of tumor growth kinetics in an LLC1 syngeneic tumor model upon treatment with Compound 174 dosed orally (PO) alone or in combination with an anti-PD-1 antibody.

FIG. 10 provides a graph comparing IV and PO dosing of Compound 174 on tumor growth kinetics in an LLC1 syngeneic tumor model when provided alone or in combination with an anti-PD-1 antibody.

### DETAILED DESCRIPTION

All definitions, as defined and used herein, should be understood to control over dictionary definitions, definitions in documents incorporated by reference, and/or ordinary meanings of the defined terms. Use of flow diagrams is not meant to be limiting with respect to the order of operations performed for all embodiments. The indefinite articles “a” and “an,” as used herein in the specification and in the claims, unless clearly indicated to the contrary, should be understood to mean “at least one.”

Reference throughout this specification to “one embodiment” or “an embodiment,” etc. means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

“Alkyl” or “alkyl group” refers to a fully saturated, straight or branched hydrocarbon chain radical, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical. Alkylenes comprising any number of carbon atoms from 1 to 12 are included. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Examples of C₁-C₃ alkyl includes methyl, ethyl, n-propyl, and i-propyl. Examples of C₁-C₄ alkyl includes methyl, ethyl, n-propyl, i-propyl, n-butyl, i-butyl, and sec-butyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl groups comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 5 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Alkylenearyl” refers to a radical of the formula —R_{b}—R_{c} where R_{b} is an alkylene, as defined above and R_{c} is one or more aryl radicals as defined above. Examples include benzyl, diphenylmethyl, and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include cycloalkyl. cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, for example having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, for example having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, for example having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Alkyleneheterocyclyl” refers to a radical of the formula —R_{b}—Rₑ where R_{b} is an alkylene as defined above and Rₑ is a heterocyclyl radical as defined above, and if the heterocyclyl is a nitrogen-containing heterocyclyl, the heterocyclyl can be attached to the alkyl, alkenyl, alkynyl radical at the nitrogen atom. Unless stated otherwise specifically in the specification, a heterocyclylalkyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in this disclosure, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Alkyleneheteroaryl” refers to a radical of the formula —R_{b}—R_{f} where R_{b} is an alkylene as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

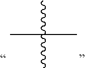

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

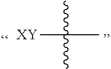

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

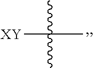

infers that when R³ is “XY”, the point of attachment bond is the same bond as the bond by which R³ is depicted as being bonded to CH₃.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

“Geminal” refers to any two substituents (e.g., those described herein such as alkyl, alkenyl, alkynyl, cycloalkyl, heterocycloalkyl, aryl, heteroaryl, etc.) that are attached to the same atom. In some embodiments, geminal substitution refers to substitution on the same carbon atom. The structure

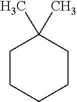

exemplifies geminal methyl substitution on cyclohexane. In some embodiments, the optional substitution is geminal substitution.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present invention includes tautomers of any said compounds.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. In some embodiments, inorganic salts include ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In particular embodiments, organic bases include isopropylamine, diethylamine, ethanolamine, trimethylamine, dicyclohexylamine, choline and caffeine.

Crystallization is a method commonly used to isolate a reaction product, for example one of the compounds disclosed herein, in purified form. Often, crystallization produces a solvate of the compound of the invention. As used herein, the term “solvate” refers to an aggregate that comprises one or more molecules of a compound of the invention with one or more molecules of solvent, typically in co-crystallized form. The solvent can be water, in which case the solvate can be a hydrate. Alternatively, the solvent can be an organic solvent. Thus, the compounds of the present invention can exist as a hydrate, including a monohydrate, dihydrate, hemihydrate, sesquihydrate, trihydrate, tetrahydrate and the like, as well as the corresponding solvated forms. The compound of the invention can be true solvates, while in other cases, the compound of the invention can merely retain adventitious water or be a mixture of water plus some adventitious solvent.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 and/or ChemDraw Professional 16.0.0.82 software naming program (CambridgeSoft), or the like. For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for some carbon atoms, which are assumed to be bonded to sufficient hydrogen atoms to complete the valency.

The invention disclosed herein is also meant to encompass the in vivo metabolic products of the disclosed compounds. Such products can result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabeled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

As used herein, a “subject” can be a human, non-human primate, mammal, rat, mouse, cow, horse, pig, sheep, goat, dog, cat, insect and the like. The subject can be suspected of having or at risk for having a cancer, such as a blood cancer, or another disease or condition. Diagnostic methods for various cancers, and the clinical delineation of cancer, are known to those of ordinary skill in the art. The subject can also be suspected of having an infection or abnormal cardiovascular function.

“Mammal” includes humans and both domestic animals such as laboratory animals and household pets (e.g., cats, dogs, swine, cattle, sheep, goats, horses, rabbits), and non-domestic animals such as wildlife and the like.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“An “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to a castration-resistant form. Typically, a prophylactic dose is used in subjects prior to or at an earlier stage of disease, so that a prophylactically effective amount can be less than a therapeutically effective amount.

“Treating” or “treatment” as used herein covers the treatment of the disease or condition of interest in a mammal, for example in a human, having the disease or condition of interest, and includes (but is not limited to):
1. preventing the disease or condition from occurring in a mammal, in particular, when such mammal is predisposed to the condition but has not yet been diagnosed as having it;
2. inhibiting the disease or condition, i.e., arresting its development;
3. relieving the disease or condition, i.e., causing regression of the disease or condition (ranging from reducing the severity of the disease or condition to curing the disease of condition); or
4. relieving the symptoms resulting from the disease or condition, i.e., relieving pain without addressing the underlying disease or condition. As used herein, the terms “disease” and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

Throughout the present specification, the terms “about” and/or “approximately” can be used in conjunction with numerical values and/or ranges. The term “about” is understood to mean those values near to a recited value. For example, “about 40 [units]” can mean within ±25% of 40 (e.g., from 30 to 50), within ±20%, +15%, +10%, ±9%, ±8%, ±7%, ±6%, +5%, ±4%, ±3%, ±2%, ±1%, less than ±1%, or any other value or range of values herein. Furthermore, the phrases “less than about [a value]” or “greater than about [a value]” should be understood in view of the definition of the term “about” provided herein. The terms “about” and “approximately” can be used interchangeably.

Numerical ranges may be provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-75, etc.).

Following below are more detailed descriptions of various concepts related to, and embodiments of inventive compounds and methods for the treatment of liver diseases and abnormal conditions of the liver. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

### Compounds and Compositions

In various embodiments, the present disclosure provides a compound of Formula (A1), Formula (A2) or a pharmaceutically acceptable salt, hydrate, or tautomer thereof:

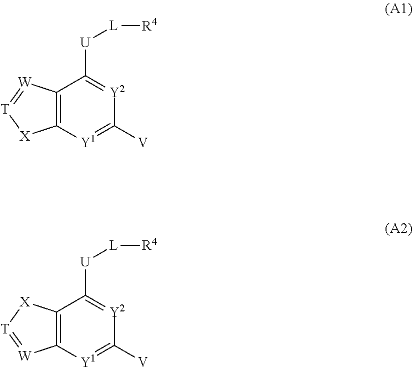

wherein:

L is a linker selected from alkylene, alkenylene, optionally substituted alkylene-S—, optionally substituted alkylene-O—, optionally substituted -alkylene-(NR⁵)—, optionally substituted

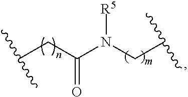

optionally substituted

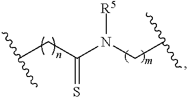

optionally substituted

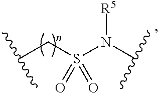

optionally substituted

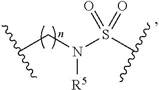

optionally substituted

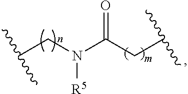

optionally substituted

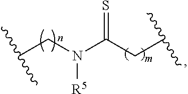

optionally substituted

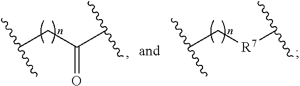

T is CR¹ or N;

U is S, S(O)₂, or NH;

V is H, OH, NR²N³ or V and Y¹ taken together with the atoms to which they are attached form an optionally substituted phenyl or pyridinyl ring;

W is CH or N;

X is O, S, NR⁶, —CH═CH—, or —CH═N—;
wherein:
when W is CH, T is N and X is O, S, or NR⁶;

Y¹ and Y² are each independently CH or N;

R¹ is H, OH, O-alkyl, alkyl or carbocyclyl;

R² and R³ are each independently H, alkyl, alkylenearyl, or —C(O)alkyl;

R⁴ is carbocyclyl, heterocyclyl, aryl, or heteroaryl, each of which is optionally substituted;

R⁵ is H, alkyl, —C(O)alkyl, carbocyclyl, alkylenecarbocyclyl, or alkylenearyl;

R⁶ is H, alkyl, carbocyclyl, alkylenecarbocyclyl, alkylenearyl, —C(O)alkyl, or —C(O)Oalkylenearyl;

R⁷ is carbocyclyl, heterocyclyl, or heteroaryl;

m is 0, 1, or 2; and

n is 1, 2, or 3.

In some embodiments, the present disclosure provides a compound of Formula (A1) or a pharmaceutically acceptable salt, hydrate, or tautomer thereof:

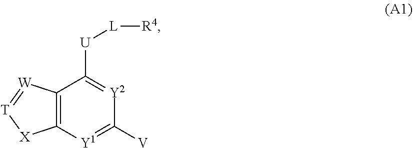

wherein L, T, U, V, W, X, Y¹, Y², and R⁴ are as defined herein.

In some embodiments of the present disclosure provides a compound of Formula (A2) or a pharmaceutically acceptable salt, hydrate, or tautomer thereof:

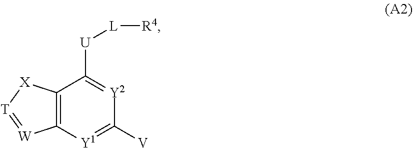

wherein L, T, U, V, W, X, Y¹, Y², and R⁴ are as defined herein.

In some embodiments, L is an alkylene, alkenylene, alkylene-(NR⁵)—,

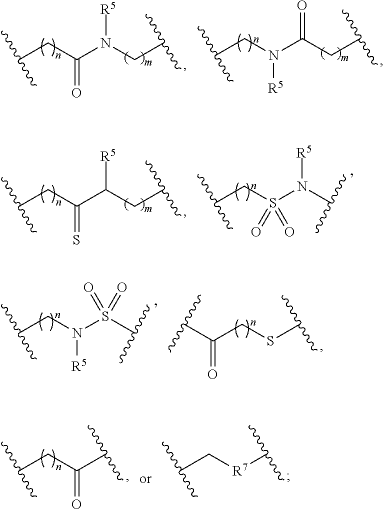

each of which is optionally substituted. In some embodiments, L is an alkylene, an alkylene-(NR⁵)—,

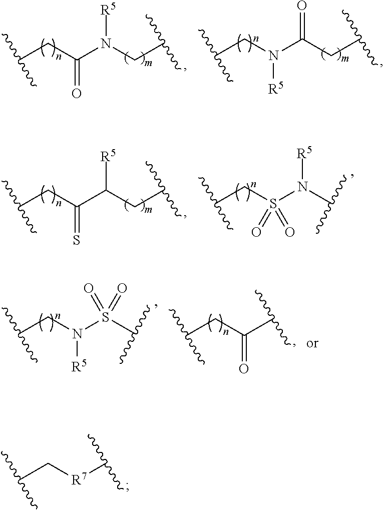

each of which is optionally substituted. In some embodiments of Formula (A1) and Formula (A2), L is an alkylene, analkenylene, an alkylene-(NR⁵)—,

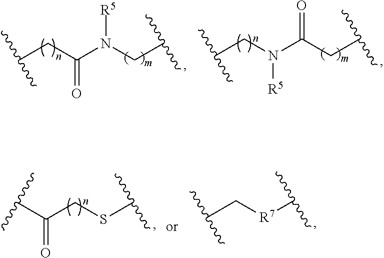

each of which is optionally substituted. In some embodiments, L is an alkylene-(NR⁵)—,

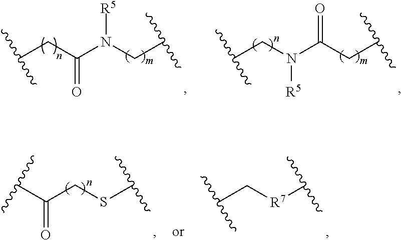

each of which is optionally substituted. In some embodiments, L is an alkylene-(NR⁵)—,

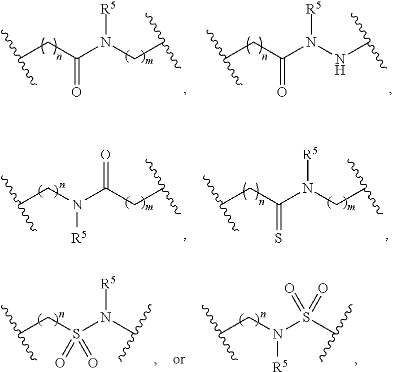

each of which is optionally substituted. In some embodiments, L is an alkylene-(NR⁵)—,

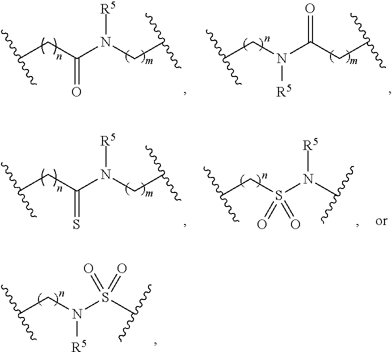

each of which is optionally substituted. In some embodiments, L is an alkylene-(NR⁵)—,

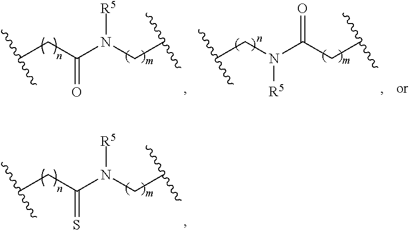

each of which is optionally substituted. In some embodiments, L is analkenylene, an alkylene-(NR⁵)—, an optionally substituted

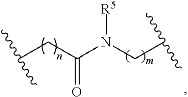

an optionally substituted

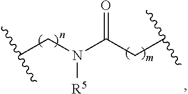

an optionally substituted

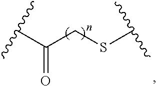

or an optionally substituted

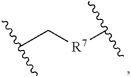

In some embodiments, L is an alkylene-(NR⁵)—,

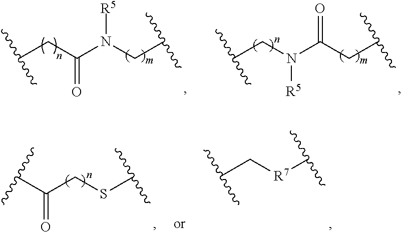

each of which is optionally substituted. In some embodiments, L is optionally substituted

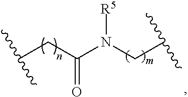

optionally substituted

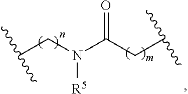

optionally substituted

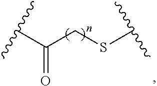

or optionally substituted

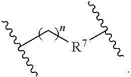

In some embodiments, L is optionally substituted

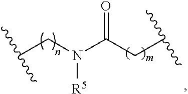

optionally substituted

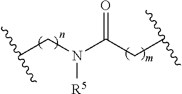

or optionally substituted

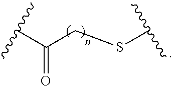 [SMILES: *SCC(*)=O]

In some embodiments, L is optionally substituted

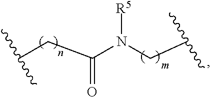

optionally substituted

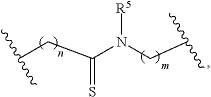

or optionally substituted

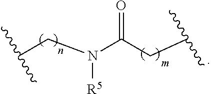

In some embodiments, L is optionally substituted

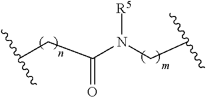

or optionally substituted

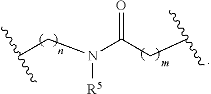

In some embodiments, L is optionally substituted

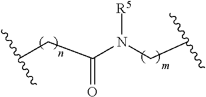

or optionally substituted

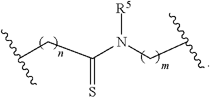

In some embodiments, L is optionally substituted

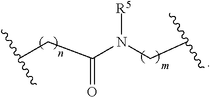

In other embodiments, L is optionally substituted

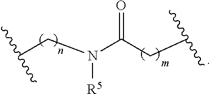

In some embodiments, L is optionally substituted

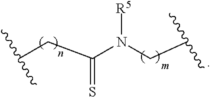

In still other embodiments, L is optionally substituted

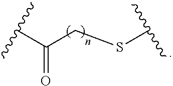 [SMILES: *SCC(*)=O]

In some embodiments, L is

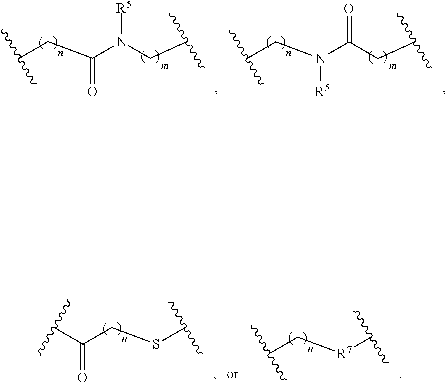

In some embodiments, L is

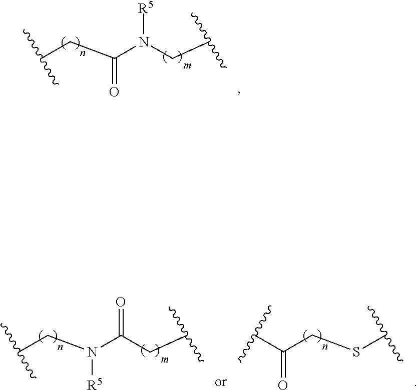

In some embodiments, L is

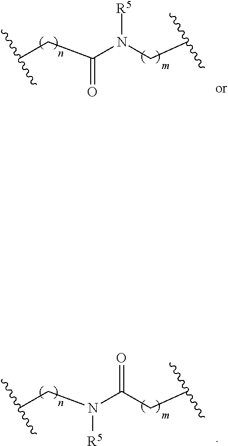

In some embodiments, L is

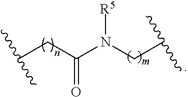

In other embodiments, L is

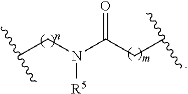

In still other embodiments, L is

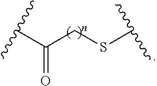 [SMILES: *SCC(*)=O]

In some embodiments, m is 0 and n is 1. In some embodiments, m is 0 and n is 2. In other embodiments, m is 1 and n is 1.

In some embodiments of Formula (A1) and Formula (A2), optionally substituted

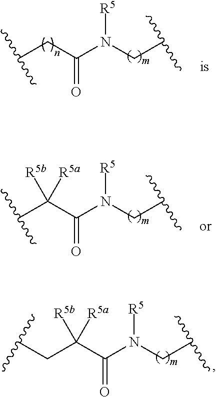

wherein:

R⁵ is H, alkyl, —C(O)alkyl, carbocyclyl, alkylenecarbocyclyl, or alkylenearyl;

R^{5a} and R^{5b} are each independently selected from the group consisting of H, halogen, C₁₋₅alkyl, C₃₋₆carbocyclyl, alkylene-C₃₋₆carbocyclyl, aryl, alkylenearyl, or NH₂; wherein two —C₁₋₅alkyl taken together with the carbon atom to which they are attached form a C₃₋₆carbocyclyl; and

m is 0 or 1.

In some embodiments, R⁵ is H, methyl, or —C(O)Me. In some embodiments, R⁵ is H. In some embodiments, R^{5a} is alkyl or carbocyclyl and R^{5b} is H.

In some embodiments, when L is

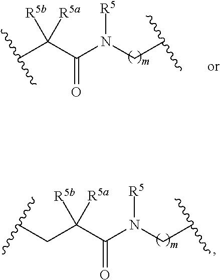

an R⁵ and an R^{5a} taken together with the carbon atoms to which they are attached form a heterocyclyl ring. In some embodiments, when L is

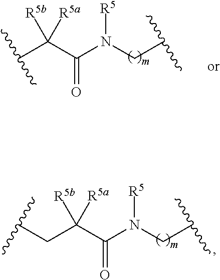

an R⁵ and an R^{5a} taken together with the carbon atoms to which they are attached form a 4-, 5- or 6-membered heterocyclyl ring. In some embodiments, the heterocyclyl ring is

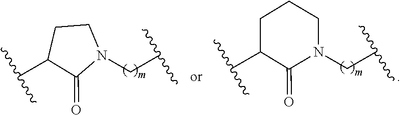 [SMILES: *CN1CCC(*)C1=O ; *CN1CCCC(*)C1=O]

In some embodiments of Formula (A1) and Formula (A2),

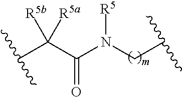

is selected from the group consisting of:

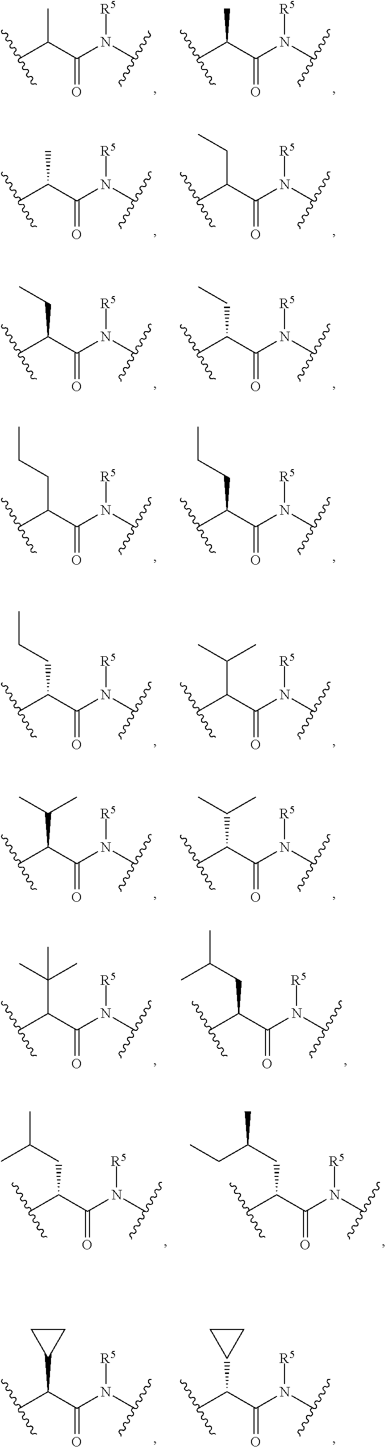

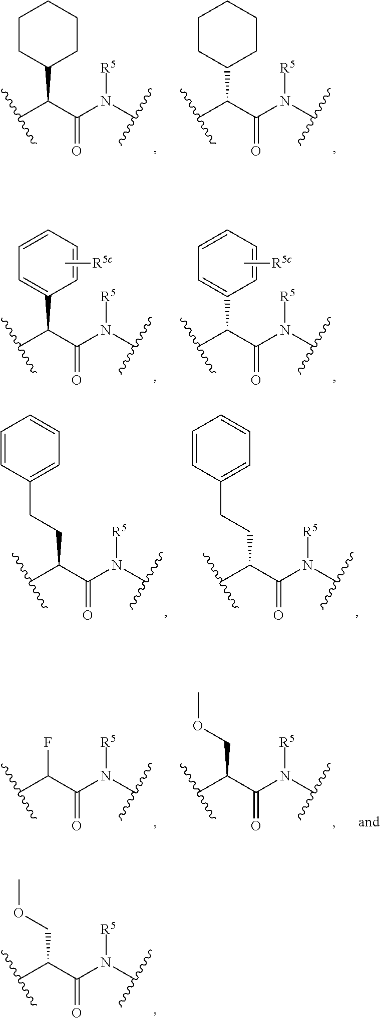

wherein R^{5c} is halogen, alkyl, haloalkyl, hydroxy, or alkoxy. In some embodiments, R^{5c} is in the para position of the phenyl ring.

In some embodiments,

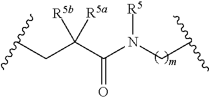

is selected from the group consisting of:

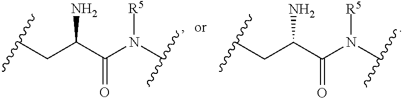

In some embodiments of Formula (A1) and Formula (A2),

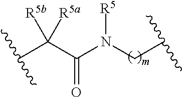

is selected from the group consisting of:

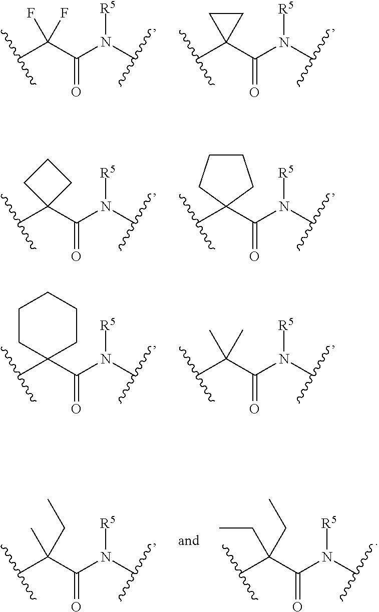

In some embodiments of Formula (A1) and Formula (A2), optionally substituted

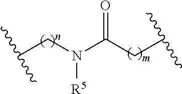

is selected from the group consisting of:

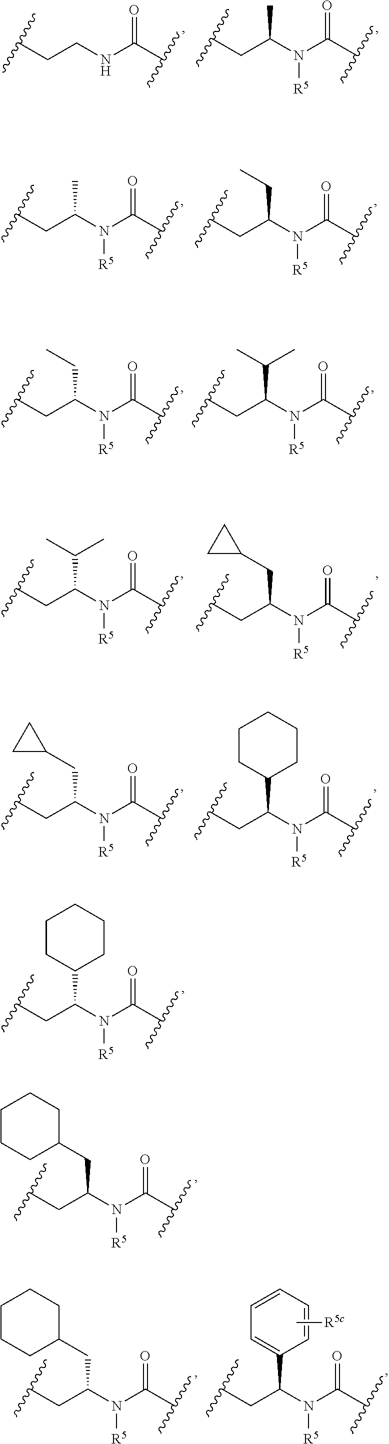

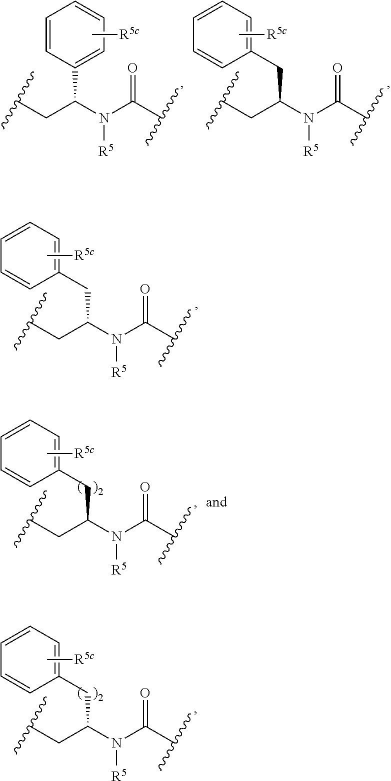

wherein R^{5c} is halogen, alkyl, haloalkyl, hydroxy, or alkoxy. In some embodiments, R^{5c} is in the para position of the phenyl ring.

In some embodiments of Formula (A1) and Formula (A2), optionally substituted

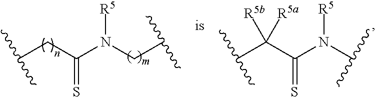

wherein:

R⁵ is H, alkyl, —C(O)alkyl, carbocyclyl, alkylenecarbocyclyl, or alkylenearyl; and

R^{5a} and R^{5b} are each independently selected from the group consisting of H, halogen, C₁₋₅alkyl, C₃₋₆carbocyclyl, alkylene-C₃₋₆carbocyclyl, aryl, alkylenearyl, or NH₂; wherein two. C₁₋₅alkyl taken together with the carbon atom to which they are attached form a C₃₋₆carbocyclyl.

In some embodiments, R⁵ is H, methyl, or —C(O)Me. In some embodiments, R⁵ is H, R^{5a} is alkyl or carbocyclyl, and R^{5b} is H. In some embodiments, R⁵ is H, R^{5a} is alkyl, and R^{5b} is H.

In some embodiments,

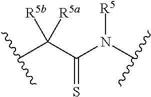

is selected from the group consisting of:

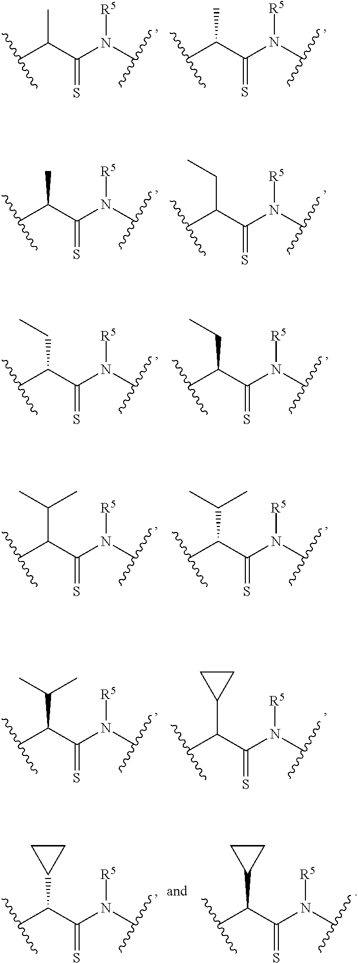

In some embodiments of Formula (A1) and Formula (A2), L comprises an alkylene. In some embodiments, the alkylene is an optionally substituted C₁₋₄alkylene. In some embodiments, the alkylene is an optionally substituted C₁₋₃alkylene. In some embodiments, the alkylene is an optionally substituted C₁₋₂alkylene. In some embodiments, the alkylene is an optionally substituted C₂₋₄alkylene. In some embodiments, the alkylene is an optionally substituted C₂₋₃alkylene. In some embodiments, the alkylene is an optionally substituted C₃₋₄alkylene. In some embodiments, when L comprises an alkylene, the alkylene is a C₁₋₄alkylene. In some embodiments, the alkylene is a C₁₋₃alkylene. In some embodiments, the alkylene is a C₁₋₂alkylene. In some embodiments, the alkylene is a C₂₋₄alkylene. In some embodiments, the alkylene is a C₂₋₃alkylene. In some embodiments, the alkylene is a C₃₋₄alkylene. In some embodiments, the alkylene is a methylene, an ethylene, a propylene, or a butylene, each of which is optionally substituted. In some embodiments, the alkylene is an ethylene, a propylene, or a butylene, each of which is optionally substituted. In some embodiments, the alkylene is an optionally substituted methylene. In some embodiments, the alkylene is an optionally substituted ethylene. In some embodiments, the alkylene is an optionally substituted propylene. In some embodiments, the alkylene is an optionally substituted butylene. In some embodiments, the alkylene is a methylene, an ethylene, a propylene, or a butylene. In some embodiments, the alkylene is a methylene. In some embodiments, the alkylene is an ethylene. In some embodiments, the alkylene is a propylene. In some embodiments, the alkylene is a butylene.

In some embodiments of Formula (A1) and Formula (A2), L is alkylene-(NR⁵)—. In some embodiments, the alkylene is optionally substituted ethylene. In some embodiments, the optionally substituted ethylene is selected from the group consisting of:

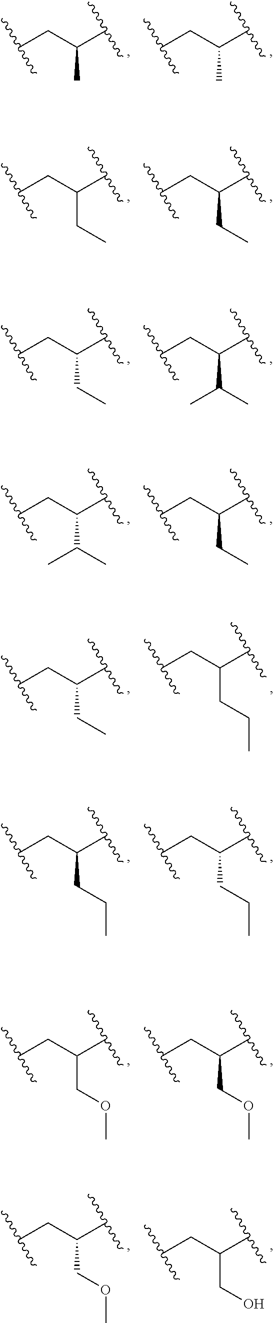

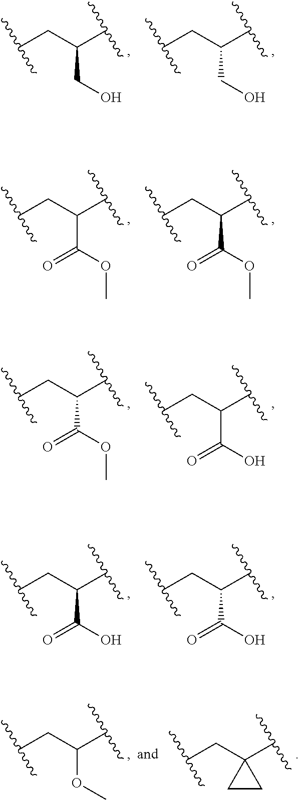

In some embodiments of Formula (A1) and Formula (A2), L is alkylene-(NR⁵)—. In some embodiments, the alkylene is optionally substituted propylene. In some embodiments, the optionally substituted propylene is selected from the group consisting of:

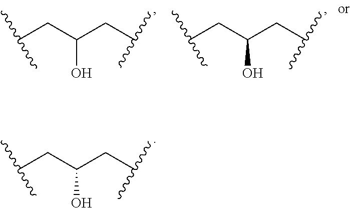

In some embodiments of Formula (A1) and Formula (A2), L comprises an alkenylene. In some embodiments, the alkenylene is an optionally substituted C₂₋₄alkenylene. In some embodiments, the alkenylene is an optionally substituted C₂₋₃alkenylene. In some embodiments, the alkenylene is an optionally substituted C₃₋₄alkenylene. In some embodiments, when L comprises an alkenylene, the alkenylene is a C₂₋₄alkenylene. In some embodiments, the alkenylene is a C₂₋₃alkenylene. In some embodiments, the alkenylene is a C₃₋₄alkenylene. In some embodiments, the alkenylene is an ethenylene, a propenylene, or a butenylene, each of which is optionally substituted. In some embodiments, the alkenylene is an optionally substituted ethenylene. In some embodiments, the alkenylene is an optionally substituted propenylene. In some embodiments, the alkenylene is an optionally substituted butenylene. In some embodiments, the alkenylene is an ethenylene, a propenylene, or a butenylene. In some embodiments, the alkenylene is an ethenylene. In some embodiments, the alkenylene is a propenylene. In some embodiments, the alkenylene is a butenylene.

In some embodiments of Formula (A1) and Formula (A2), the optional substituent is selected from the group consisting of oxo, halogen, C₁₋₅alkyl, C₃₋₆carbocyclyl, alkylenecarbocyclyl, aryl, heteroaryl, alkylenearyl, and alkyleneheteroaryl. In some embodiments, the optional substituent is selected from the group consisting of oxo, C₁₋₅alkyl, and C₃₋₆cycloalkyl. In some embodiments, the optional substituent is selected from the group consisting of oxo and C₁₋₅alkyl. In some embodiments, the optional substituent is oxo. In other embodiments, the optional substituent is C₁₋₅alkyl. In some embodiments, the C₁₋₅alkyl is methyl, ethyl, propyl or isopropyl. In some embodiments, the C₁₋₅alkyl is methyl, ethyl, or isopropyl. In other embodiments, the C₁₋₅alkyl is methyl. In some embodiments, the C₃₋₆cycloalkyl is cyclopropyl or cyclohexyl. In some embodiments, the aryl is phenyl. In some embodiments, the alkylenecarbocyclyl is methylenecyclopropyl or methylenecyclohexyl. In some embodiments, the alkylenearyl is methylenephenyl.

In some embodiments of Formula (A1) and Formula (A2), m is 0 or 1. In some embodiments, m is 1 or 2. In some embodiments, m is 0 or 2. In some embodiments, m is 0. In some embodiments, m is 1. In some embodiments, m is 2.

In some embodiments of Formula (A1) and Formula (A2), n is 1 or 2. In some embodiments, n is 2 or 3. In some embodiments, n is 1 or 3. In some embodiments, n is 1. In some embodiments, n is 2. In some embodiments, n is 3.

In some embodiments of Formula (A1) and Formula (A2), m is 0 and n is 1. In other embodiments, m is 1 and n is 1. In still other embodiments, m is 0 and n is 2. In yet another embodiment, m is 2 and n is 1.

In some embodiments of Formula (A1) and Formula (A2), T is N. In other embodiments, T is CR¹.

In some embodiments of Formula (A1) and Formula (A2), U is S. In other embodiments, U is NH.

In some embodiments of Formula (A1) and Formula (A2), V is H, OH, NR²N³, or N═CR²R³. In some embodiments of Formula (A1) and Formula (A2), V is H, OH, or NR²N³. In some embodiments, V and Y¹ taken together with the atoms to which they are attached form an optionally substituted phenyl or pyridinyl ring. In some embodiments, V is NR²N³. In other embodiments, V is OH. In some embodiments, V is H.

In some embodiments of Formula (A1) and Formula (A2), W is N. In other embodiments, W is CH.

In some embodiments of Formula (A1) and Formula (A2), X is O, S, or NR⁶. In some embodiments, X is O or NR⁶. In some embodiments, X is NR⁶. In some embodiments, X is O. In some embodiments, X is S. In some embodiments, X is —CH═CH— or —CH═N—.

In some embodiments of Formula (A1) and Formula (A2), Y¹ or Y² is N. In some embodiments, Y¹ and Y² are both N. In some embodiments, Y¹ is N and Y² is CH. In some embodiments, Y¹ is CH and Y² is N.

In some embodiments of Formula (A1) and Formula (A2), U is S, W is N, and X is NR⁶. In certain embodiments, V is NR²NR³.

In some embodiments of Formula (A1) and Formula (A2), U is S, W is N, and X is NR⁶. In certain embodiments, Y¹ and Y² are each N.

In some embodiments of Formula (A1) and Formula (A2), U is S, W is N, and X is NR⁶. In certain embodiments, V is NR²NR³.

In some embodiments of Formula (A1) and Formula (A2), U is S, W is N, X is NR⁶, and Y¹ and Y² are each N. In certain embodiments, V is NR²NR³.

In some embodiments of Formula (A1) and Formula (A2), U is S, W is N, X is NR⁶, and V is NR²NR³. In certain embodiments, Y¹ and Y² are each N.

In some embodiments of Formula (A1) and Formula (A2), when W is CH, T is N, U is S, V is H or NR²R³, and X is NR⁶. In certain embodiments, Y¹ and Y² are each N.

In some embodiments of Formula (A1) and Formula (A2), R¹ is H, OH, or C₁₋₅alkyl. In other embodiments, R¹ is H. In some embodiments, R¹ is OH. In some embodiments, R¹ is C₁₋₅alkyl. In some embodiments, the C₁₋₅alkyl is selected from the group consisting of methyl, ethyl, propyl, isopropyl, isoamyl, and isobutyl. In other embodiments, the C₁₋₅alkyl is selected from the group consisting of methyl, ethyl, and isopropyl.

In some embodiments of Formula (A1) and Formula (A2), R² and R³ are independently H, —C₁₋₅alkyl, —CH₂Ph, or —C(O)(C₁₋₅alkyl). In some embodiments, R² and R³ are independently H, —C₁₋₅alkyl, —CH₂Ph, or —C(O)(CH₃). In some embodiments, one of R² and R³ is H. In some embodiments, R² and R³ are H. In some embodiments, one of R² and R³ is —C₁₋₅alkyl. In some embodiments, one of R² and R³ is —CH₂Ph. In some embodiments, one of R² and R³ is —C(O)(CH₃). In some embodiments, the C₁₋₅alkyl is selected from the group consisting of methyl, ethyl, and isopropyl.

In some embodiments of Formula (A1) and Formula (A2), R⁴ is aryl or heteroaryl, each of which is optionally substituted. In some embodiments, R⁴ is optionally substituted aryl. In some embodiments, R⁴ is optionally substituted heteroaryl. In some embodiments, the heteroaryl is oxazolyl, thiazolyl, triazolyl, oxadiazolyl, thiadiazolyl, imidazolyl, isoxazolyl, indolyl, oxindolyl, isatinyl, benzothiazolyl, benzoxazolyl, benzimidazolyl, benzotriazolyl, benzofuranyl, benzothiophenyl, pyrazolyl, pyridinyl, pyrazinyl, pyrimidinyl, quinolinyl, isoquinolinyl, cinnolinyl, quinazolinyl, or quinoxalinyl. In some embodiments, the aryl is a 6- to 12-membered aryl and the heteroaryl is a 5- to 12-membered heteroaryl with 1, 2, or 3 heteroatoms selected from the group consisting of N, O, and S. In some embodiments, the 5- to 12-membered heteroaryl with 1, 2, or 3 heteroatoms selected from the group consisting of N, O, and S is oxazolyl, thiazolyl, triazolyl, oxadiazolyl, thiadiazolyl, imidazolyl, isoxazolyl, tetrazolyl, or pyrazolyl. In some embodiments, the 5- to 12-membered heteroaryl with 1, 2, or 3 heteroatoms selected from the group consisting of N, O, and S is pyridinyl, pyrazinyl, or pyrimidinyl. In some embodiments, the 5- to 12-membered heteroaryl with 1, 2, or 3 heteroatoms selected from the group consisting of N, O, and S is indolinyl, benzothiazolyl, benzoxazolyl, benzimidazolyl, benzofuranyl, quinolinyl, isoquinolinyl, cinnolinyl, quinazolinyl, and quinoxalinyl.

In some embodiments, R⁴ is an aryl or heteroaryl, each of which is optionally substituted with one or more H, halogen, alkyl, alkene, alkyne, haloalkyl, carbocyclyl, heterocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN. In other embodiments, the aryl or heteroaryl is optionally substituted with one or more H, halogen, —C₁₋₅alkyl, —CF₃, —OH, —O(C₁₋₅alkyl), —OCF₃, —OSO₂Me, —COOH, —C(O)OMe, or —SO₂Me. In some embodiments, the aryl is an optionally substituted phenyl. In some embodiments, the heteroaryl is an optionally substituted pyridinyl. In certain embodiments, the optionally substituted pyridinyl is selected from the group consisting of

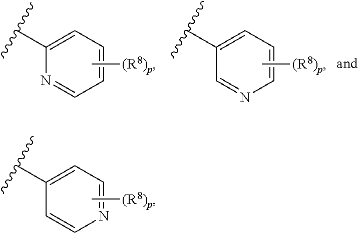

wherein p is 0, 1, or 2. In some embodiments, the heteroaryl is an optionally substituted pyrimidinyl. In certain embodiments, the optionally substituted pyrimidinyl is

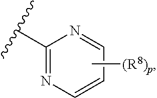

wherein p is 0, 1, or 2. In some embodiments, each R⁸ is independently halogen, alkyl, —OH, —Oalkyl, —CO₂H, or —CO₂alkyl.

In some embodiments of Formula (A1) and Formula (A2), R⁴ is an optionally substituted aryl selected from the group consisting of:

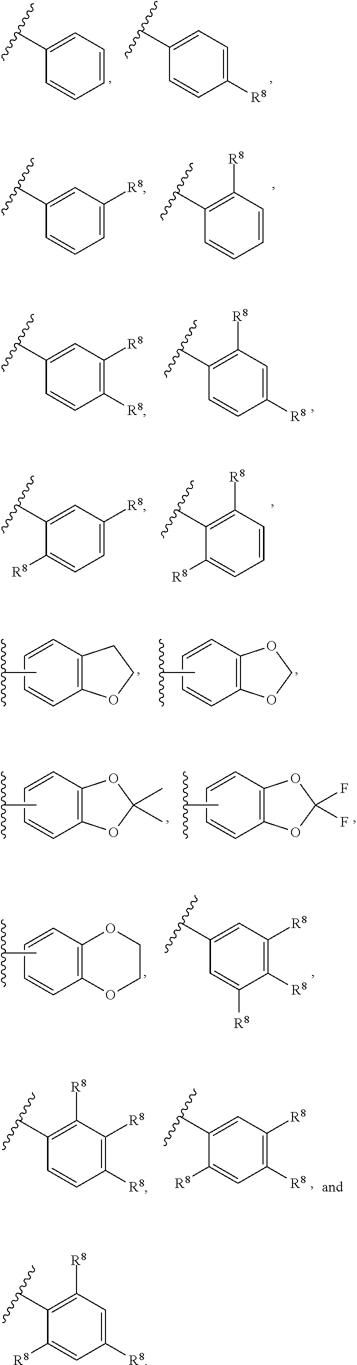

In some embodiments, R⁴ is an optionally substituted heteroaryl selected from the group consisting of:

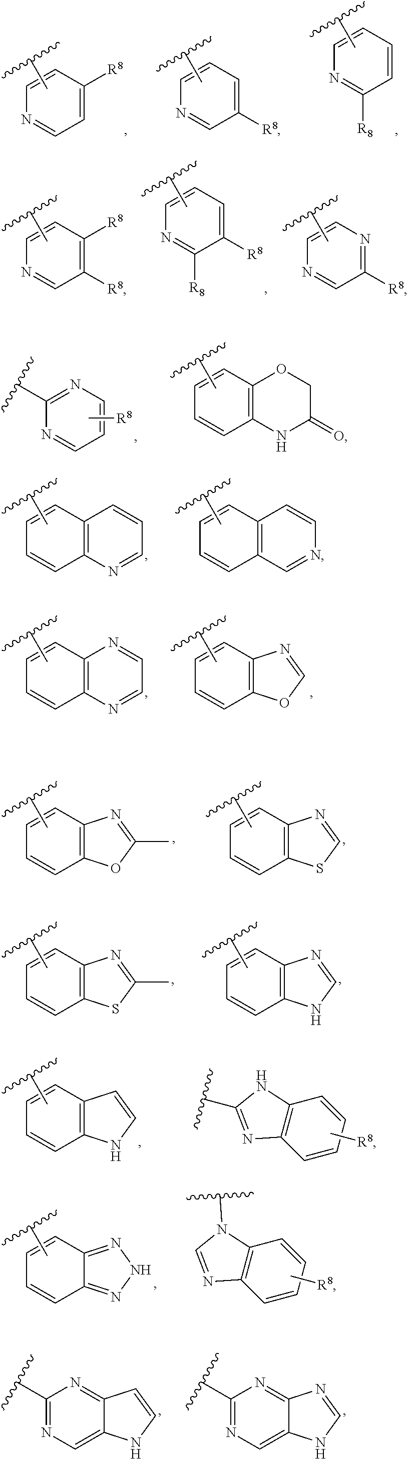

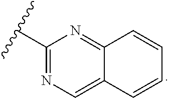 [SMILES: *c1ncc2ccccc2n1]

In some embodiments, R⁴ is selected from the group consisting of:

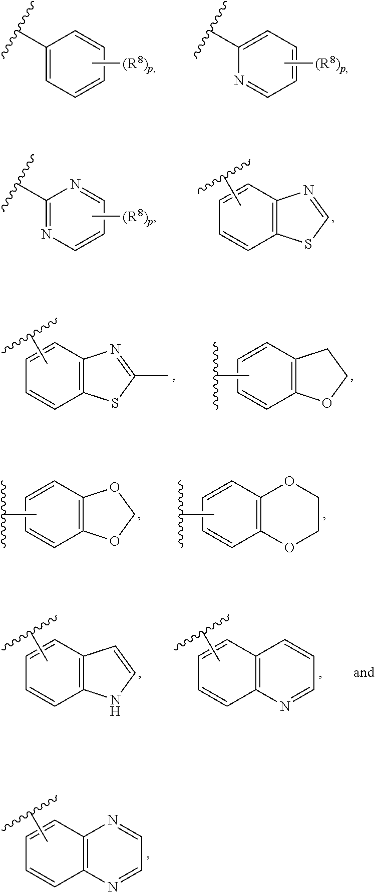

wherein p is an integer from 0-3. In some embodiments, each R⁸ is independently halogen, alkyl, haloalkyl, alkenyl, —OH, —Oalkyl, —N(alkyl)₂, —CO₂H, —CO₂alkyl, or —CN.

In some embodiments, R⁴ is selected from the group consisting of:

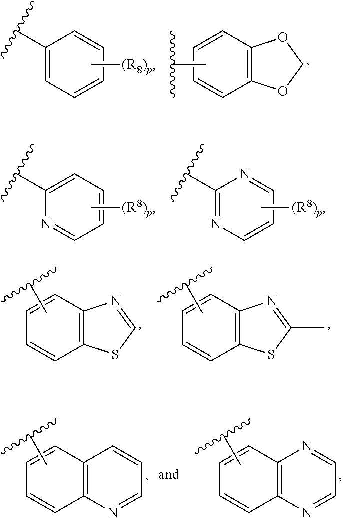

wherein:

each R⁸ is independently halogen, C₁₋₅ alkyl, —OH, —OC₁₋₅alkyl, —COOH, or —CO₂C₁₋₅alkyl; and

p is an integer from 0-3.

In some embodiments of Formula (A1) and Formula (A2), R⁴ is carbocyclyl. In some embodiments, the carbocyclyl is an optionally substituted C₃₋₆carbocyclyl. In some embodiments, the carbocyclyl is cyclopropyl, cyclobutyl, cyclopentyl, or cyclohexyl. In some embodiments, the carbocyclyl is cyclohexyl.

In some embodiments of Formula (A1) and Formula (A2), R⁴ is heterocyclyl. In some embodiments, the heterocyclyl is an optionally substituted 4- to 6-membered heterocyclyl containing 1 or 2 heteroatoms selected from N, O, and S. In some embodiments, the heterocyclyl is azetidinyl, pyrrolidinyl, piperidinyl, piperazinyl, morpholinyl, or thiomorpholinyl.

In some embodiments of Formula (A1) and Formula (A2), R⁵ is H, C₁₋₅alkyl, —C(O)C₁₋₄alkyl, C₃₋₆carbocyclyl, —CH₂-aryl, or CH₂—(C₃₋₆carbocyclyl). In some embodiments, R⁵ is H, C₁₋₅alkyl, —C(O)Me, or C₃₋₆carbocyclyl. In some embodiments, R⁵ is H or C₁₋₅alkyl. In some embodiments, the C₁₋₅alkyl is selected from the group consisting of methyl, ethyl, and isopropyl. In some embodiments, the C₃₋₆carbocyclyl is cyclopropyl or cyclohexyl. In some embodiments, R⁵ is H, Me, or —C(O)Me. In some embodiments, R⁵ is H, Me, or CH₂Ph. In some embodiments, R⁵ is H. In some embodiments, R⁵ is Me. In some embodiments, R⁵ is —C(O)Me.

In some embodiments of Formula (A1) and Formula (A2), R⁶ is H, C₁₋₅alkyl, CH₂aryl, or CH₂—(C₃₋₆carbocyclyl). In some embodiments, R⁶ is H, C₁₋₅alkyl, or CH₂Ph. In some embodiments, C₁₋₅alkyl is selected from the group consisting of methyl, ethyl, and isopropyl. In some embodiments, R⁶ is H.

In some embodiments of Formula (A1) and Formula (A2), R⁷ is a C₃₋₆carbocyclyl, a 3- to 6-membered heterocyclyl, or a 5- to 6-membered heteroaryl. In some embodiments, R⁷ is a C₃₋₆carbocyclyl. In some embodiments, the C₃₋₆carbocyclyl is cyclopropyl or cyclohexyl. In some embodiments, R⁷ is a 5- to 6-membered heteroaryl. In some embodiments, the 5- to 6-membered heteroaryl is selected from the group consisting of oxazolyl, thiazolyl, triazolyl, oxadiazolyl, thiadiazolyl, imidazolyl, isoxazolyl, pyrazolyl, pyridinyl, pyrimidinyl, and pyrazinyl. In some embodiments, the 5- to 6-membered heteroaryl is selected from the group consisting of

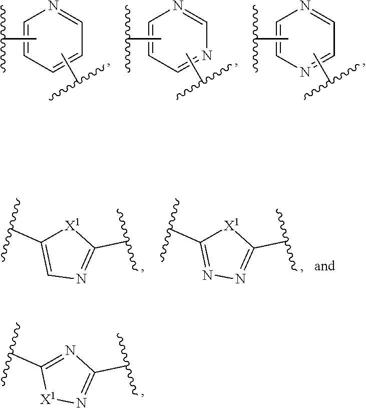

wherein X¹ is NR⁶, S, or O and R⁶ is H or alkyl. In some embodiments, R⁷ is a 5-membered heteroaryl. In some embodiments, the 5-membered heteroaryl is selected from the group consisting of

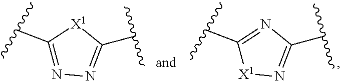

wherein X¹ is NR⁶, S, or O. In some embodiments, the 5-membered heteroaryl is selected from the group consisting of

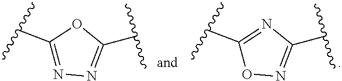 [SMILES: *c1nnc(*)o1 ; *c1noc(*)n1]

In some embodiments, R⁷ is a 3- to 6-membered heterocyclyl. In some embodiments, the 3- to 6-membered heterocyclyl is selected from the group consisting of azetidinyl, pyrrolidinyl, piperidinyl, piperazinyl, morpholinyl, and thiomorpholinyl.

In some embodiments, each R⁸ is independently halogen, alkyl, haloalkyl, alkenyl, —OH, —Oalkyl, —N(alkyl)₂, —CO₂H, —CO₂alkyl, or —CN. In some embodiments, each R⁸ is independently halogen, alkyl, haloalkyl, —OH, —Oalkyl, —Ohaloalkyl, or —CO₂H. In some embodiments, each R⁸ is independently halogen, alkyl, —OH, —Oalkyl, or —CO₂H.

In some embodiments, the compound of Formula (A1) or Formula (A2) has a structure according to one of the following

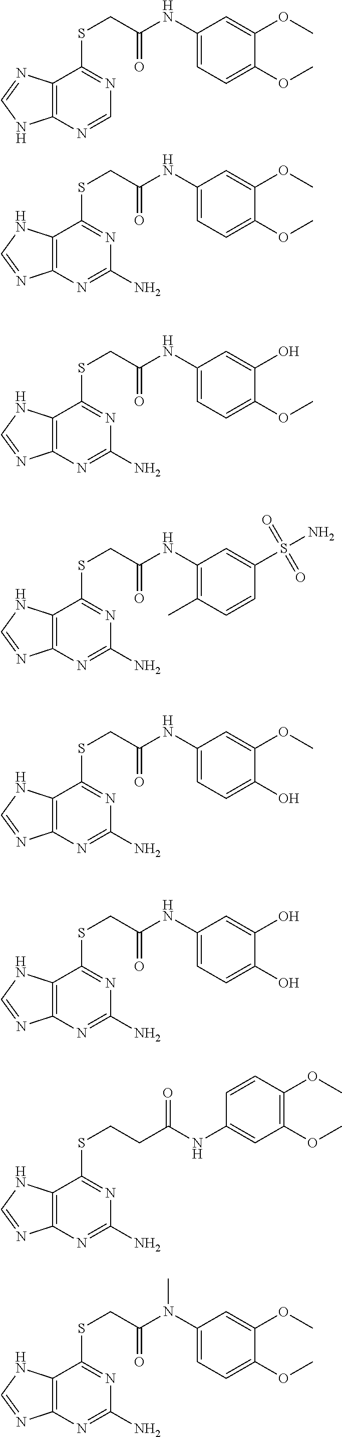

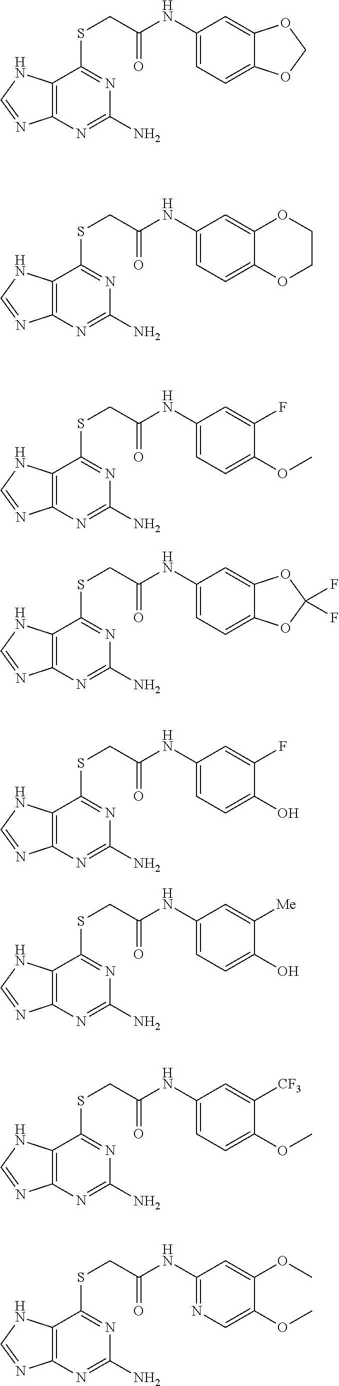

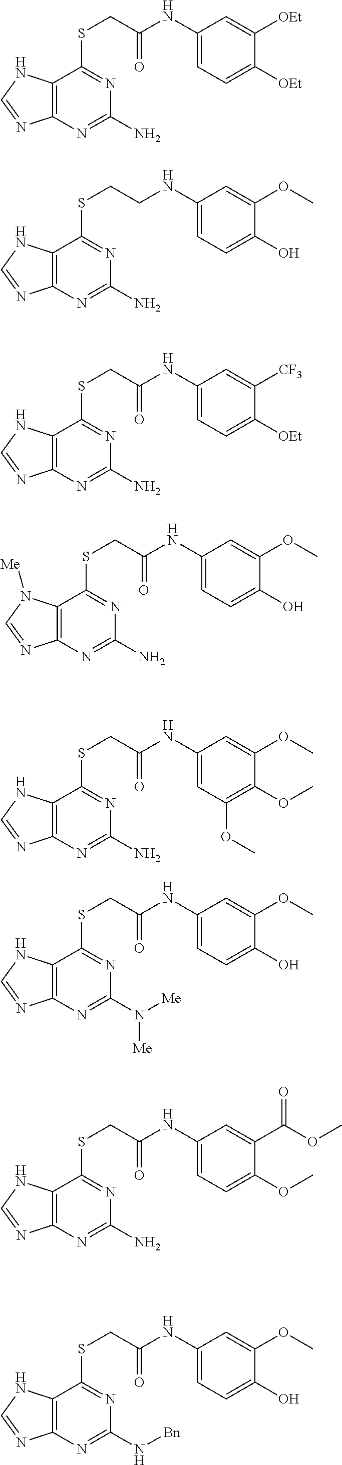

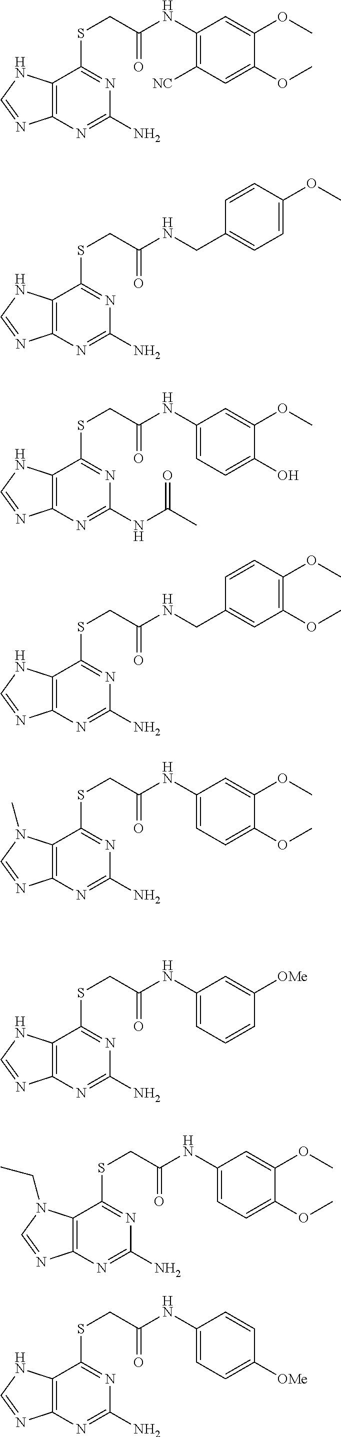

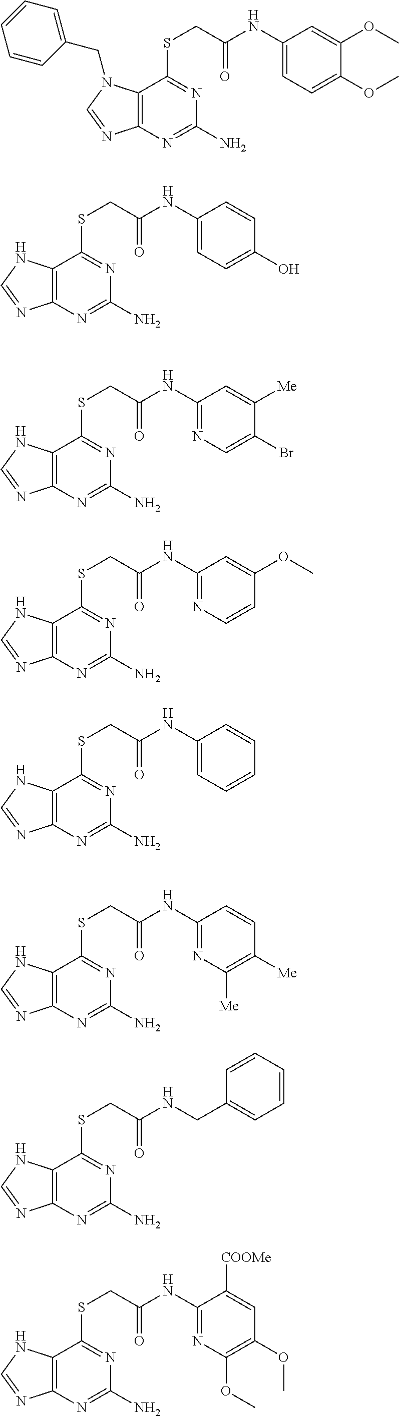

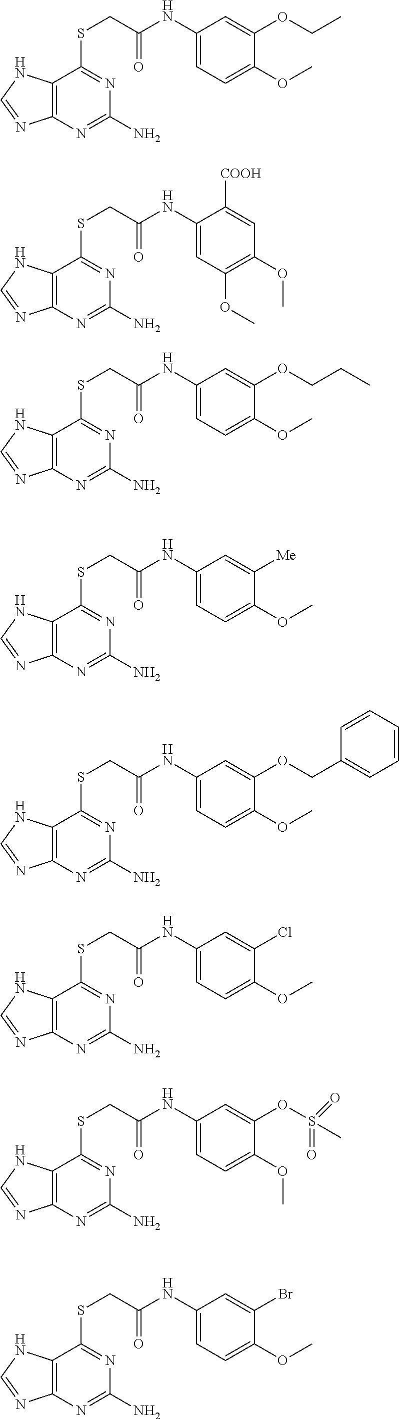

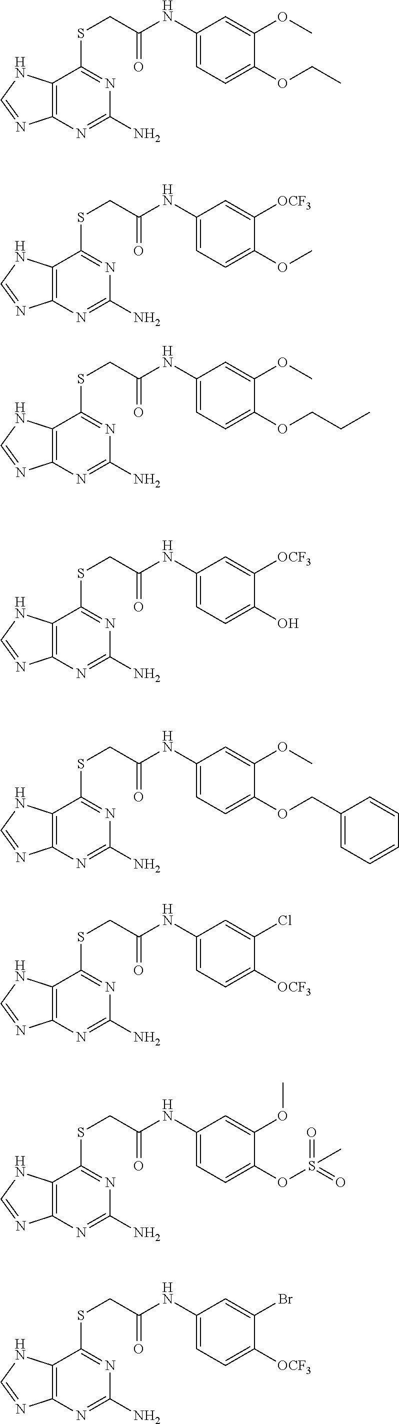

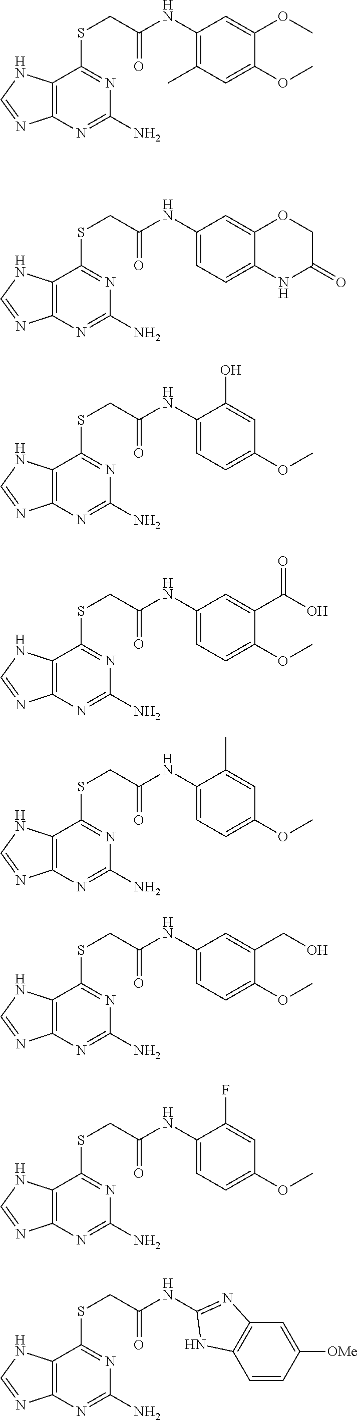

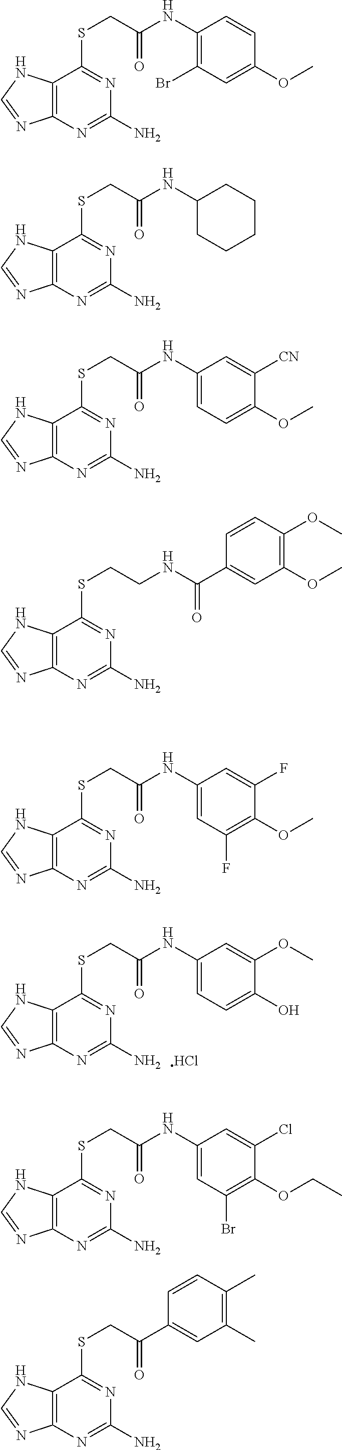

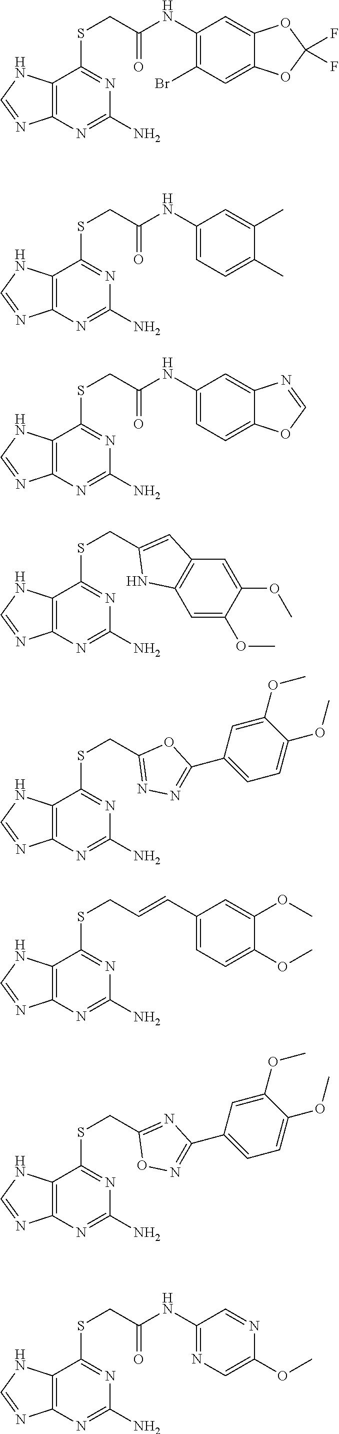

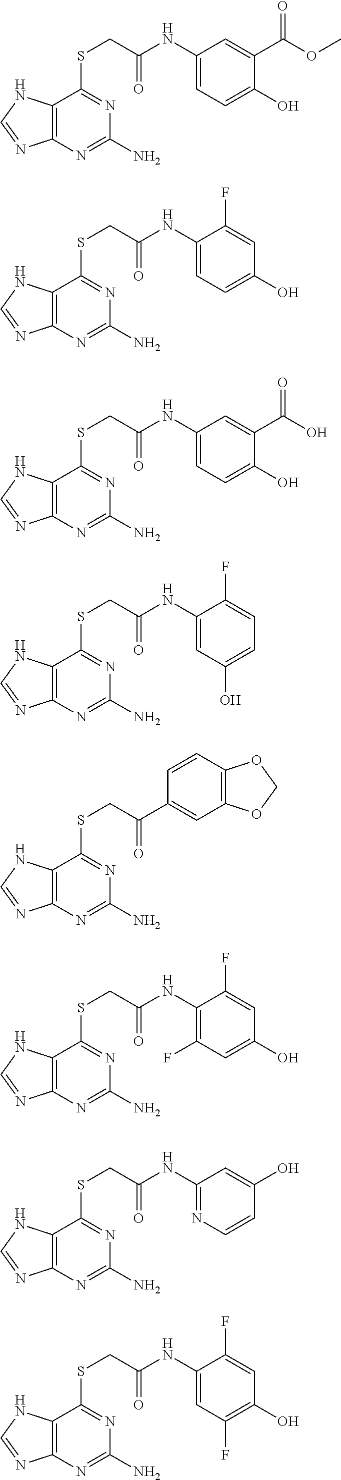

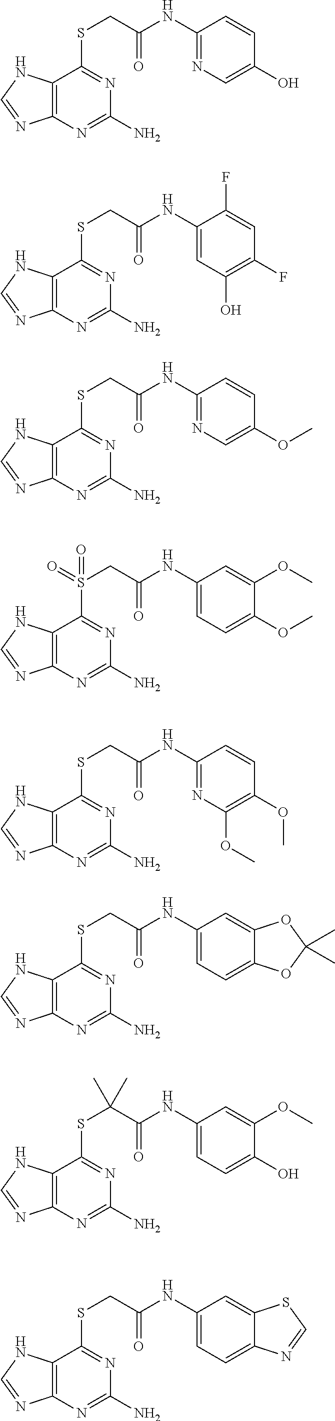

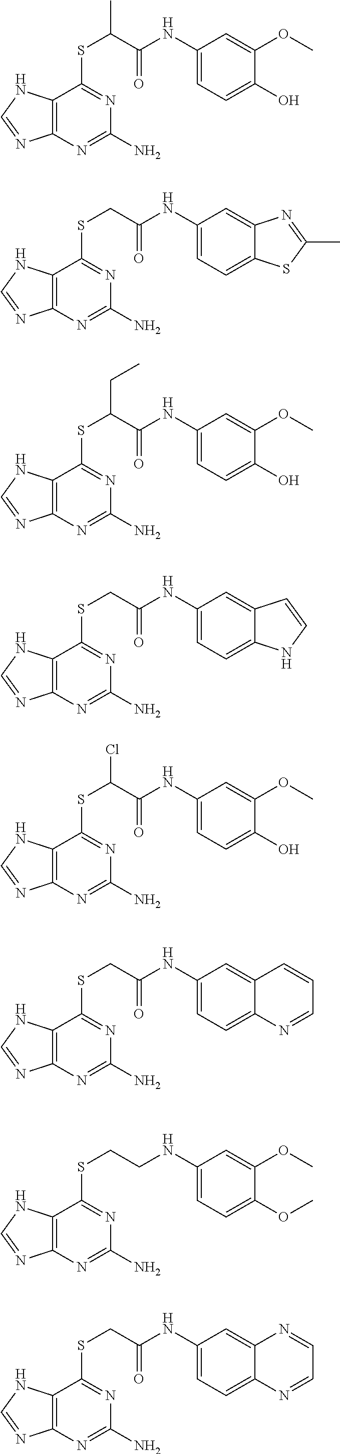

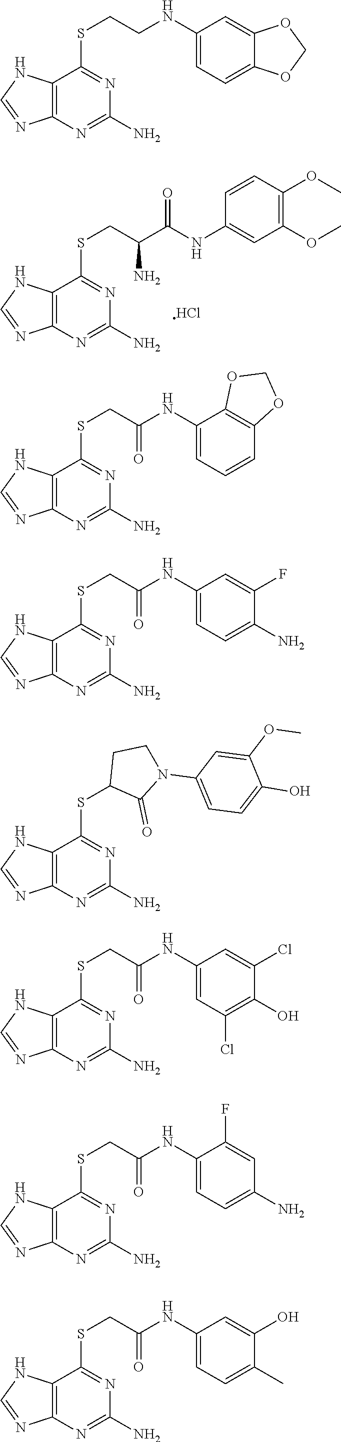

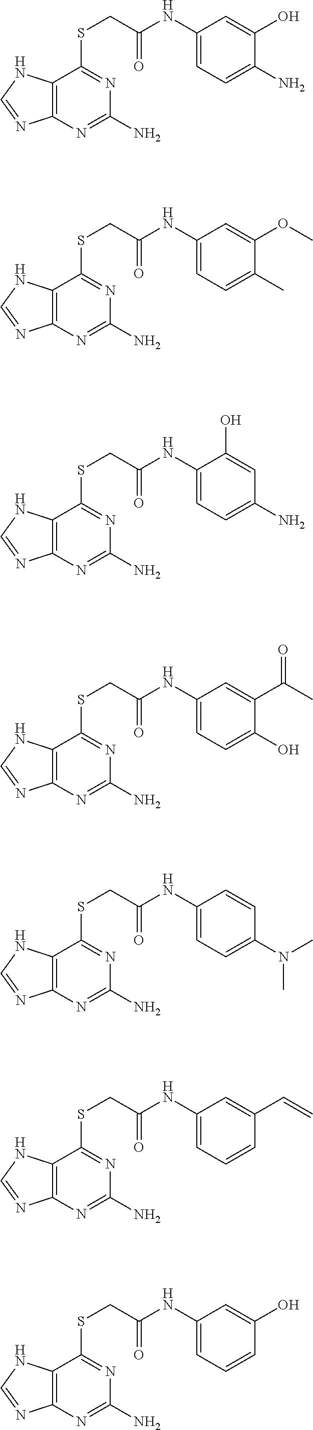

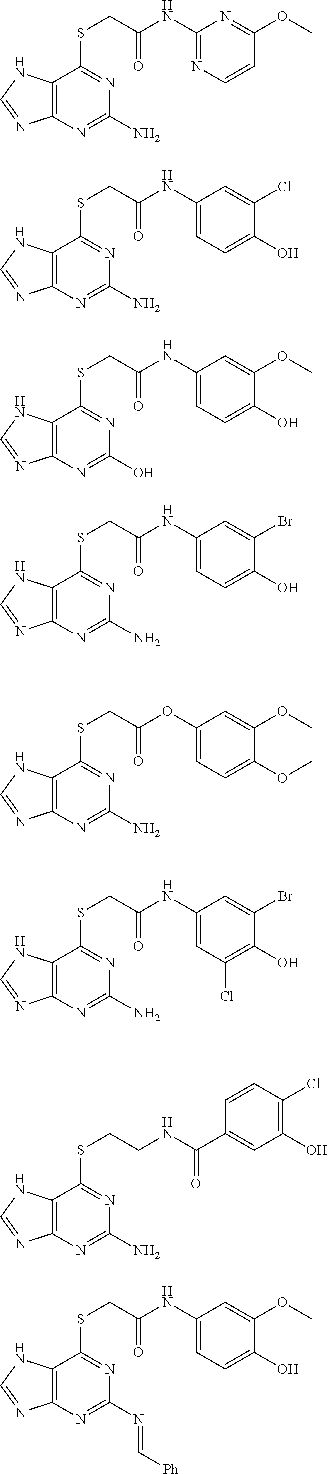

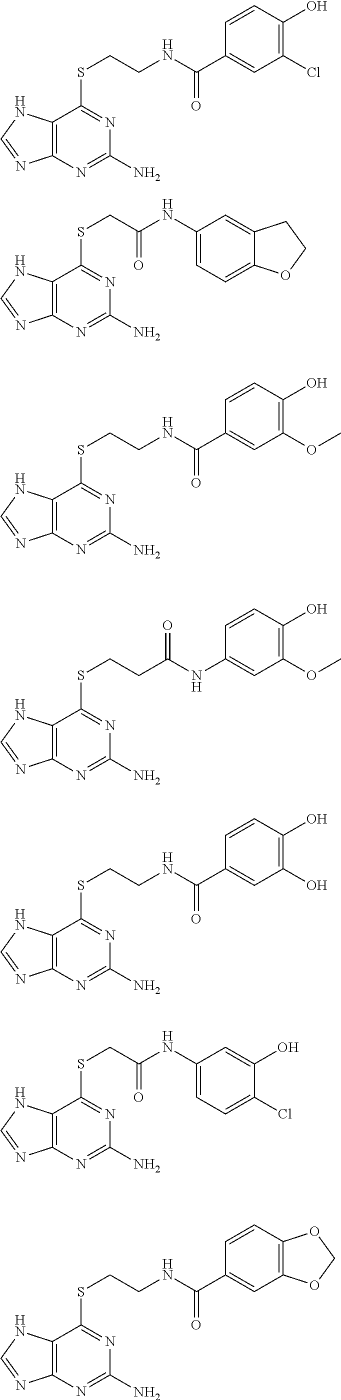

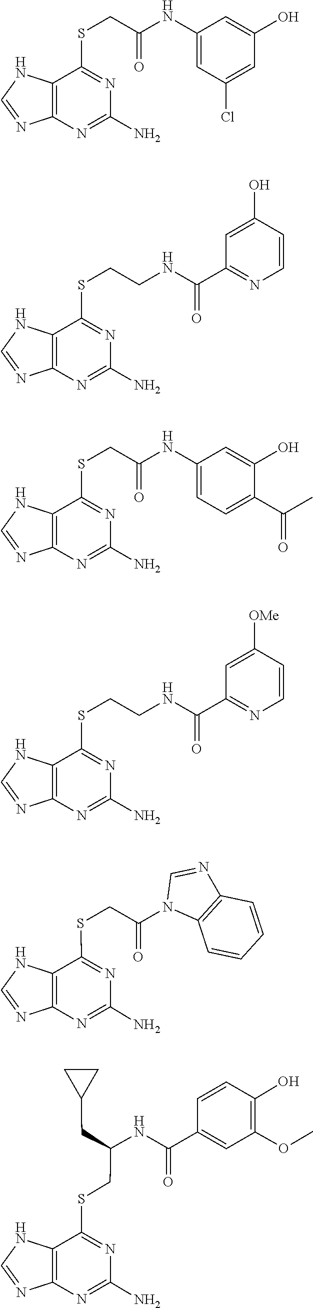 [SMILES: Nc1nc(SCC(=O)Nc2cc(O)cc(Cl)c2)c2[nH]cnc2n1 ; Nc1nc(SCCNC(=O)c2cc(O)ccn2)c2[nH]cnc2n1 ; CC(=O)c1ccc(NC(=O)CSc2nc(N)nc3nc[nH]c23)cc1O ; COc1ccnc(C(=O)NCCSc2nc(N)nc3nc[nH]c23)c1 ; Nc1nc(SCC(=O)n2cnc3ccccc32)c2[nH]cnc2n1 ; COc1cc(C(=O)N[C@@H](CSc2nc(N)nc3nc[nH]c23)CC2CC2)ccc1O]

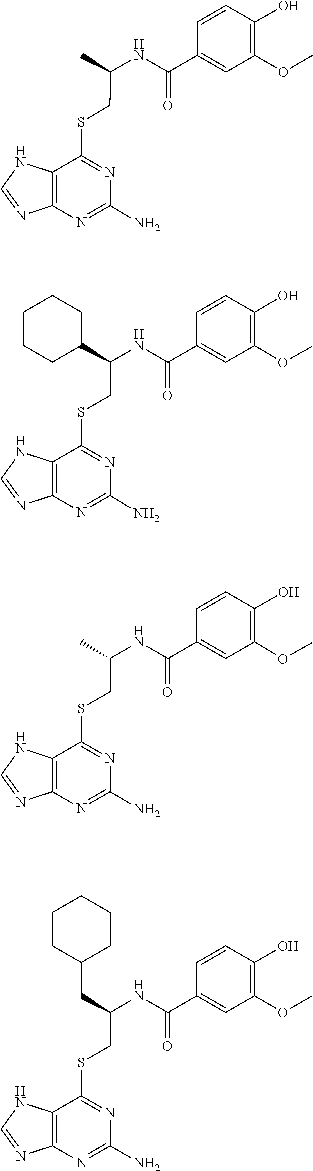 [SMILES: COc1cc(C(=O)N[C@H](C)CSc2nc(N)nc3nc[nH]c23)ccc1O ; COc1cc(C(=O)N[C@@H](CSc2nc(N)nc3nc[nH]c23)C2CCCCC2)ccc1O ; COc1cc(C(=O)N[C@@H](C)CSc2nc(N)nc3nc[nH]c23)ccc1O ; COc1cc(C(=O)N[C@@H](CSc2nc(N)nc3nc[nH]c23)CC2CCCCC2)ccc1O]

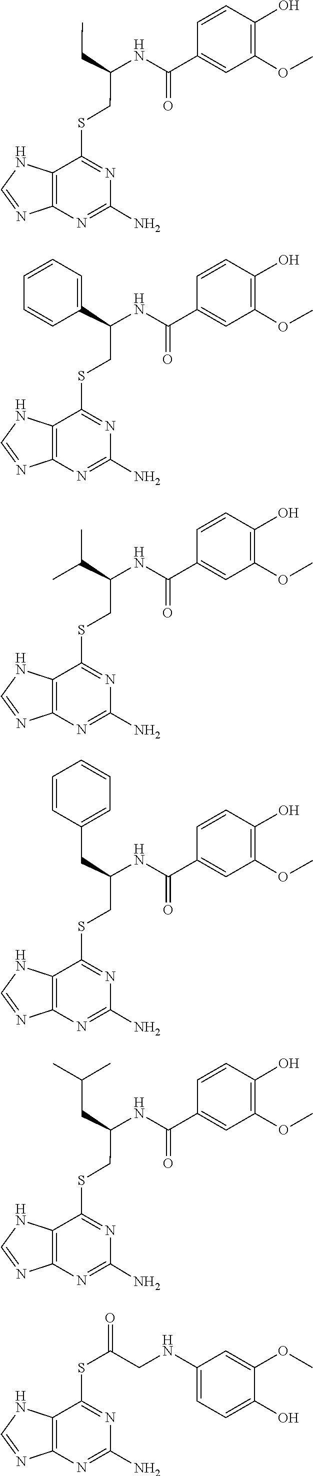

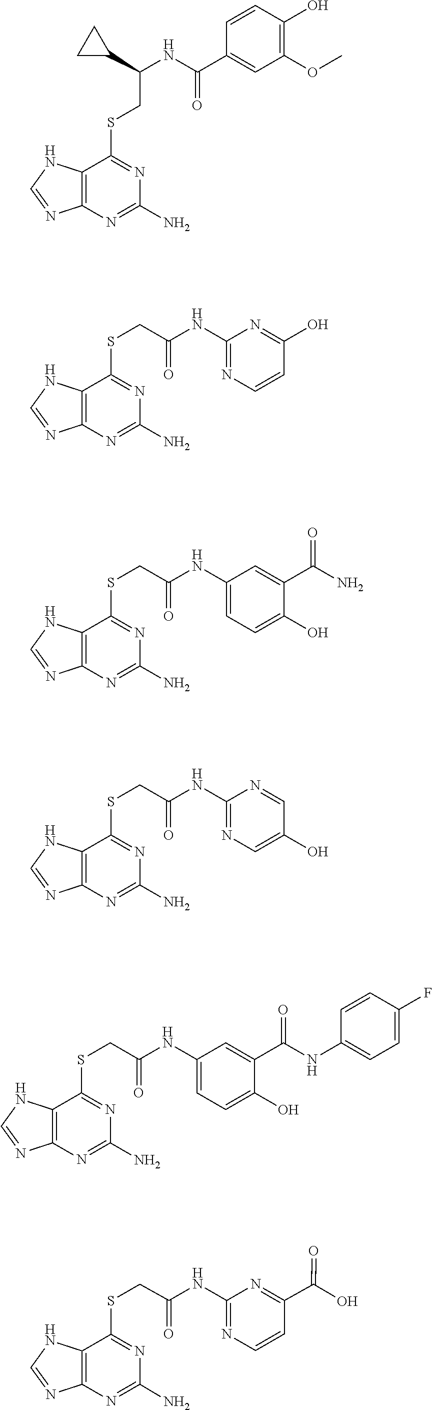 [SMILES: COc1cc(C(=O)N[C@@H](CSc2nc(N)nc3nc[nH]c23)C2CC2)ccc1O ; Nc1nc(SCC(=O)Nc2nccc(O)n2)c2[nH]cnc2n1 ; NC(=O)c1cc(NC(=O)CSc2nc(N)nc3nc[nH]c23)ccc1O ; Nc1nc(SCC(=O)Nc2ncc(O)cn2)c2[nH]cnc2n1 ; Nc1nc(SCC(=O)Nc2ccc(O)c(C(=O)Nc3ccc(F)cc3)c2)c2[nH]cnc2n1 ; Nc1nc(SCC(=O)Nc2nccc(C(=O)O)n2)c2[nH]cnc2n1]

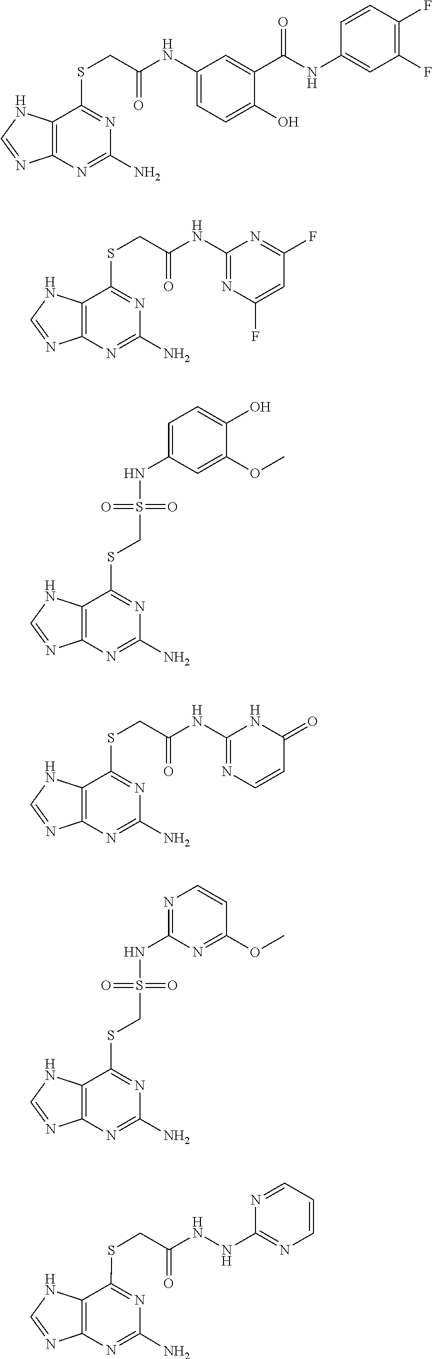 [SMILES: Nc1nc(SCC(=O)Nc2ccc(O)c(C(=O)Nc3ccc(F)c(F)c3)c2)c2[nH]cnc2n1 ; Nc1nc(SCC(=O)Nc2nc(F)cc(F)n2)c2[nH]cnc2n1 ; COc1cc(NS(=O)(=O)CSc2nc(N)nc3nc[nH]c23)ccc1O ; Nc1nc(SCC(=O)Nc2nccc(=O)[nH]2)c2[nH]cnc2n1 ; COc1ccnc(NS(=O)(=O)CSc2nc(N)nc3nc[nH]c23)n1 ; Nc1nc(SCC(=O)NNc2ncccn2)c2[nH]cnc2n1]

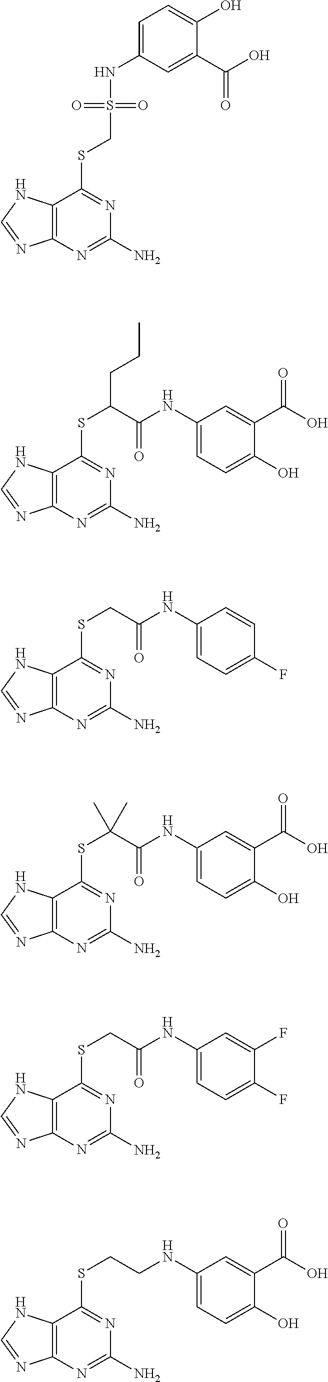 [SMILES: Nc1nc(SCS(=O)(=O)Nc2ccc(O)c(C(=O)O)c2)c2[nH]cnc2n1 ; CCCC(Sc1nc(N)nc2nc[nH]c12)C(=O)Nc1ccc(O)c(C(=O)O)c1 ; Nc1nc(SCC(=O)Nc2ccc(F)cc2)c2[nH]cnc2n1 ; CC(C)(Sc1nc(N)nc2nc[nH]c12)C(=O)Nc1ccc(O)c(C(=O)O)c1 ; Nc1nc(SCC(=O)Nc2ccc(F)c(F)c2)c2[nH]cnc2n1 ; Nc1nc(SCCNc2ccc(O)c(C(=O)O)c2)c2[nH]cnc2n1]

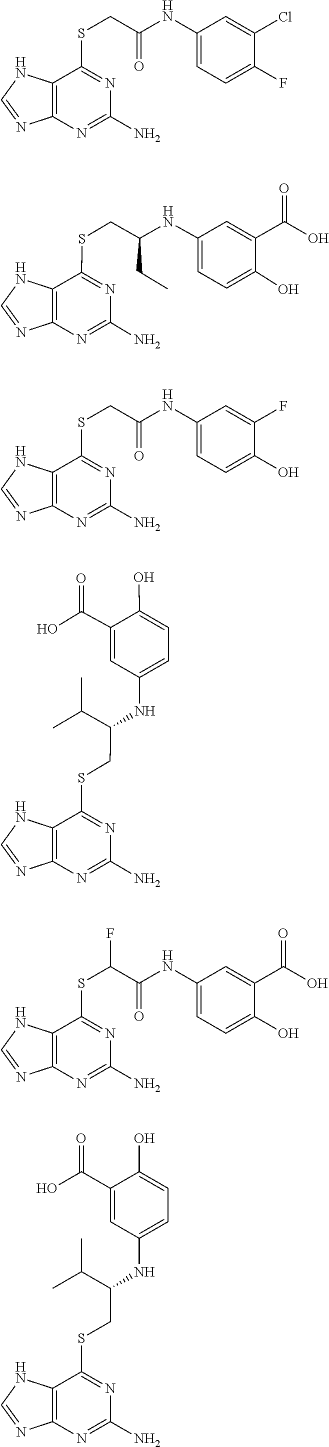 [SMILES: Nc1nc(SCC(=O)Nc2ccc(F)c(Cl)c2)c2[nH]cnc2n1 ; CC[C@@H](CSc1nc(N)nc2nc[nH]c12)Nc1ccc(O)c(C(=O)O)c1 ; Nc1nc(SCC(=O)Nc2ccc(O)c(F)c2)c2[nH]cnc2n1 ; CC(C)[C@@H](CSc1nc(N)nc2nc[nH]c12)Nc1ccc(O)c(C(=O)O)c1 ; Nc1nc(SC(F)C(=O)Nc2ccc(O)c(C(=O)O)c2)c2[nH]cnc2n1 ; CC(C)[C@@H](CSc1nc(N)nc2nc[nH]c12)Nc1ccc(O)c(C(=O)O)c1]

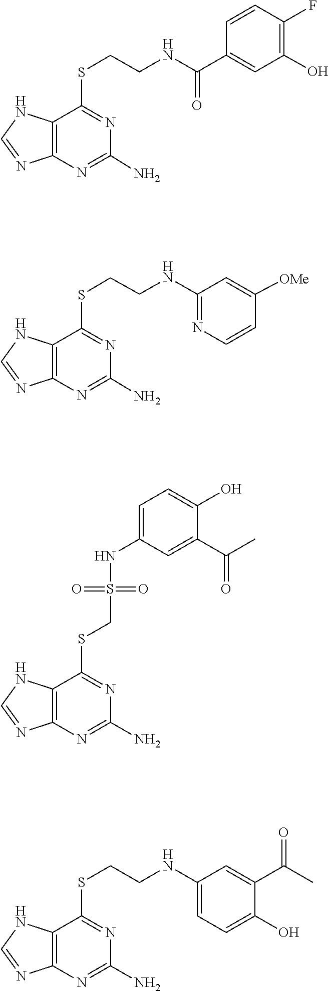 [SMILES: Nc1nc(SCCNC(=O)c2ccc(F)c(O)c2)c2[nH]cnc2n1 ; COc1ccnc(NCCSc2nc(N)nc3nc[nH]c23)c1 ; CC(=O)c1cc(NS(=O)(=O)CSc2nc(N)nc3nc[nH]c23)ccc1O ; CC(=O)c1cc(NCCSc2nc(N)nc3nc[nH]c23)ccc1O]

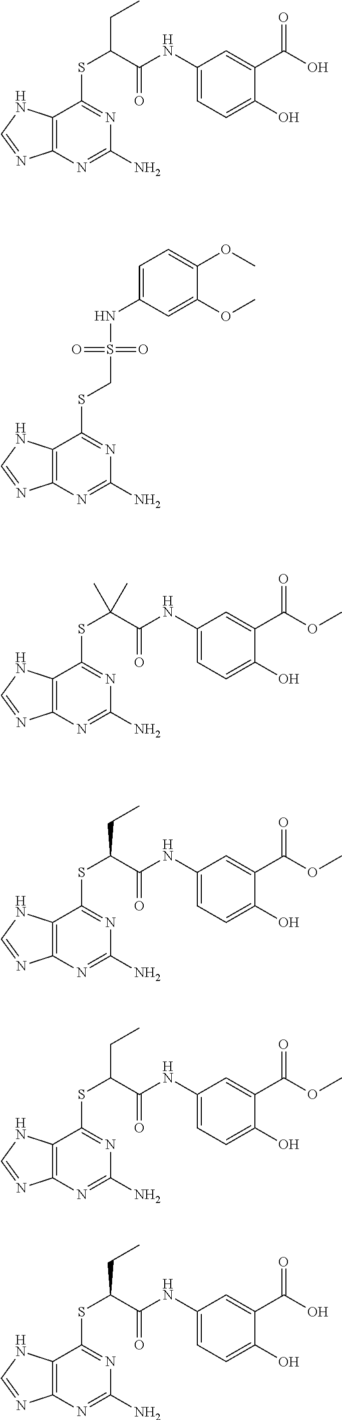 [SMILES: CCC(Sc1nc(N)nc2nc[nH]c12)C(=O)Nc1ccc(O)c(C(=O)O)c1 ; COc1ccc(NS(=O)(=O)CSc2nc(N)nc3nc[nH]c23)cc1OC ; COC(=O)c1cc(NC(=O)C(C)(C)Sc2nc(N)nc3nc[nH]c23)ccc1O ; CC[C@H](Sc1nc(N)nc2nc[nH]c12)C(=O)Nc1ccc(O)c(C(=O)OC)c1 ; CCC(Sc1nc(N)nc2nc[nH]c12)C(=O)Nc1ccc(O)c(C(=O)OC)c1 ; CC[C@H](Sc1nc(N)nc2nc[nH]c12)C(=O)Nc1ccc(O)c(C(=O)O)c1]

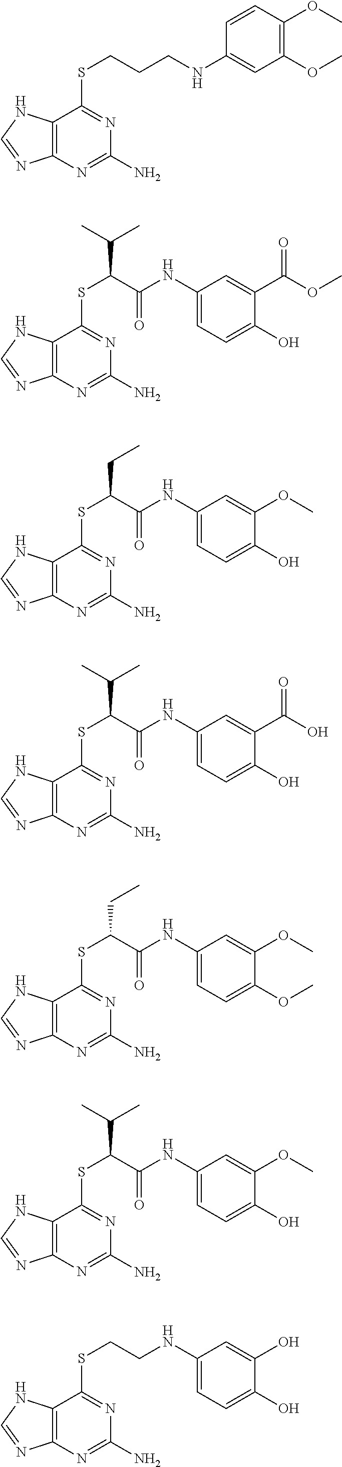

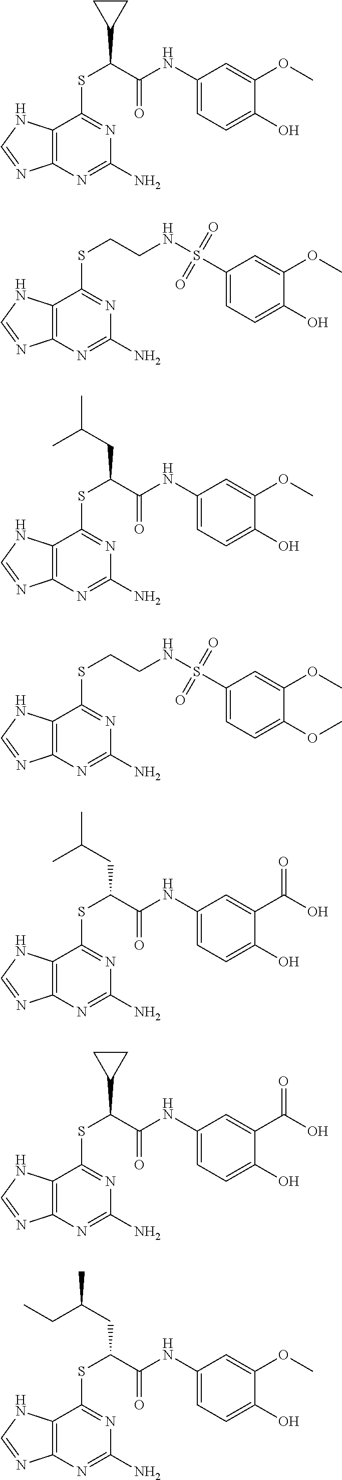

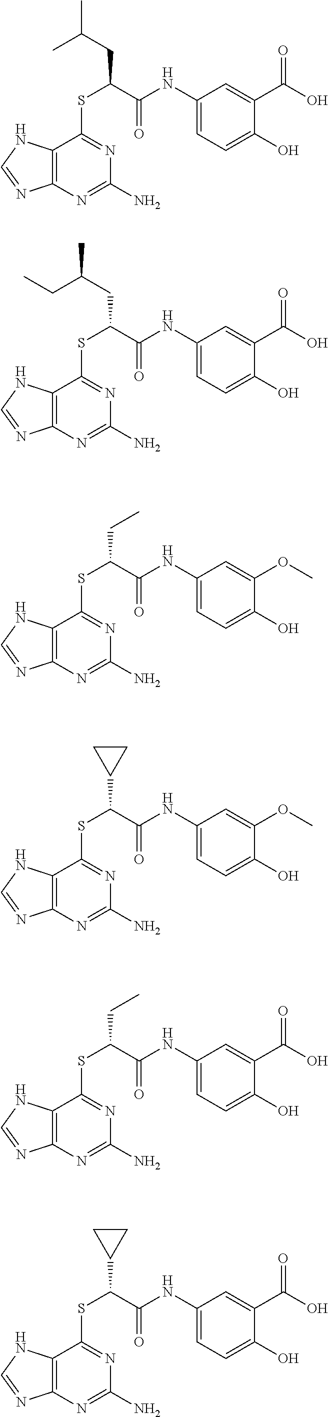

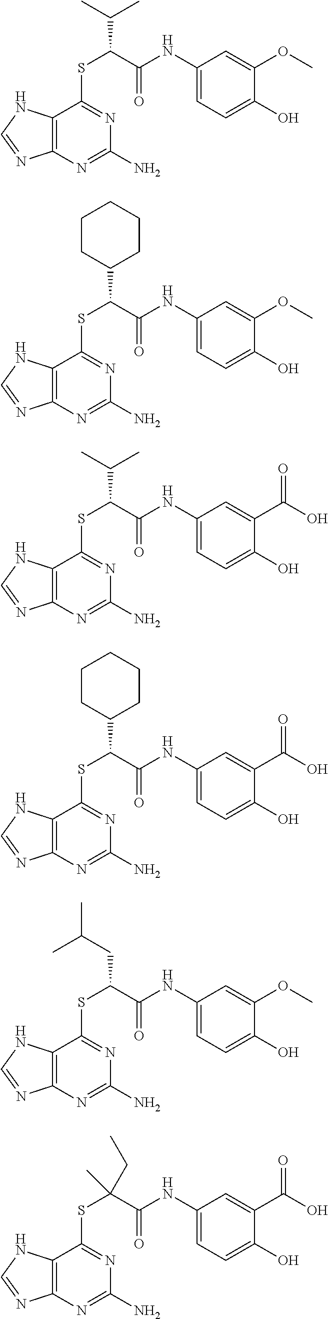

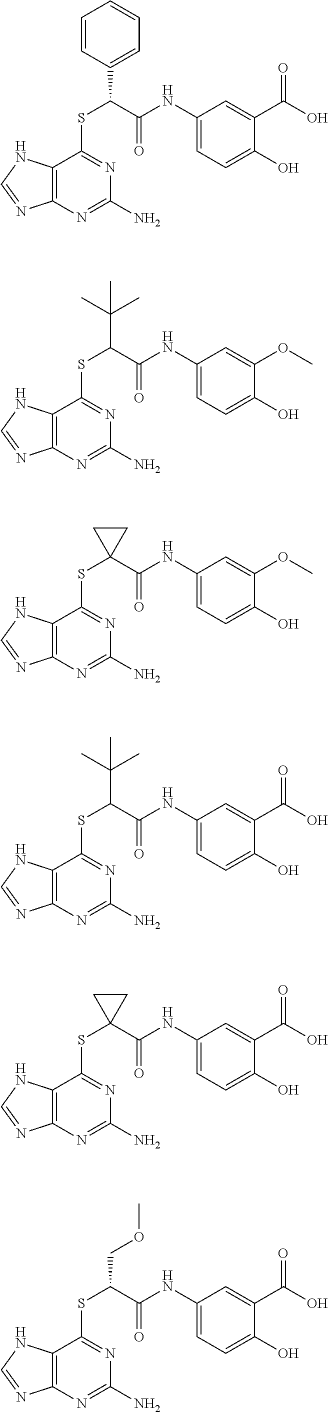 [SMILES: Nc1nc(S[C@@H](C(=O)Nc2ccc(O)c(C(=O)O)c2)c2ccccc2)c2[nH]cnc2n1 ; COc1cc(NC(=O)C(Sc2nc(N)nc3nc[nH]c23)C(C)(C)C)ccc1O ; COc1cc(NC(=O)C2(Sc3nc(N)nc4nc[nH]c34)CC2)ccc1O ; CC(C)(C)C(Sc1nc(N)nc2nc[nH]c12)C(=O)Nc1ccc(O)c(C(=O)O)c1 ; Nc1nc(SC2(C(=O)Nc3ccc(O)c(C(=O)O)c3)CC2)c2[nH]cnc2n1 ; COC[C@@H](Sc1nc(N)nc2nc[nH]c12)C(=O)Nc1ccc(O)c(C(=O)O)c1]

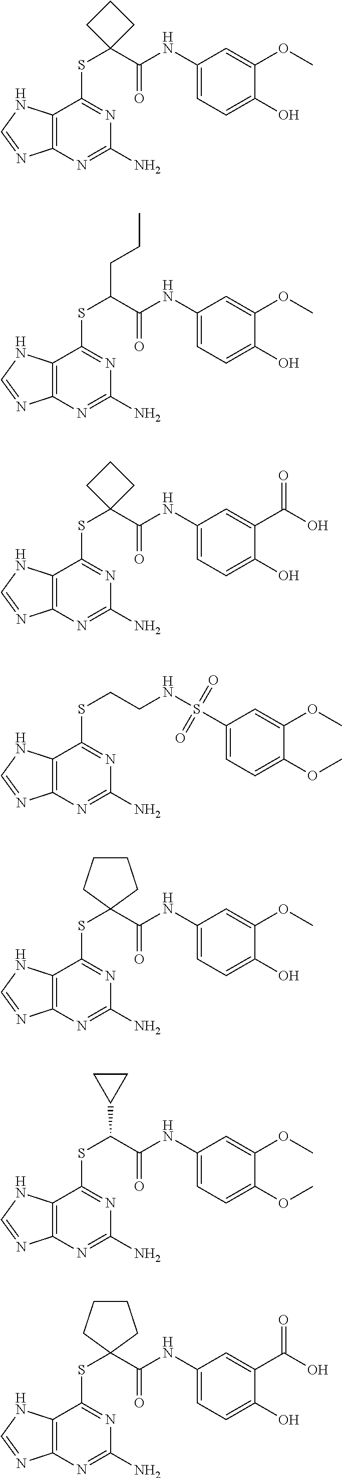

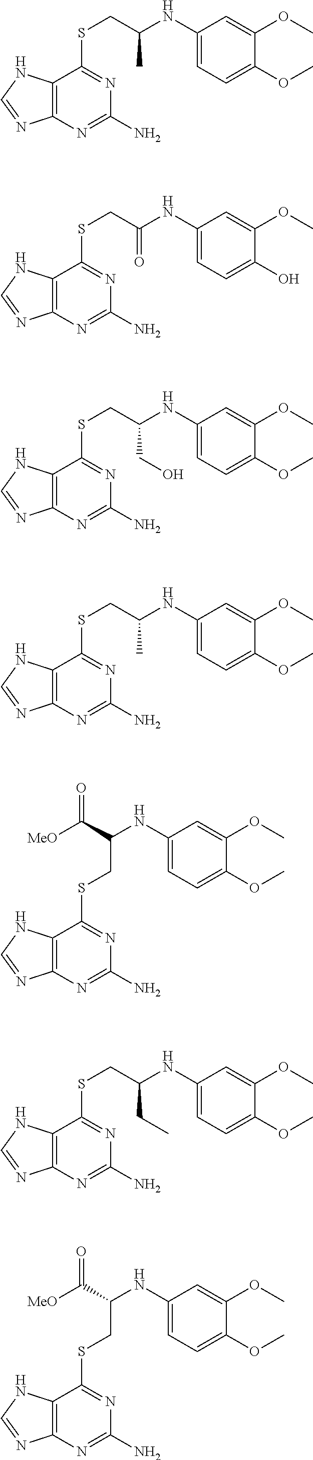

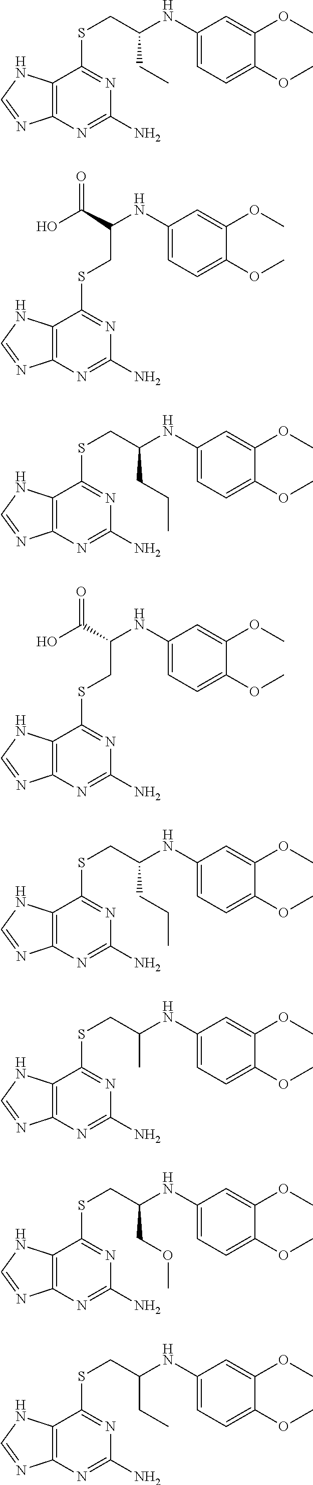

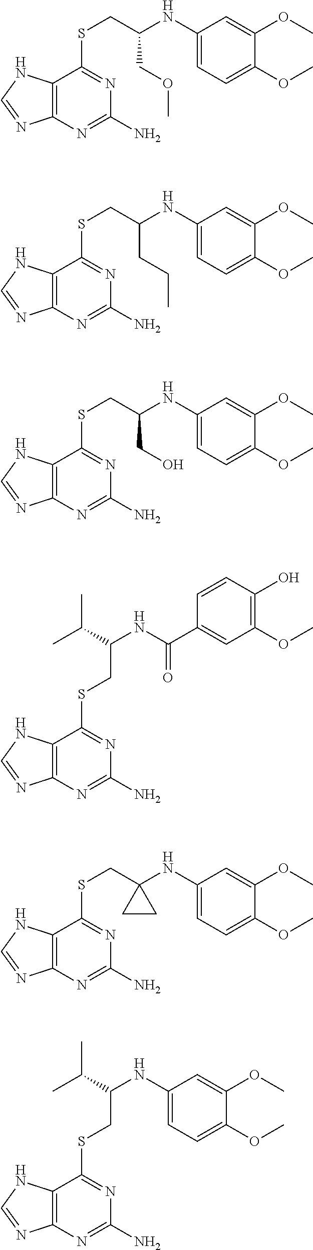

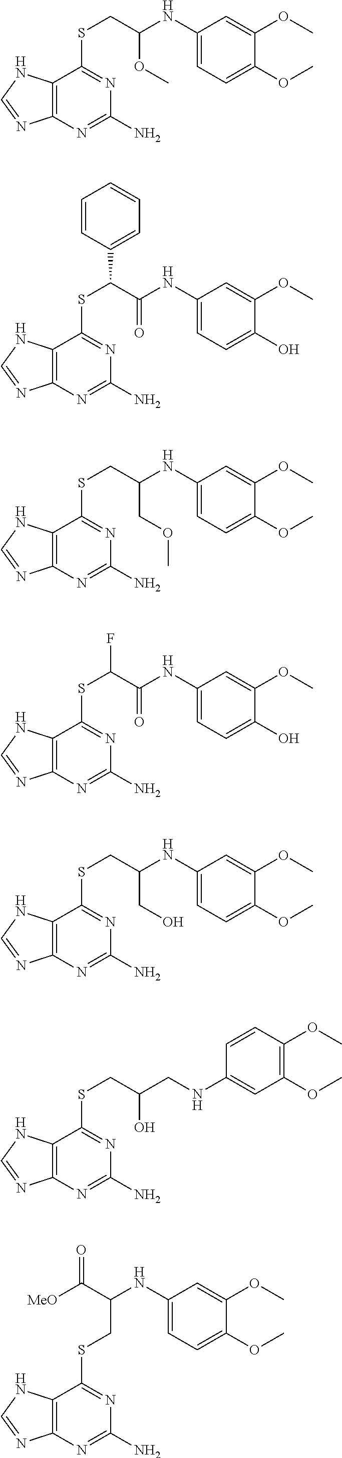

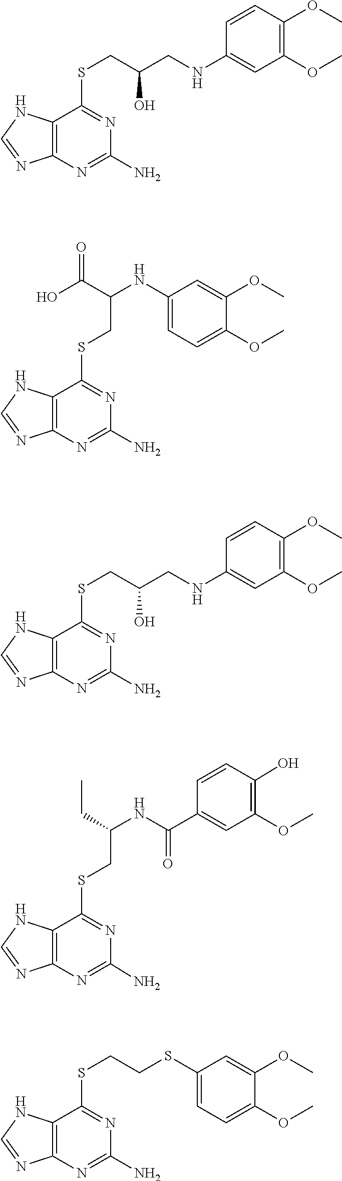 [SMILES: COc1ccc(NC[C@@H](O)CSc2nc(N)nc3nc[nH]c23)cc1OC ; COc1ccc(NC(CSc2nc(N)nc3nc[nH]c23)C(=O)O)cc1OC ; COc1ccc(NC[C@H](O)CSc2nc(N)nc3nc[nH]c23)cc1OC ; CC[C@@H](CSc1nc(N)nc2nc[nH]c12)NC(=O)c1ccc(O)c(OC)c1 ; COc1ccc(SCCSc2nc(N)nc3nc[nH]c23)cc1OC]

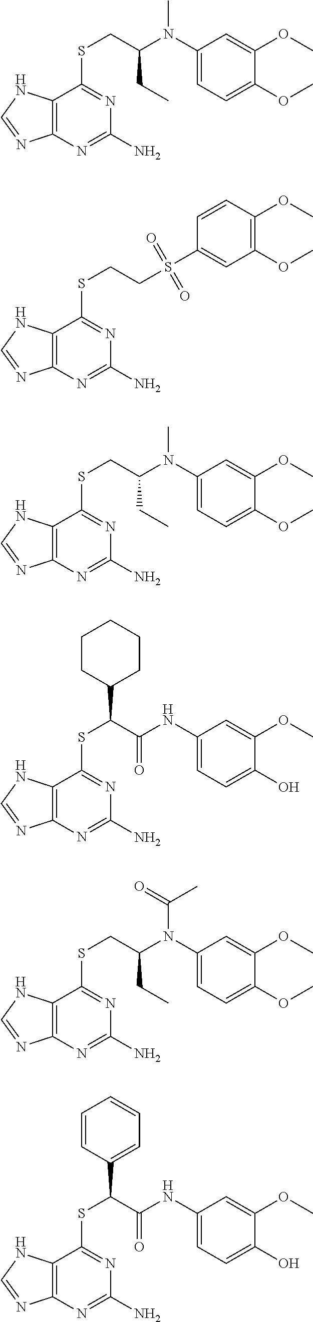 [SMILES: CC[C@@H](CSc1nc(N)nc2nc[nH]c12)N(C)c1ccc(OC)c(OC)c1 ; COc1ccc(S(=O)(=O)CCSc2nc(N)nc3nc[nH]c23)cc1OC ; CC[C@H](CSc1nc(N)nc2nc[nH]c12)N(C)c1ccc(OC)c(OC)c1 ; COc1cc(NC(=O)[C@@H](Sc2nc(N)nc3nc[nH]c23)C2CCCCC2)ccc1O ; CC[C@@H](CSc1nc(N)nc2nc[nH]c12)N(C(C)=O)c1ccc(OC)c(OC)c1 ; COc1cc(NC(=O)[C@@H](Sc2nc(N)nc3nc[nH]c23)c2ccccc2)ccc1O]

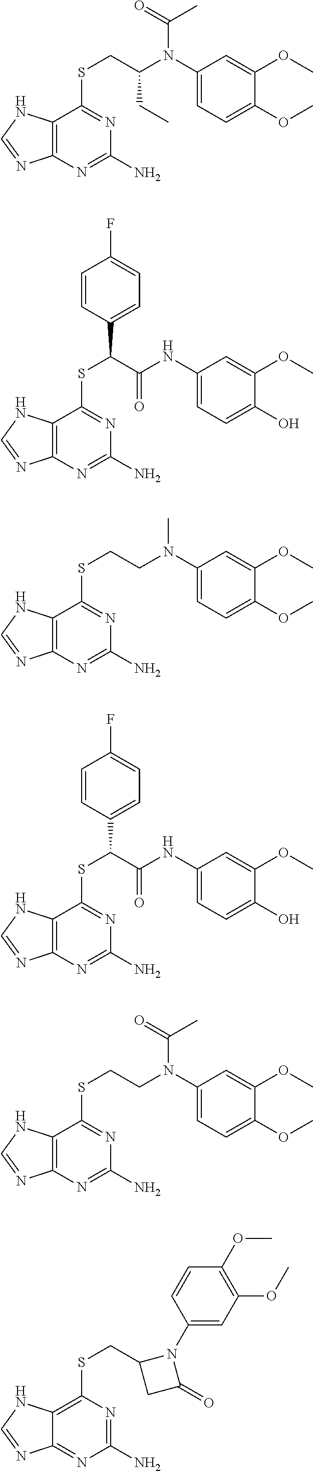 [SMILES: CC[C@H](CSc1nc(N)nc2nc[nH]c12)N(C(C)=O)c1ccc(OC)c(OC)c1 ; COc1cc(NC(=O)[C@@H](Sc2nc(N)nc3nc[nH]c23)c2ccc(F)cc2)ccc1O ; COc1ccc(N(C)CCSc2nc(N)nc3nc[nH]c23)cc1OC ; COc1cc(NC(=O)[C@H](Sc2nc(N)nc3nc[nH]c23)c2ccc(F)cc2)ccc1O ; COc1ccc(N(CCSc2nc(N)nc3nc[nH]c23)C(C)=O)cc1OC ; COc1ccc(N2C(=O)CC2CSc2nc(N)nc3nc[nH]c23)cc1OC]

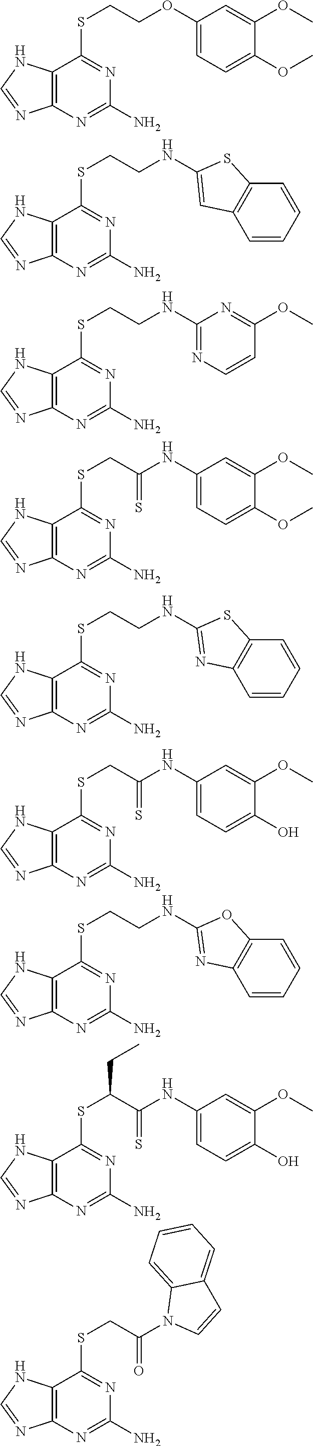

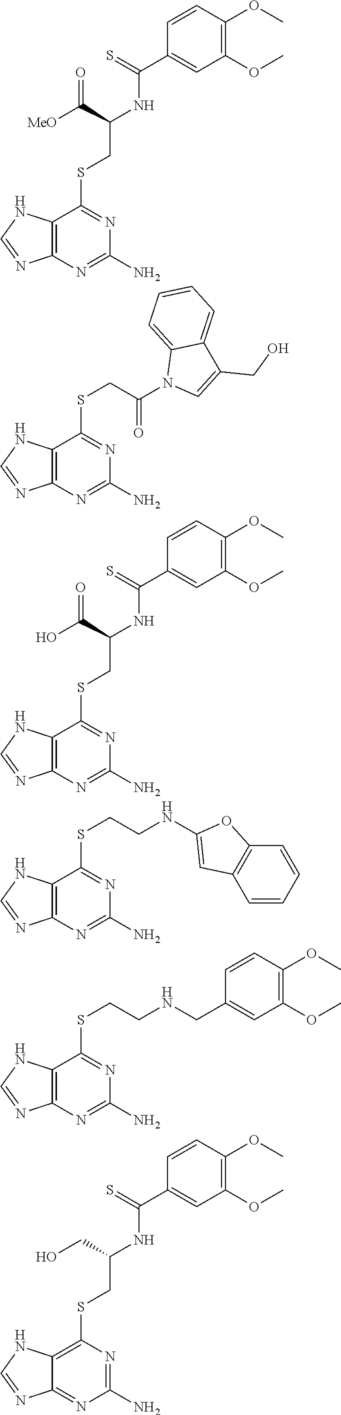

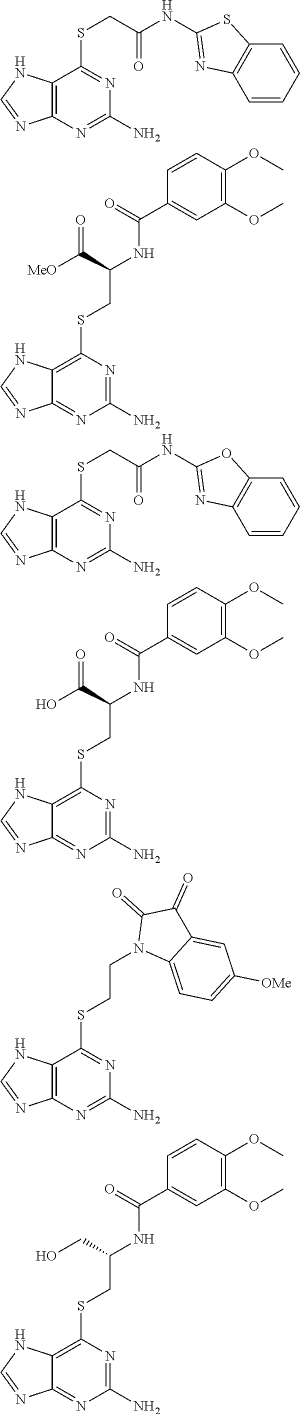

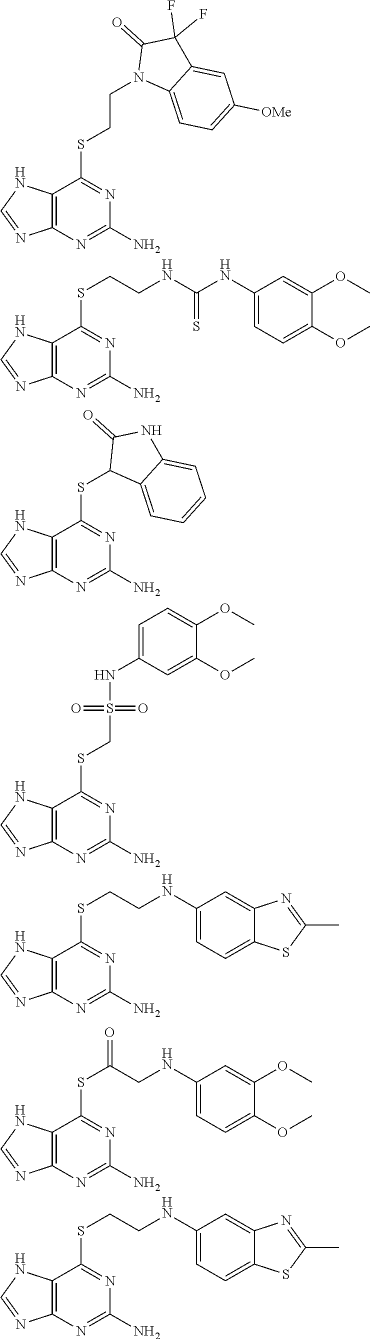

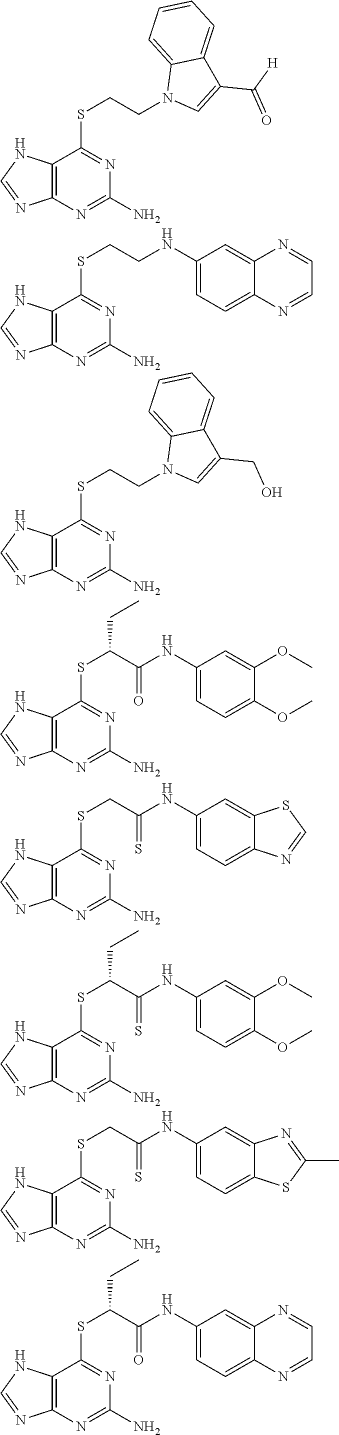

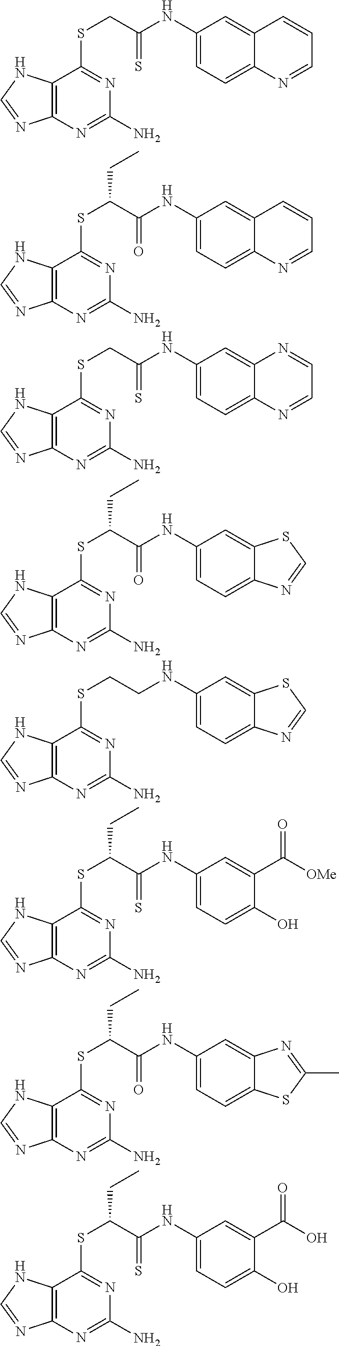

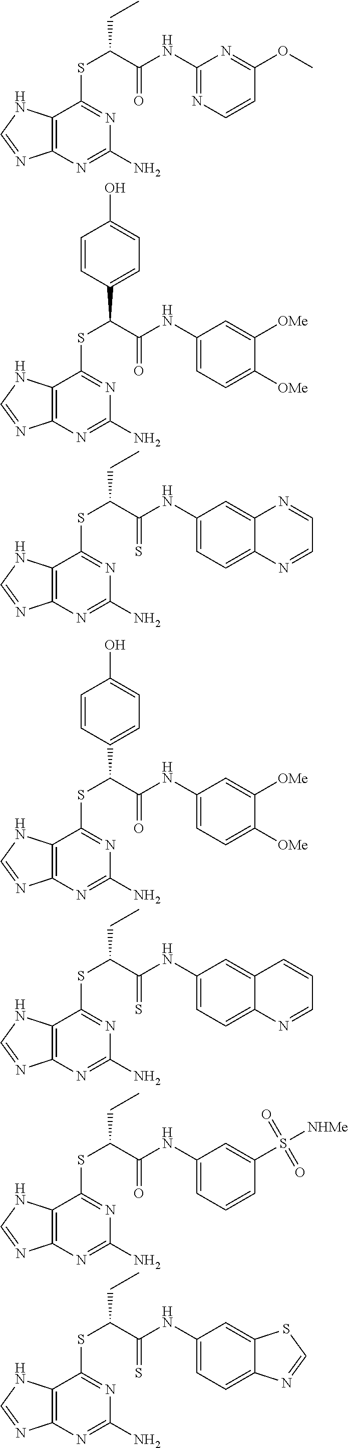

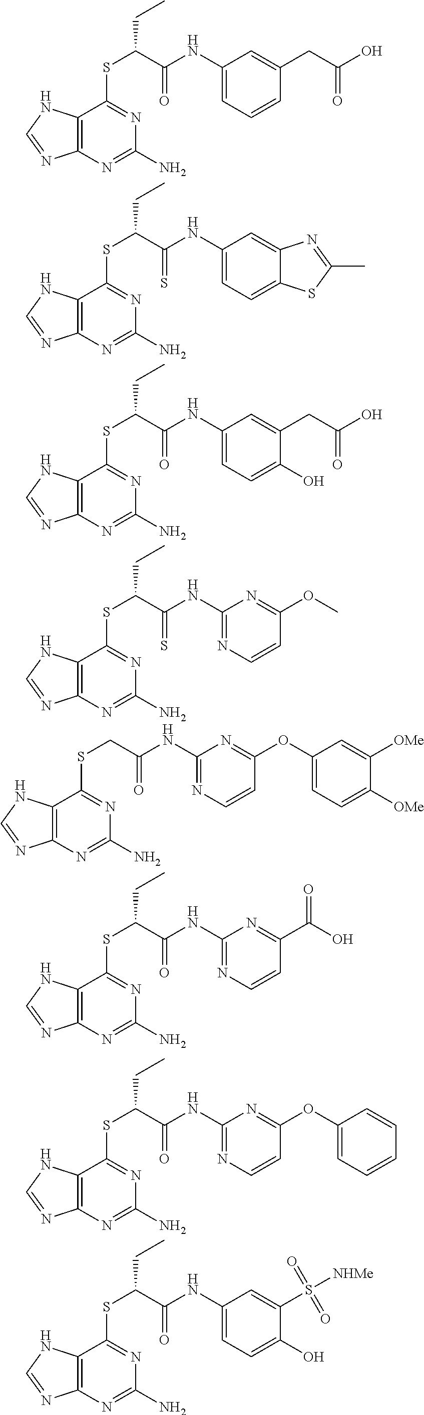

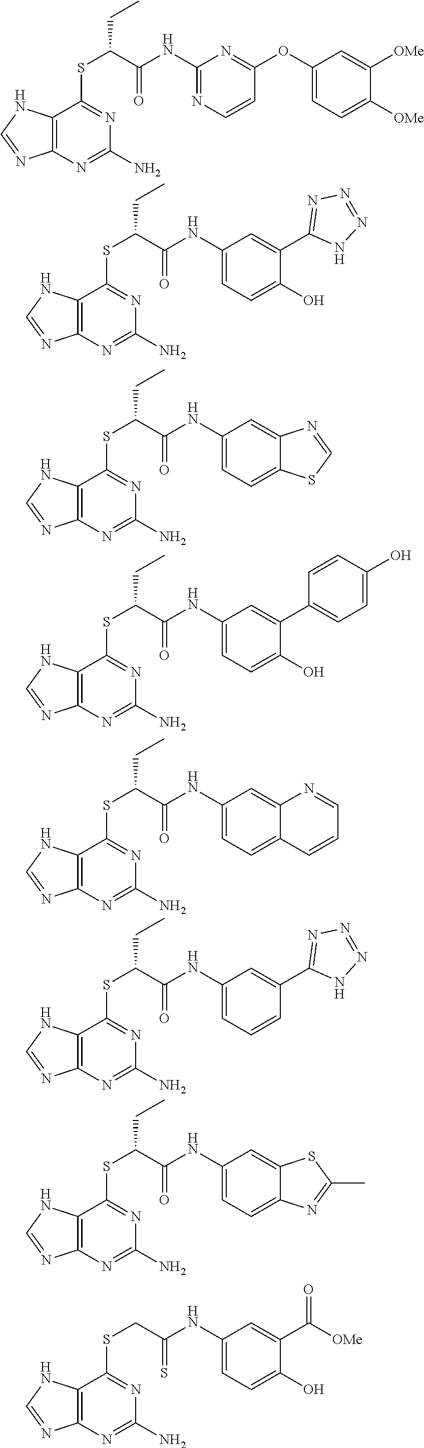

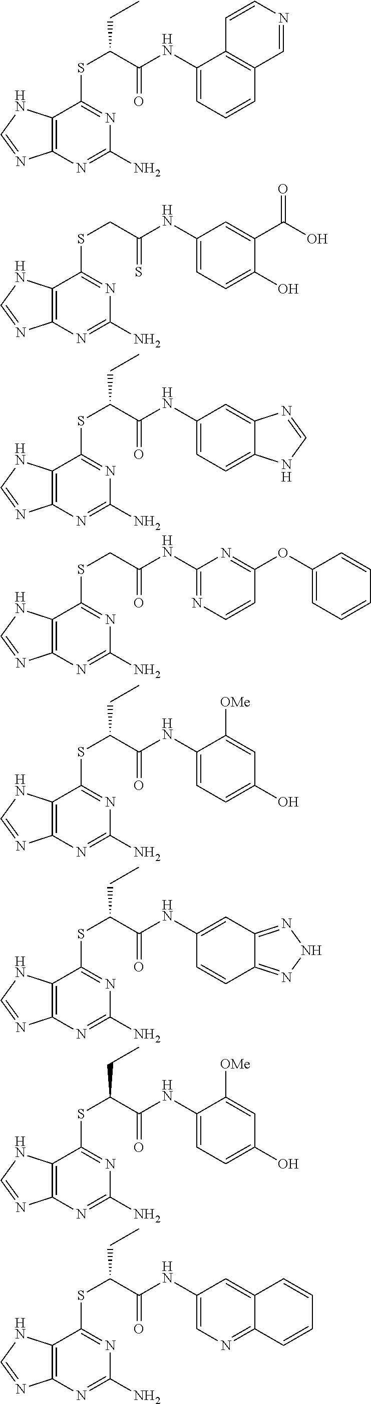

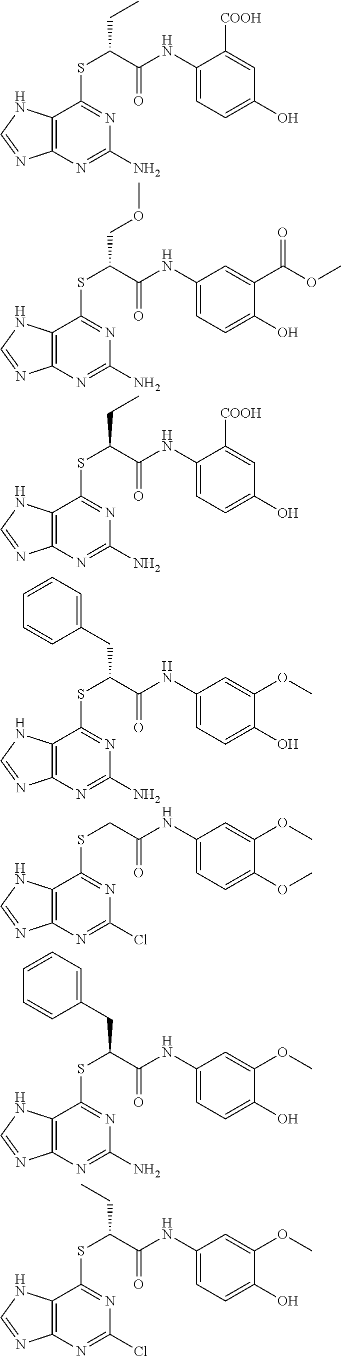

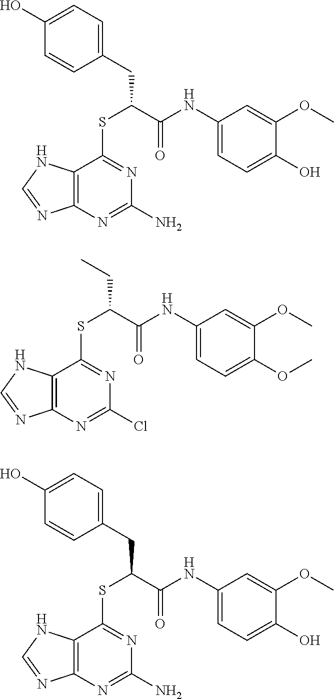

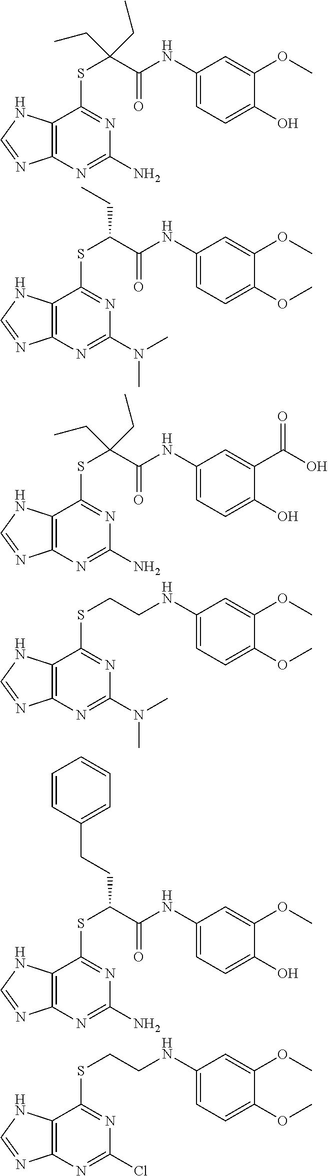

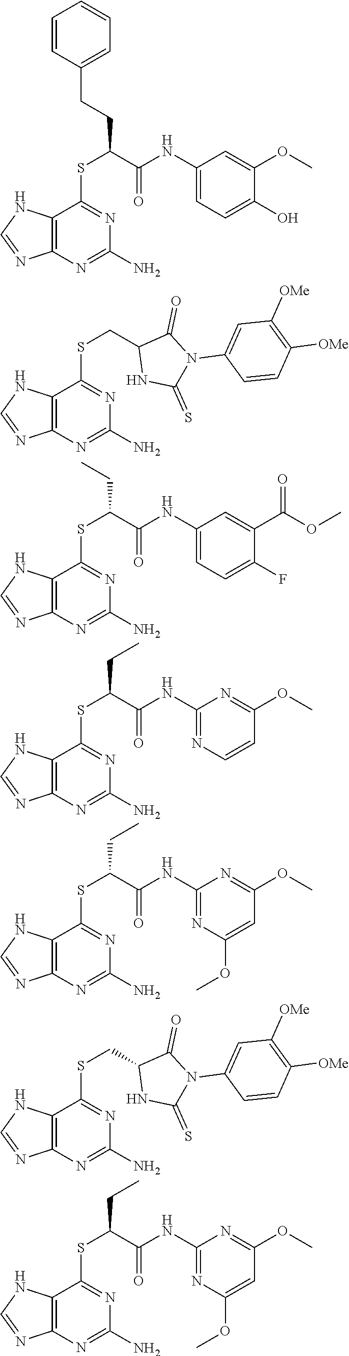

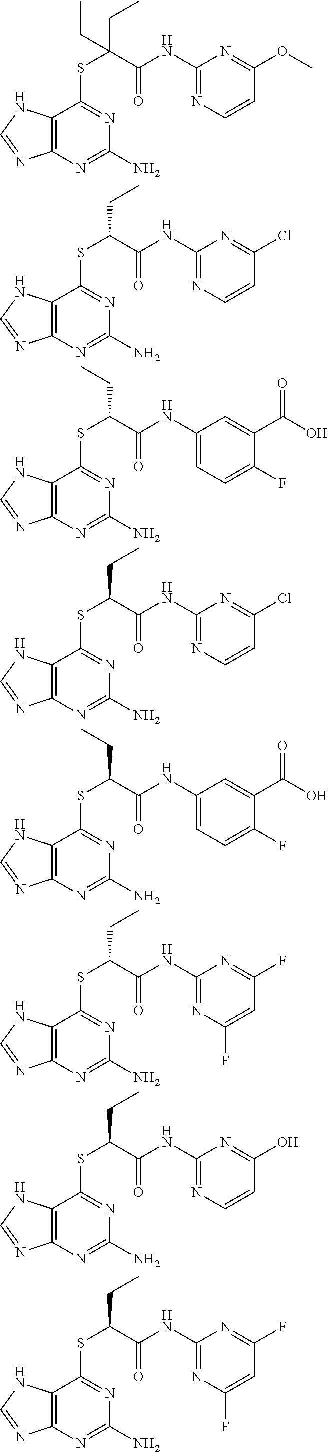

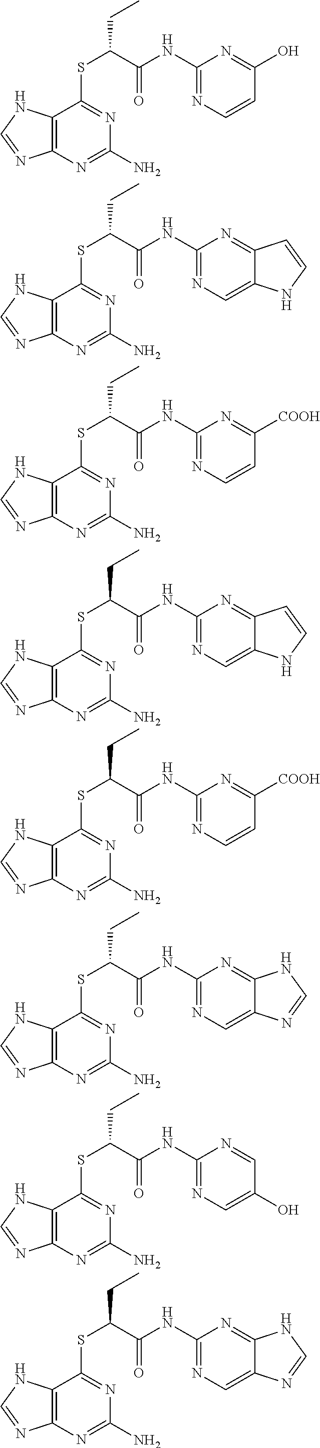

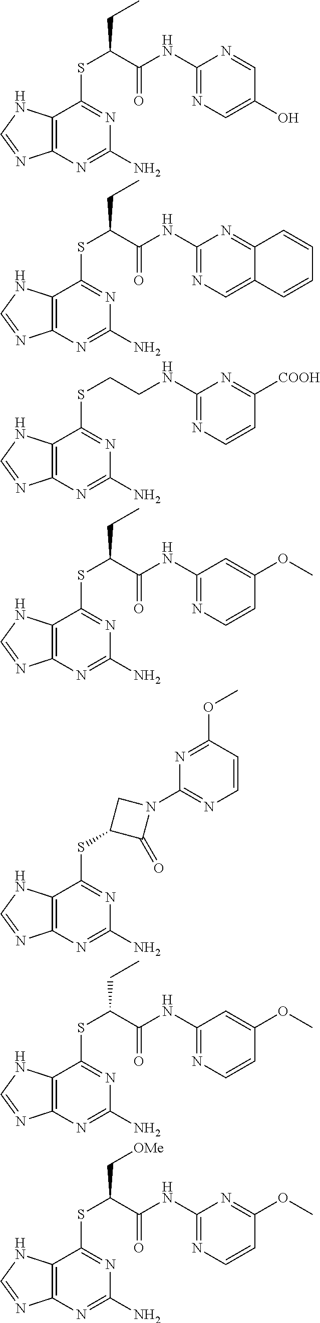

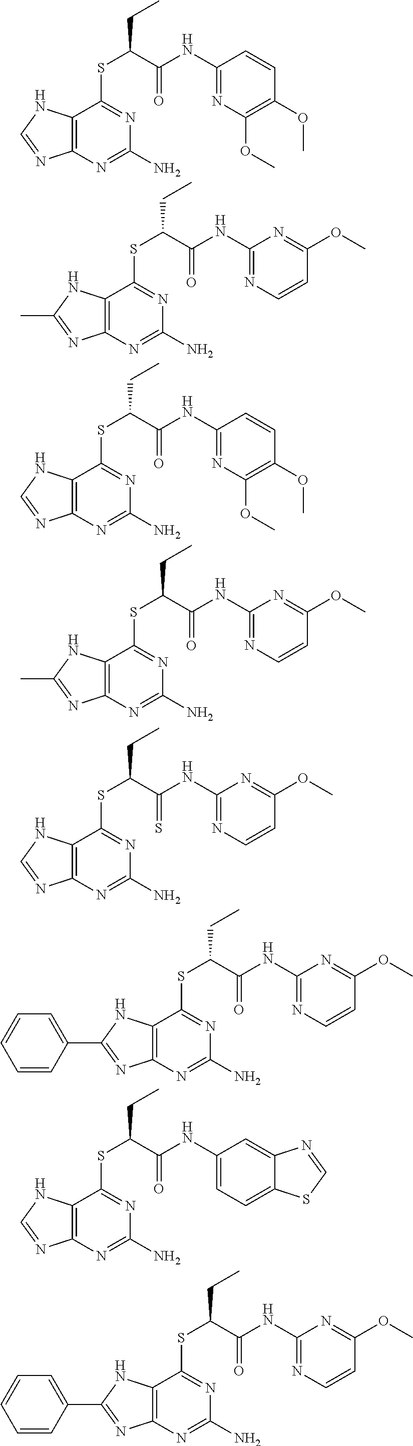

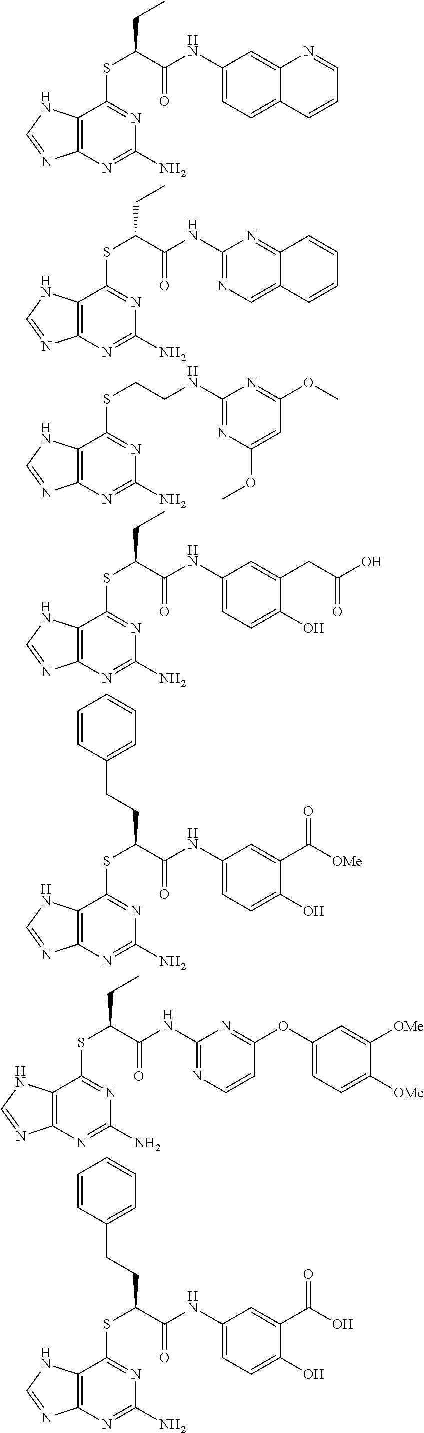

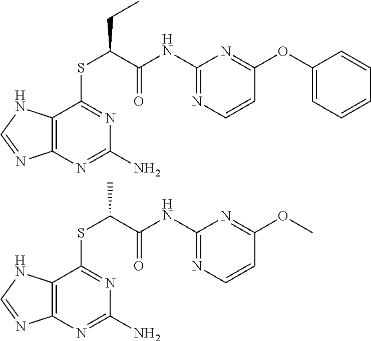

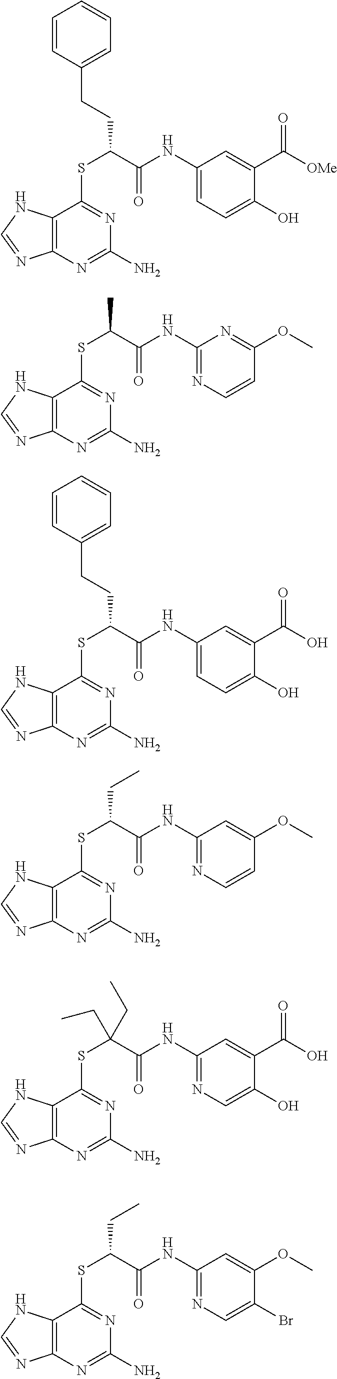

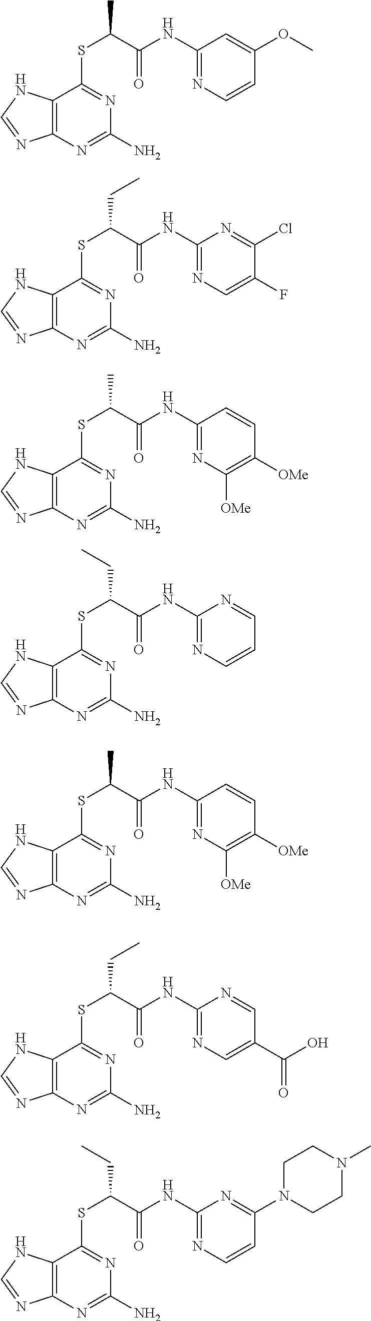

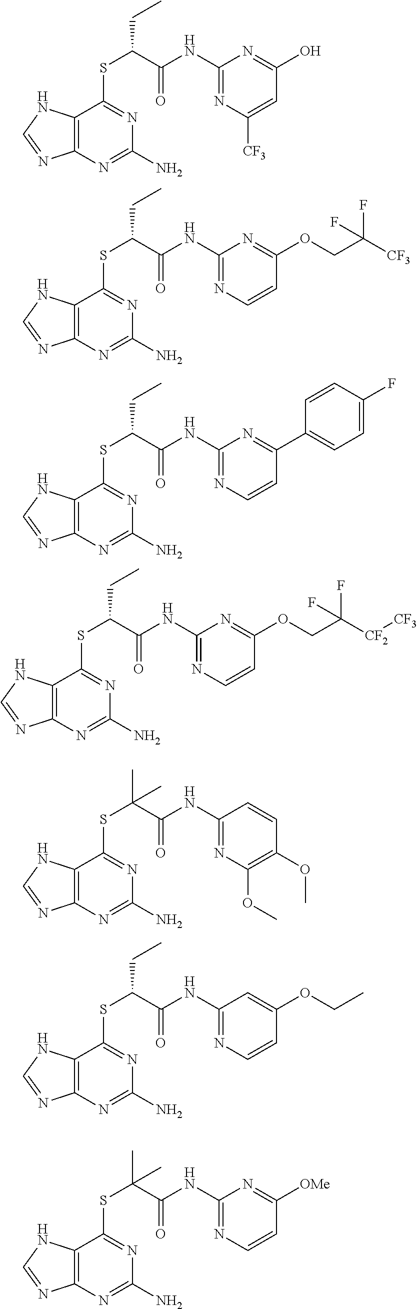

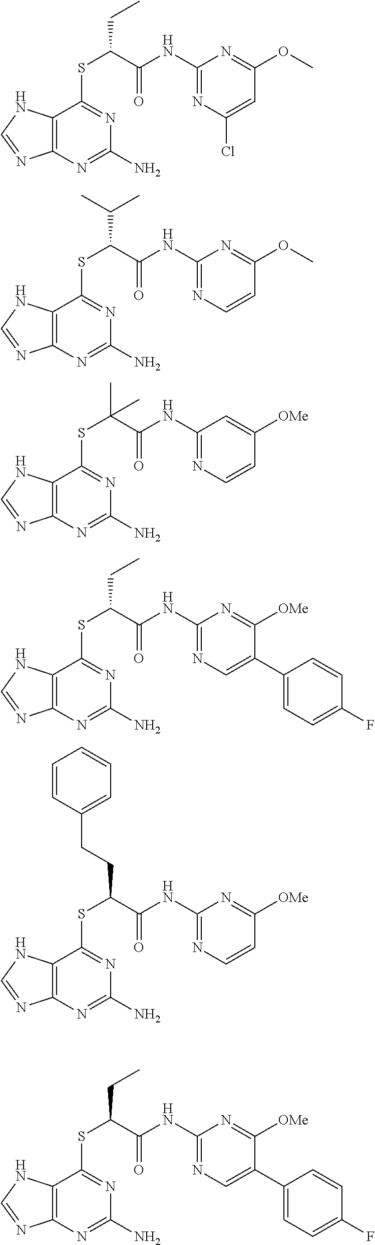

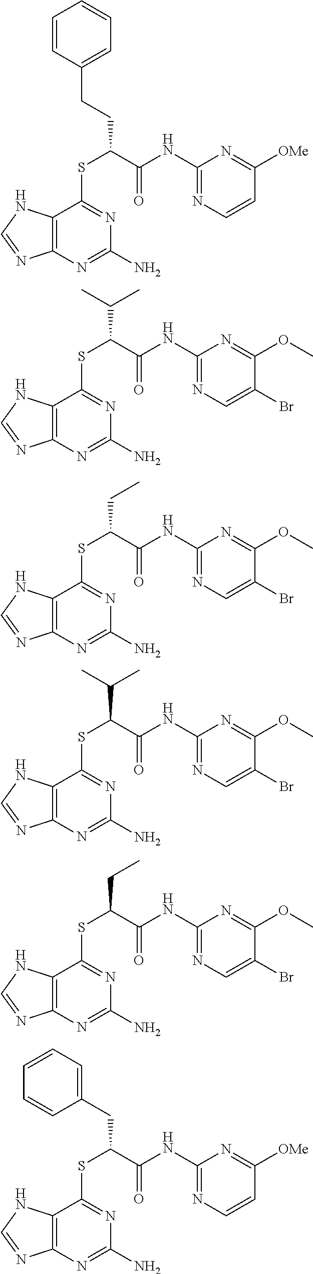

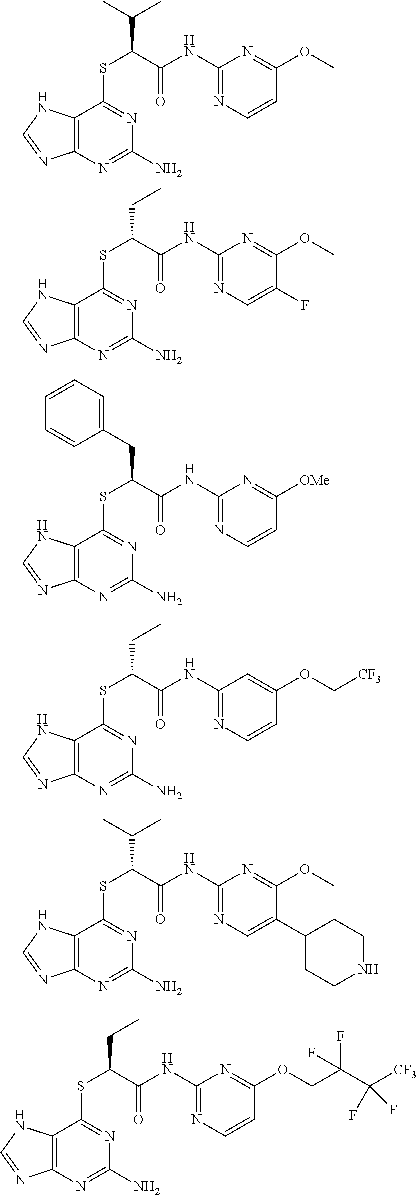

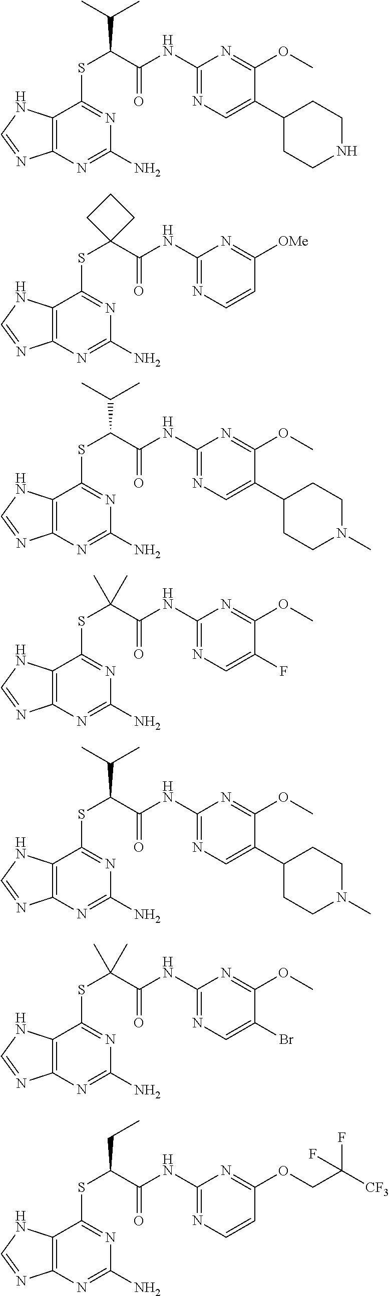

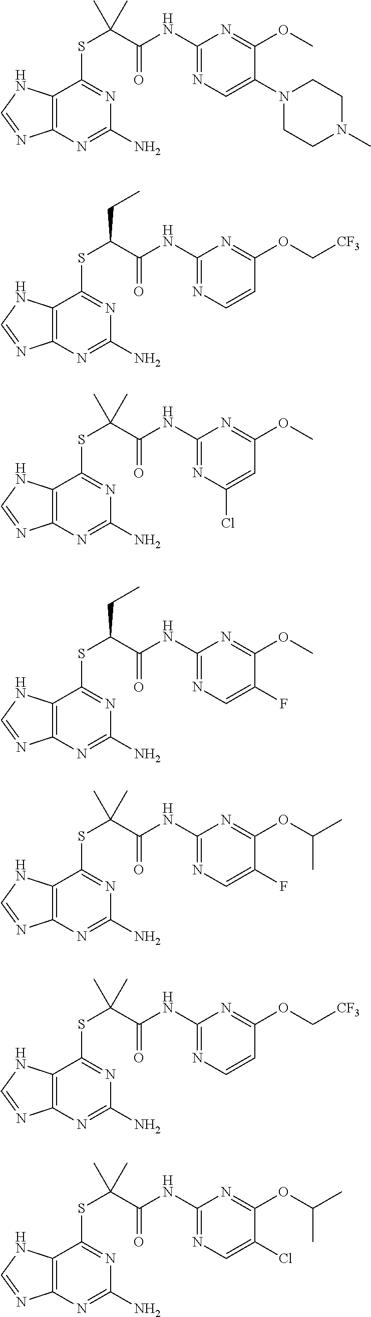

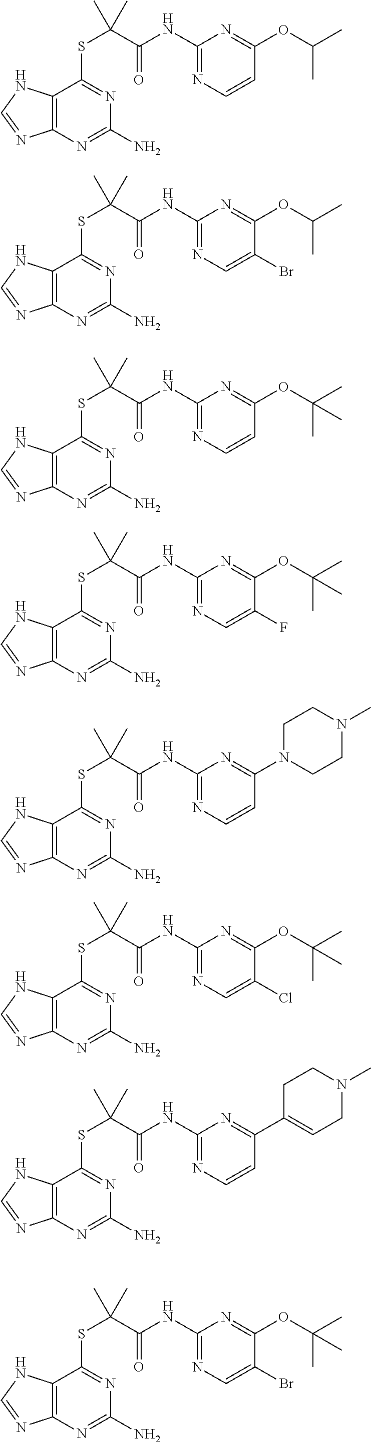

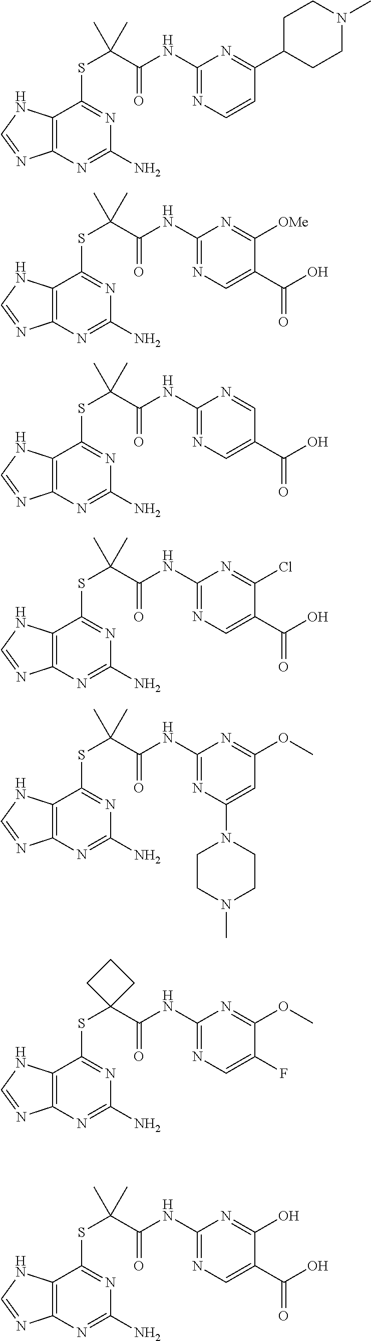

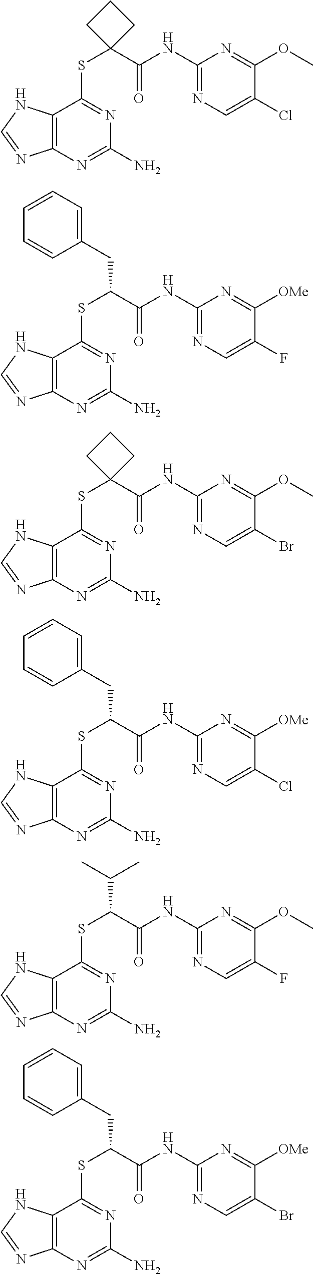

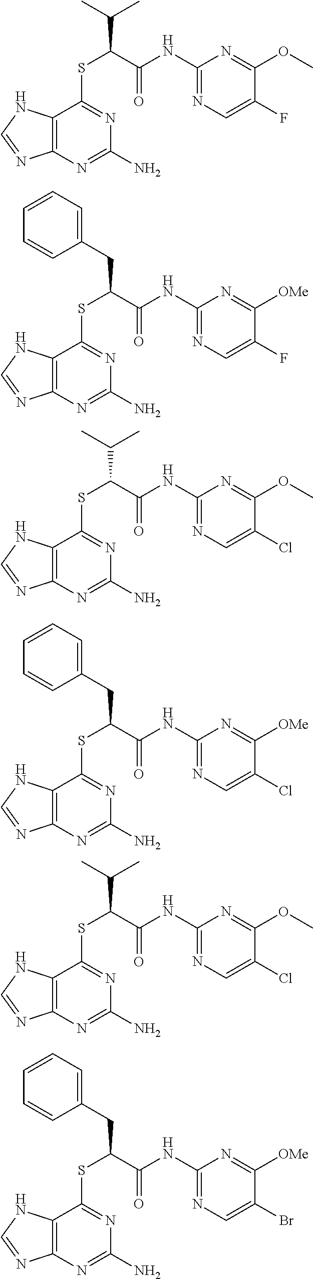

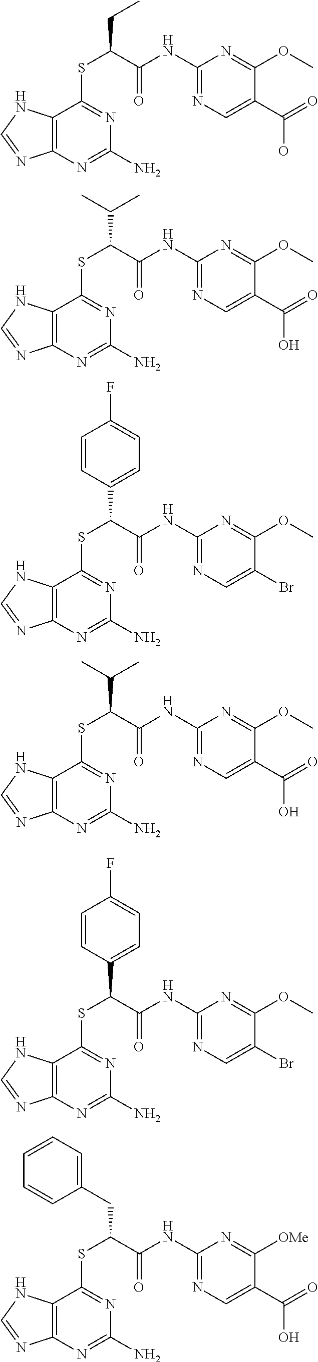

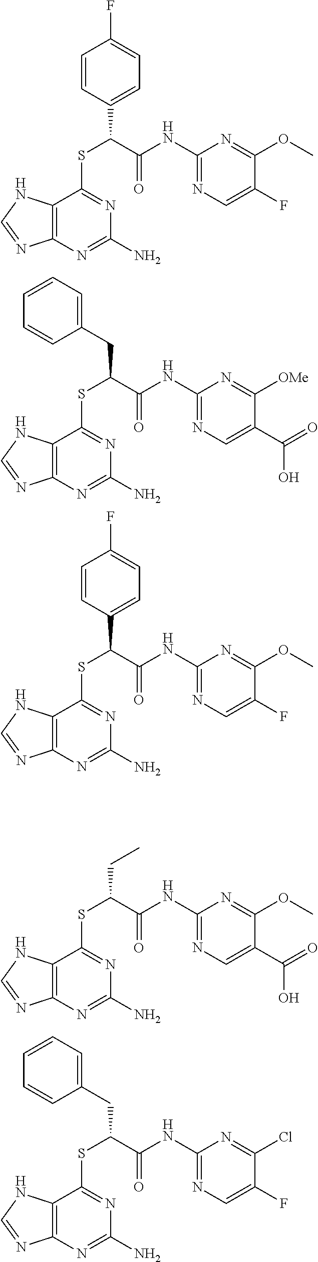

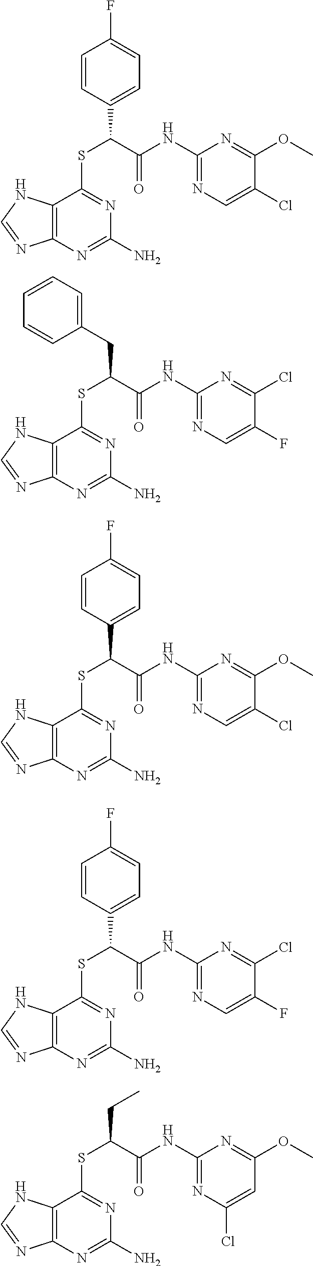

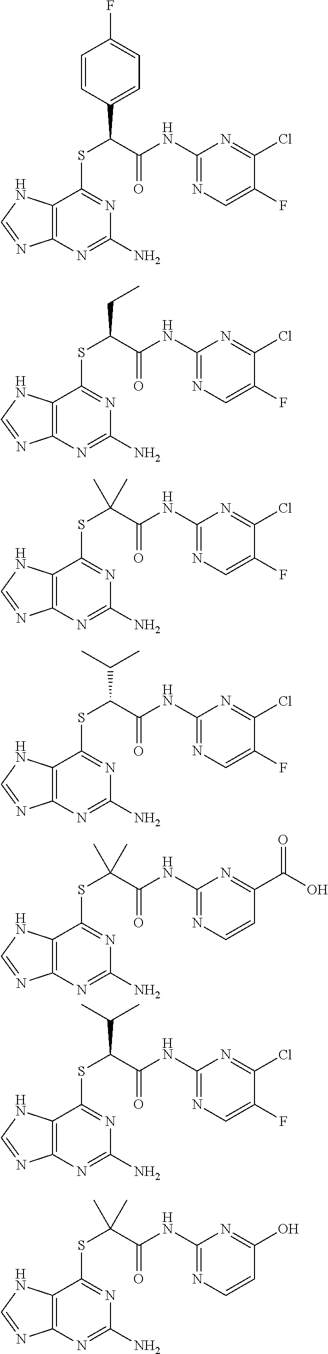

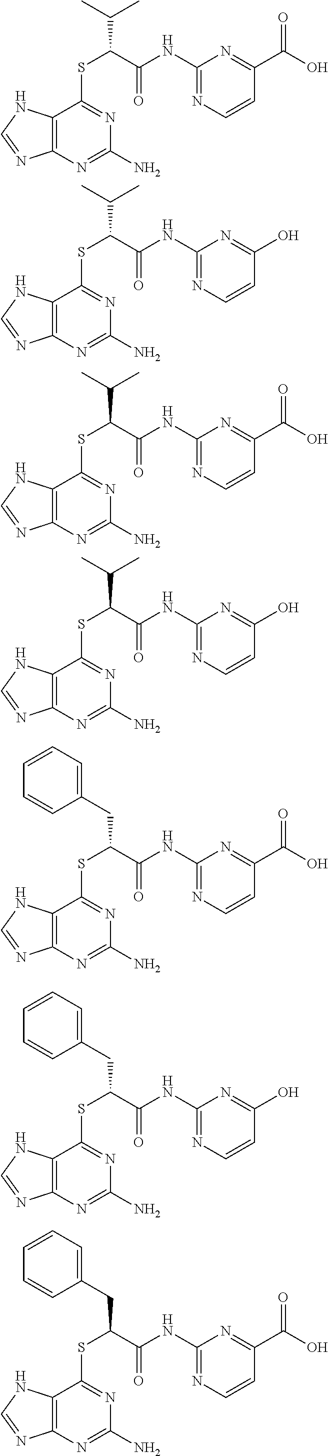

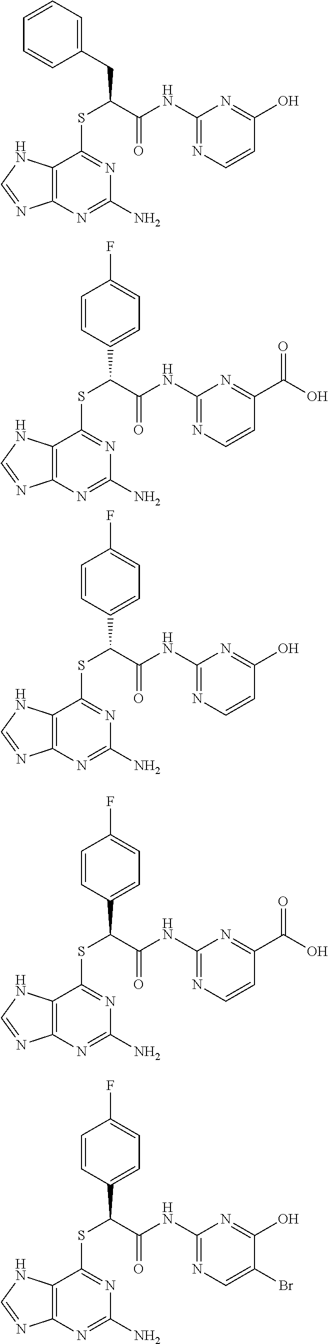

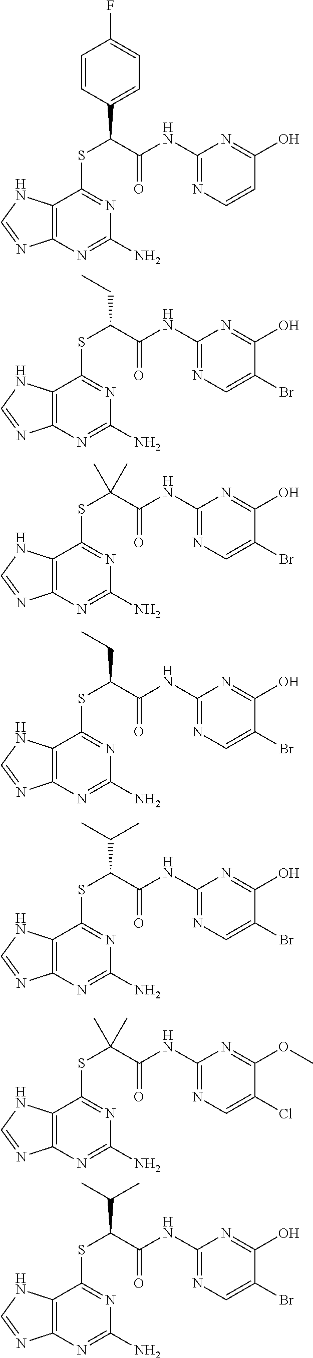

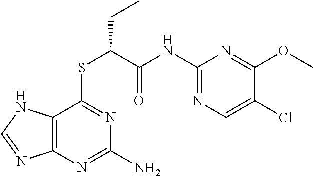 [SMILES: CC[C@@H](Sc1nc(N)nc2nc[nH]c12)C(=O)Nc1ncc(Cl)c(OC)n1]

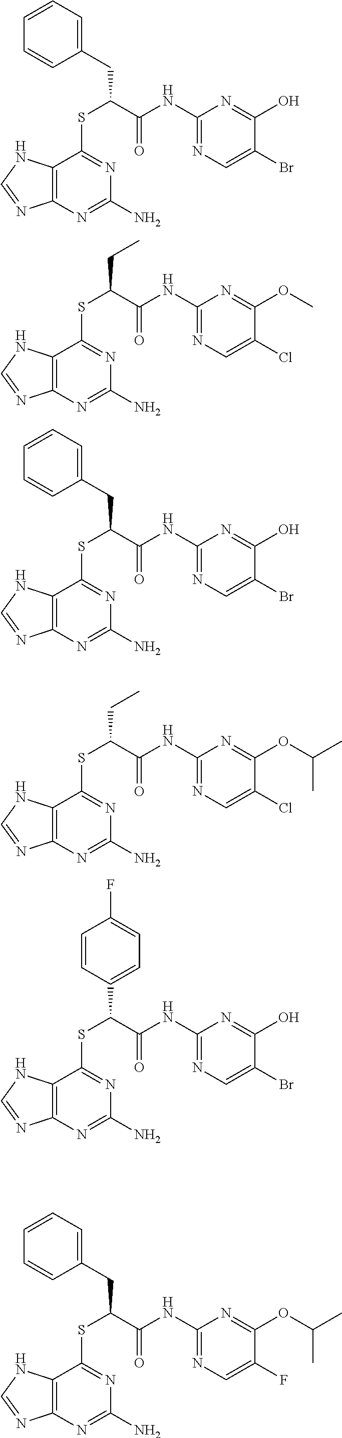

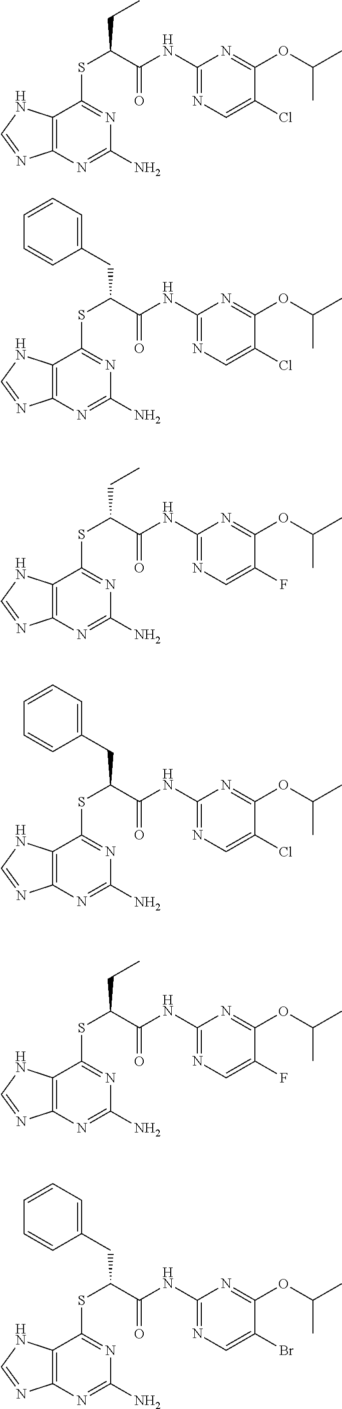

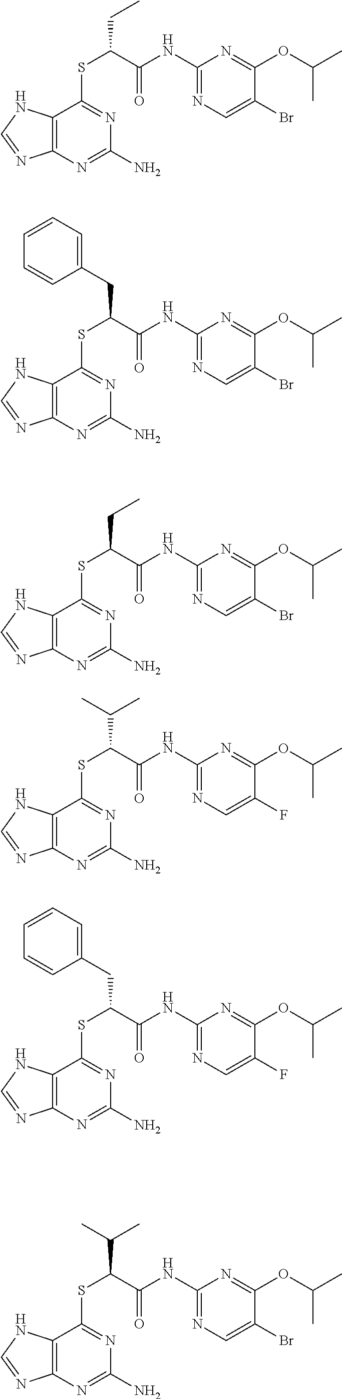

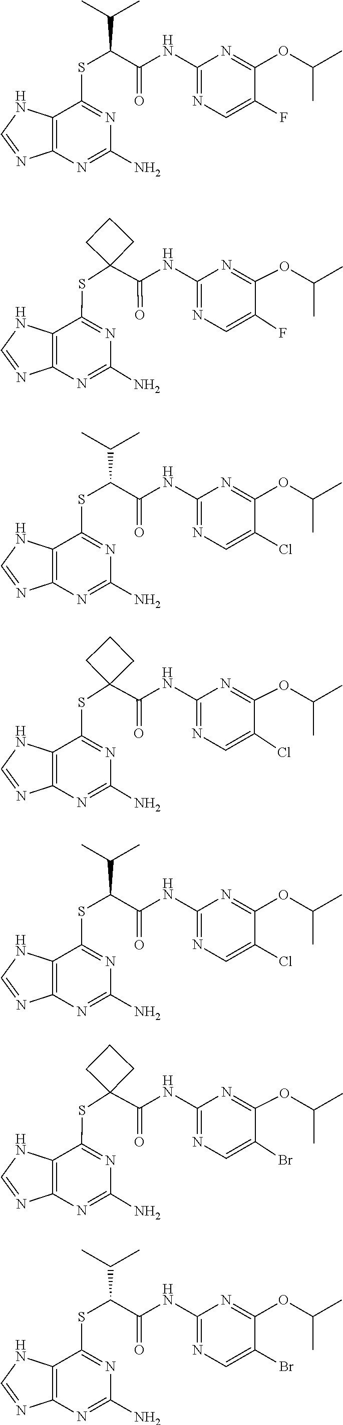

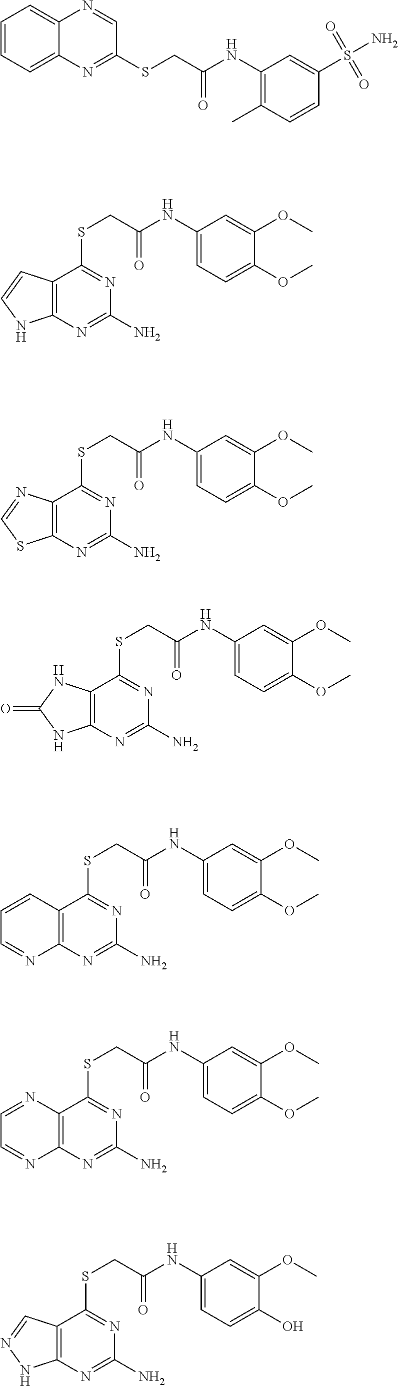

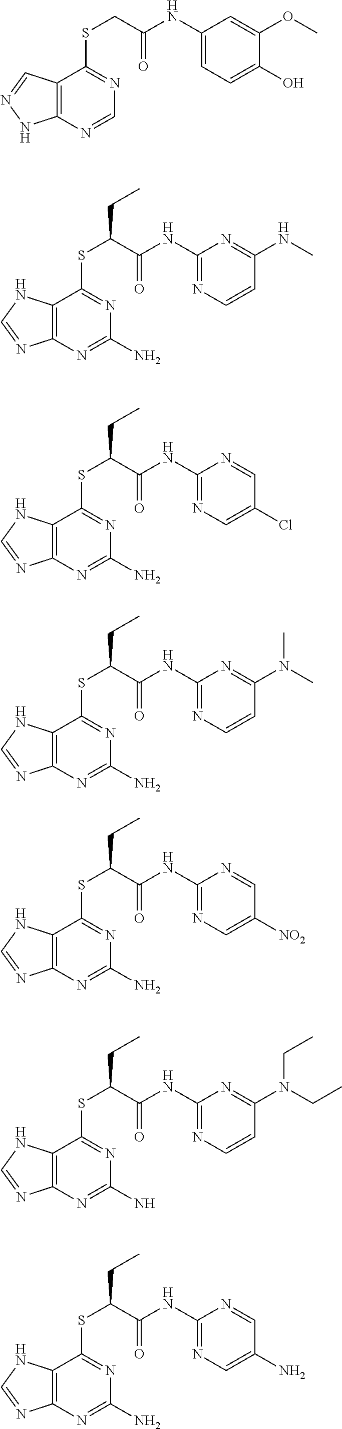

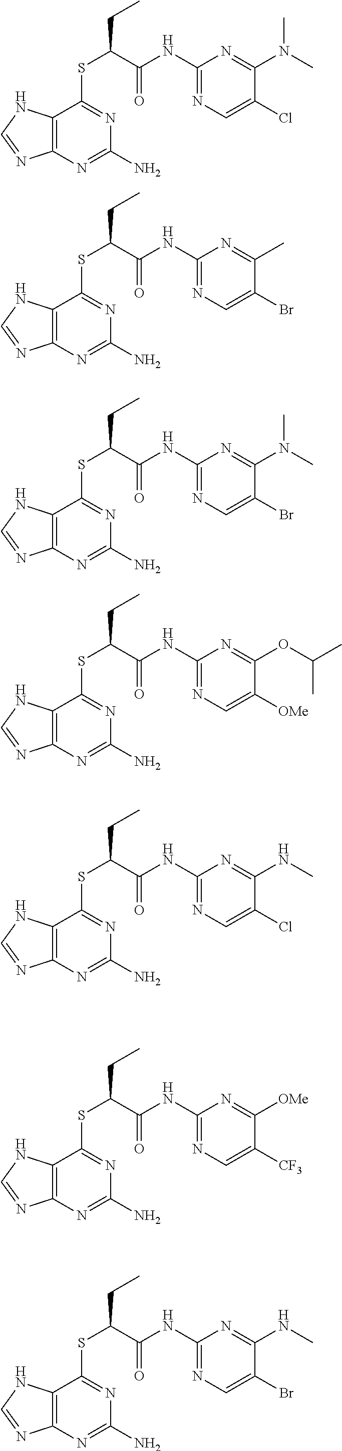

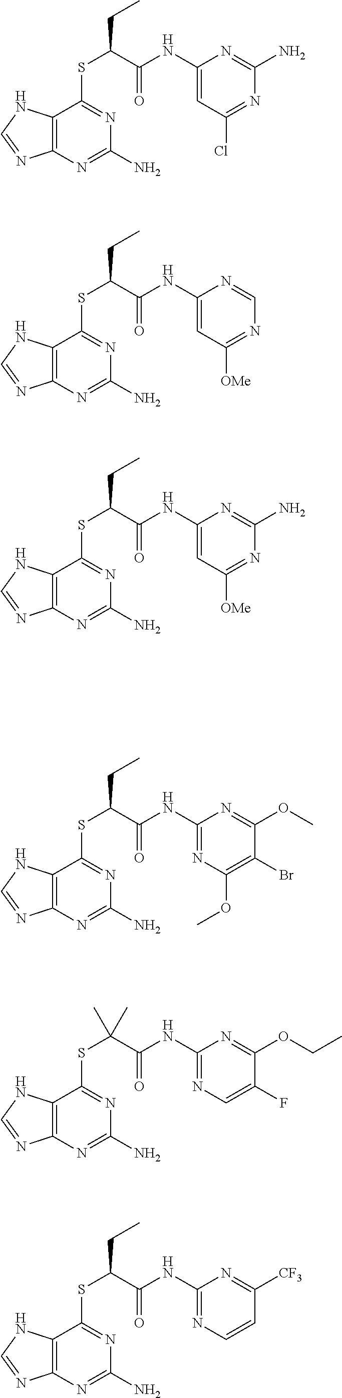

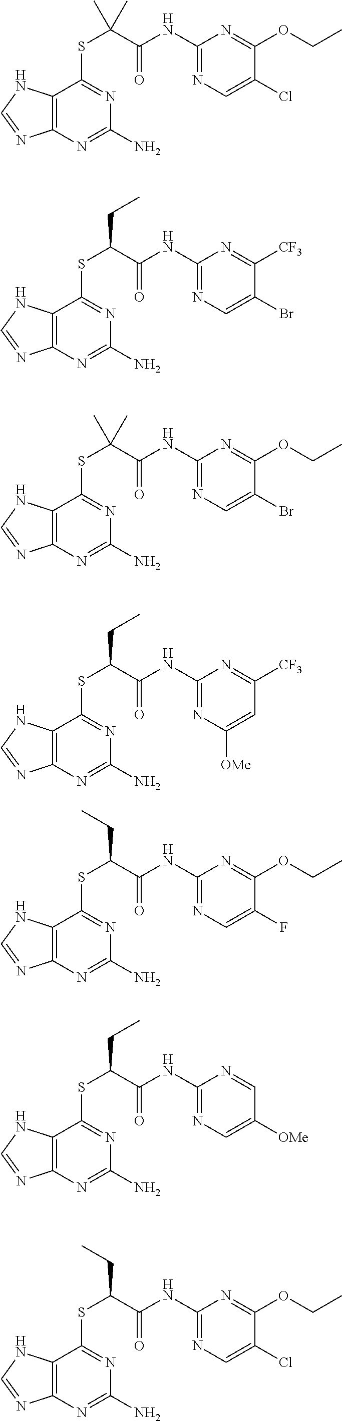

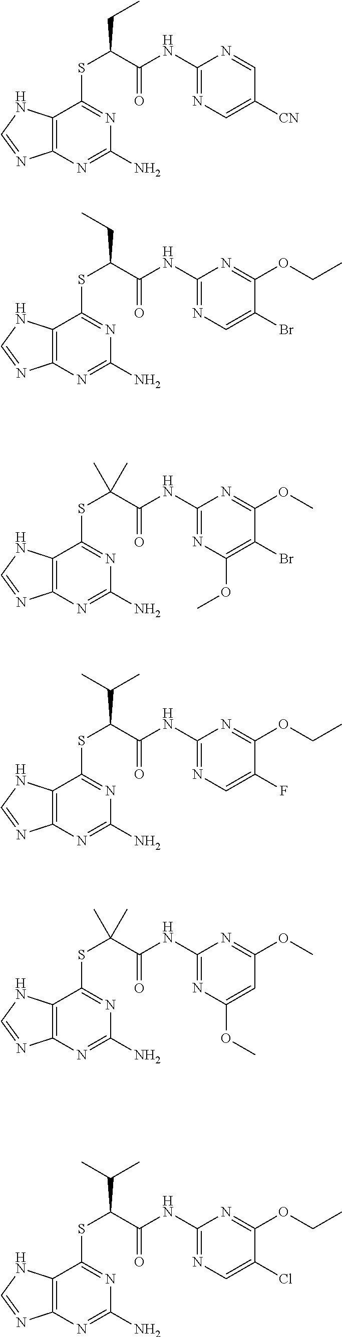

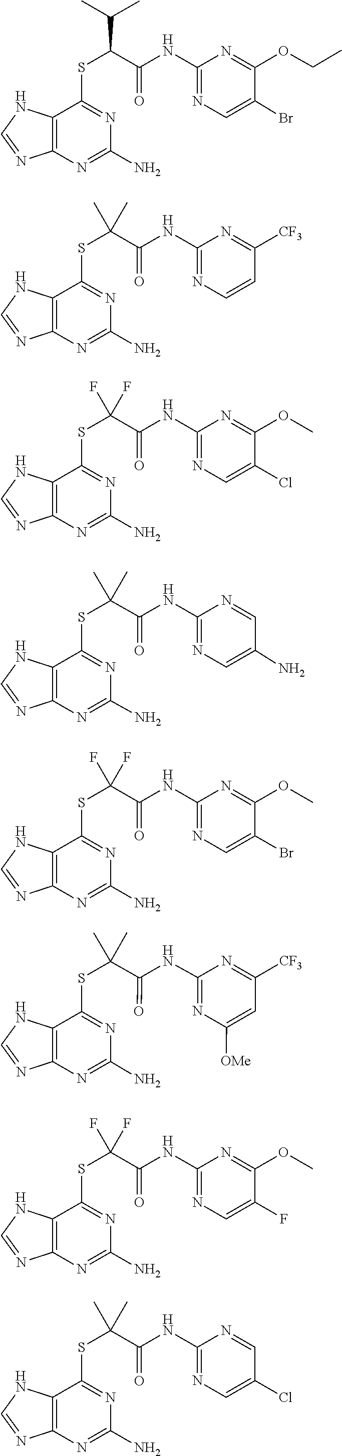

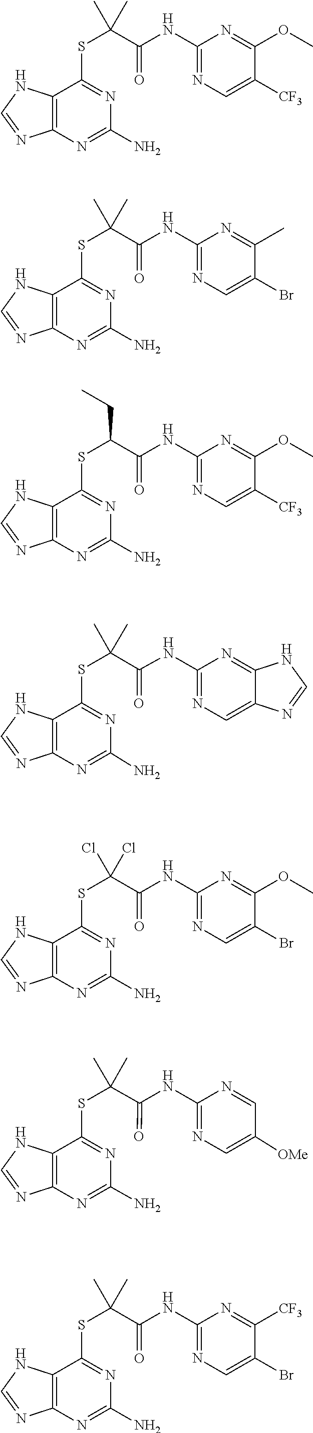

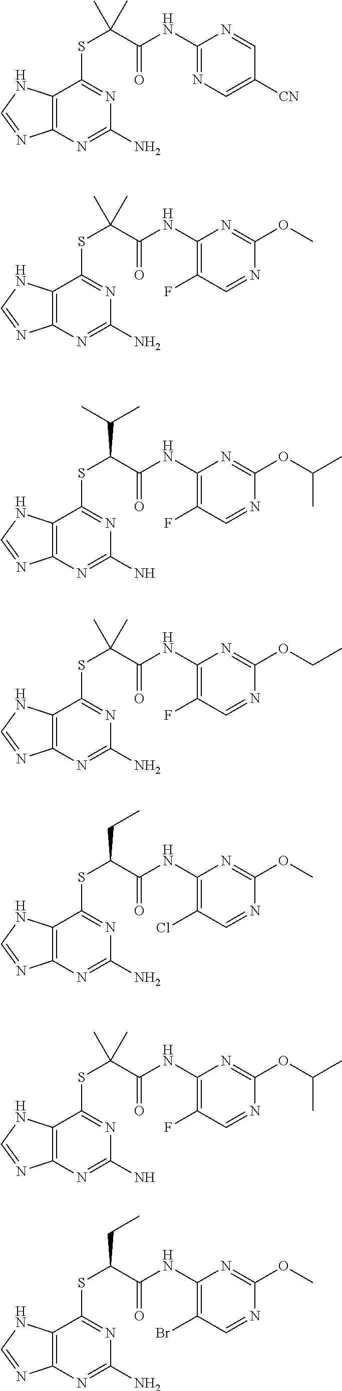

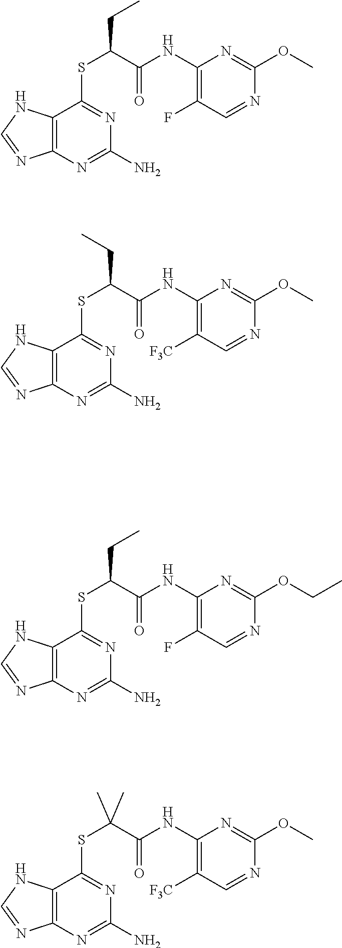 [SMILES: CC[C@H](Sc1nc(N)nc2nc[nH]c12)C(=O)Nc1nc(OC)ncc1F ; CC[C@H](Sc1nc(N)nc2nc[nH]c12)C(=O)Nc1nc(OC)ncc1C(F)(F)F ; CCOc1ncc(F)c(NC(=O)[C@H](CC)Sc2nc(N)nc3nc[nH]c23)n1 ; COc1ncc(C(F)(F)F)c(NC(=O)C(C)(C)Sc2nc(N)nc3nc[nH]c23)n1]

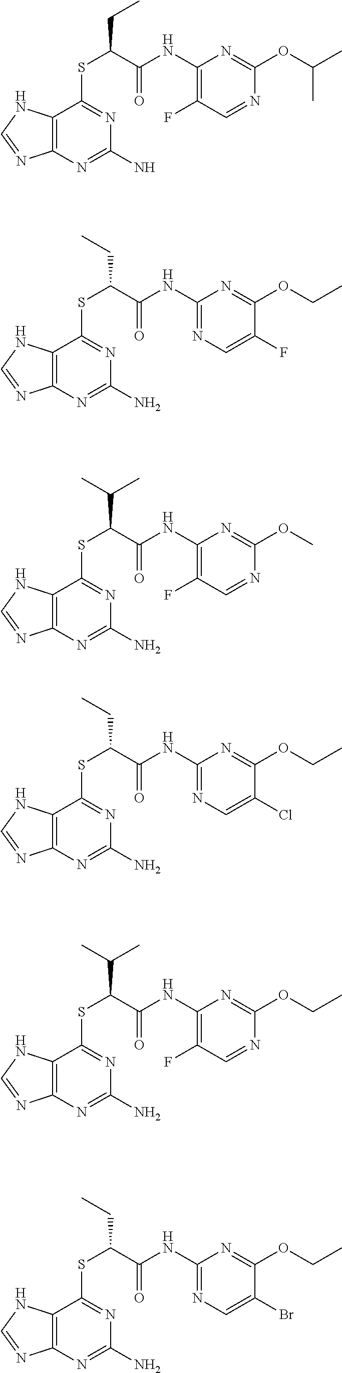 [SMILES: CC[C@H](Sc1nc(N)nc2nc[nH]c12)C(=O)Nc1nc(OC(C)C)ncc1F ; CCOc1nc(NC(=O)[C@@H](CC)Sc2nc(N)nc3nc[nH]c23)ncc1F ; COc1ncc(F)c(NC(=O)[C@@H](Sc2nc(N)nc3nc[nH]c23)C(C)C)n1 ; CCOc1nc(NC(=O)[C@@H](CC)Sc2nc(N)nc3nc[nH]c23)ncc1Cl ; CCOc1ncc(F)c(NC(=O)[C@@H](Sc2nc(N)nc3nc[nH]c23)C(C)C)n1 ; CCOc1nc(NC(=O)[C@@H](CC)Sc2nc(N)nc3nc[nH]c23)ncc1Br]

or a pharmaceutically acceptable salt, tautomer, hydrate or solvate thereof.

In some embodiments, the compound of Formula (A1) or Formula (A2) is a compound provided in Table 2, Table 3, or Table 4, below.

In some embodiments, the compound of Formula (A2) is a compound of Formula (X).

In various embodiments, the present disclosure provides a compound of Formula (X) or a pharmaceutically acceptable salt, hydrate, or tautomer thereof:

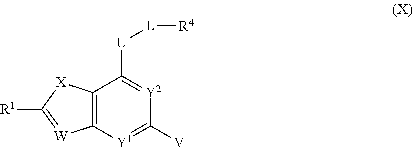

wherein:

L is a linker selected from alkylene, alkenylene, optionally substituted alkylene-S—, optionally substituted alkylene-O—, optionally substituted -alkylene-(NR⁵)—, optionally substituted

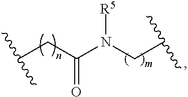

optionally substituted

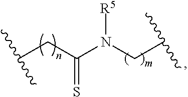

optionally substituted

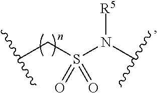

optionally substituted

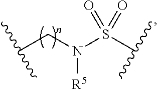

optionally substituted

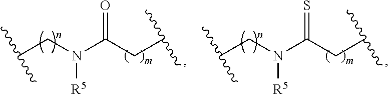

optionally substituted

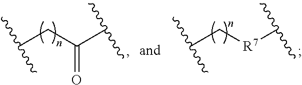

U is S, S(O)₂, or NH;

V is OH, NR²N³ or V and Y¹ taken together with the atoms to which they are attached form an optionally substituted phenyl or pyridinyl ring;

W is CH or N;

X is O, S, NR⁶, —CH═CH—, or —CH═N—;

Y¹ and Y² are each independently CH or N;

R¹ is H, OH, O-alkyl, alkyl or carbocyclyl;

R² and R³ are each independently H, alkyl, alkylenearyl, or —C(O)alkyl;

R⁴ is carbocyclyl, heterocyclyl, aryl, or heteroaryl, each of which is optionally substituted;

R⁵ is H, alkyl, —C(O)alkyl, carbocyclyl, alkylenecarbocyclyl, or alkylenearyl;

R⁶ is H, alkyl, carbocyclyl, alkylenecarbocyclyl, alkylenearyl, —C(O)alkyl, or —C(O)Oalkylenearyl;

R⁷ is carbocyclyl, heterocyclyl, or heteroaryl;

m is 0, 1, or 2; and

n is 1, 2, or 3.

In various embodiments, the present disclosure provides a compound of Formula (X) or a pharmaceutically acceptable salt, hydrate, or tautomer thereof:

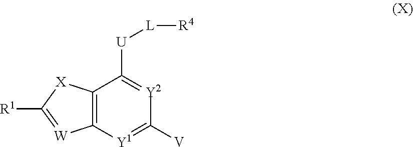

wherein:

L is a linker selected from alkylene, alkenylene, optionally substituted alkylene-S—, optionally substituted alkylene-O—, optionally substituted -alkylene-(NR⁵)—, optionally substituted

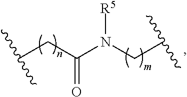

optionally substituted

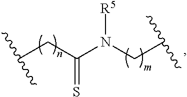

optionally substituted

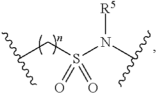

optionally substituted

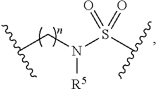

optionally substituted

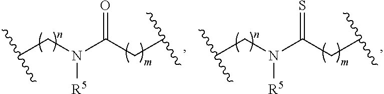

optionally substituted

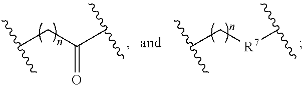

U is S, S(O)₂, or NH;

V is OH, NR²N³ or V and Y¹ taken together with the atoms to which they are attached form an optionally substituted phenyl or pyridinyl ring;

W is CH or N;

X is O, S, NR⁶, —CH═CH—, or —CH═N—;

Y¹ and Y² are each independently CH or N;

R¹ is H, OH, O-alkyl, alkyl or carbocyclyl;

R² and R³ are each independently H, alkyl, alkylenearyl, or —C(O)alkyl;

R⁴ is carbocyclyl, heterocyclyl, aryl, or heteroaryl, each of which is optionally substituted;

R⁵ is H, alkyl, —C(O)alkyl, carbocyclyl, alkylenecarbocyclyl, or alkylenearyl;

R⁶ is H, alkyl, carbocyclyl, alkylenecarbocyclyl, alkylenearyl, —C(O)alkyl, or —C(O)Oalkylenearyl;

R⁷ is carbocyclyl, heterocyclyl, or heteroaryl;

m is 0, 1, or 2; and

n is 1, 2, or 3,

provided that the compound of Formula (X) is not one or more of the following:

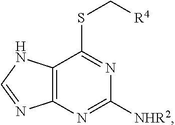

wherein:

(a) R² is H and R⁴ is

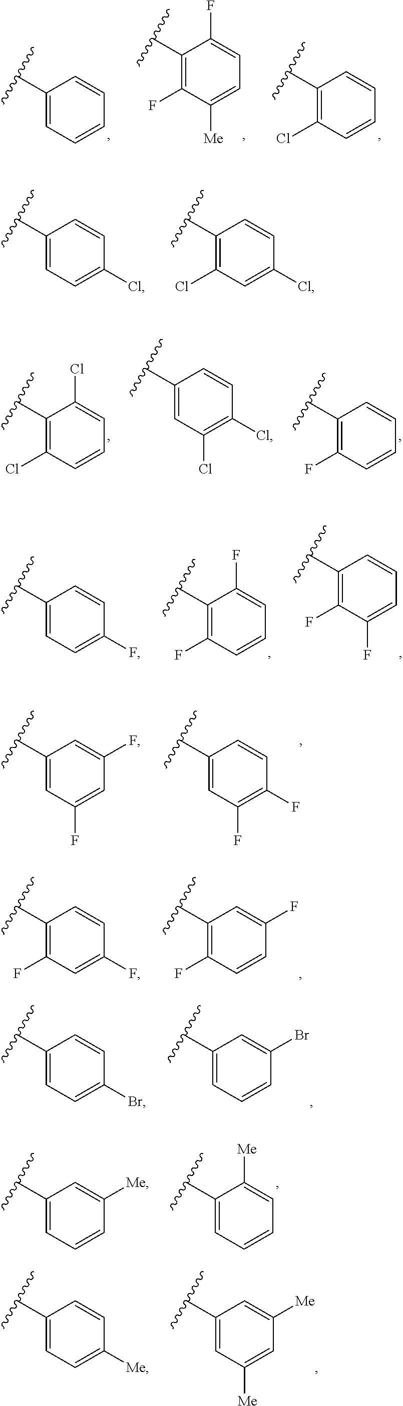

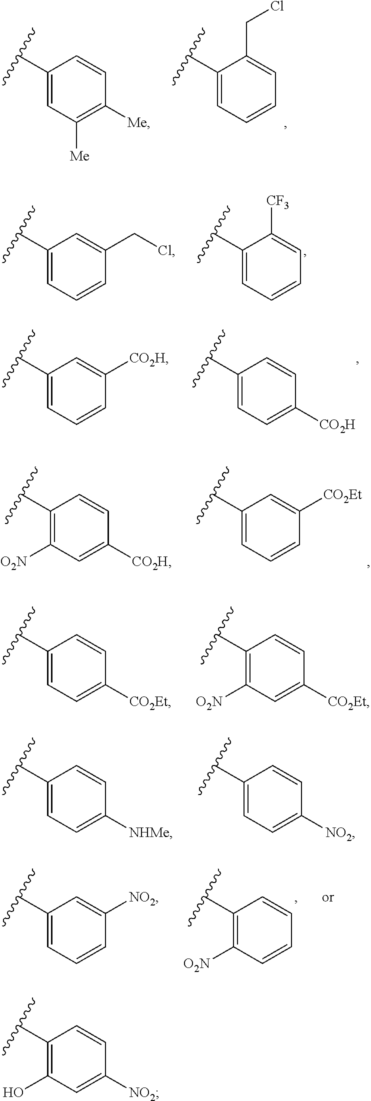

or

(b) R² is Me and R⁴ is

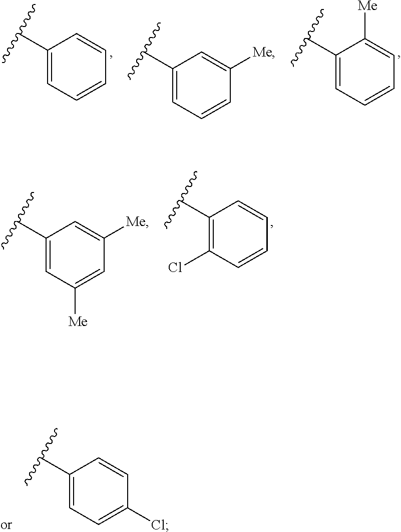

or

(c) R² is Et and R⁴ is

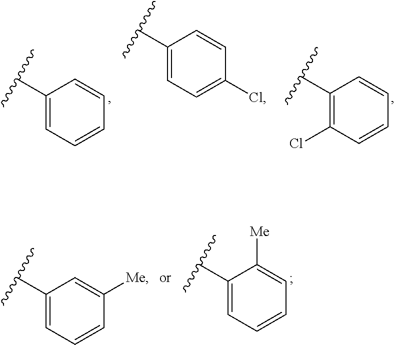 [SMILES: *c1ccccc1 ; *c1ccc(Cl)cc1 ; *c1ccccc1Cl ; *c1cccc(C)c1 ; "or" ; *c1ccccc1C]

(d) R² is nPr, C(O)Me or CO₂nBu and R⁴ is

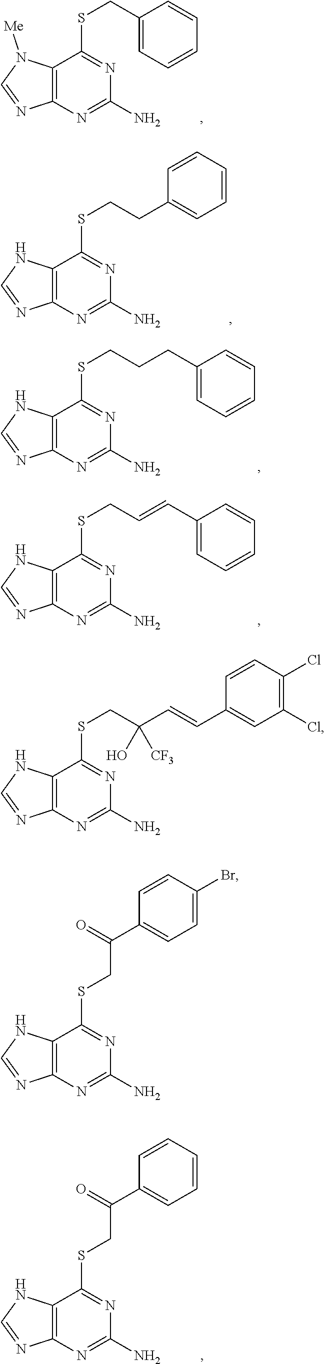

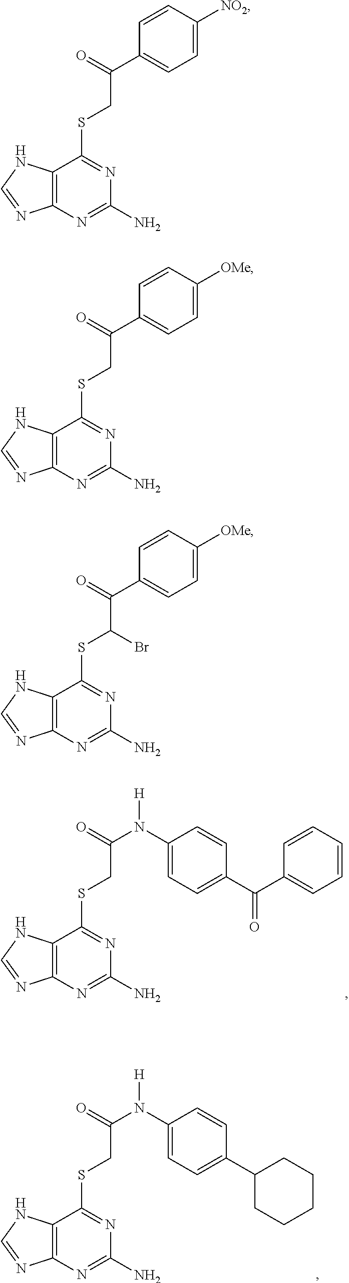

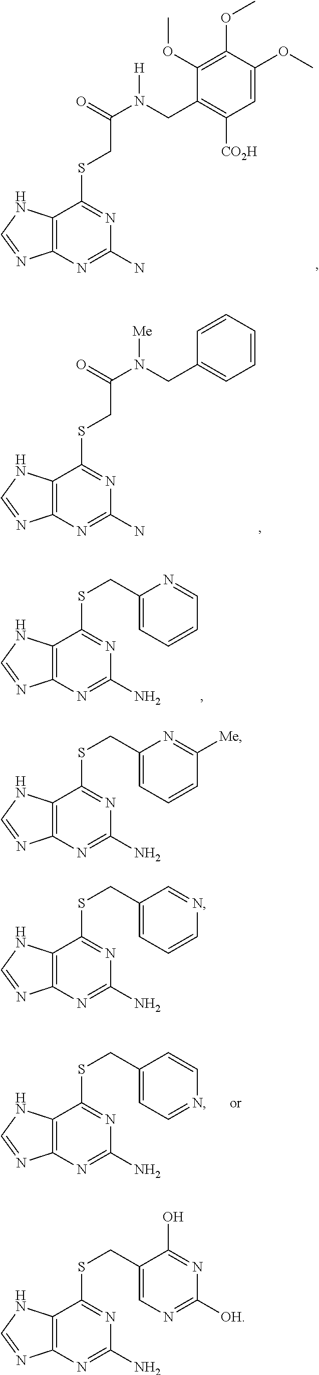

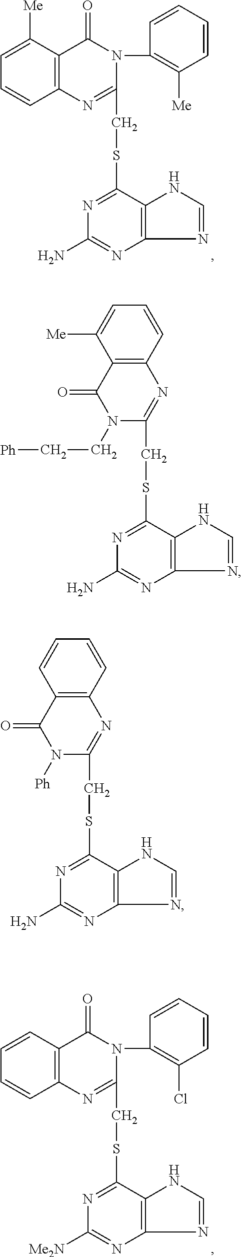 [SMILES: Cc1ccccc1-n1c(CSc2nc(N)nc3nc[nH]c23)nc2cccc(C)c2c1=O ; Cc1cccc2nc(CSc3nc(N)nc4nc[nH]c34)n(CCc3ccccc3)c(=O)c12 ; Nc1nc(SCc2nc3ccccc3c(=O)n2-c2ccccc2)c2[nH]cnc2n1 ; CN(C)c1nc(SCc2nc3ccccc3c(=O)n2-c2ccccc2Cl)c2[nH]cnc2n1]

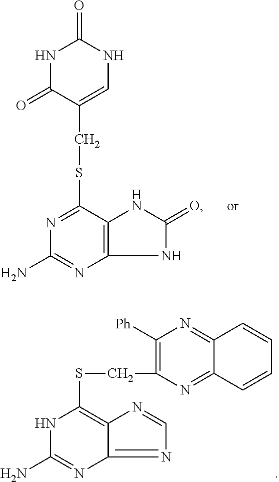 [SMILES: Nc1nc(SCc2c[nH]c(=O)[nH]c2=O)c2[nH]c(=O)[nH]c2n1 ; Nc1nc2ncnc-2c(SCc2nc3ccccc3nc2-c2ccccc2)[nH]1]

In some embodiments of Formula (X), L is an alkylene, alkenylene, alkylene-(NR⁵)—,

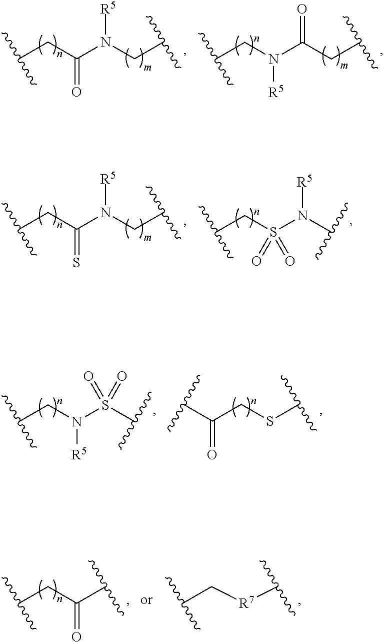

each of which is optionally substituted. In some embodiments, L is an alkylene, an alkylene-(NR⁵)—,

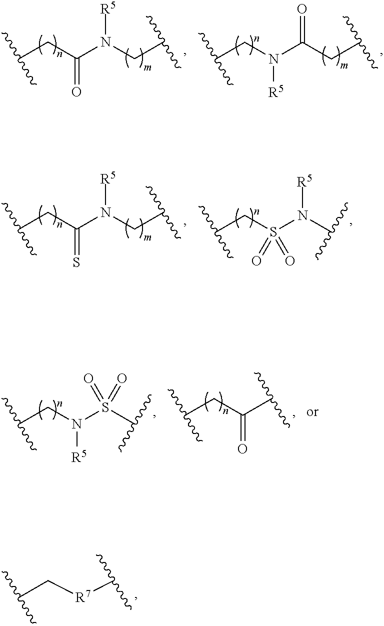

each of which is optionally substituted. In some embodiments of Formula (A1) and Formula (A2), L is an alkylene, analkenylene, an alkylene-(NR⁵)—,

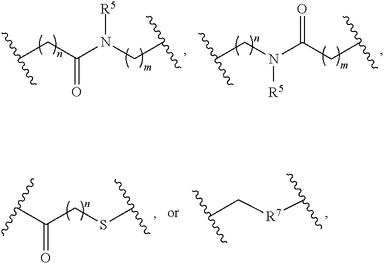

each of which is optionally substituted. In some embodiments, L is an alkylene-(NR⁵)—,

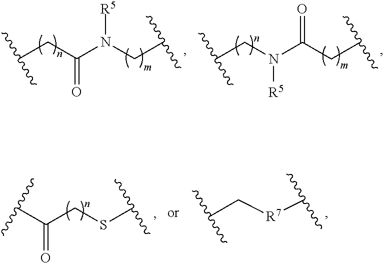

each of which is optionally substituted. In some embodiments, L is an alkylene-(NR⁵)—,

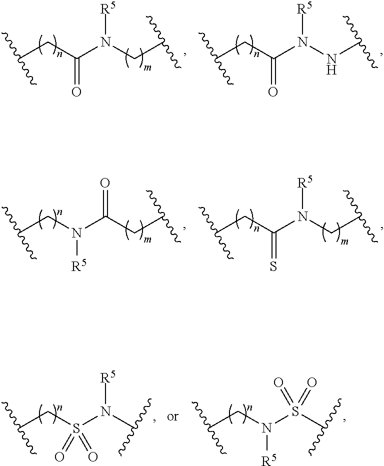

each of which is optionally substituted. In some embodiments L is an alkylene-(NR⁵)—,

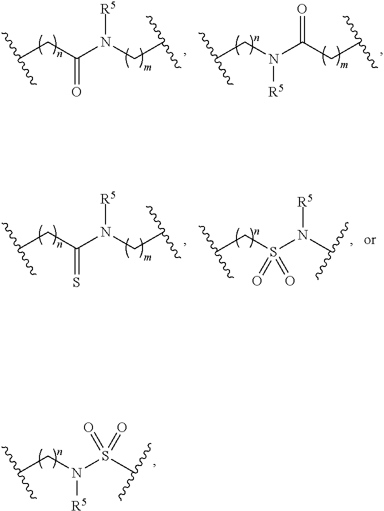

each of which is optionally substituted. In some embodiments, L is an alkylene-(NR⁵)—,

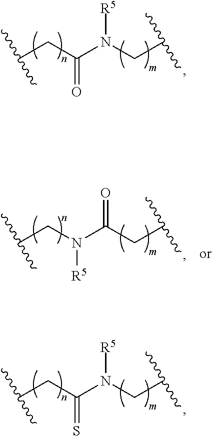

each of which is optionally substituted. In some embodiments, L is analkenylene, an alkylene-(NR⁵)—, an optionally substituted

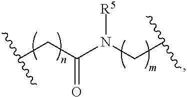

an optionally substituted

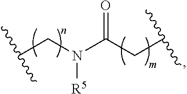

an optionally substituted

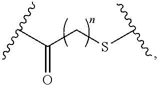

or an optionally substituted

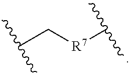

In some embodiments, L is an alkylene-(NR⁵)—,

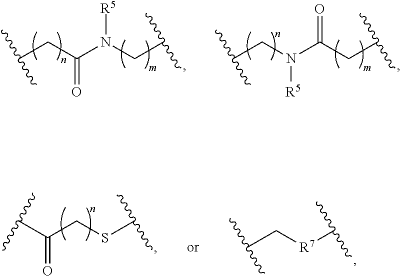

each of which is optionally substituted. In some embodiments, L is optionally substituted

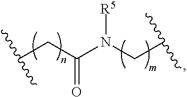

optionally substituted

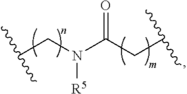

optionally substituted O

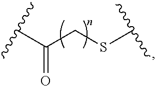

optionally substituted

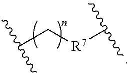

In some embodiments, L is optionally substituted

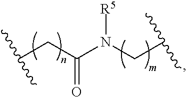

optionally substituted

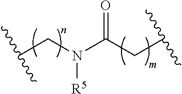

or optionally substituted

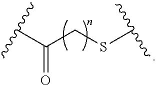

In some embodiments, L is optionally substituted

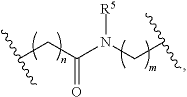

optionally substituted

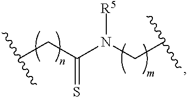

or optionally substituted

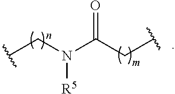

In some embodiments, L is optionally substituted

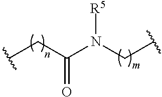

or optionally substituted

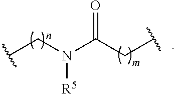

In some embodiments, L is optionally substituted

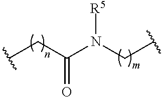

or optionally substituted

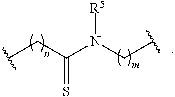

In some embodiments, L is optionally substituted

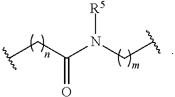

In other embodiments, L is optionally substituted

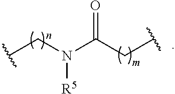

In some embodiments, L is optionally substituted

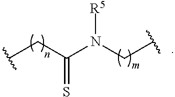

In still other embodiments, L is optionally substituted

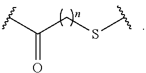

In some embodiments, L is

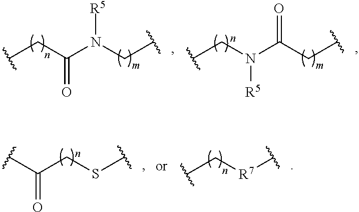

In some embodiments, L is

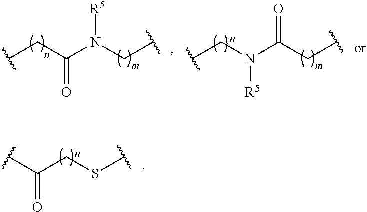

In some embodiments, L is

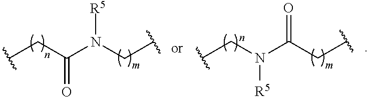

In some embodiments, L is

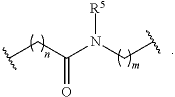

In other embodiments, L is

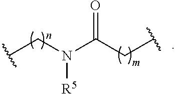

In still other embodiments, L is

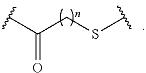

In some embodiments, m is 0 and n is 1. In some embodiments, m is 0 and n is 2. In other embodiments, m is 1 and n is 1.

In some embodiments of Formula (X), optionally substituted

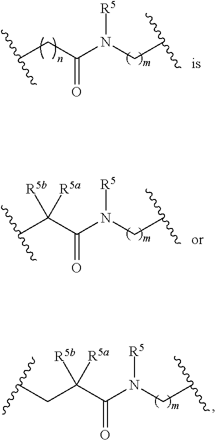

wherein:

R⁵ is H, alkyl, —C(O)alkyl, carbocyclyl, alkylenecarbocyclyl, or alkylenearyl;

R^{5a} and R^{5b} are each independently selected from the group consisting of H, halogen, C₁₋₅alkyl, C₃₋₆carbocyclyl, alkylene-C₃₋₆carbocyclyl, aryl, alkylenearyl, or NH₂; wherein two. C₁₋₅alkyl taken together with the carbon atom to which they are attached form a C₃₋₆carbocyclyl; and m is 0 or 1.

In some embodiments of Formula (X), optionally substituted

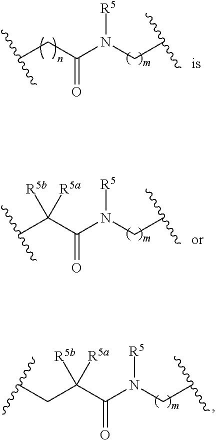

wherein:

R⁵ is H, methyl, or —C(O)Me;

R^{5a} is alkyl or carbocyclyl; and

R^{5b} is H,

wherein two C₁₋₅alkyl taken together with the carbon atom to which they are attached form a C₃₋₆carbocyclyl; and

m is 0 or 1.

In some embodiments of Formula (X), optionally substituted

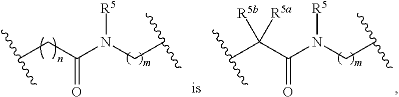

wherein:

R⁵ is H or methyl;

R^{5a} is fluoro or alkyl; and

R^{5b} is H,

wherein two C₁₋₅alkyl taken together with the carbon atom to which they are attached form a C₃₋₆carbocyclyl; and

m is 0.

In some embodiments, when L is

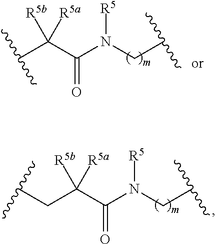

an R⁵ and an R^{5a} taken together with the carbon atoms to which they are attached form a heterocyclyl ring. In some embodiments, when L is

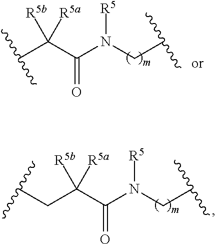

an R⁵ and an R^{5a} taken together with the carbon atoms to which they are attached form a 4-, 5- or 6-membered heterocyclyl ring. In some embodiments, the heterocyclyl ring is

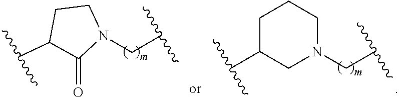

In some embodiments of Formula (X),

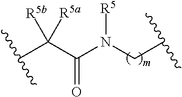

is selected from the group consisting of:

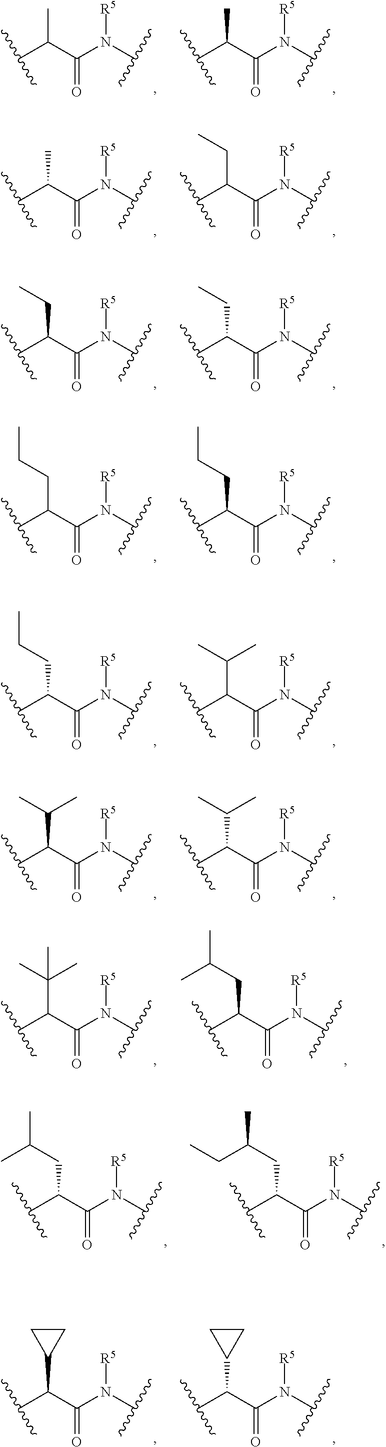

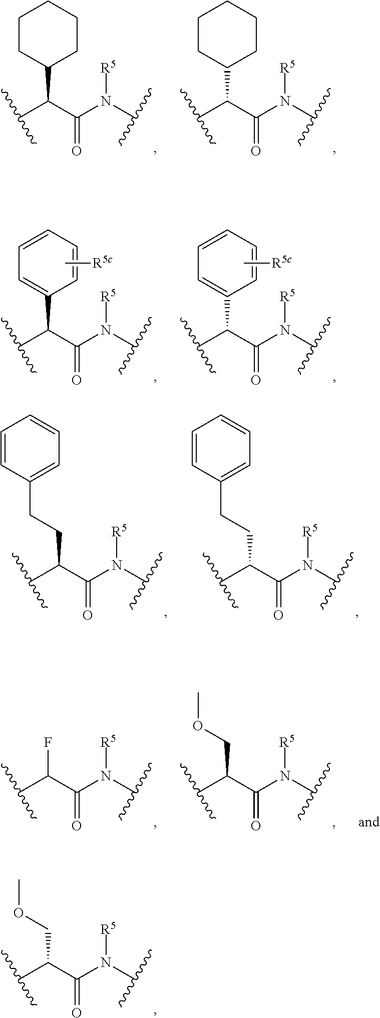

wherein R^{5c} is halogen, alkyl, haloalkyl, hydroxy, or alkoxy. In some embodiments, R^{5c} is in the para position of the phenyl ring.

In some embodiments,

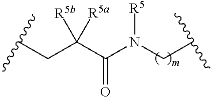

is selected from the group consisting of:

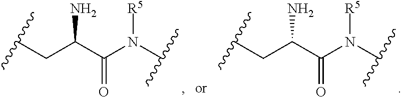

In some embodiments of Formula (X),

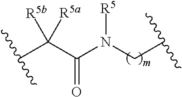

is selected from the group consisting of:

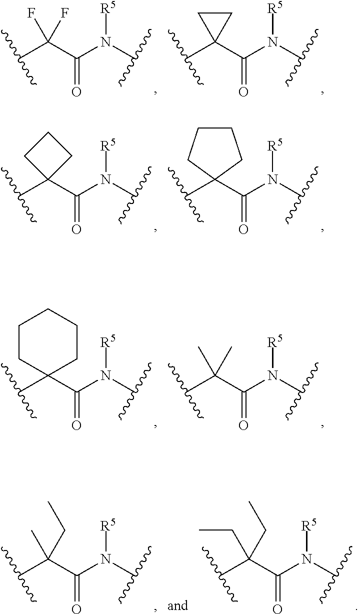

In some embodiments of Formula (X), optionally substituted selected from the group consisting of:

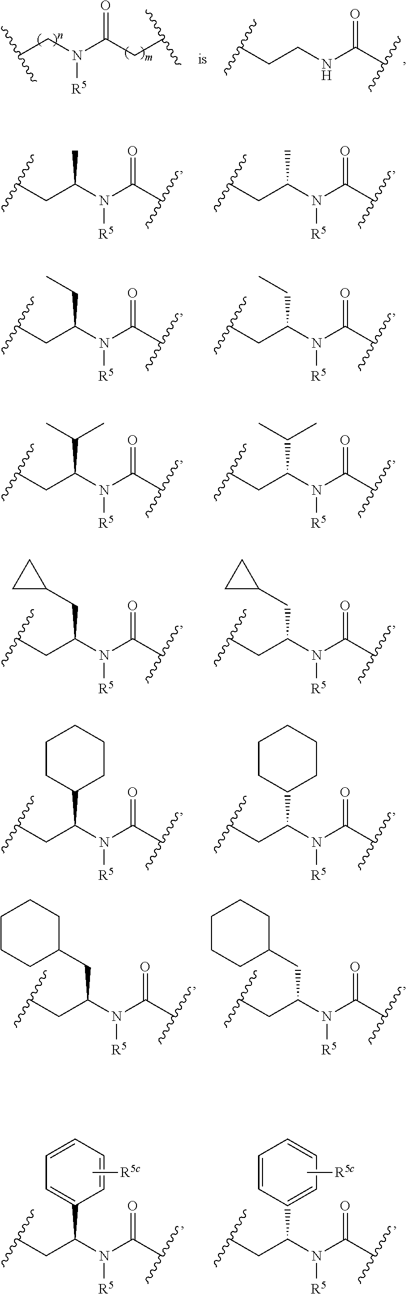

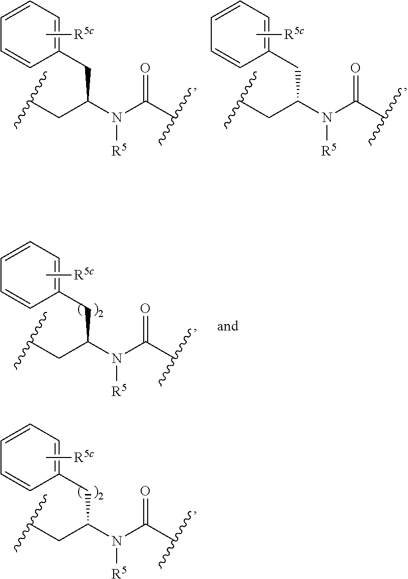

wherein R^{5c} is halogen, alkyl, haloalkyl, hydroxy, or alkoxy. In some embodiments, R^{5c} is in the para position of the phenyl ring.

In some embodiments of Formula (X), optionally substituted

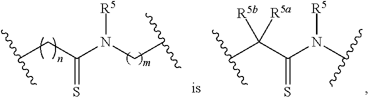

wherein:

R⁵ is H, alkyl, —C(O)alkyl, carbocyclyl, alkylenecarbocyclyl, or alkylenearyl; and

R^{5a} and R^{5b} are each independently selected from the group consisting of H, halogen, C₁₋₅alkyl, C₃₋₆carbocyclyl, alkylene-C₃₋₆carbocyclyl, aryl, alkylenearyl, or NH₂; wherein two —C₁₋₅alkyl taken together with the carbon atom to which they are attached form a C₃₋₆carbocyclyl.

In some embodiments, R⁵ is H, methyl, or —C(O)Me. In some embodiments, R⁵ is H, R^{5a} is alkyl or carbocyclyl, and R^{5b} is H. In some embodiments, R⁵ is H, R^{5a} is alkyl, and R^{5b} is H.

In some embodiments,

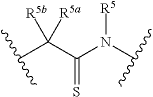

is selected from the group consisting of:

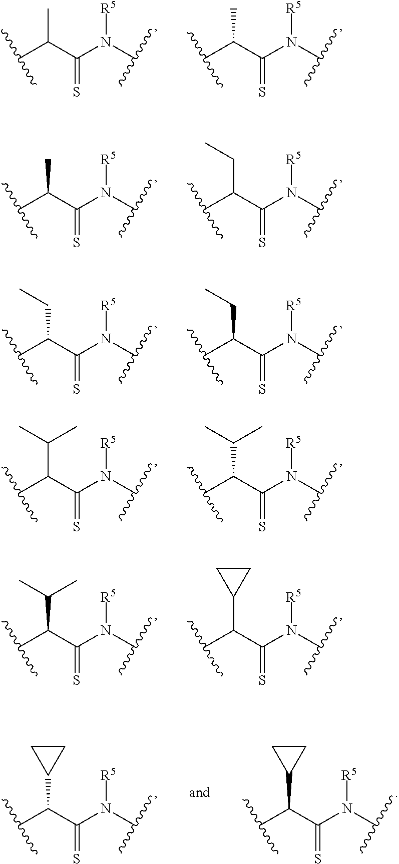

In some embodiments of Formula (X), when L comprises an alkylene, the alkylene is an optionally substituted C₁₋₄alkylene. In some embodiments, the alkylene is an optionally substituted C₁₋₃alkylene. In some embodiments, the alkylene is an optionally substituted C₁₋₂alkylene. In some embodiments, the alkylene is an optionally substituted C₂₋₄alkylene. In some embodiments, the alkylene is an optionally substituted C₂₋₃alkylene. In some embodiments, the alkylene is an optionally substituted C₃₋₄alkylene. In some embodiments, when L comprises an alkylene, the alkylene is a C₁₋₄alkylene. In some embodiments, the alkylene is a C₁₋₃alkylene. In some embodiments, the alkylene is a C₁₋₂alkylene. In some embodiments, the alkylene is a C₂₋₄alkylene. In some embodiments, the alkylene is a C₂₋₃alkylene. In some embodiments, the alkylene is a C₃₋₄alkylene. In some embodiments, the alkylene is a methylene, an ethylene, a propylene, or a butylene, each of which is optionally substituted. In some embodiments, the alkylene is an ethylene, a propylene, or a butylene, each of which is optionally substituted. In some embodiments, the alkylene is an optionally substituted methylene. In some embodiments, the alkylene is an optionally substituted ethylene. In some embodiments, the alkylene is an optionally substituted propylene. In some embodiments, the alkylene is an optionally substituted butylene. In some embodiments, the alkylene is a methylene, an ethylene, a propylene, or a butylene. In some embodiments, the alkylene is a methylene. In some embodiments, the alkylene is an ethylene. In some embodiments, the alkylene is a propylene. In some embodiments, the alkylene is a butylene.

In some embodiments of Formula (X), L is alkylene-(NR⁵)—. In some embodiments, the alkylene is optionally substituted ethylene. In some embodiments, the optionally substituted ethylene is selected from the group consisting of:

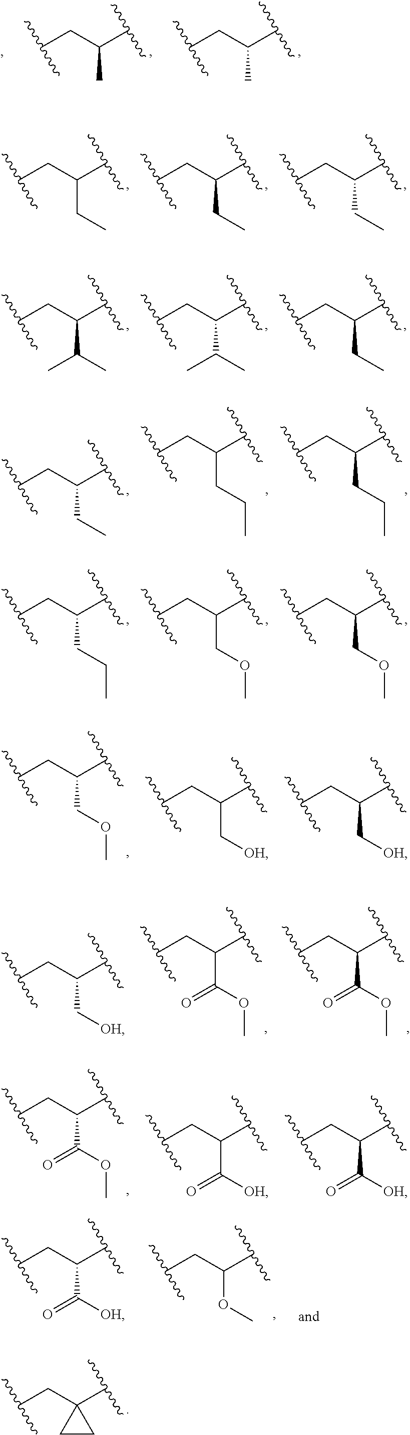

In some embodiments of Formula (X), L is alkylene-(NR⁵)—. In some embodiments, the alkylene is optionally substituted propylene. In some embodiments, the optionally substituted propylene is selected from the group consisting of:

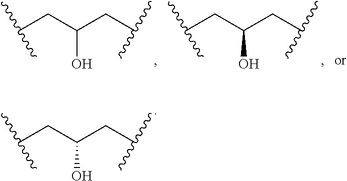

In some embodiments of Formula (X), when L comprises an alkenylene, the alkenylene is an optionally substituted C₂₋₄alkenylene. In some embodiments, the alkenylene is an optionally substituted C₂₋₃alkenylene. In some embodiments, the alkenylene is an optionally substituted C₃₋₄alkenylene. In some embodiments, when L comprises an alkenylene, the alkenylene is a C₂₋₄alkenylene. In some embodiments, the alkenylene is a C₂₋₃alkenylene. In some embodiments, the alkenylene is a C₃₋₄alkenylene. In some embodiments, the alkenylene is an ethenylene, a propenylene, or a butenylene, each of which is optionally substituted. In some embodiments, the alkenylene is an optionally substituted ethenylene. In some embodiments, the alkenylene is an optionally substituted propenylene. In some embodiments, the alkenylene is an optionally substituted butenylene. In some embodiments, the alkenylene is an ethenylene, a propenylene, or a butenylene. In some embodiments, the alkenylene is an ethenylene. In some embodiments, the alkenylene is a propenylene. In some embodiments, the alkenylene is a butenylene.

In some embodiments of Formula (X), the optional substituent is selected from the group consisting of oxo, halogen, C₁₋₅alkyl, C₃₋₆carbocyclyl, alkylenecarbocyclyl, aryl, heteroaryl, alkylenearyl, and alkyleneheteroaryl. In some embodiments, the optional substituent is selected from the group consisting of oxo, C₁₋₅alkyl, and C₃₋₆cycloalkyl. In some embodiments, the optional substituent is selected from the group consisting of oxo and C₁₋₅alkyl. In some embodiments, the optional substituent is oxo. In other embodiments, the optional substituent is C₁₋₅alkyl. In some embodiments, the C₁₋₅alkyl is methyl, ethyl, propyl or isopropyl. In some embodiments, the C₁₋₅alkyl is methyl, ethyl, or isopropyl. In other embodiments, the C₁₋₅alkyl is methyl. In some embodiments, the C₃₋₆cycloalkyl is cyclopropyl or cyclohexyl. In some embodiments, the aryl is phenyl. In some embodiments, the alkylenecarbocyclyl is methylenecyclopropyl or methylenecyclohexyl. In some embodiments, the alkylenearyl is methylenephenyl.

In some embodiments of Formula (X), m is 0 or 1. In some embodiments, m is 1 or 2. In some embodiments, m is 0 or 2. In some embodiments, m is 0. In some embodiments, m is 1. In some embodiments, m is 2.

In some embodiments of Formula (X), n is 1 or 2. In some embodiments, n is 2 or 3. In some embodiments, n is 1 or 3. In some embodiments, n is 1. In some embodiments, n is 2. In some embodiments, n is 3.

In some embodiments of Formula (X), m is 0 and n is 1. In other embodiments, m is 1 and n is 1. In still other embodiments, m is 0 and n is 2. In yet another embodiment, m is 2 and n is 1.

In some embodiments of Formula (X), U is S. In other embodiments, U is NH.

In some embodiments of Formula (X), V is H, OH, NR²N³, or N═CR²R³. In some embodiments of Formula (X), V is H, OH, or NR²N³. In some embodiments, V and Y¹ taken together with the atoms to which they are attached form an optionally substituted phenyl or pyridinyl ring. In some embodiments, V is NR²N³. In other embodiments, V is OH. In some embodiments, V is H.

In some embodiments of Formula (X), W is N. In other embodiments, W is CH.

In some embodiments of Formula (X), X is O, S, or NR⁶. In some embodiments, X is O or NR⁶. In some embodiments, X is NR⁶. In some embodiments, X is O. In some embodiments, X is S. In some embodiments, X is —CH═CH— or —CH═N—.

In some embodiments of Formula (X), Y¹ or Y² is N. In some embodiments, Y¹ and Y² are both N. In some embodiments, Y¹ is N and Y² is CH. In some embodiments, Y¹ is CH and Y² is N.

In some embodiments of Formula (X), U is S, W is N, and X is NR⁶. In certain embodiments, V is NR²NR³.

In some embodiments of Formula (X), U is S, W is N, and X is NR⁶. In certain embodiments, Y¹ and Y² are each N.

In some embodiments of Formula (X), U is S, W is N, and X is NR⁶. In certain embodiments, V is NR²NR³.

In some embodiments of Formula (X), U is S, W is N, X is NR⁶, and Y¹ and Y² are each N. In certain embodiments, V is NR²NR³.

In some embodiments of Formula (X), U is S, W is N, X is NR⁶, and V is NR²NR³. In certain embodiments, Y¹ and Y² are each N.

In some embodiments of Formula (X), R¹ is H, OH, or C₁₋₅alkyl. In other embodiments, R¹ is H. In some embodiments, R¹ is OH. In some embodiments, R¹ is C₁₋₅alkyl. In some embodiments, the C₁₋₅alkyl is selected from the group consisting of methyl, ethyl, propyl, isopropyl, isoamyl, and isobutyl. In other embodiments, the C₁₋₅alkyl is selected from the group consisting of methyl, ethyl, and isopropyl.

In some embodiments of Formula (X), R² and R³ are independently H, —C₁₋₅alkyl, —CH₂Ph, or —C(O)(C₁₋₅alkyl). In some embodiments, R² and R³ are independently H, —C₁₋₅alkyl, —CH₂Ph, or —C(O)(CH₃). In some embodiments, one of R² and R³ is H. In some embodiments, R² and R³ are H. In some embodiments, one of R² and R³ is —C₁₋₅alkyl. In some embodiments, one of R² and R³ is —CH₂Ph. In some embodiments, one of R² and R³ is —C(O)(CH₃). In some embodiments, the C₁₋₅alkyl is selected from the group consisting of methyl, ethyl, and isopropyl.

In some embodiments of Formula (X), R⁴ is aryl or heteroaryl, each of which is optionally substituted. In some embodiments, R⁴ is optionally substituted aryl. In some embodiments, R⁴ is optionally substituted heteroaryl. In some embodiments, the heteroaryl is oxazolyl, thiazolyl, triazolyl, oxadiazolyl, thiadiazolyl, imidazolyl, isoxazolyl, indolyl, oxindolyl, isatinyl, benzothiazolyl, benzoxazolyl, benzimidazolyl, benzotriazolyl, benzofuranyl, benzothiophenyl, pyrazolyl, pyridinyl, pyrazinyl, pyrimidinyl, quinolinyl, isoquinolinyl, cinnolinyl, quinazolinyl, or quinoxalinyl. In some embodiments, the aryl is a 6- to 12-membered aryl and the heteroaryl is a 5- to 12-membered heteroaryl with 1, 2, or 3 heteroatoms selected from the group consisting of N, O, and S. In some embodiments, the 5- to 12-membered heteroaryl with 1, 2, or 3 heteroatoms selected from the group consisting of N, O, and S is oxazolyl, thiazolyl, triazolyl, oxadiazolyl, thiadiazolyl, imidazolyl, isoxazolyl, tetrazolyl, or pyrazolyl. In some embodiments, the 5- to 12-membered heteroaryl with 1, 2, or 3 heteroatoms selected from the group consisting of N, O, and S is pyridinyl, pyrazinyl, or pyrimidinyl. In some embodiments, the 5- to 12-membered heteroaryl with 1, 2, or 3 heteroatoms selected from the group consisting of N, O, and S is indolinyl, benzothiazolyl, benzoxazolyl, benzimidazolyl, benzofuranyl, quinolinyl, isoquinolinyl, cinnolinyl, quinazolinyl, and quinoxalinyl.

In some embodiments of Formula (X), the aryl or heteroaryl is optionally substituted with one or more H, halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN. In other embodiments, the aryl or heteroaryl is optionally substituted with one or more H, halogen, —C₁₋₅alkyl, CF₃, —OH, —O(C₁₋₅alkyl), —OCF₃, —OSO₂Me, —COOH, —C(O)OMe, or —SO₂Me. In some embodiments, the aryl is an optionally substituted phenyl. In some embodiments, the heteroaryl is an optionally substituted pyridinyl. In certain embodiments, the optionally substituted pyridinyl is selected from the group consisting of

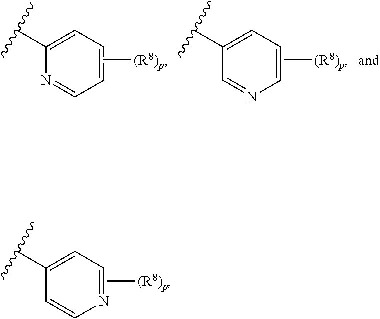

wherein p is 0, 1, or 2. In some embodiments, the heteroaryl is an optionally substituted pyrimidinyl. In certain embodiments, the optionally substituted pyrimidinyl is

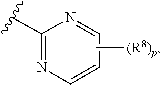

wherein p is 0, 1, or 2. In some embodiments, each R⁸ is independently halogen, alkyl, —OH, —Oalkyl, —CO₂H, or —CO₂alkyl.

In some embodiments of Formula (X), R⁴ is an optionally substituted aryl selected from the group consisting of:

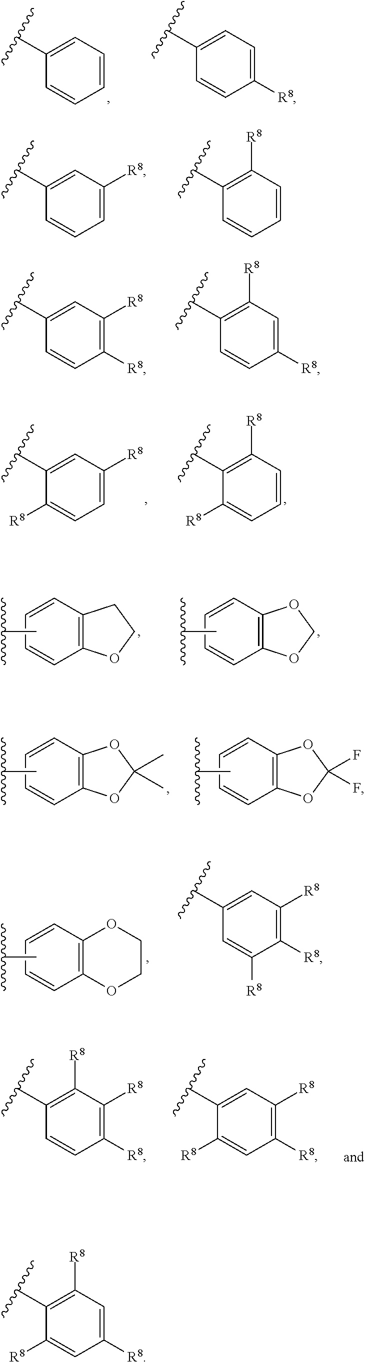

In some embodiments, R⁴ is an optionally substituted heteroaryl selected from the group consisting of:

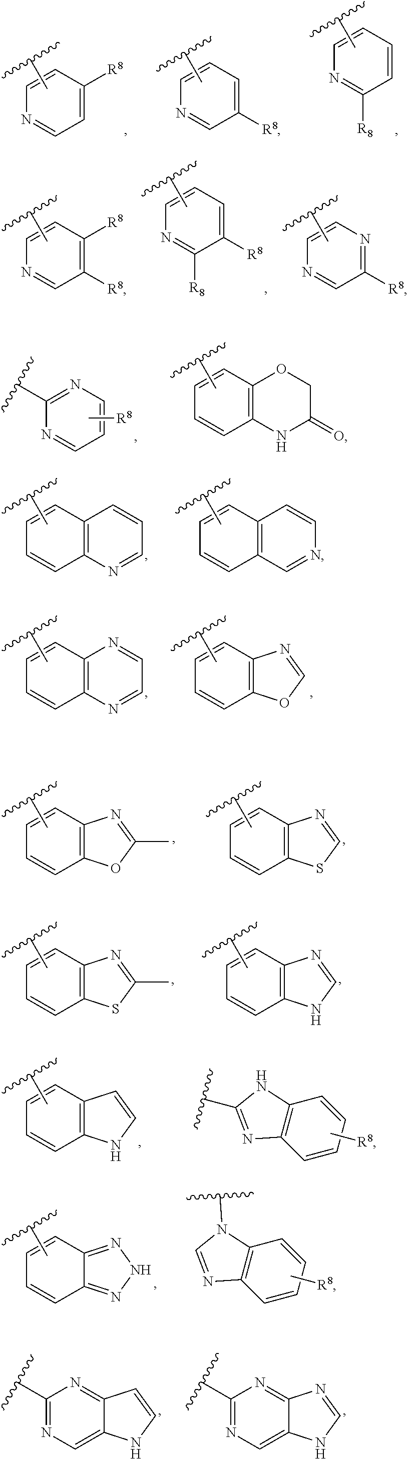

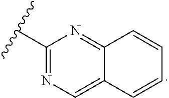 [SMILES: *c1ncc2ccccc2n1]

In some embodiments, R⁴ is selected from the group consisting of:

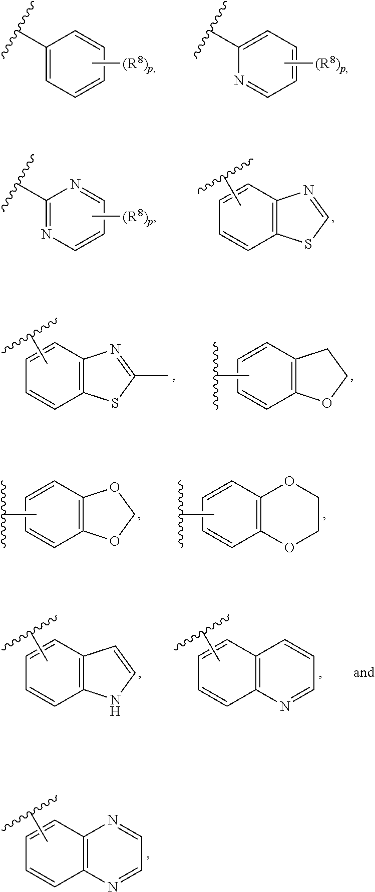

wherein p is an integer from 0-3. In some embodiments, each R⁸ is independently halogen, alkyl, haloalkyl, alkenyl, —OH, —Oalkyl, —N(alkyl)₂, —CO₂H, —CO₂alkyl, or —CN.

In some embodiments, R⁴ is selected from the group consisting of:

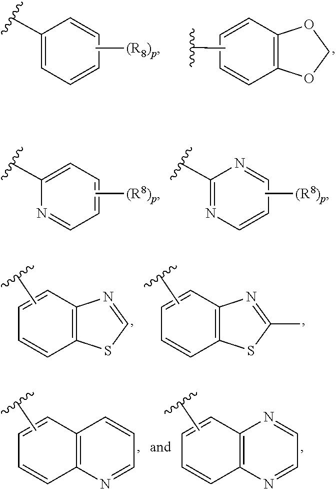

wherein:

each R⁸ is independently halogen, C₁₋₅ alkyl, —OH, —OC₁₋₅alkyl, —COOH, or —CO₂C₁₋₅alkyl; and

p is an integer from 0-3.

In some embodiments of Formula (X), R⁴ is carbocyclyl. In some embodiments, the carbocyclyl is an optionally substituted C₃₋₆carbocyclyl. In some embodiments, the carbocyclyl is cyclopropyl, cyclobutyl, cyclopentyl, or cyclohexyl. In some embodiments, the carbocyclyl is cyclohexyl.

In some embodiments of Formula (X), R⁴ is heterocyclyl. In some embodiments, the heterocyclyl is an optionally substituted 4- to 6-membered heterocyclyl containing 1 or 2 heteroatoms selected from N, O, and S. In some embodiments, the heterocyclyl is azetidinyl, pyrrolidinyl, piperidinyl, piperazinyl, morpholinyl, or thiomorpholinyl.

In some embodiments of Formula (X), R⁵ is H, —C(O)C₁₋₅alkyl, C₁₋₅alkyl, C₃₋₆carbocyclyl, —CH₂-aryl, or CH₂—(C₃₋₆carbocyclyl). In some embodiments, R⁵ is H, —C(O)C₁₋₅-alkyl, C₁₋₅alkyl, C₃₋₆carbocyclyl. In some embodiments, R⁵ is H, —C(O)C₁₋₅alkyl, or C₁₋₅alkyl. In some embodiments, R⁵ is H or C₁₋₅alkyl. In some embodiments, the C₁₋₅alkyl is selected from the group consisting of methyl, ethyl, and isopropyl. In some embodiments, the C₃₋₆carbocyclyl is cyclopropyl or cyclohexyl. In some embodiments, R⁵ is H, Me, or CH₂Ph. In some embodiments, R⁵ is H.

In some embodiments of Formula (X), R⁶ is H, C₁₋₅alkyl, CH₂aryl, or CH₂—(C₃₋₆carbocyclyl). In some embodiments, R⁶ is H, C₁₋₅alkyl, or CH₂Ph. In some embodiments, C₁₋₅alkyl is selected from the group consisting of methyl, ethyl, and isopropyl. In some embodiments, R⁶ is H.

In some embodiments of Formula (X), R⁷ is a C₃₋₆carbocyclyl, a 3- to 6-membered heterocyclyl, or a 5- to 6-membered heteroaryl. In some embodiments, R⁷ is a C₃₋₆carbocyclyl. In some embodiments, the C₃₋₆carbocyclyl is cyclopropyl or cyclohexyl. In some embodiments, R⁷ is a 5- to 6-membered heteroaryl. In some embodiments, the 5- to 6-membered heteroaryl is selected from the group consisting of oxazolyl, thiazolyl, triazolyl, oxadiazolyl, thiadiazolyl, imidazolyl, isoxazolyl, pyrazolyl, pyridinyl, pyrimidinyl, and pyrazinyl. In some embodiments, the 5- to 6-membered heteroaryl is selected from the group consisting of

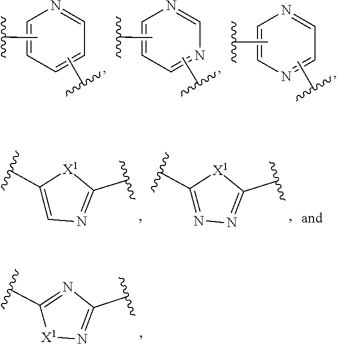

wherein X¹ is NR⁶, S, or O and R⁶ is H or alkyl. In some embodiments, R⁷ is a 5-membered heteroaryl. In some embodiments, the 5-membered heteroaryl is selected from the group consisting of

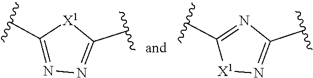

wherein X¹ is NR⁶, S, or O. In some embodiments, the 5-membered heteroaryl is selected from the group consisting of

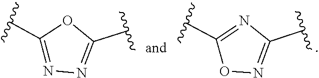 [SMILES: *c1nnc(*)o1 ; *c1noc(*)n1]

In some embodiments, R⁷ is a 3- to 6-membered heterocyclyl. In some embodiments, the 3- to 6-membered heterocyclyl is selected from the group consisting of azetidinyl, pyrrolidinyl, piperidinyl, piperazinyl, morpholinyl, and thiomorpholinyl.

In some embodiments, each R⁸ is independently halogen, alkyl, haloalkyl, alkenyl, —OH, —Oalkyl, —N(alkyl)₂, —CO₂H, —CO₂alkyl, or —CN. In some embodiments, each R⁸ is independently halogen, alkyl, haloalkyl, —OH, —Oalkyl, —Ohaloalkyl, or —CO₂H. In some embodiments, each R⁸ is independently halogen, alkyl, —OH, —Oalkyl, or —CO₂H.

In some embodiments, the compound of Formula (X) has a structure according to one of the following:

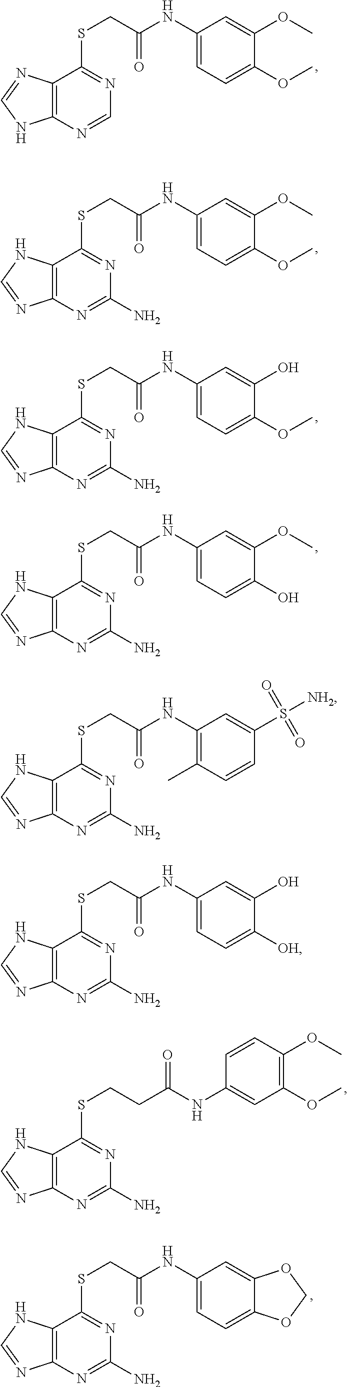

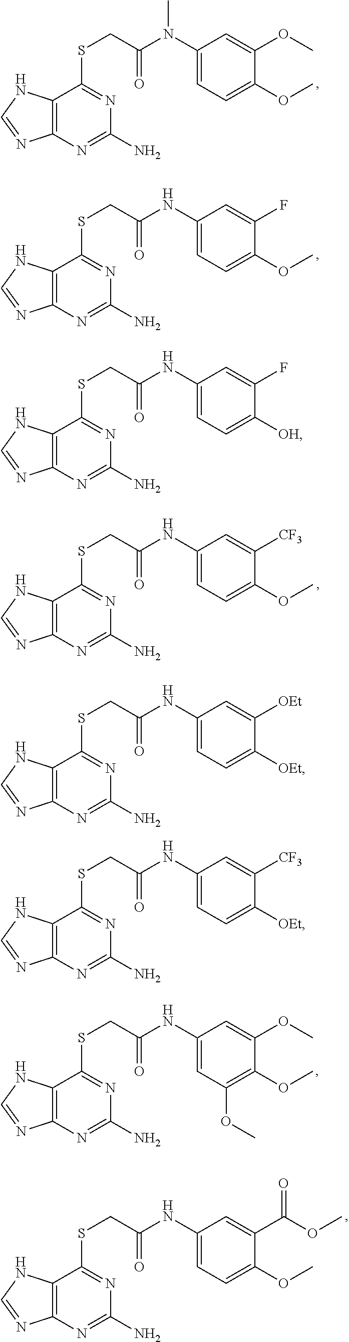

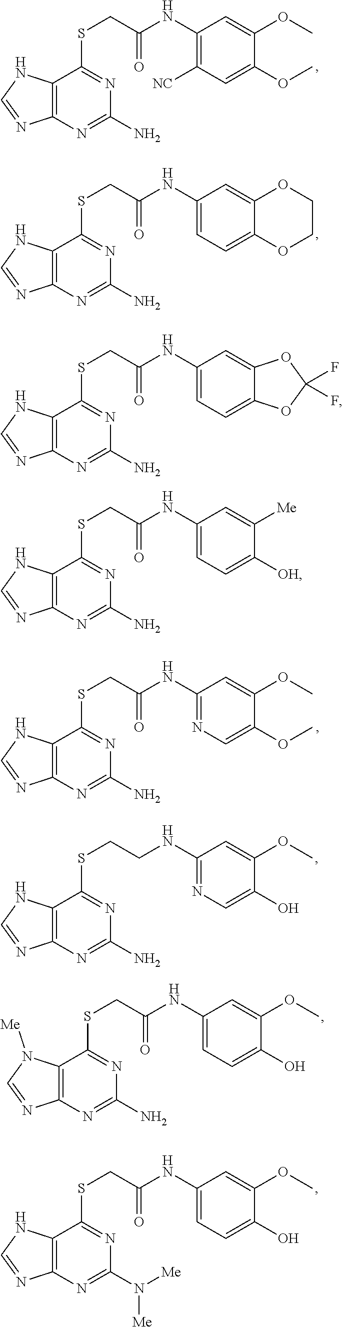

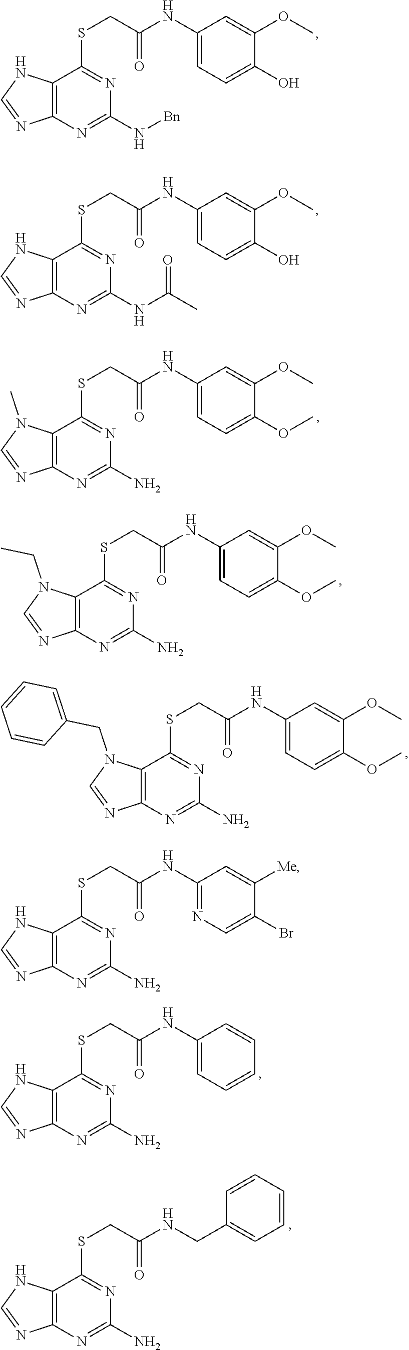

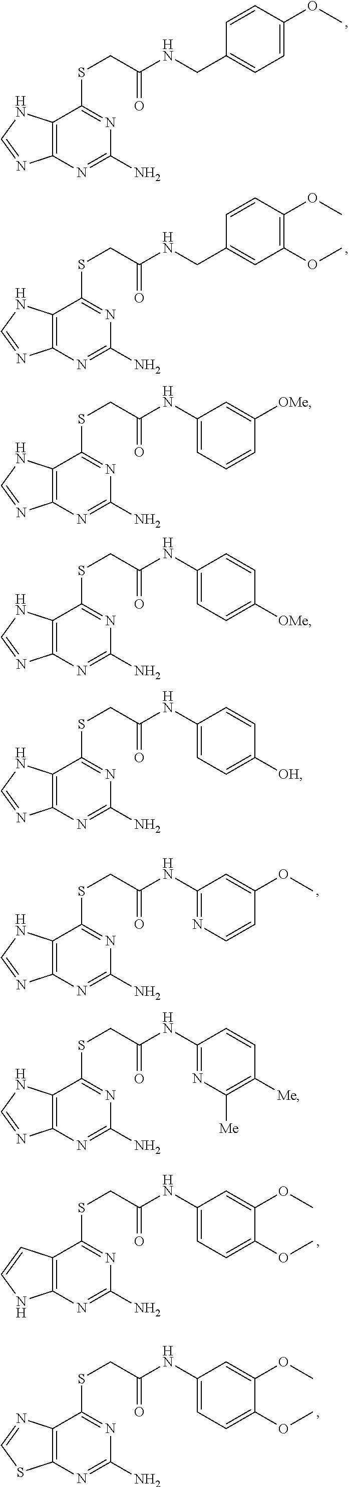

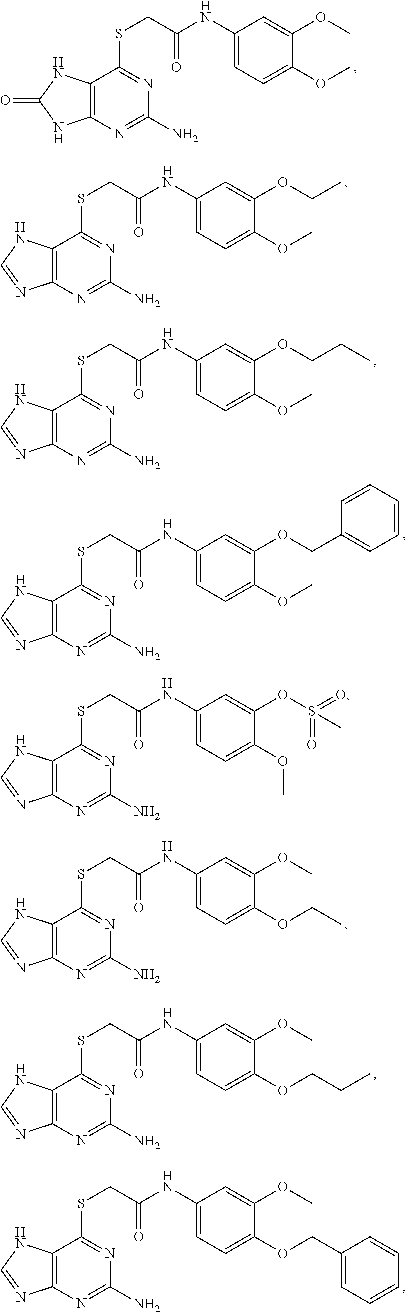

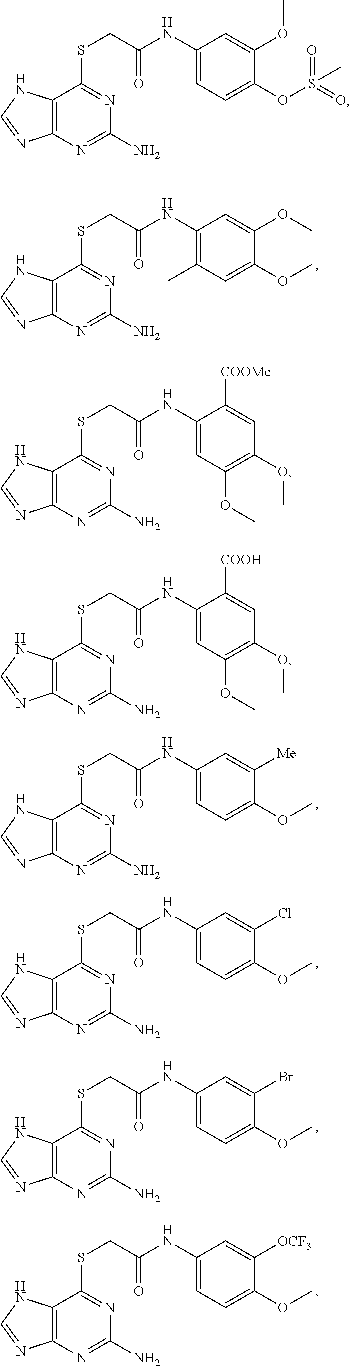

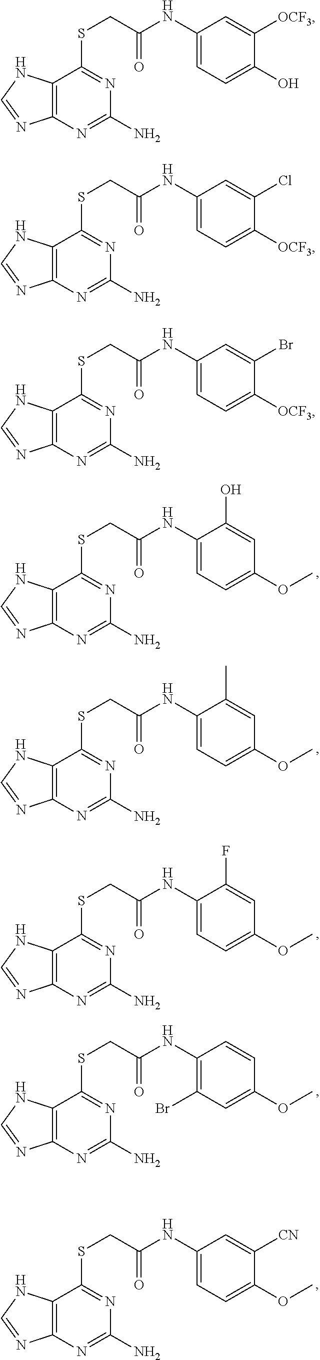

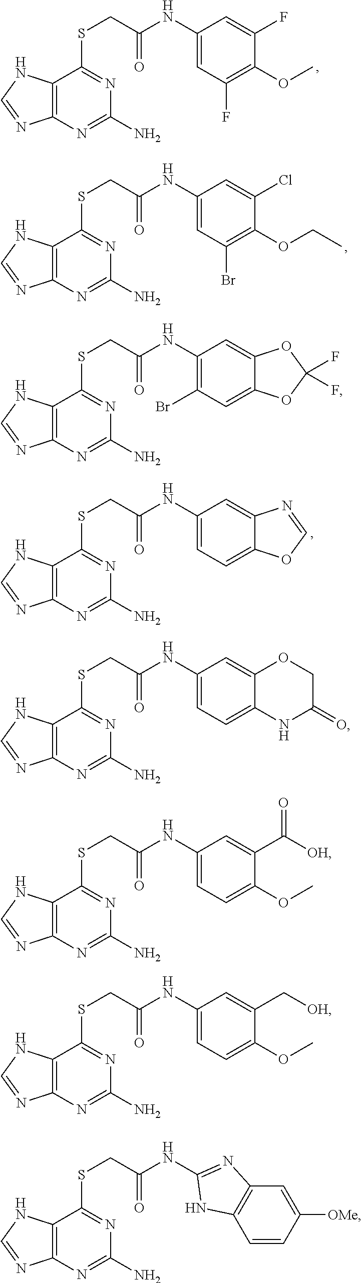

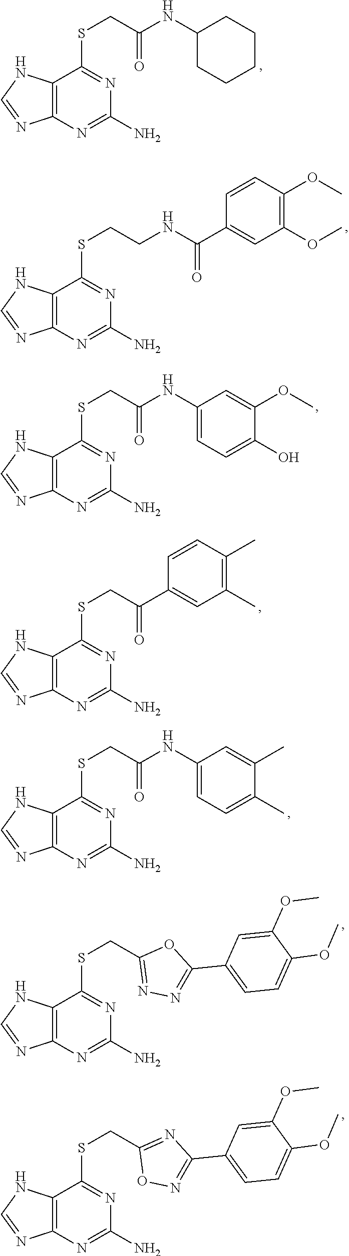

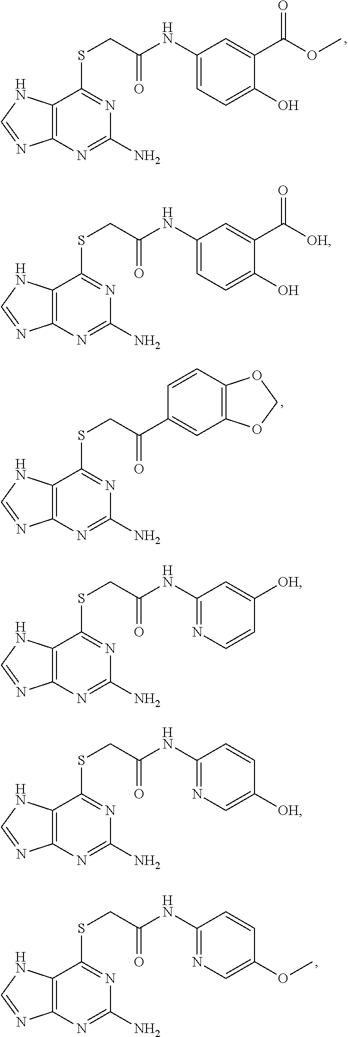 [SMILES: COC(=O)c1cc(NC(=O)CSc2nc(N)nc3nc[nH]c23)ccc1O ; Nc1nc(SCC(=O)Nc2ccc(O)c(C(=O)O)c2)c2[nH]cnc2n1 ; Nc1nc(SCC(=O)c2ccc3c(c2)OCO3)c2[nH]cnc2n1 ; Nc1nc(SCC(=O)Nc2cc(O)ccn2)c2[nH]cnc2n1 ; Nc1nc(SCC(=O)Nc2ccc(O)cn2)c2[nH]cnc2n1 ; COc1ccc(NC(=O)CSc2nc(N)nc3nc[nH]c23)nc1]

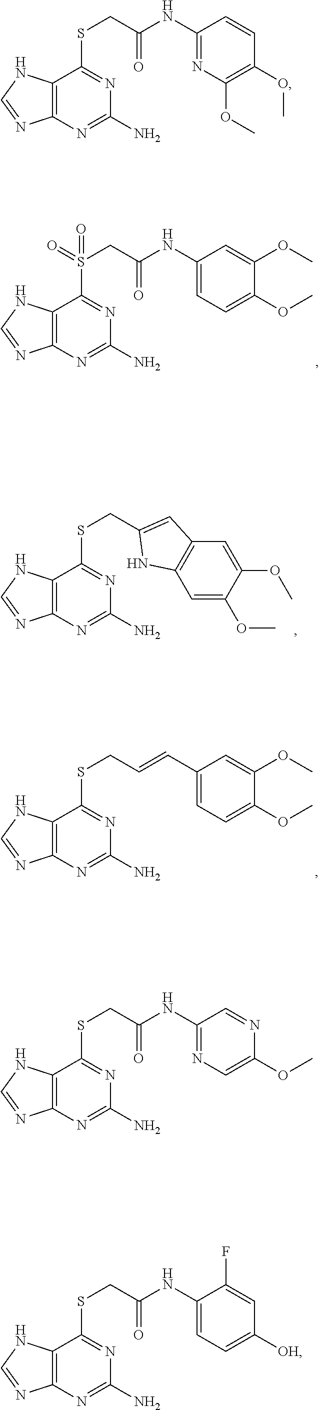 [SMILES: COc1ccc(NC(=O)CSc2nc(N)nc3nc[nH]c23)nc1OC ; COc1ccc(NC(=O)CS(=O)(=O)c2nc(N)nc3nc[nH]c23)cc1OC ; COc1cc2cc(CSc3nc(N)nc4nc[nH]c34)[nH]c2cc1OC ; COc1ccc(/C=C/CSc2nc(N)nc3nc[nH]c23)cc1OC ; COc1cnc(NC(=O)CSc2nc(N)nc3nc[nH]c23)cn1 ; Nc1nc(SCC(=O)Nc2ccc(O)cc2F)c2[nH]cnc2n1]

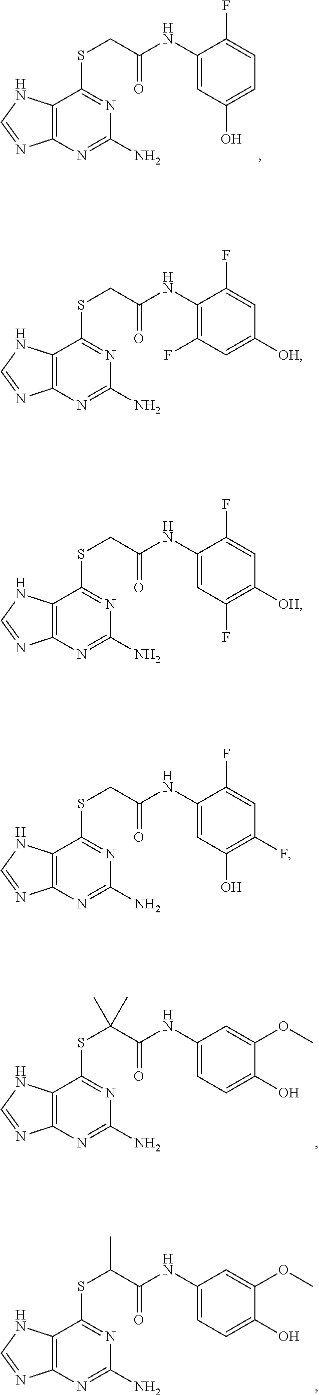 [SMILES: Nc1nc(SCC(=O)Nc2cc(O)ccc2F)c2[nH]cnc2n1 ; Nc1nc(SCC(=O)Nc2c(F)cc(O)cc2F)c2[nH]cnc2n1 ; Nc1nc(SCC(=O)Nc2cc(F)c(O)cc2F)c2[nH]cnc2n1 ; Nc1nc(SCC(=O)Nc2cc(O)c(F)cc2F)c2[nH]cnc2n1 ; COc1cc(NC(=O)C(C)(C)Sc2nc(N)nc3nc[nH]c23)ccc1O ; COc1cc(NC(=O)C(C)Sc2nc(N)nc3nc[nH]c23)ccc1O]

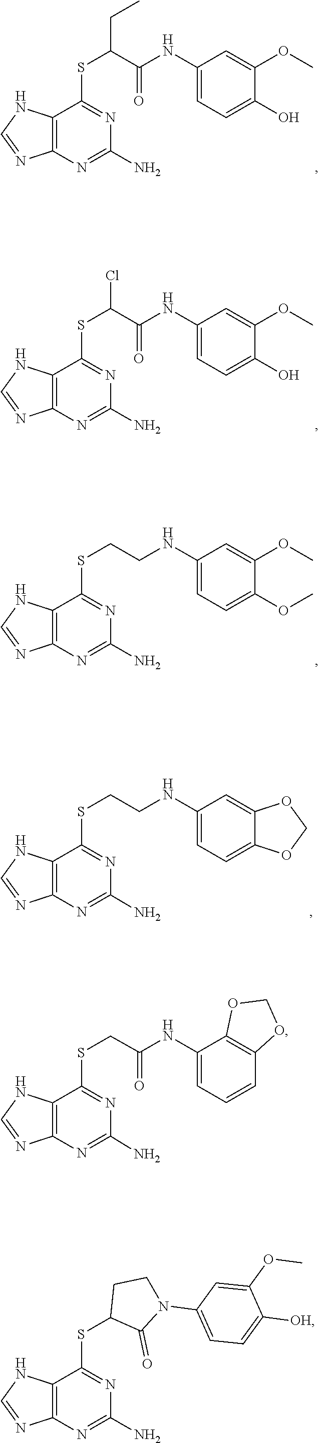 [SMILES: CCC(Sc1nc(N)nc2nc[nH]c12)C(=O)Nc1ccc(O)c(OC)c1 ; COc1cc(NC(=O)C(Cl)Sc2nc(N)nc3nc[nH]c23)ccc1O ; COc1ccc(NCCSc2nc(N)nc3nc[nH]c23)cc1OC ; Nc1nc(SCCNc2ccc3c(c2)OCO3)c2[nH]cnc2n1 ; Nc1nc(SCC(=O)Nc2cccc3c2OCO3)c2[nH]cnc2n1 ; COc1cc(N2CCC(Sc3nc(N)nc4nc[nH]c34)C2=O)ccc1O]

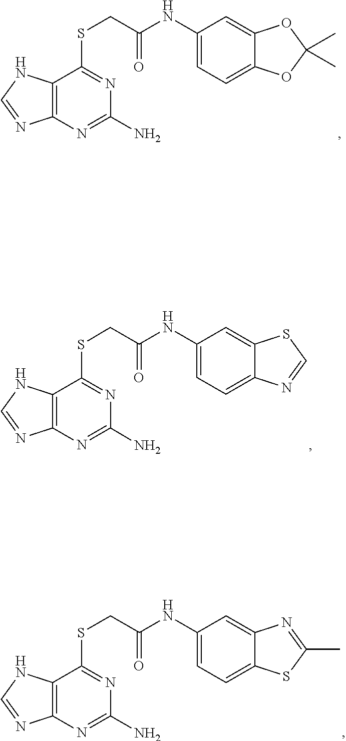 [SMILES: CC1(C)Oc2ccc(NC(=O)CSc3nc(N)nc4nc[nH]c34)cc2O1 ; Nc1nc(SCC(=O)Nc2ccc3ncsc3c2)c2[nH]cnc2n1 ; Cc1nc2cc(NC(=O)CSc3nc(N)nc4nc[nH]c34)ccc2s1]

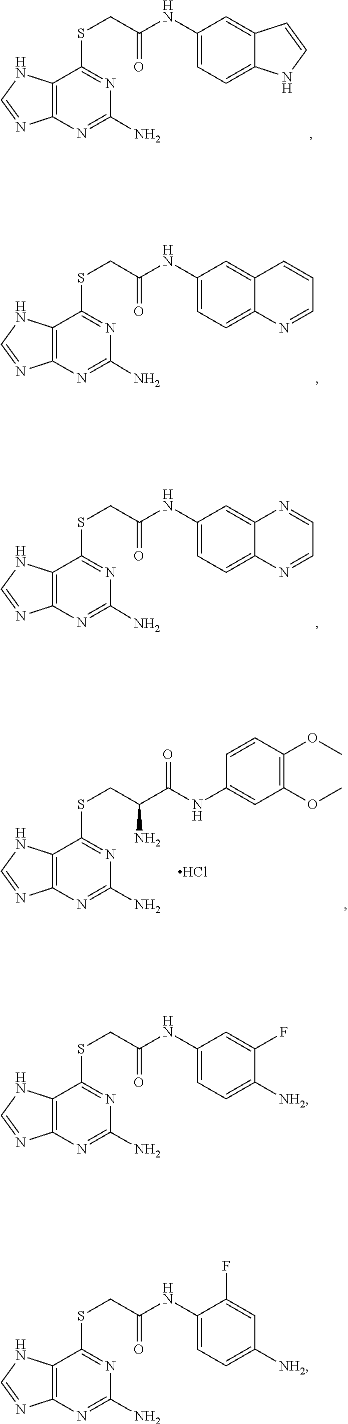

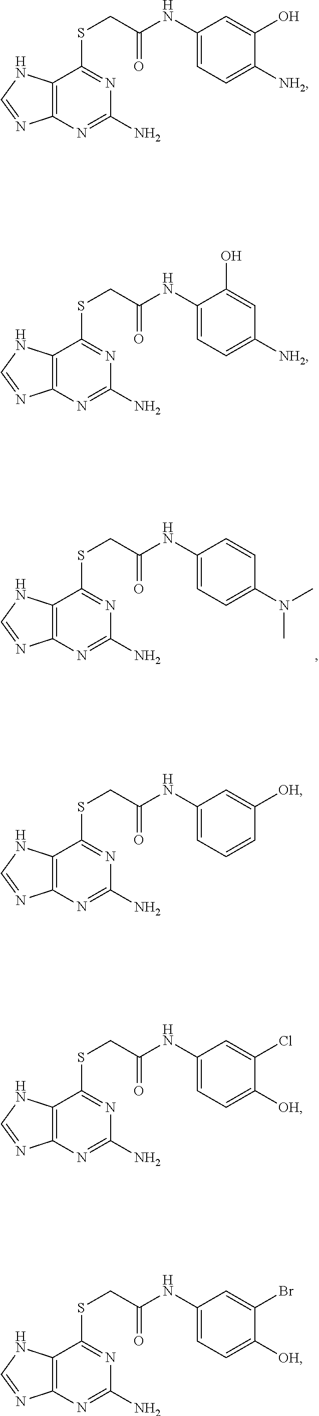 [SMILES: Nc1nc(SCC(=O)Nc2ccc(N)c(O)c2)c2[nH]cnc2n1 ; Nc1ccc(NC(=O)CSc2nc(N)nc3nc[nH]c23)c(O)c1 ; CN(C)c1ccc(NC(=O)CSc2nc(N)nc3nc[nH]c23)cc1 ; Nc1nc(SCC(=O)Nc2cccc(O)c2)c2[nH]cnc2n1 ; Nc1nc(SCC(=O)Nc2ccc(O)c(Cl)c2)c2[nH]cnc2n1 ; Nc1nc(SCC(=O)Nc2ccc(O)c(Br)c2)c2[nH]cnc2n1]

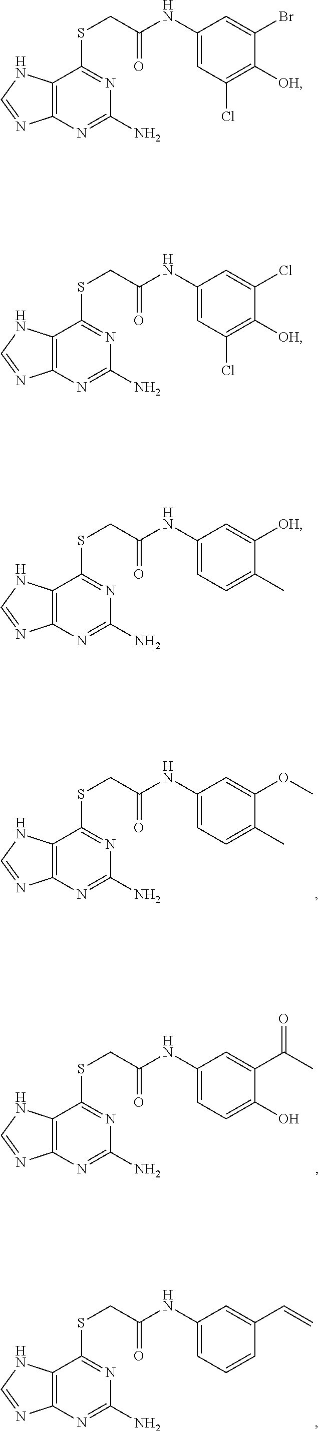 [SMILES: Nc1nc(SCC(=O)Nc2cc(Cl)c(O)c(Br)c2)c2[nH]cnc2n1 ; Nc1nc(SCC(=O)Nc2cc(Cl)c(O)c(Cl)c2)c2[nH]cnc2n1 ; Cc1ccc(NC(=O)CSc2nc(N)nc3nc[nH]c23)cc1O ; COc1cc(NC(=O)CSc2nc(N)nc3nc[nH]c23)ccc1C ; CC(=O)c1cc(NC(=O)CSc2nc(N)nc3nc[nH]c23)ccc1O ; C=Cc1cccc(NC(=O)CSc2nc(N)nc3nc[nH]c23)c1]

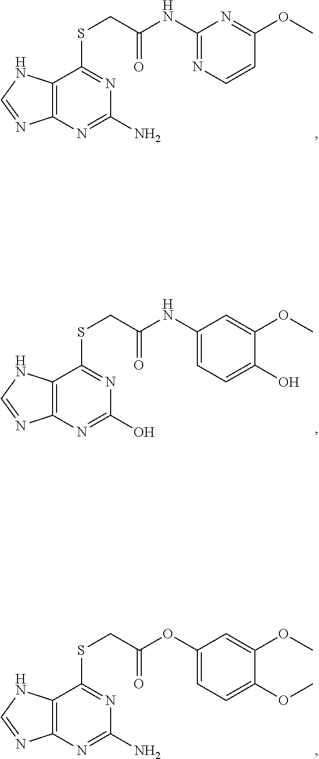 [SMILES: COc1ccnc(NC(=O)CSc2nc(N)nc3nc[nH]c23)n1 ; COc1cc(NC(=O)CSc2nc(O)nc3nc[nH]c23)ccc1O ; COc1ccc(OC(=O)CSc2nc(N)nc3nc[nH]c23)cc1OC]

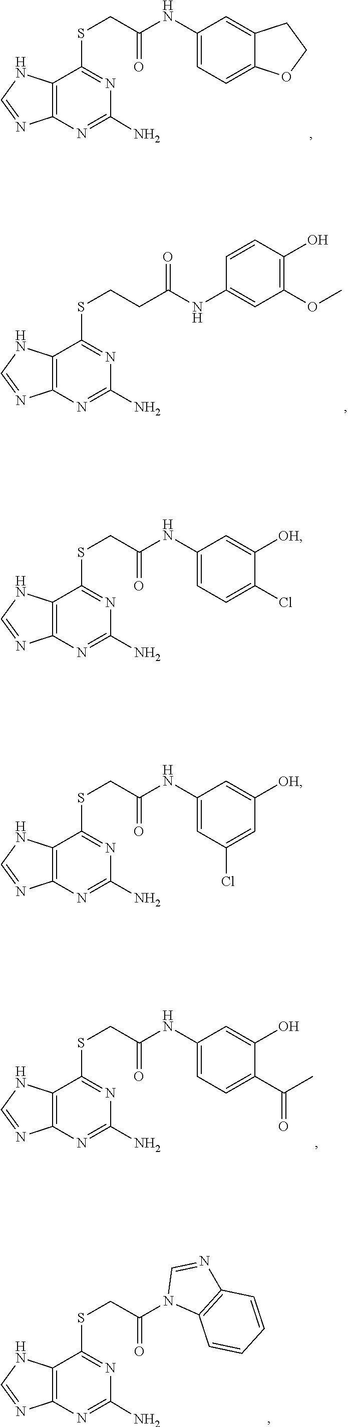 [SMILES: Nc1nc(SCC(=O)Nc2ccc3c(c2)CCO3)c2[nH]cnc2n1 ; COc1cc(NC(=O)CCSc2nc(N)nc3nc[nH]c23)ccc1O ; Nc1nc(SCC(=O)Nc2ccc(Cl)c(O)c2)c2[nH]cnc2n1 ; Nc1nc(SCC(=O)Nc2cc(O)cc(Cl)c2)c2[nH]cnc2n1 ; CC(=O)c1ccc(NC(=O)CSc2nc(N)nc3nc[nH]c23)cc1O ; Nc1nc(SCC(=O)n2cnc3ccccc32)c2[nH]cnc2n1]

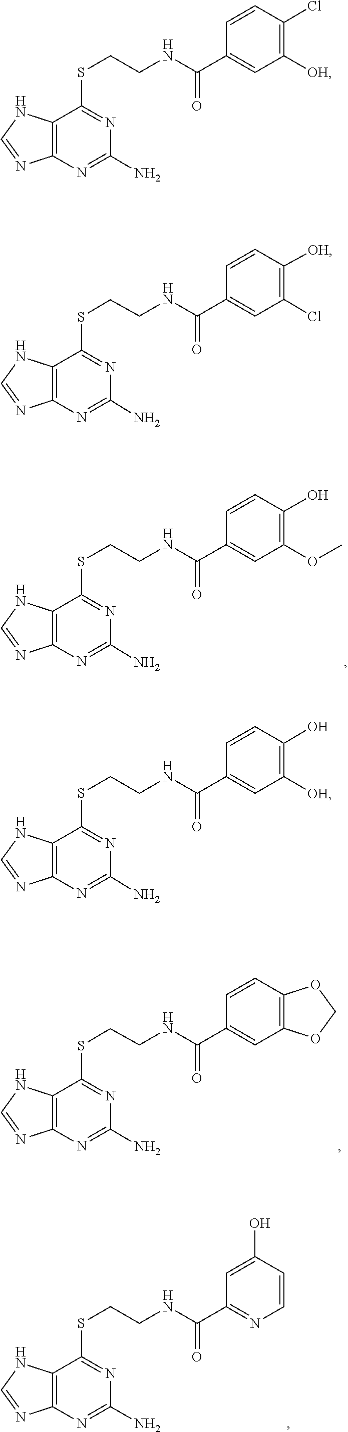 [SMILES: Nc1nc(SCCNC(=O)c2ccc(Cl)c(O)c2)c2[nH]cnc2n1 ; Nc1nc(SCCNC(=O)c2ccc(O)c(Cl)c2)c2[nH]cnc2n1 ; COc1cc(C(=O)NCCSc2nc(N)nc3nc[nH]c23)ccc1O ; Nc1nc(SCCNC(=O)c2ccc(O)c(O)c2)c2[nH]cnc2n1 ; Nc1nc(SCCNC(=O)c2ccc3c(c2)OCO3)c2[nH]cnc2n1 ; Nc1nc(SCCNC(=O)c2cc(O)ccn2)c2[nH]cnc2n1]

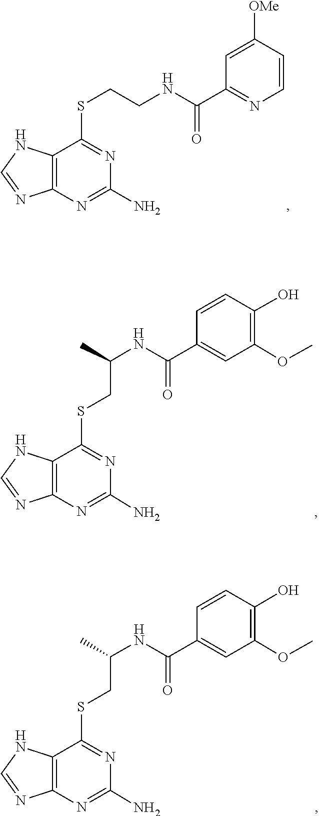 [SMILES: COc1ccnc(C(=O)NCCSc2nc(N)nc3nc[nH]c23)c1 ; COc1cc(C(=O)N[C@H](C)CSc2nc(N)nc3nc[nH]c23)ccc1O ; COc1cc(C(=O)N[C@@H](C)CSc2nc(N)nc3nc[nH]c23)ccc1O]

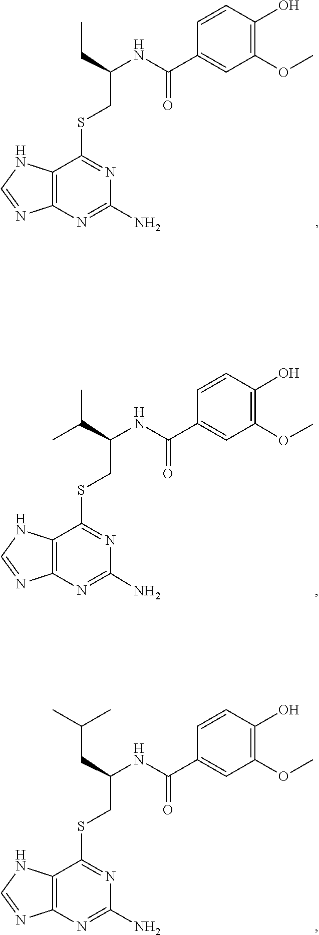 [SMILES: CC[C@H](CSc1nc(N)nc2nc[nH]c12)NC(=O)c1ccc(O)c(OC)c1 ; COc1cc(C(=O)N[C@@H](CSc2nc(N)nc3nc[nH]c23)C(C)C)ccc1O ; COc1cc(C(=O)N[C@@H](CSc2nc(N)nc3nc[nH]c23)CC(C)C)ccc1O]

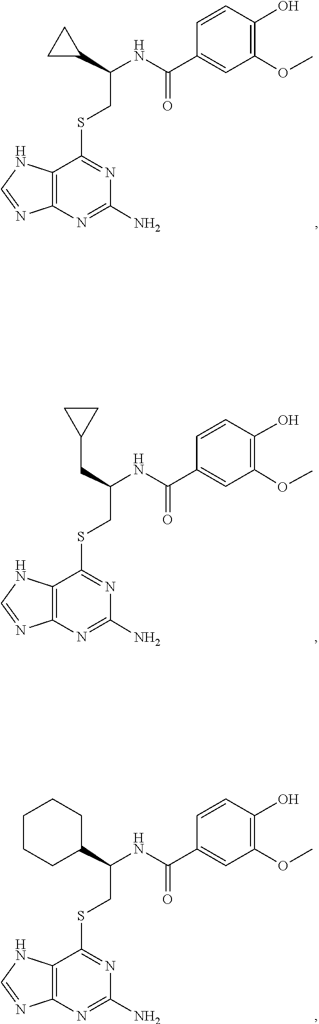 [SMILES: COc1cc(C(=O)N[C@@H](CSc2nc(N)nc3nc[nH]c23)C2CC2)ccc1O ; COc1cc(C(=O)N[C@@H](CSc2nc(N)nc3nc[nH]c23)CC2CC2)ccc1O ; COc1cc(C(=O)N[C@@H](CSc2nc(N)nc3nc[nH]c23)C2CCCCC2)ccc1O]

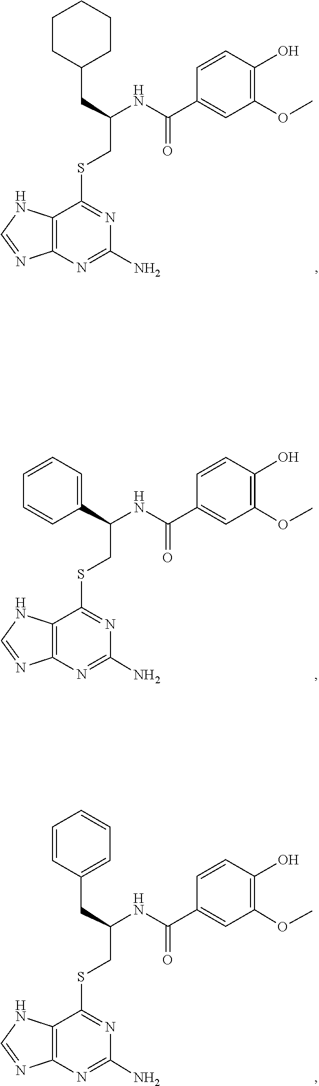 [SMILES: COc1cc(C(=O)N[C@@H](CSc2nc(N)nc3nc[nH]c23)CC2CCCCC2)ccc1O ; COc1cc(C(=O)N[C@@H](CSc2nc(N)nc3nc[nH]c23)c2ccccc2)ccc1O ; COc1cc(C(=O)N[C@@H](CSc2nc(N)nc3nc[nH]c23)Cc2ccccc2)ccc1O]

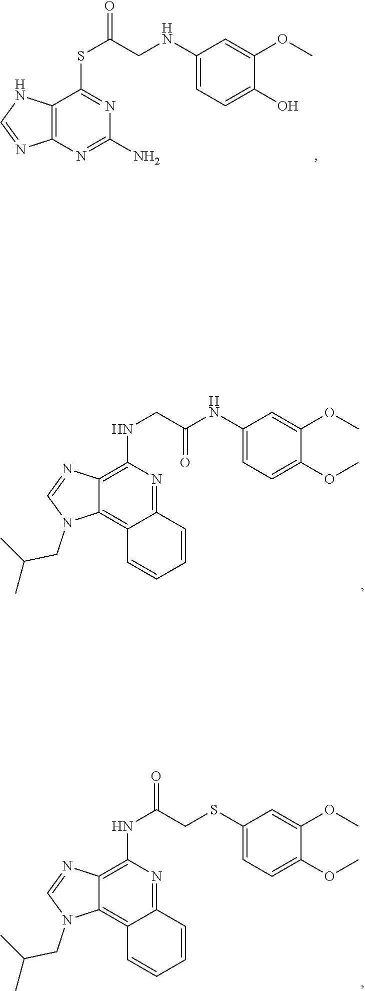

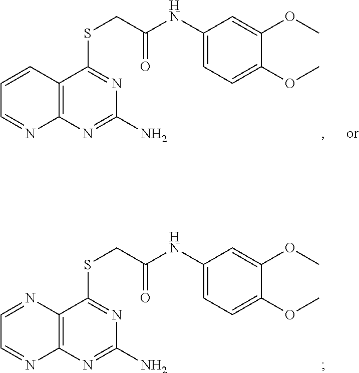 [SMILES: COc1ccc(NC(=O)CSc2nc(N)nc3ncccc23)cc1OC ; COc1ccc(NC(=O)CSc2nc(N)nc3nccnc23)cc1OC]

or a pharmaceutically acceptable salt, tautomer, hydrate or solvate thereof.

In some embodiments, the compound of Formula (X) has a structure according to one of the following:

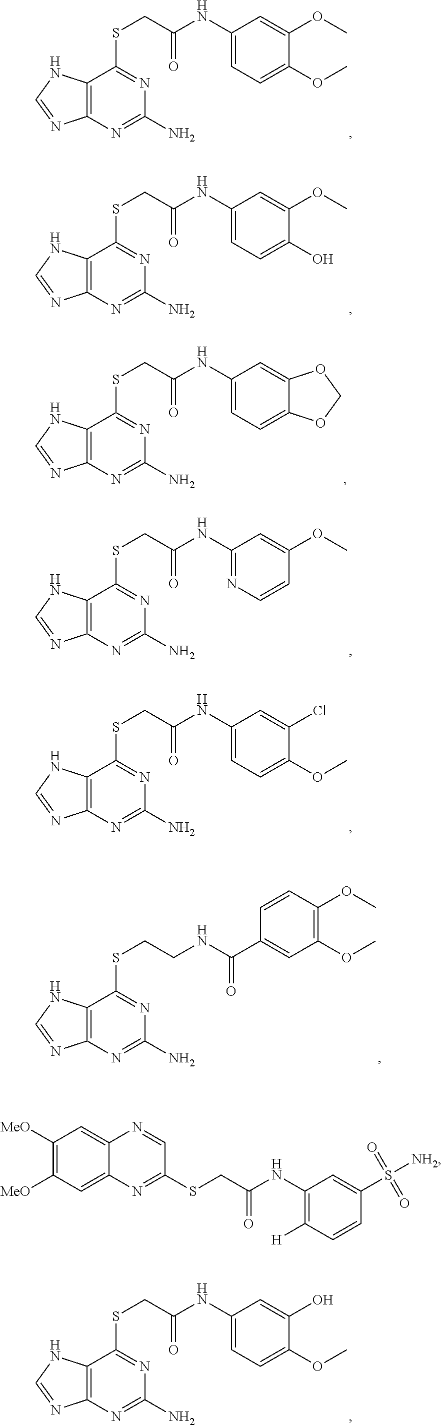

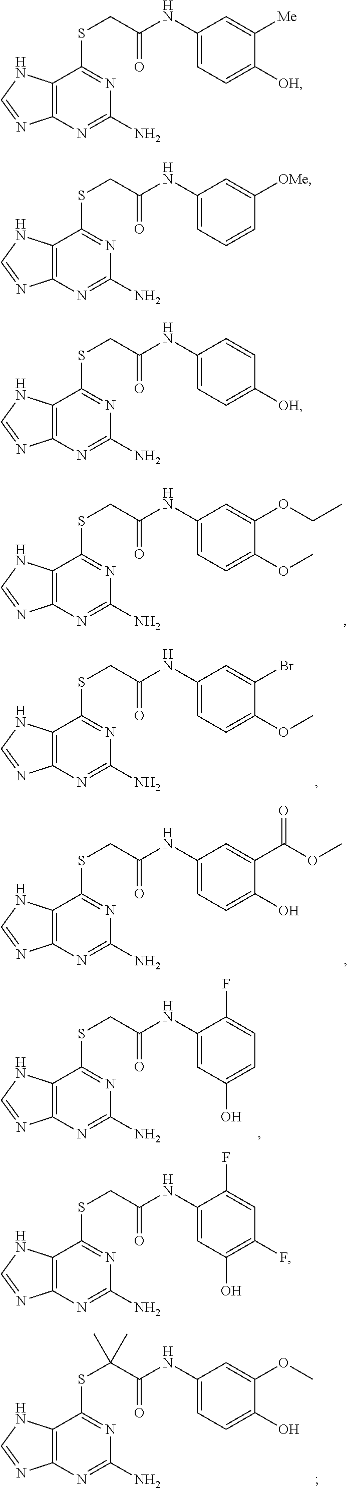

or a pharmaceutically acceptable salt, tautomer, hydrate or solvate thereof.

In some embodiments, the compound of Formula (X) has a structure according to one of the following:

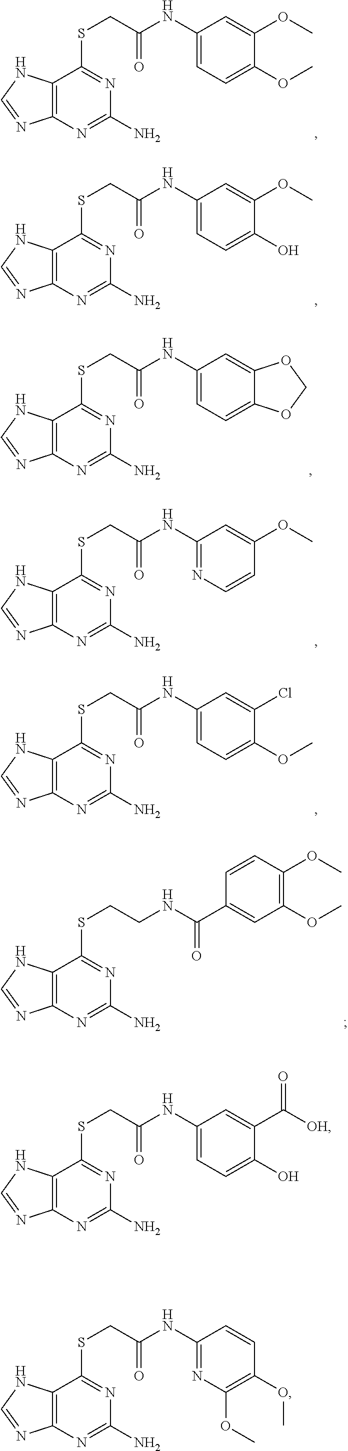

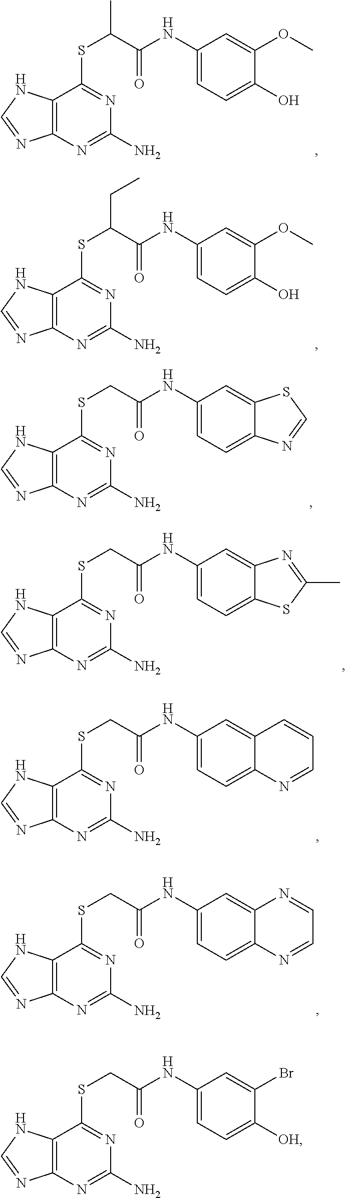

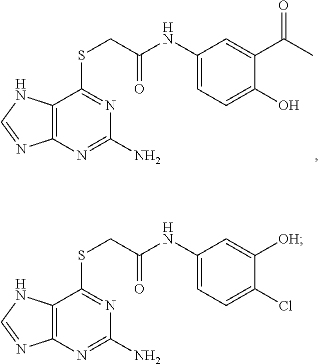 [SMILES: CC(=O)c1cc(NC(=O)CSc2nc(N)nc3nc[nH]c23)ccc1O ; Nc1nc(SCC(=O)Nc2ccc(Cl)c(O)c2)c2[nH]cnc2n1]

or a pharmaceutically acceptable salt, tautomer, hydrate or solvate thereof.

In some embodiments, the compound of Formula (X) has a structure according to the following:

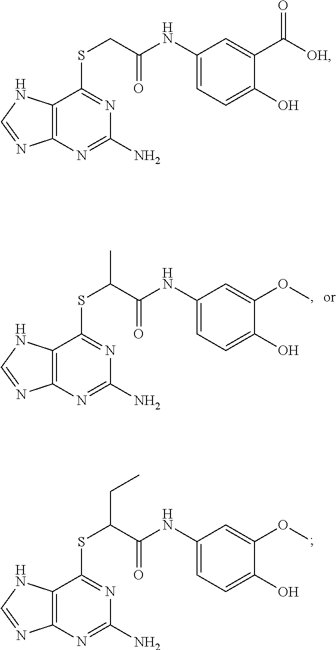

or a pharmaceutically acceptable salt, tautomer, hydrate or solvate thereof.

In some embodiments, the compound of Formula (X) is a compound provided in Table 2, Table 3, or Table 4, below. In some embodiments, the compound of Formula (X) is a compound provided in Table 2. In some embodiments, the compound of Formula (X) is a compound provided in Table 3.

In some embodiments of the present disclosure, the compound of Formula (X) is not one or more of:

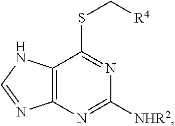

wherein:

(a) R² is H and R⁴ is

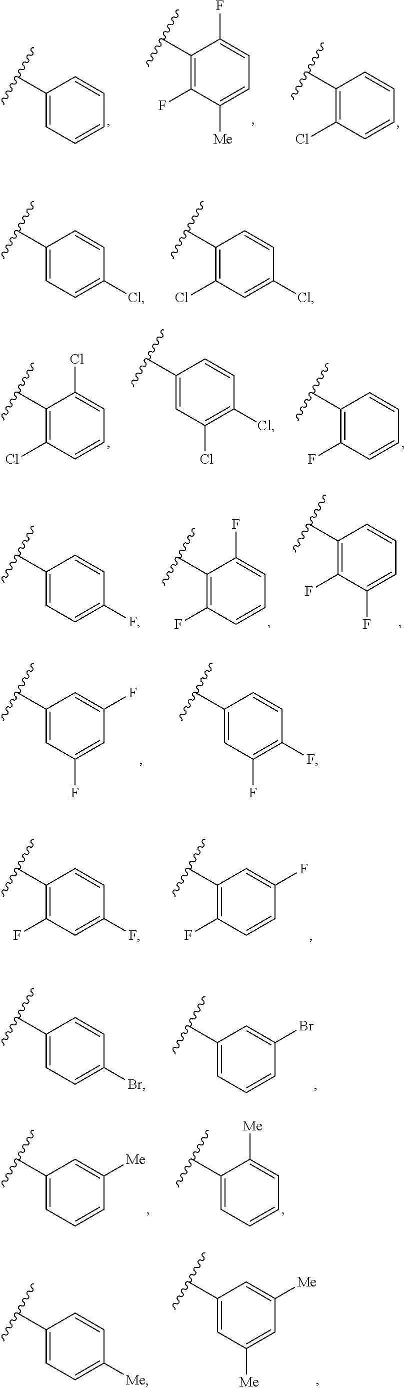

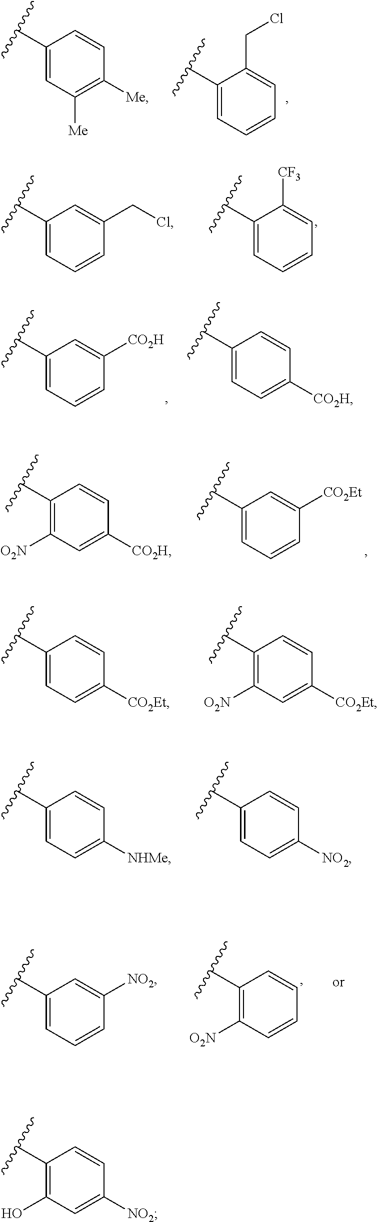

or

(b) R² is Me and R⁴ is

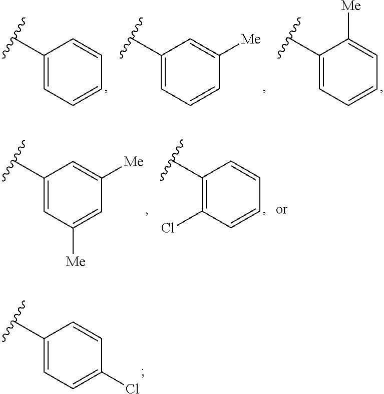

or

(c) R² is Et and R⁴ is

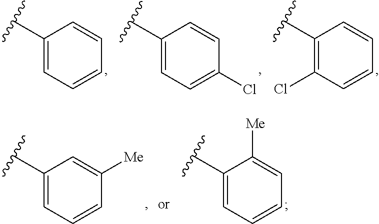

(d) R² is nPr, C(O)Me or CO₂nBu and R⁴ is

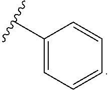 [SMILES: *c1ccccc1]

In some embodiments of the present disclosure, the compound of Formula (X) is not one or more of:

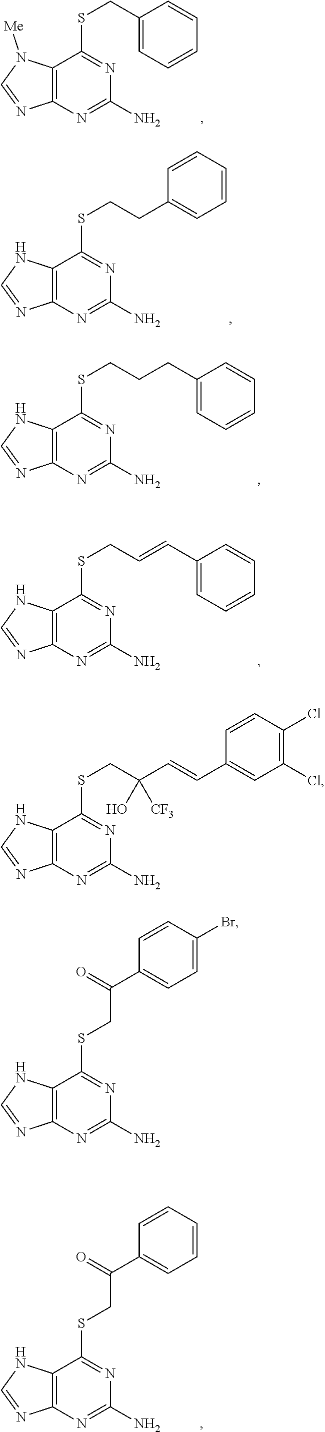

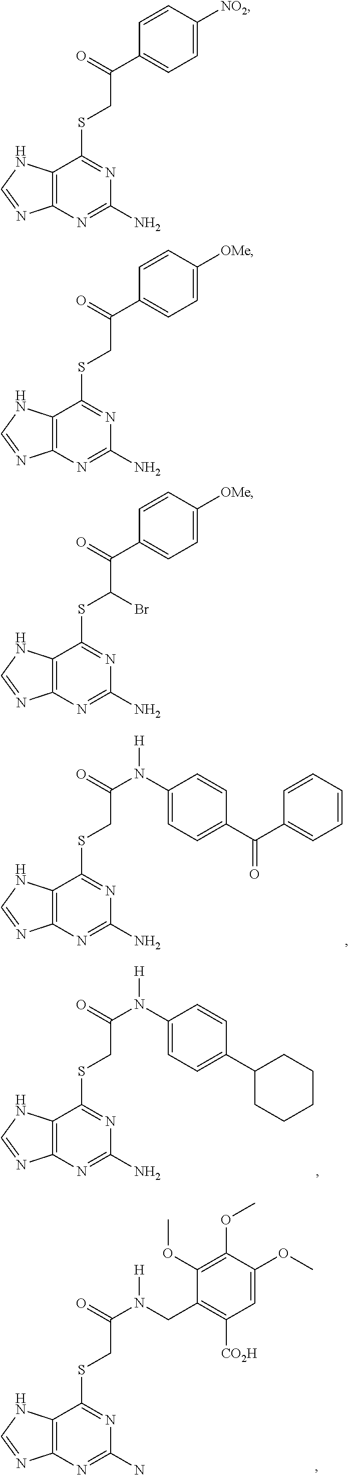

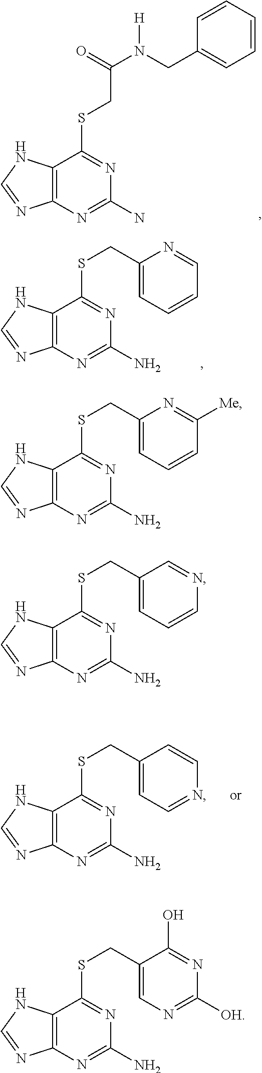

In some embodiments of the present disclosure, the compound of Formula (X) is not:

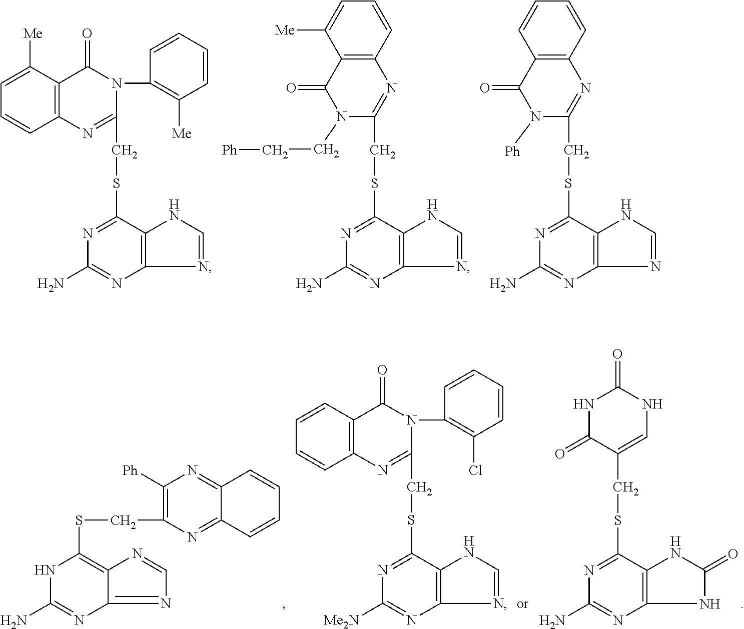

In some embodiments of Formula (X), when -L-R⁴ is -alkylene-aryl, the compound of Formula (X) is not:

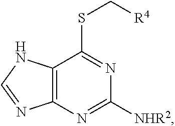

wherein:

(a) R² is H and R⁴ is

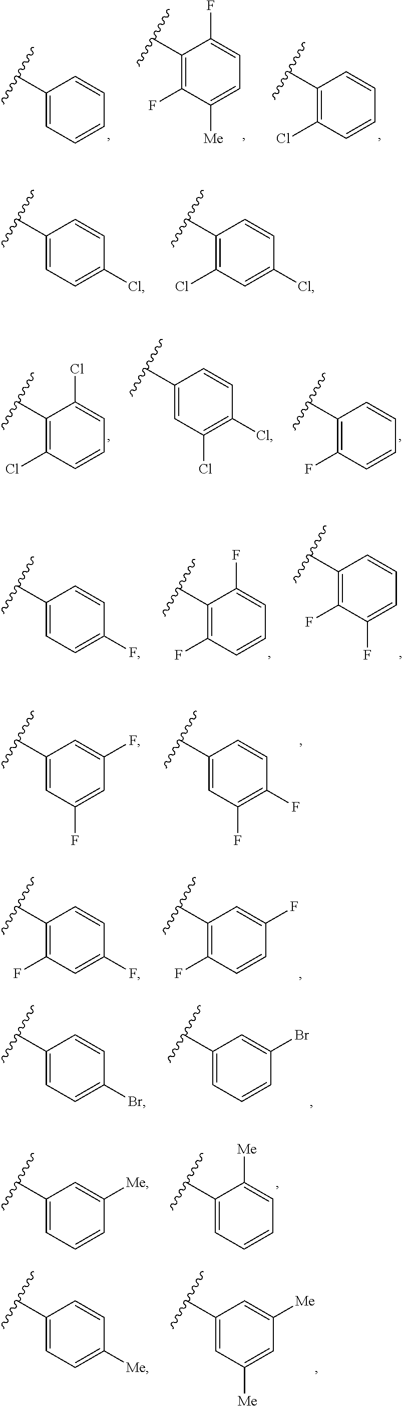

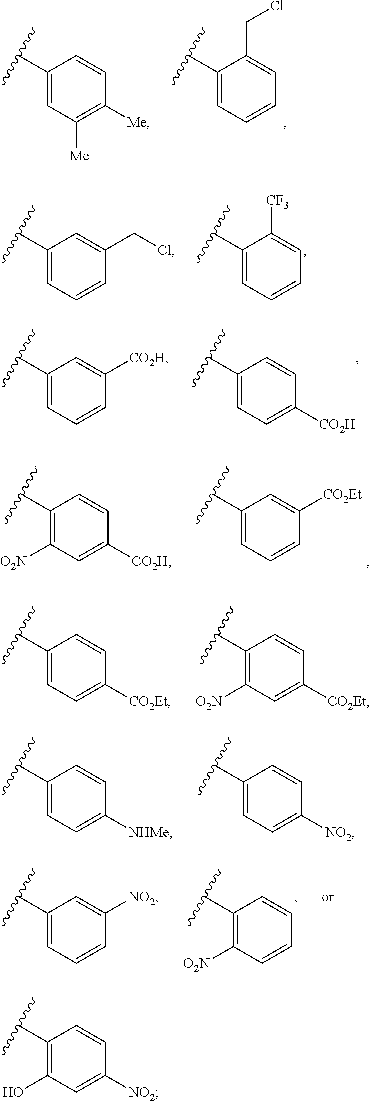

(b) R² is Me and R⁴ is

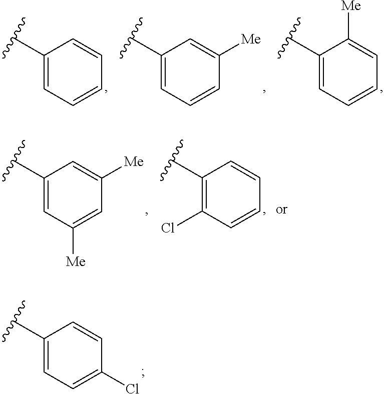

or

(c) R² is Et and R⁴ is

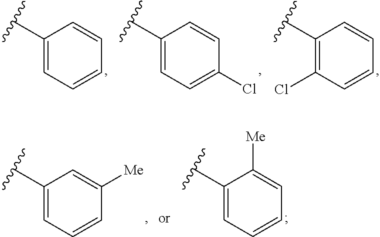

(d) R² is nPr, C(O)Me or CO₂nBu and R⁴ is

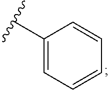 [SMILES: *c1ccccc1]

or

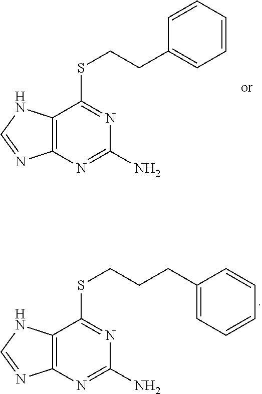 [SMILES: Nc1nc(SCCc2ccccc2)c2[nH]cnc2n1 ; Nc1nc(SCCCc2ccccc2)c2[nH]cnc2n1]

In some embodiments of Formula (X), when -L-R⁴ is -alkylene-aryl, the compound of Formula (X) is not:

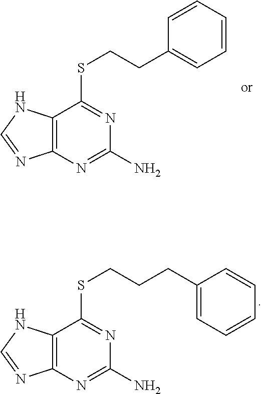 [SMILES: Nc1nc(SCCc2ccccc2)c2[nH]cnc2n1 ; Nc1nc(SCCCc2ccccc2)c2[nH]cnc2n1]

In some embodiments of Formula (X), when -L-R⁴ is -alkenylene-aryl, the compound of Formula (X) is not:

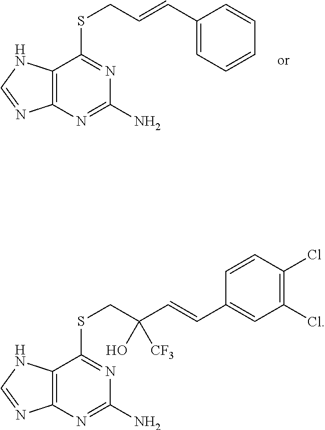 [SMILES: Nc1nc(SC/C=C/c2ccccc2)c2[nH]cnc2n1 ; Nc1nc(SCC(O)(/C=C/c2ccc(Cl)c(Cl)c2)C(F)(F)F)c2[nH]cnc2n1]

In some embodiments of Formula (X), when -L-R⁴ is —CH₂C(O)-aryl, the compound of Formula (X) is not:

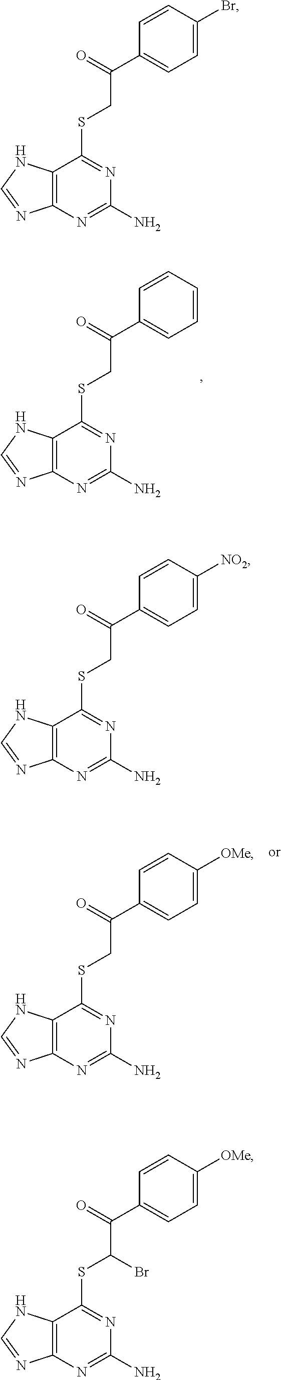 [SMILES: Nc1nc(SCC(=O)c2ccc(Br)cc2)c2[nH]cnc2n1 ; Nc1nc(SCC(=O)c2ccccc2)c2[nH]cnc2n1 ; Nc1nc(SCC(=O)c2ccc([N+](=O)[O-])cc2)c2[nH]cnc2n1 ; COc1ccc(C(=O)CSc2nc(N)nc3nc[nH]c23)cc1 ; "or" ; COc1ccc(C(=O)C(Br)Sc2nc(N)nc3nc[nH]c23)cc1]

In some embodiments of Formula (X), when -L-R⁴ is —CH₂C(O)N(R⁵)-aryl, the compound of Formula (X) is not:

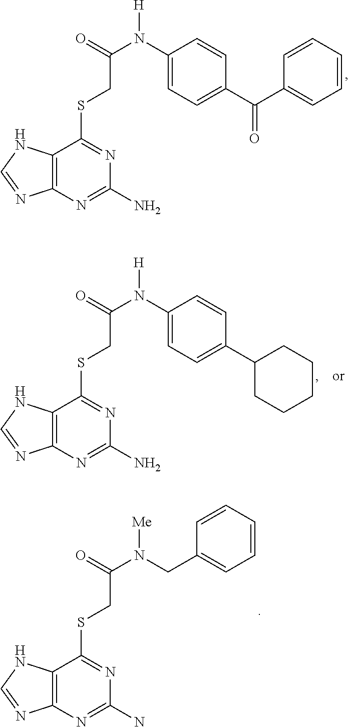 [SMILES: Nc1nc(SCC(=O)Nc2ccc(C(=O)c3ccccc3)cc2)c2[nH]cnc2n1 ; Nc1nc(SCC(=O)Nc2ccc(C3CCCCC3)cc2)c2[nH]cnc2n1 ; CN(Cc1ccccc1)C(=O)CSc1nc(N)nc2nc[nH]c12]

In some embodiments of Formula (X), when -L-R⁴ is -alkylene-heteroaryl, the compound of Formula (X) is not:

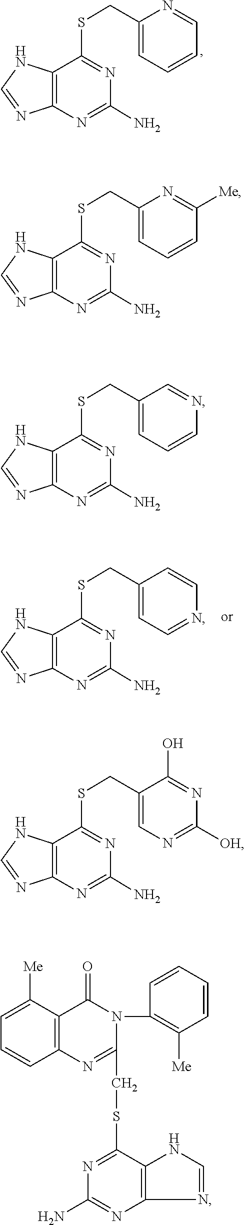

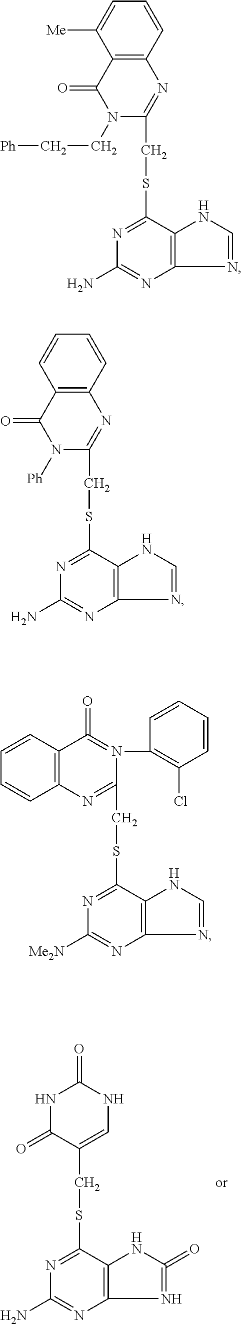

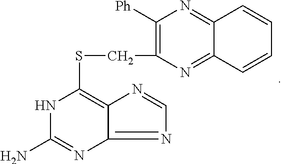 [SMILES: Nc1nc2ncnc-2c(SCc2nc3ccccc3nc2-c2ccccc2)[nH]1]

In various embodiments of the present disclosure, the compound of Formula (X) is not a compound disclosed in WO 2019/051269 or WO 2019/046778.

In some embodiments of the present disclosure, the compound of Formula (X) is a compound of Formula (XX):

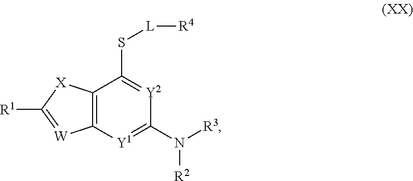

or a pharmaceutically acceptable salt, hydrate, or tautomer thereof, wherein L, W, X, Y¹, Y², R¹, R², R³, and R⁴ are as defined above for Formula (X).

In some embodiments, the compound of Formula (XX) is selected from the group consisting of

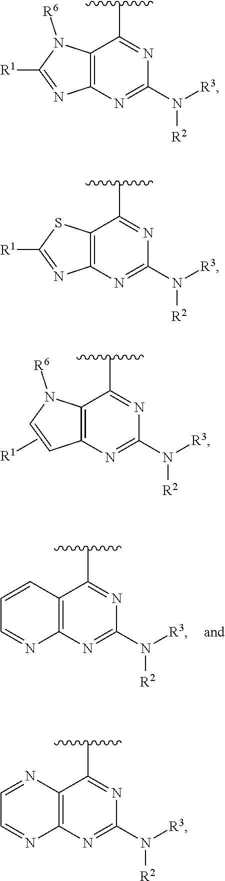

wherein R¹, R², R³, and R⁶ are as defined above for Formula (X). In some embodiments, the compound of Formula (XX) is selected from the group consisting of

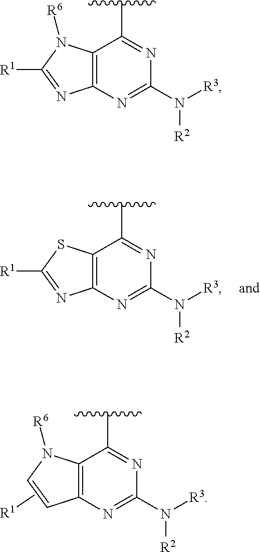

In some embodiments, the compound of Formula (XX) is selected from the group consisting of

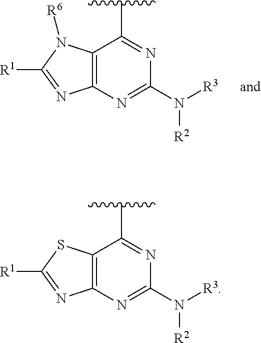

In some embodiments, the compound of Formula (XX) is

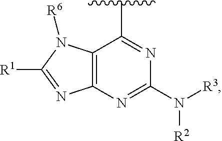

wherein R¹, R², R³, and R⁶ are as defined above for Formula (X).

In some embodiments of the present disclosure, the compound of Formula (X) is a compound of Formula (XXa):

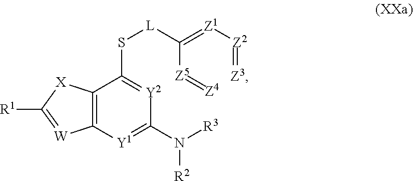

or a pharmaceutically acceptable salt, hydrate, or tautomer thereof, wherein L, W, X, Y¹, Y², R¹, R², and R³ are as defined above for Formula (X), and Z¹, Z², Z³, Z⁴, and Z⁵ are each independently CR⁸ or N.

In some embodiments of Formula (XXa), each of Z¹, Z², Z³, Z⁴, and Z⁵ is CR⁸. In some embodiments, at least one of Z¹, Z², Z³, Z⁴, and Z⁵ is N. In other embodiments, one of Z¹, Z², Z³, Z⁴, and Z⁵ is N. In still other embodiments, two of Z¹, Z², Z³, Z⁴, and Z⁵ are N. In some embodiments, Z¹ and Z⁵ are N and Z²-Z⁴ are CR⁸. In some embodiments. Z⁵ is N and Z¹-Z⁴ are CR⁸.

In some embodiments of Formula (XXa), each R⁸ is independently halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN. In some embodiments, each R⁸ is independently H, halogen, —C₁₋₅alkyl, CF₃, —OH, —O(C₁₋₅alkyl), —OCF₃, —OSO₂Me, —COOH, —C(O)OMe, or —SO₂Me.

In some embodiments of Formula (XXa),

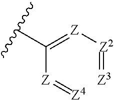

is selected from the group consisting of

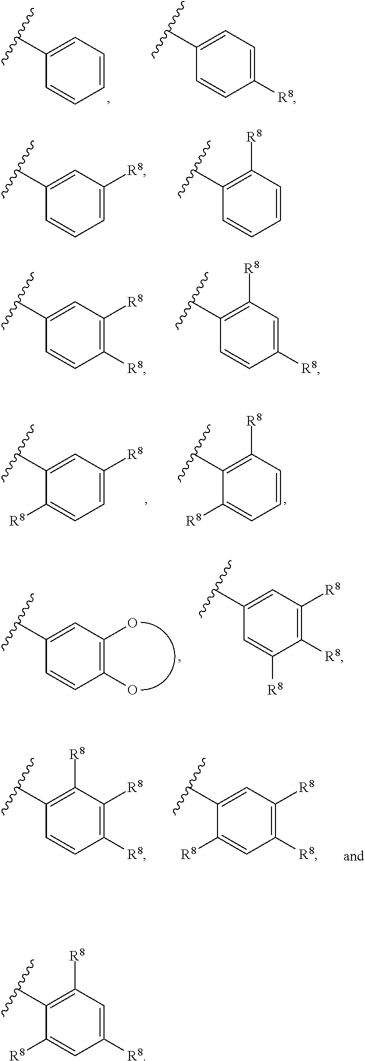

In some embodiments of Formula (XXa),

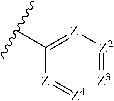

is selected from the group consisting of

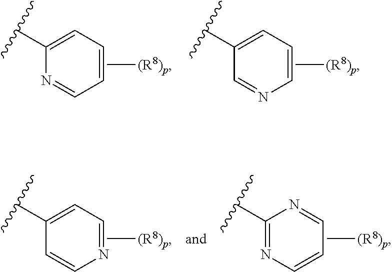

wherein p is 0, 1, or 2.

In some embodiments of Formula (XXa),

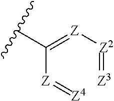

is selected from the group consisting of

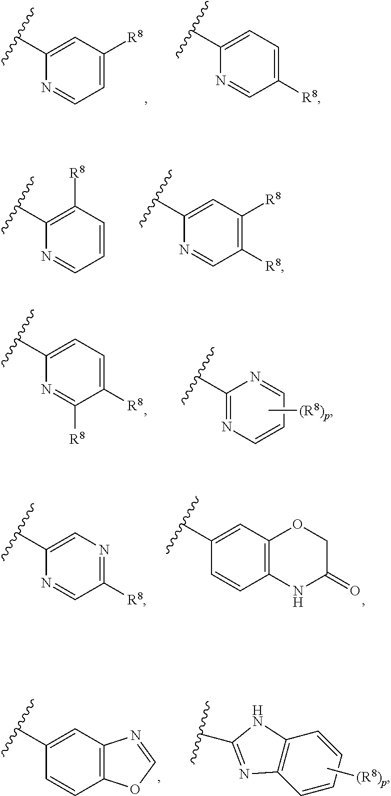

wherein p is 0, 1, or 2.

In some embodiments of the present disclosure, the compound of Formula (X) is a compound of Formula (XXb):

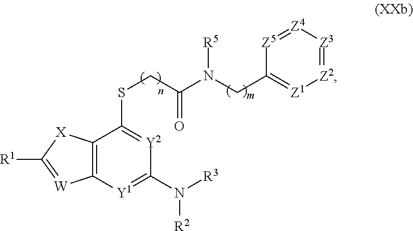

or a pharmaceutically acceptable salt, hydrate, or tautomer thereof, wherein L, W, X, Y¹, Y², R¹, R², R³, R⁵, m and n are as defined above for Formula (X), and Z¹, Z², Z³, Z⁴, and Z⁵ are each independently CR⁸ or N.

In some embodiments of Formula (XXb), each of Z¹, Z², Z³, Z⁴, and Z⁵ is CR⁸. In some embodiments, at least one of Z¹, Z², Z³, Z⁴, and Z⁵ is N. In other embodiments, one of Z¹, Z², Z³, Z⁴, and Z⁵ is N. In still other embodiments, two of Z¹, Z², Z³, Z⁴, and Z⁵ are N. In some embodiments, Z¹ and Z⁵ are N and Z²-Z⁴ are CR⁸. In some embodiments. Z⁵ is N and Z¹-Z⁴ are CRg.

In some embodiments of Formula (XXb), each R⁸ is independently halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN. In some embodiments, each R⁸ is independently H, halogen, —C₁₋₅alkyl, CF₃, —OH, —O(C₁₋₅alkyl), —OCF₃, —OSO₂Me, —COOH, —C(O)OMe, or —SO₂Me.

In some embodiments of Formula (XXb),

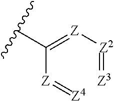

is selected from the group consisting of

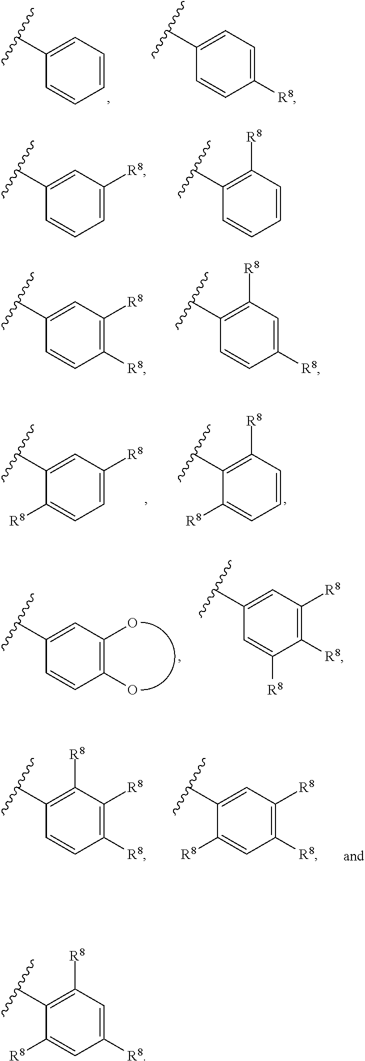

In some embodiments of Formula (XXb),

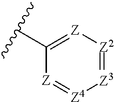

is selected from the group consisting of

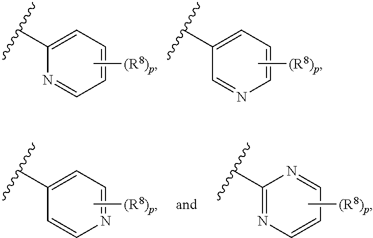

wherein p is 0, 1, or 2.

In some embodiments of Formula (XXb),

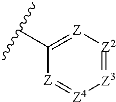

is selected from the group consisting of

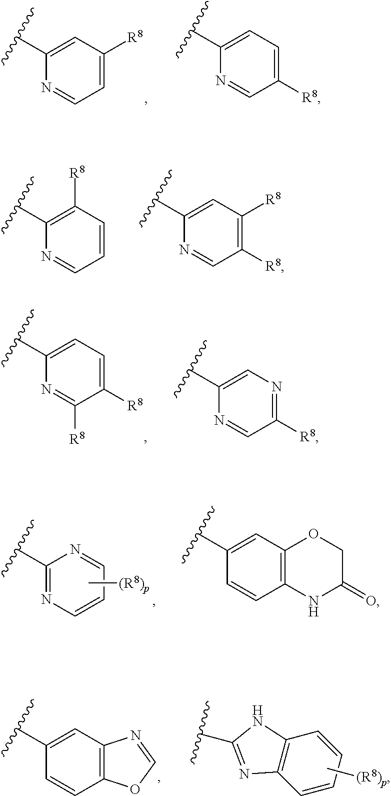

wherein p is 0, 1, or 2.

In some embodiments of the present disclosure, the compound of Formula (X), Formula (XXa), or Formula (XXb) is selected from the group consisting of:

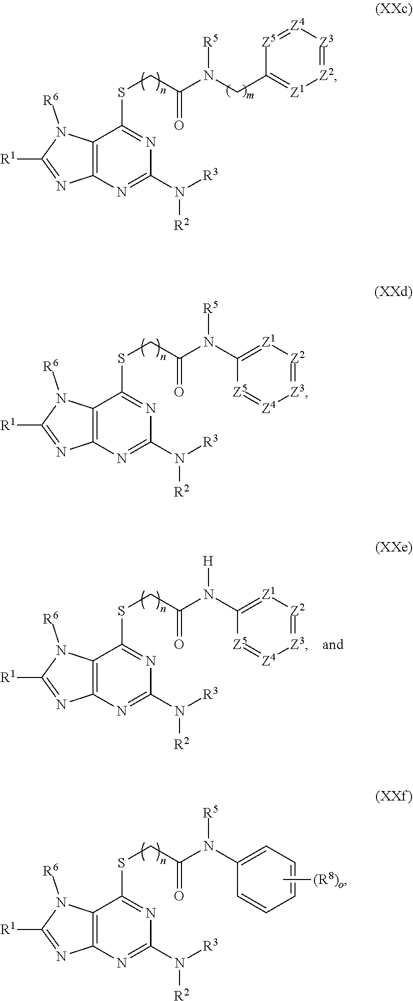

wherein R¹, R², R³, R⁵, R⁶, m and nare as defined above for Formula (X), and Z¹, Z², Z³, Z⁴, Z⁵ are each independently CR⁸ or N as defined above for formula (XXa).

In some embodiments of the present disclosure, the compound of Formula (X), Formula (XXa), or Formula (XXb) is selected from the group consisting of:

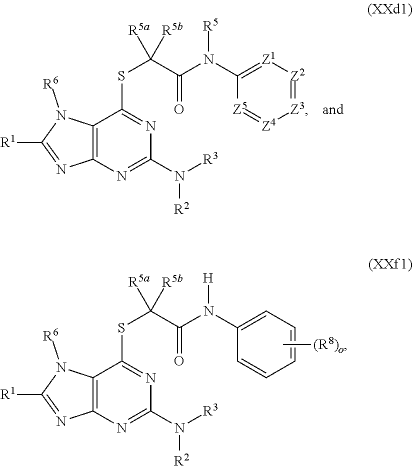

wherein R¹, R², R³, R⁵, R^{5a}, R^{5b} and R⁶ are as defined above for Formula (A1), (A2), and(X), and Z¹, Z², Z³, Z⁴, Z⁵ are each independently CR⁸ or N as defined above for Formula (XXa).

In some embodiments of Formula (XXc), Formula (XXd), Formula (XXe), and Formula (XXf), each of Z¹, Z², Z³, Z⁴, and Z⁵ is CR⁸. In some embodiments, at least one of Z¹, Z², Z³, Z⁴, and Z⁵ is N. In other embodiments, one of Z¹, Z², Z³, Z⁴, and Z⁵ is N. In still other embodiments, two of Z¹, Z², Z³, Z⁴, and Z⁵ are N. In some embodiments, Z¹ and Z⁵ are N and Z²-Z⁴ are CR⁸. In some embodiments. Z⁵ is N and Z¹-Z⁴ are CR⁸.

In some embodiments of Formula (XXc), Formula (XXd), Formula (XXe), and Formula (XXf), each R⁸ is independently halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN. In some embodiments, each R⁸ is independently H, halogen, —C₁₋₅alkyl, CF₃, —OH, —O(C₁₋₅alkyl), —OCF₃, —OSO₂Me, —COOH, —C(O)OMe, or —SO₂Me.

In some embodiments of Formula (XXc), Formula (XXd), Formula (XXe), and Formula (XXf),

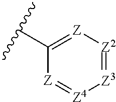

is selected from the group consisting of

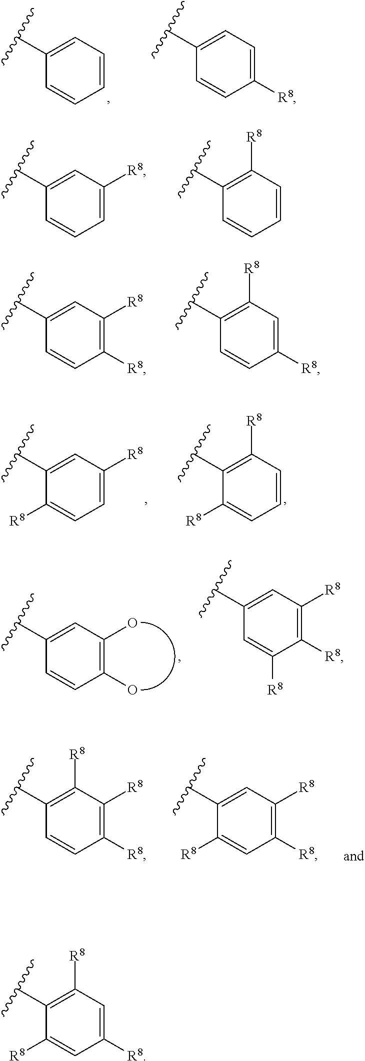

In some embodiments of Formula (XXc), Formula (XXd), Formula (XXe), and Formula (XXf),

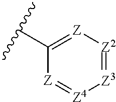

is selected from the group consisting of

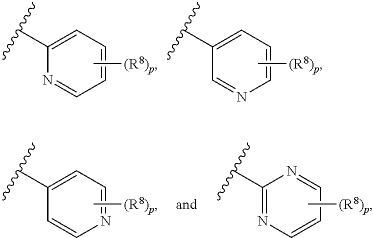

wherein p is 0, 1, or 2.

In some embodiments of Formula (XXc), Formula (XXd), Formula (XXe), and Formula (XXf)

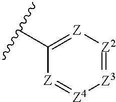

is selected from the group consisting of

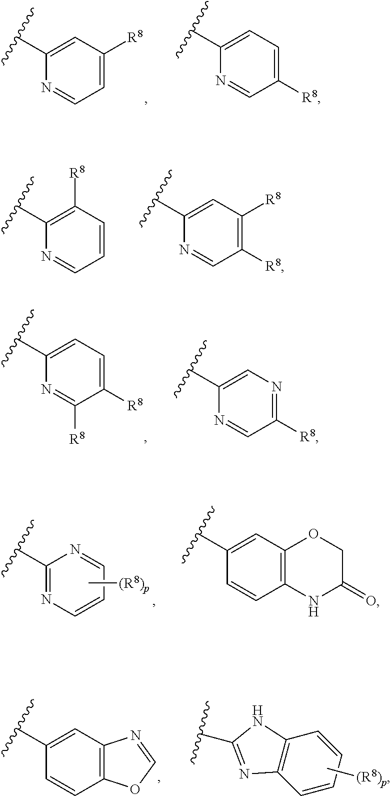

wherein p is 0, 1, or 2.

In some embodiments, the present disclosure provides a compound of Formula (Y) or a pharmaceutically acceptable salt, hydrate, or tautomer thereof:

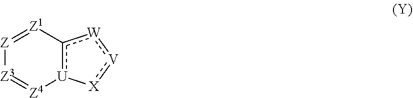

wherein:

U is C or N;
wherein
when U is C, Y is

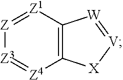

or
when U is N, Y is

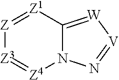

V is N or CR¹⁰;

W is CH or N;

X is S, O, N-L-R¹¹, or NR¹²;

L is selected from alkylene, alkenylene, optionally substituted -alkylene-(NR¹²)—, optionally substituted

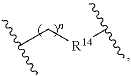

optionally substituted

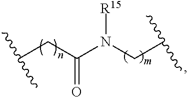

optionally substituted

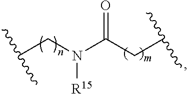

optionally substituted

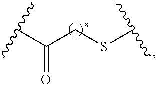 [SMILES: *SCC(*)=O]

optionally substituted

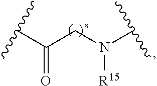

and

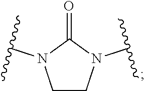 [SMILES: *N1CCN(*)C1=O]

R¹⁰ is H, alkyl, —O-alkyl, —S-alkyl, carbocyclyl, alkylenecarbocyclyl, —O-L-R¹¹, —S-L-R¹¹, —N(R¹²)-L-R¹¹, -L-R¹¹;

R¹¹ is alkyl, carbocyclyl, heterocyclyl, aryl, or heteroaryl, each of which is optionally substituted; and

R¹² is each independently H, alkyl, alkylenecarbocyclyl, or carbocyclyl, wherein two R¹² groups taken together with the carbon atom to which they are attached can form a heterocyclyl;

R¹⁴ is carbocyclyl, heterocyclyl, or heteroaryl;

R¹⁵ is H, alkyl, carbocyclyl, alkylenecarbocyclyl, or alkylenearyl;

Z¹, Z², Z³, and Z⁴ are each independently CR¹³ or N;

R¹³ is H, halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN, wherein two R¹³ taken together with the atoms to which they are attached can form carbocyclyl, heterocyclyl, or heteroaryl, each of which is optionally substituted;

m is 0, 1, or 2; and

n is 1, 2, or 3;

provided that either X is N-L-R¹¹ or R¹⁰ is —O-L-R¹¹, —S-L-R¹¹, —N(R¹²)-L-R¹¹, or -L-R¹¹.

In some embodiments, X is N-L-R¹¹ and R¹⁰ is H, alkyl, —O-alkyl, —S-alkyl, carbocyclyl, or alkylenecarbocyclyl. In other embodiments, X is S, O, or NR¹² and R¹⁰ is —O-L-R¹¹, —S-L-R¹¹, —N(R¹²)-L-R¹¹, or -L-R¹¹.

In some embodiments, the present disclosure provides a compound of Formula (Y) or a pharmaceutically acceptable salt, hydrate, or tautomer thereof:

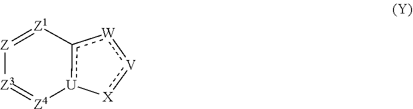

wherein:

U is C or N;
wherein
when U is C, Y is

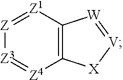

when U is N, Y is

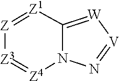

V is N or CR¹⁰;

W is CH or N;

X is S, O, N-L-R¹¹, or NR¹²;

L is selected from alkylene, alkenylene, optionally substituted -alkylene-(NR¹²)—, optionally substituted

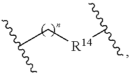

optionally substituted

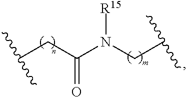

optionally substituted

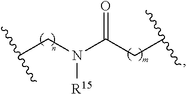

optionally substituted

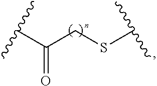 [SMILES: *SCC(*)=O]

optionally substituted

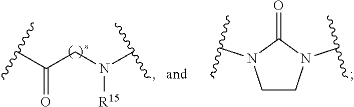

R¹⁰ is H, alkyl, —O-alkyl, —S-alkyl, carbocyclyl, alkylenecarbocyclyl, —O-L-R¹¹, —S-L-R¹¹, —N(R¹²)-L-R¹¹, -L-R¹¹;

R¹¹ is alkyl, carbocyclyl, heterocyclyl, aryl, or heteroaryl, each of which is optionally substituted; and

R¹² is each independently H, alkyl, alkylenecarbocyclyl, or carbocyclyl, wherein two R¹² groups taken together with the carbon atom to which they are attached can form a heterocyclyl;

R¹⁴ is carbocyclyl, heterocyclyl, or heteroaryl;

R¹⁵ is H, alkyl, carbocyclyl, alkylenecarbocyclyl, or alkylenearyl;
wherein:
   when X is N-L-R¹¹, V is N or CR¹⁰, wherein R¹⁰ is H, alkyl, —O-alkyl, —S-alkyl, carbocyclyl, or alkylenecarbocyclyl;
   when X is S, O, NR¹²; V is CR¹⁰, wherein R¹⁰ is —O-L-R¹¹, —S-L-R¹¹, —N(R¹²)-L-R¹¹, or -L-R¹¹; or
   when U is N, V is CR¹⁰, wherein R¹⁰ is —O-L-R¹¹, —S-L-R¹¹, —N(R¹²)-L-R¹¹, or -L-R¹¹;

Z¹, Z², Z³, and Z⁴ are each independently CR¹³ or N;

R¹³ is H, halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN, wherein two R¹³ taken together with the atoms to which they are attached can form carbocyclyl, heterocyclyl, or heteroaryl, each of which is optionally substituted;

m is 0, 1, or 2; and

n is 1, 2, or 3.

In some embodiments of Formula (Y), U is C. In other embodiments, U is N.

In some embodiments of Formula (Y), when U is C, Y is

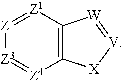

In other embodiments, when U is N, Y is

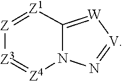

In some embodiments of Formula (Y), V is N. In other embodiments, V is CR¹⁰.

In some embodiments of Formula (Y), W is N. In other embodiments, W is CH

In some embodiments of Formula (Y), when X is S, O, or NH, V is CR¹⁰, wherein R¹⁰ is —O-L-R¹¹, —S-L-R¹¹, —N(R¹²)-L-R¹¹, or -L-R¹¹. In some embodiments, when X is S or O, V is CR¹⁰, wherein R¹⁰ is —O-L-R¹¹, —S-L-R¹¹, —N(R¹²)-L-R¹¹, or -L-R¹¹. In some embodiments when X is S, O, or NH, V is CR¹⁰, wherein R¹⁰ is —S-L-R¹¹ or —N(R¹²)-L-R¹¹. In some embodiments when X is S, O, or NH, V is CR¹⁰, wherein R¹⁰ is —S-L-R¹¹. In some embodiments when X is S or O, V is CR¹⁰, wherein R¹⁰ is —S-L-R¹¹ or —N(R¹²)-L-R¹¹. In some embodiments when X is S or O, V is CR¹⁰, wherein R¹⁰ is —S-L-R¹¹. In some embodiments when X is NH, V is CR¹⁰, wherein R¹⁰ is —O-L-R¹¹, —S-L-R¹¹, —N(R¹²)-L-R¹¹, or -L-R¹¹. In some embodiments when X is NH, V is CR¹⁰, wherein R¹⁰ is —S-L-R¹¹ or —N(R¹²)-L-R¹¹. In some embodiments when X is NH, V is CR¹⁰, wherein R¹⁰ is —S-L-R¹¹.

In some embodiments of Formula (Y), X is N-L-R¹¹ and V is N. In some embodiments, X is N-L-R¹¹ and V is CR¹⁰, wherein R¹⁰ is H, alkyl, —O-alkyl, or —S-alkyl. In some embodiments, X is N-L-R¹¹ and V is CR¹⁰, wherein R¹⁰ is H, —O-alkyl, or —S-alkyl. In some embodiments, X is N-L-R¹¹ and V is CR¹⁰, wherein R¹⁰ is H. In some embodiments of Formula (Y), X is N-L-R¹¹ and V is CR¹⁰, wherein R¹⁰ is H, alkyl, —O-alkyl, or —S-alkyl.

In some embodiments of Formula (Y), L is -alkylene-(NR¹²)—,

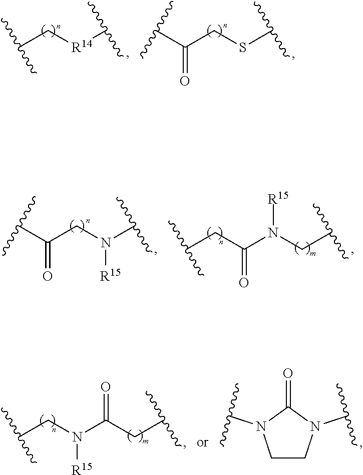

each of which is optionally substituted. In some embodiments, L is optionally substituted

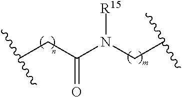

optionally substituted

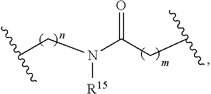

optionally substituted

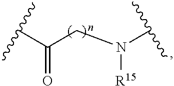

optionally substituted

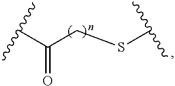 [SMILES: *SCC(*)=O]

optionally substituted

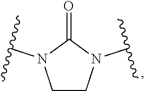 [SMILES: *N1CCN(*)C1=O]

optionally substituted

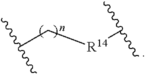

In some embodiments, L is optionally substituted

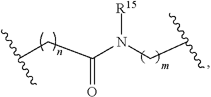

optionally substituted

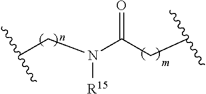

optionally substituted

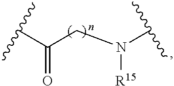

or optionally substituted

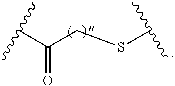 [SMILES: *SCC(*)=O]

In some embodiments, L is optionally substituted

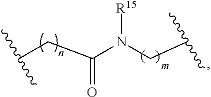

optionally substituted

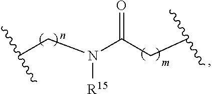

or optionally substituted

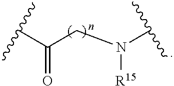

In some embodiments, L is optionally substituted

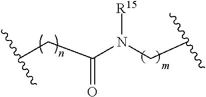

or optionally substituted

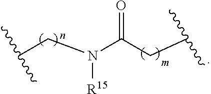

In some embodiments, L is optionally substituted

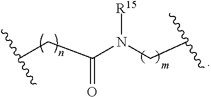

In other embodiments, L is optionally substituted

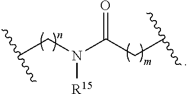

In yet other embodiments, L is optionally substituted

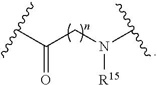

In still other embodiments, L is optionally substituted

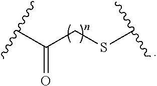

In another embodiment, L is optionally substituted

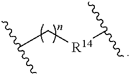

In yet another embodiment, L is optionally substituted

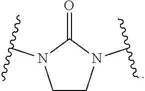 [SMILES: *N1CCN(*)C1=O]

In some embodiments, L is

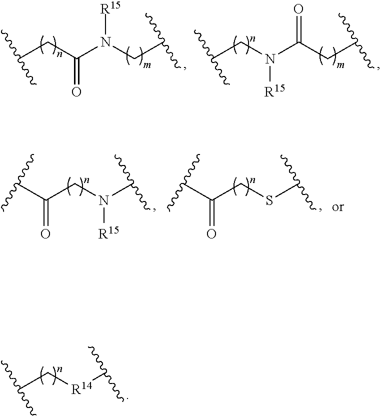

In some embodiments, L is

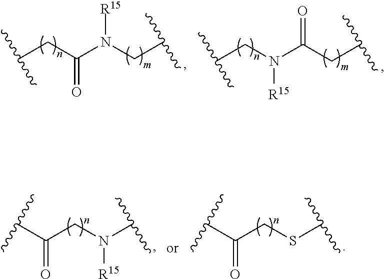

In some embodiments, L is

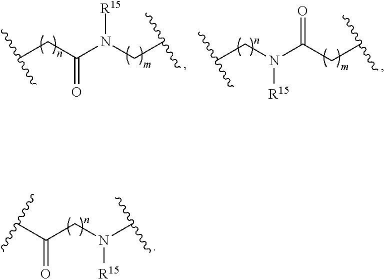

In some embodiments, L is

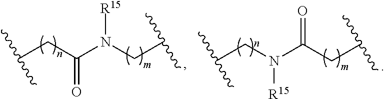

In some embodiments, L is

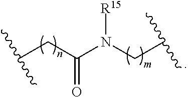

In other embodiments, L is

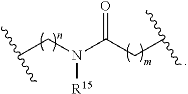

In yet other embodiments, L is

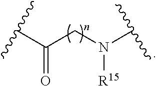

In still other embodiments, L is

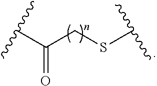

In another embodiment, L is

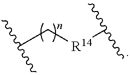

In yet another embodiment, L is

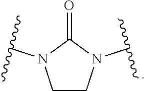 [SMILES: *N1CCN(*)C1=O]

In some embodiments of Formula (Y), L is

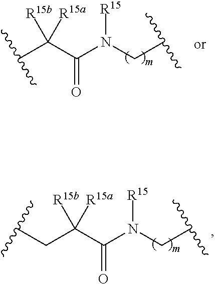

wherein:

R¹⁵ is as defined above for Formula (Y);

R^{15a} and R^{15b} are each independently selected from the group consisting of H, halogen, C₁₋₅alkyl, C₃₋₆carbocyclyl, alkylene-C₃₋₆carbocyclyl, aryl, alkylenearyl, or NH₂; wherein two —C₁₋₅alkyl taken together with the carbon atom to which they are attached form a C₃₋₆carbocyclyl; and

m is 0 or 1.

In some embodiments of Formula (Y), L is selected from the group consisting of:

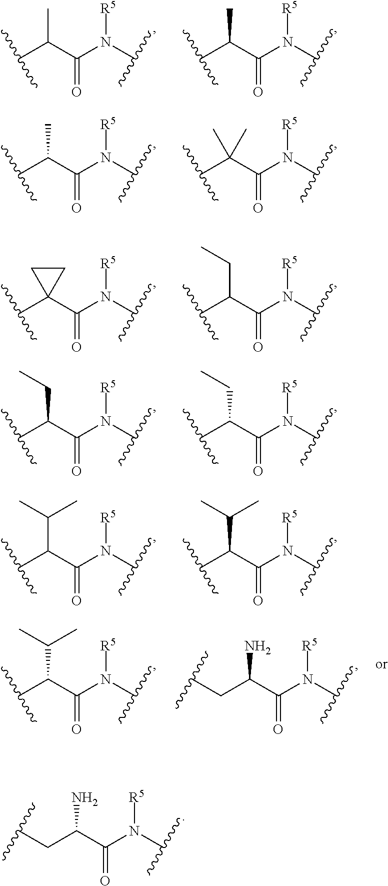

In some embodiments of Formula (Y), L is selected from the group consisting of:

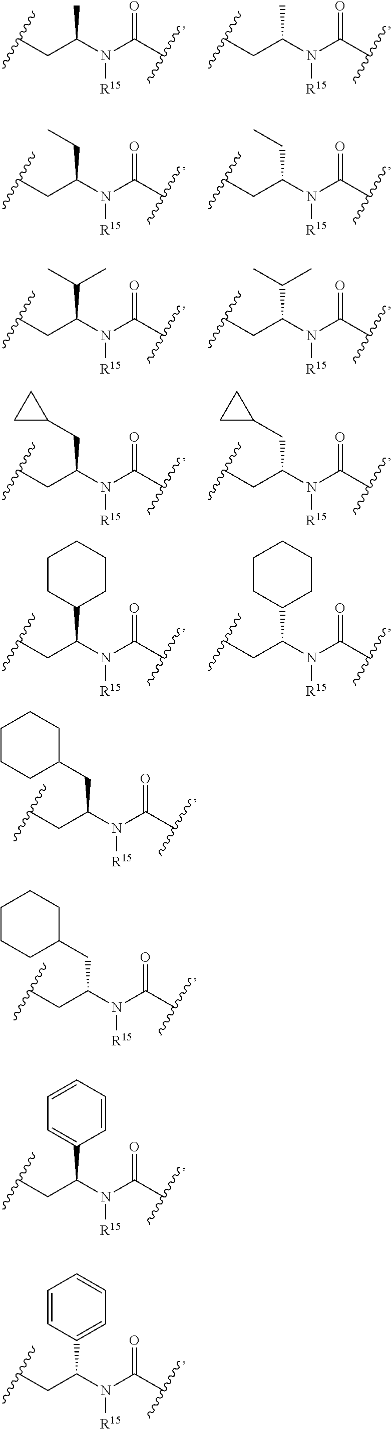

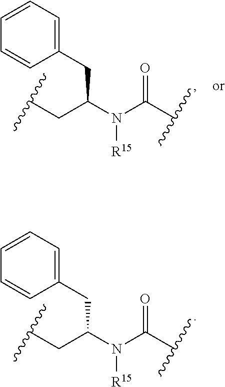

In some embodiments of Formula (Y), when L comprises an alkylene, the alkylene is an optionally substituted C₁₋₄alkylene. In some embodiments, the alkylene is an optionally substituted C₁₋₃alkylene. In some embodiments, the alkylene is an optionally substituted C₁₋₂alkylene. In some embodiments, the alkylene is an optionally substituted C₂₋₄alkylene. In some embodiments, the alkylene is an optionally substituted C₂₋₃alkylene. In some embodiments, the alkylene is an optionally substituted C₃₋₄alkylene. In some embodiments, when L comprises an alkylene, the alkylene is a C₁₋₄alkylene. In some embodiments, the alkylene is a C₁₋₃alkylene. In some embodiments, the alkylene is a C₁₋₂alkylene. In some embodiments, the alkylene is a C₂₋₄alkylene. In some embodiments, the alkylene is a C₂₋₃alkylene. In some embodiments, the alkylene is a C₃₋₄alkylene. In some embodiments, the alkylene is a methylene, an ethylene, a propylene, or a butylene, each of which is optionally substituted. In some embodiments, the alkylene is an ethylene, a propylene, or a butylene, each of which is optionally substituted. In some embodiments, the alkylene is an optionally substituted methylene. In some embodiments, the alkylene is an optionally substituted ethylene. In some embodiments, the alkylene is an optionally substituted propylene. In some embodiments, the alkylene is an optionally substituted butylene. In some embodiments, the alkylene is a methylene, an ethylene, a propylene, or a butylene. In some embodiments, the alkylene is a methylene. In some embodiments, the alkylene is an ethylene. In some embodiments, the alkylene is a propylene. In some embodiments, the alkylene is a butylene.

In some embodiments of Formula (Y), when L comprises an alkenylene, the alkenylene is an optionally substituted C₂₋₄alkenylene. In some embodiments, the alkenylene is an optionally substituted C₂₋₃alkenylene. In some embodiments, the alkenylene is an optionally substituted C₃₋₄alkenylene. In some embodiments, when L comprises an alkenylene, the alkenylene is a C₂₋₄alkenylene. In some embodiments, the alkenylene is a C₂₋₃alkenylene. In some embodiments, the alkenylene is a C₃₋₄alkenylene. In some embodiments, the alkenylene is an ethenylene, a propenylene, or a butenylene, each of which is optionally substituted. In some embodiments, the alkenylene is an optionally substituted ethenylene. In some embodiments, the alkenylene is an optionally substituted propenylene. In some embodiments, the alkenylene is an optionally substituted butenylene. In some embodiments, the alkenylene is an ethenylene, a propenylene, or a butenylene. In some embodiments, the alkenylene is an ethenylene. In some embodiments, the alkenylene is a propenylene. In some embodiments, the alkenylene is a butenylene.

In some embodiments of Formula (Y), the optional substituent is selected from the group consisting of oxo, halogen, C₁₋₅alkyl, C₃₋₆carbocyclyl, alkylenecarbocyclyl, aryl, heteroaryl, alkylenearyl, and alkyleneheteroaryl. In some embodiments, the optional substituent is selected from the group consisting of oxo, C₁₋₅alkyl, and C₃₋₆cycloalkyl. In some embodiments, the optional substituent is selected from the group consisting of oxo and C₁₋₅alkyl. In some embodiments, the optional substituent is oxo. In other embodiments, the optional substituent is C₁₋₅alkyl. In some embodiments, the C₁₋₅alkyl is methyl, ethyl, propyl or isopropyl. In some embodiments, the C₁₋₅alkyl is methyl, ethyl, or isopropyl. In other embodiments, the C₁₋₅alkyl is methyl. In some embodiments, the C₃₋₆cycloalkyl is cyclopropyl or cyclohexyl. In some embodiments, the aryl is phenyl. In some embodiments, the alkylenecarbocyclyl is methylenecyclopropyl or methylenecyclohexyl. In some embodiments, the alkylenearyl is methylenephenyl.

In some embodiments of Formula (Y), R¹¹ is heterocyclyl, aryl, or heteroaryl, each of which is optionally substituted. In some embodiments, R¹¹ is aryl or heteroaryl, each of which is optionally substituted. In some embodiments, R¹¹ is an optionally substituted aryl. In some embodiments, the aryl is an optionally substituted 6- to 12-membered aryl. In some embodiments, the aryl is an optionally substituted phenyl. In some embodiments of Formula (Y), the optionally substituted phenyl is selected from the group consisting of

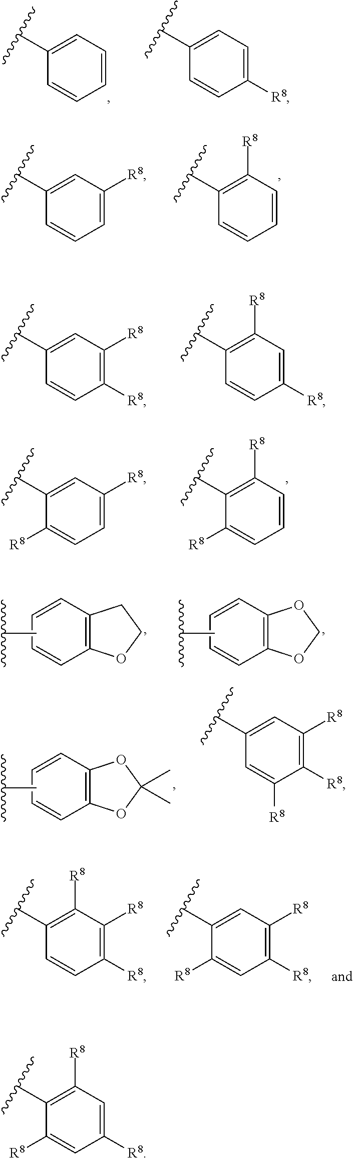

In some embodiments, R¹¹ is an optionally substituted heteroaryl. In some embodiments, the heteroaryl is a 5- to 12-membered heteroaryl with 1, 2, or 3 heteroatoms selected from the group consisting of N, O, and S. In some embodiments, the heteroaryl is an optionally substituted 5- or 6-membered heteroaryl having 1, 2, or 3 heteroatoms selected from S, O, and N. In some embodiments, the 5- or 6-membered heteroaryl with 1, 2, or 3 heteroatoms selected from the group consisting of N, O, and S is oxazolyl, thiazolyl, triazolyl, oxadiazolyl, thiadiazolyl, imidazolyl, isoxazolyl, pyrazolyl, pyridinyl, pyrimidinyl, or pyrazinyl. In some embodiments, the optionally substituted heteroaryl is selected from the group consisting of

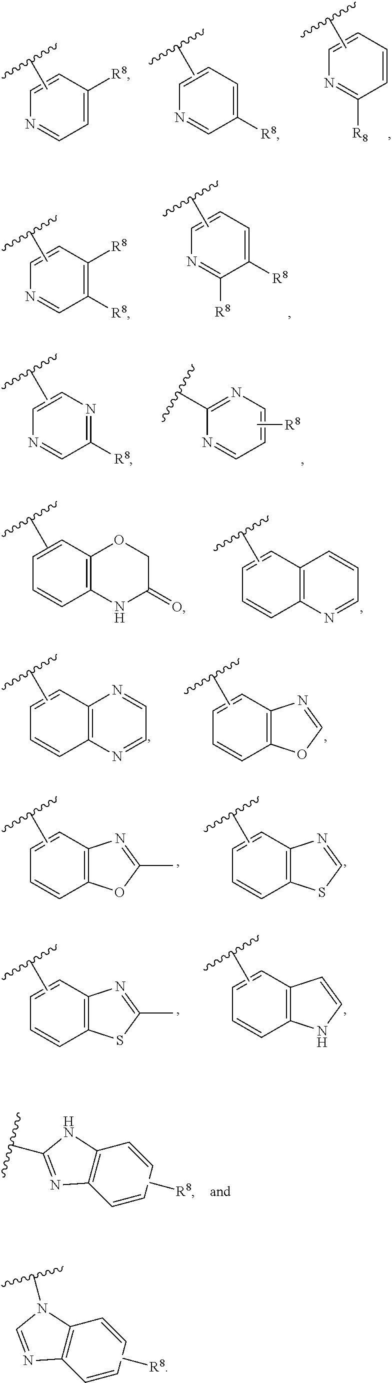

In some embodiments, the heteroaryl is an optionally substituted pyridinyl. In certain embodiments, the optionally substituted pyridinyl is selected from the group consisting of

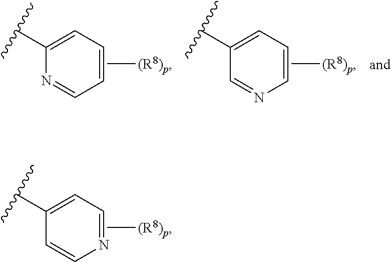

wherein p is 0, 1, or 2.

In some embodiments, the aryl or heteroaryl is optionally substituted with one or more H, halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN. In some embodiments, the aryl or heteroaryl is optionally substituted with one or more H, halogen, —C₁₋₅alkyl, CF₃, —OH, —O(C₁₋₅alkyl), —OCF₃, —OSO₂Me, —COOH, —C(O)OMe, or —SO₂Me.

In some embodiments, R¹¹ is an optionally substituted heterocyclyl. In some embodiments, the heterocyclyl is an optionally substituted 4- to 6-membered heterocyclyl having 1 or 2 heteroatoms selected from S, O, and N. In some embodiments, the heterocyclyl is an optionally substituted 3- to 6-membered heterocyclyl having up to 2 nitrogen atoms. In some embodiments, the heterocyclyl is azetidinyl, pyrrolidinyl, piperidinyl, piperazinyl, morpholinyl, or thiomorpholinyl.

In some embodiments of Formula (Y), R¹² is each independently H, C₁₋₅alkyl, CH₂aryl, or CH₂—(C₃₋₆carbocyclyl). In some embodiments, R¹² is each independently H, C₁₋₅alkyl, or CH₂Ph. In some embodiments of Formula (Y), R¹² is each independently H or C₁₋₅alkyl. In some embodiments, C₁₋₅alkyl is selected from the group consisting of methyl, ethyl, and isopropyl. In some embodiments, each R¹² is independently H.

In some embodiments of Formula (Y), R¹⁴ is heterocyclyl or heteroaryl. In some embodiments, R¹⁴ is heteroaryl. In some embodiments, the heteroaryl is an optionally substituted 5- or 6-membered heteroaryl having 1, 2, or 3 heteroatoms selected from S, O, and N. In some embodiments, the heteroaryl is selected from the group consisting of

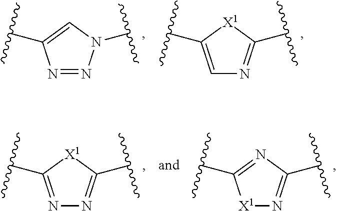

wherein X¹ is NR¹⁶, S, or O; and R¹⁶ is H or alkyl. In some embodiments of Formula (Y), R¹⁴ is heterocyclyl. In some embodiments, the heterocyclyl is an optionally substituted 3- to 12-membered heterocyclyl having 1, 2, or 3 heteroatoms selected from S, O, and N. In some embodiments, the heterocyclyl is an optionally substituted 5- or 6-membered heterocyclyl having up to 2 nitrogen atoms. In some embodiments, the heterocyclyl is selected from the group consisting of

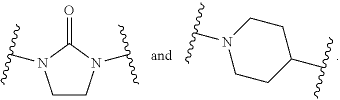 [SMILES: *N1CCN(*)C1=O ; *C1CCN(*)CC1]

In some embodiments of Formula (Y), R¹⁵ is H or alkyl. In some embodiments, the alkyl is a C₁₋₅alkyl. In certain embodiments, the C₁₋₅alkyl is selected from the group consisting of methyl, ethyl, propyl, isopropyl, isoamyl, and isobutyl. In other embodiments, the C₁₋₅alkyl is selected from the group consisting of methyl, ethyl, and isopropyl.

In some embodiments of Formula (Y), each of Z¹, Z², Z³, and Z⁴ is CR¹³. In some embodiments, at least one of Z¹, Z², Z³, and Z⁴ is N. In some embodiments, one of Z¹, Z², Z³, and Z⁴ is N. In some embodiments, two of Z¹, Z², Z³, and Z⁴are N. In certain embodiments, Z¹ is N and Z², Z³, and Z⁴ are CR¹³. In other embodiments, Z² is N and Z¹, Z³, and Z⁴ are CR¹³. In yet other embodiments, Z³ is N and Z¹, Z², and Z⁴ are CR¹³. In still other embodiments, Z⁴ is N and Z¹, Z², and Z³ are CR¹³. In another embodiment, Z¹ and Z⁴ are each N, and Z² and Z³ are CR¹³. In yet another embodiment, Z¹ and Z³ are each N, and Z² and Z⁴ are CR¹³. In still another 5 embodiment, Z² and Z⁴ are each N, and Z¹ and Z³ are CR¹³.

In some embodiments of Formula (Y), each R¹³ is independently H, halogen, alkyl, alkene, alkyne, haloalkyl, carbocyclyl, OH, O-alkyl, O-haloalkyl, O-carbocyclyl, OSO₂-alkyl, OSO₂-aryl, OSO₂NH₂, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN. In other embodiments, each R¹³ is independently H, halogen, —C₁₋₅alkyl, CF₃, —OH, —O(C₁₋₅alkyl), —OCF₃, —OSO₂Me, —COOH, —C(O)OMe, or —SO—₂Me. In some embodiments, two R¹³ taken together with the atoms to which they are attached can form carbocyclyl, heterocyclyl, or heteroaryl, each of which is optionally substituted.

In some embodiments of Formula (Y), m is 0 or 1. In some embodiments, m is 1 or 2. In some embodiments, m is 0 or 2. In some embodiments, m is 0. In some embodiments, m is 1. In some embodiments, m is 2.

In some embodiments of Formula (Y), n is 1 or 2. In some embodiments, n is 2 or 3. In some embodiments, n is 1 or 3. In some embodiments, n is 1. In some embodiments, n is 2. In some embodiments, n is 3.

In some embodiments of Formula (Y), m is 0 and n is 1. In other embodiments, m is 1 and n is 1. In still other embodiments, m is 0 and n is 2. In yet another embodiment, m is 2 and n is 1.

In some embodiments, the compound of Formula (Y) is selected from the group consisting of:

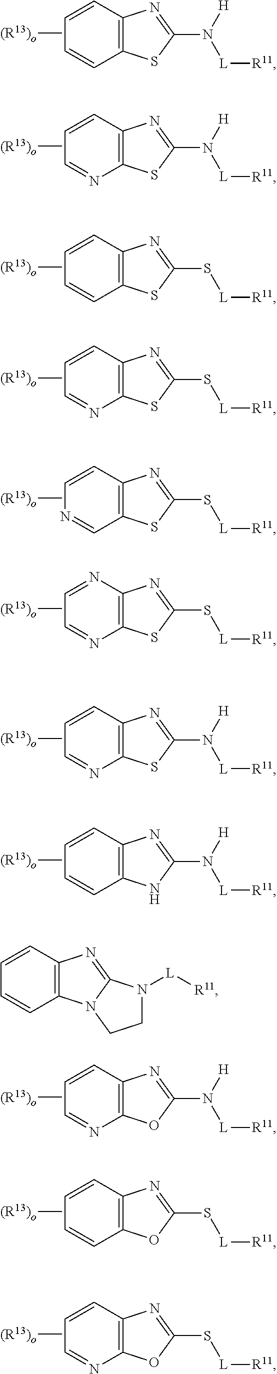

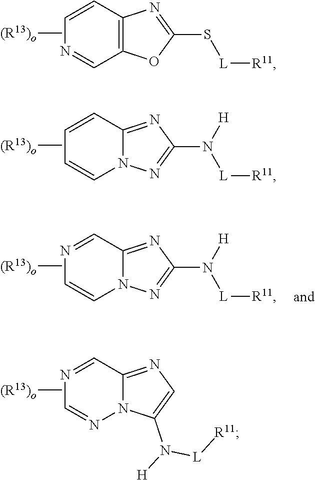

or a pharmaceutically acceptable salt, tautomer, solvate, or hydrate thereof,

wherein:

L, R¹¹, and R¹³ are as defined above for Formula (Y); and

o is an integer from 1 to 3.

In some embodiments, the compound of Formula (Y) is selected from the group consisting of:

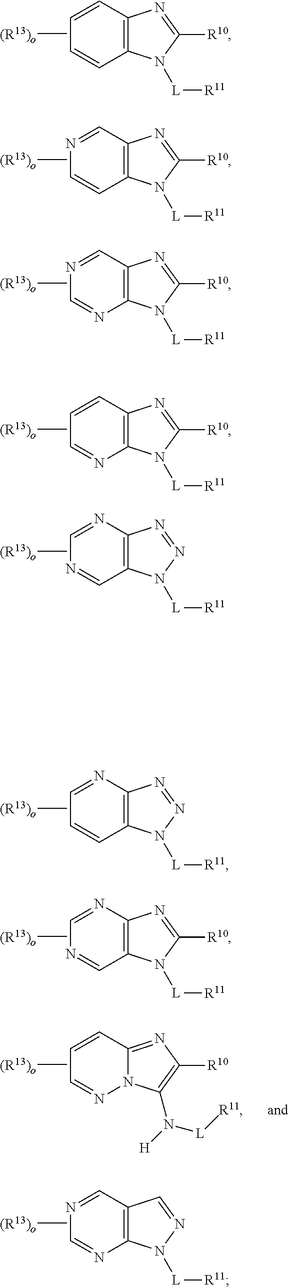

or a pharmaceutically acceptable salt, tautomer, solvate, or hydrate thereof,

wherein:

L, R¹⁰, R¹¹, and R¹³ are as defined above for Formula (Y); and

o is an integer from 1 to 3.

In some embodiments, the present disclosure provides a compound of Formula (Y) having one of the following structures:

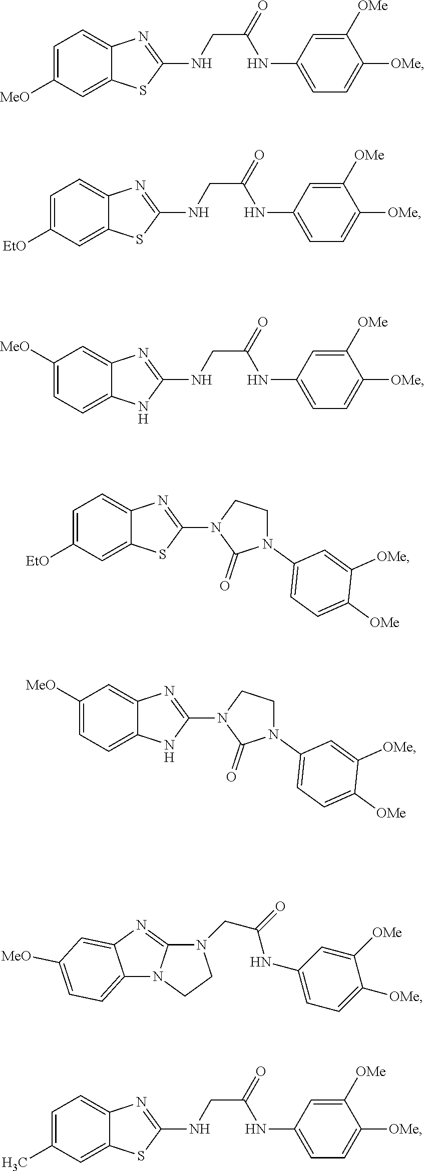

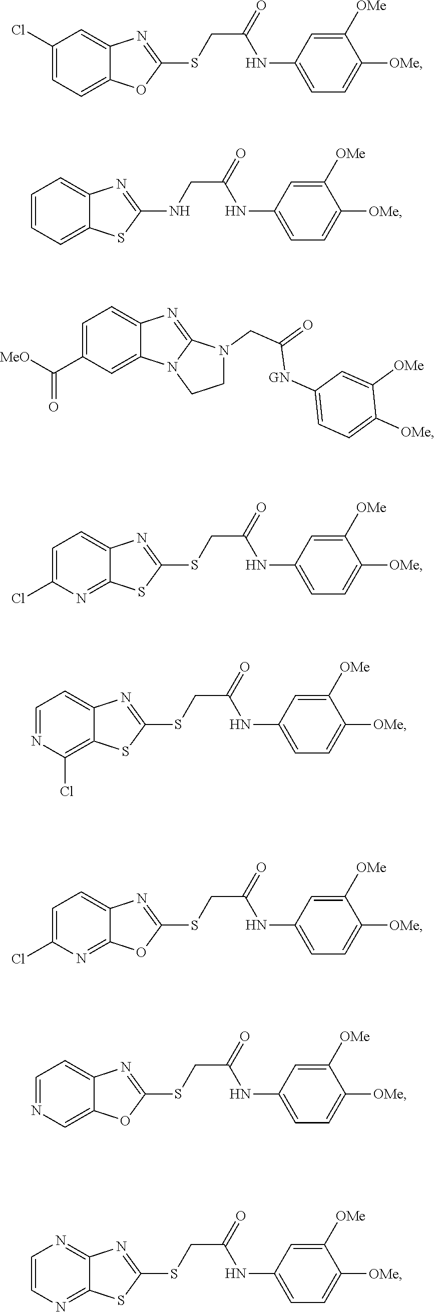

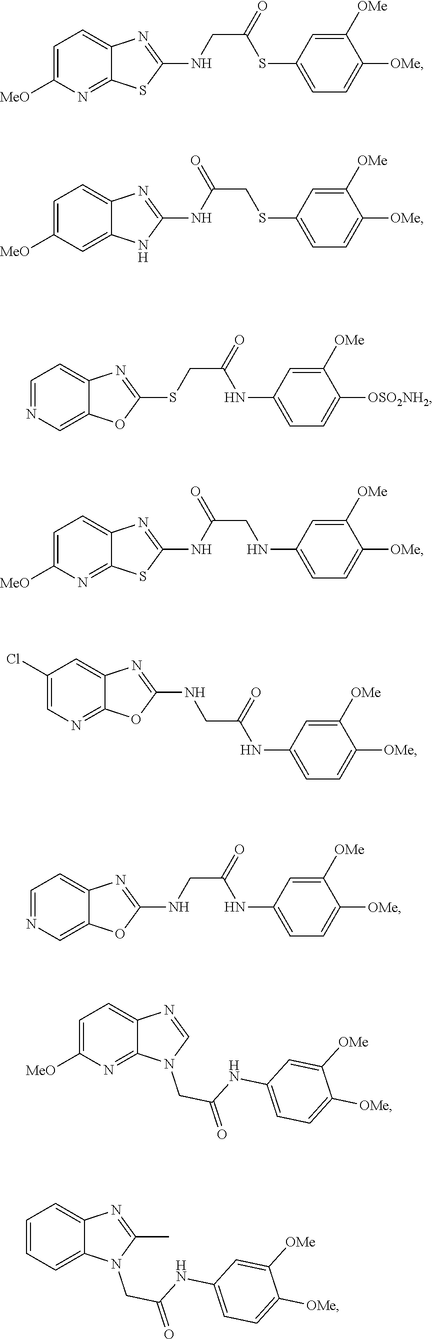

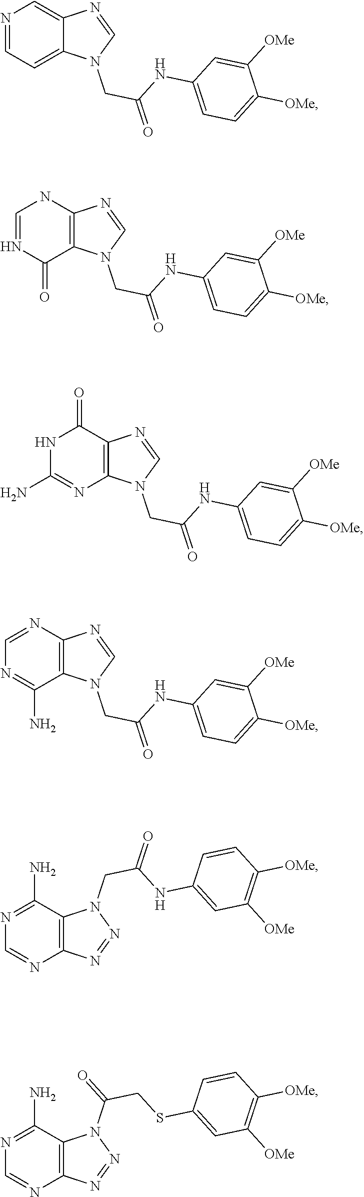 [SMILES: COc1ccc(NC(=O)Cn2cnc3cnccc32)cc1OC ; COc1ccc(NC(=O)Cn2cnc3[nH]cnc(=O)c32)cc1OC ; COc1ccc(NC(=O)Cn2cnc3c(=O)[nH]c(N)nc32)cc1OC ; COc1ccc(NC(=O)Cn2cnc3ncnc(N)c32)cc1OC ; COc1ccc(NC(=O)Cn2nnc3ncnc(N)c32)cc1OC ; COc1ccc(SCC(=O)n2nnc3ncnc(N)c32)cc1OC]

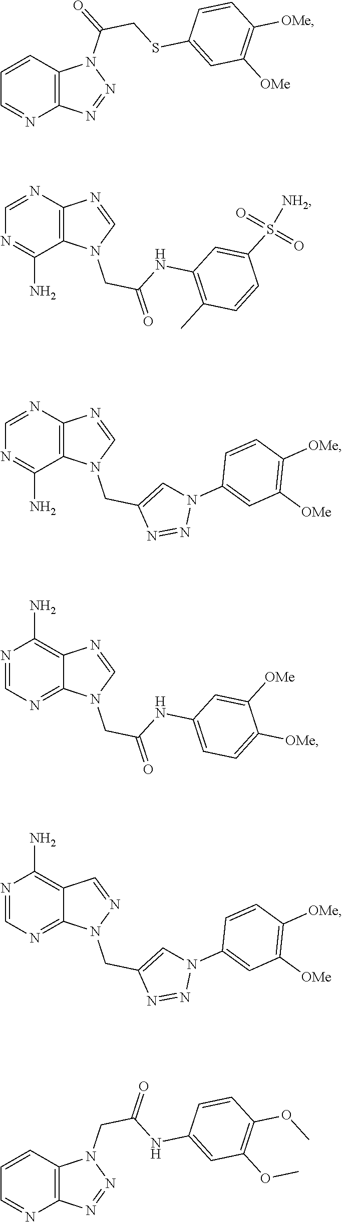 [SMILES: COc1ccc(SCC(=O)n2nnc3ncccc32)cc1OC ; Cc1ccc(S(N)(=O)=O)cc1NC(=O)Cn1cnc2ncnc(N)c21 ; COc1ccc(-n2cc(Cn3cnc4ncnc(N)c43)nn2)cc1OC ; COc1ccc(NC(=O)Cn2cnc3c(N)ncnc32)cc1OC ; COc1ccc(-n2cc(Cn3ncc4c(N)ncnc43)nn2)cc1OC ; COc1ccc(NC(=O)Cn2nnc3ncccc32)cc1OC]

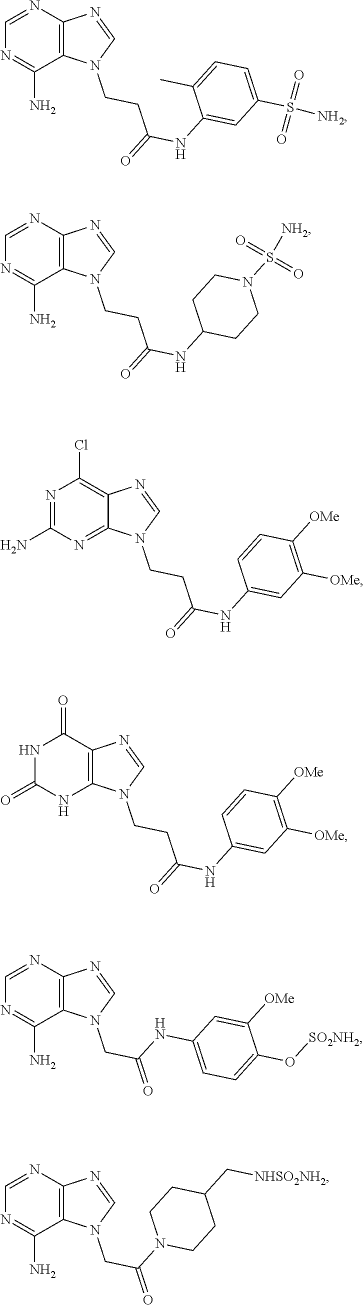

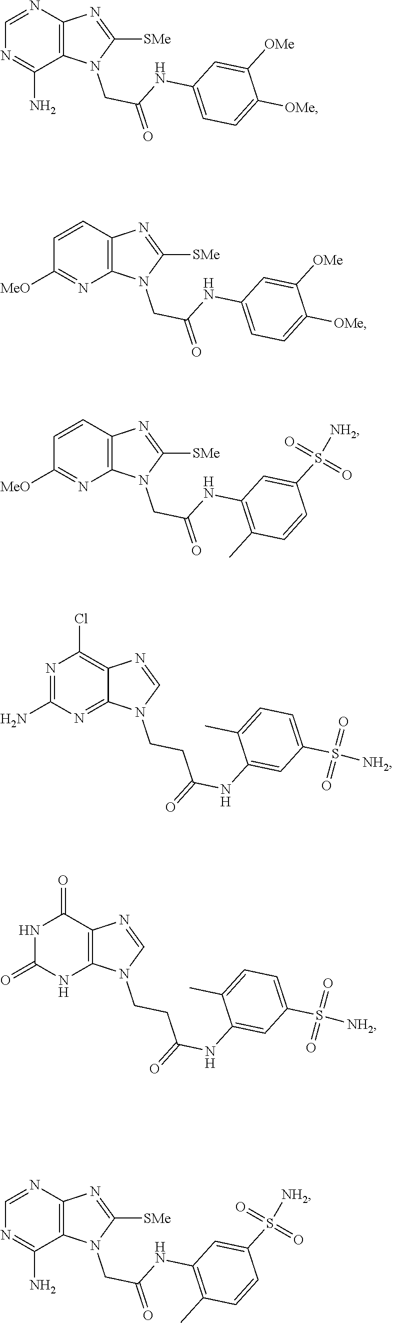 [SMILES: COc1ccc(NC(=O)Cn2c(SC)nc3ncnc(N)c32)cc1OC ; COc1ccc2nc(SC)n(CC(=O)Nc3ccc(OC)c(OC)c3)c2n1 ; COc1ccc2nc(SC)n(CC(=O)Nc3cc(S(N)(=O)=O)ccc3C)c2n1 ; Cc1ccc(S(N)(=O)=O)cc1NC(=O)CCn1cnc2c(Cl)nc(N)nc21 ; Cc1ccc(S(N)(=O)=O)cc1NC(=O)CCn1cnc2c(=O)[nH]c(=O)[nH]c21 ; CSc1nc2ncnc(N)c2n1CC(=O)Nc1cc(S(N)(=O)=O)ccc1C]

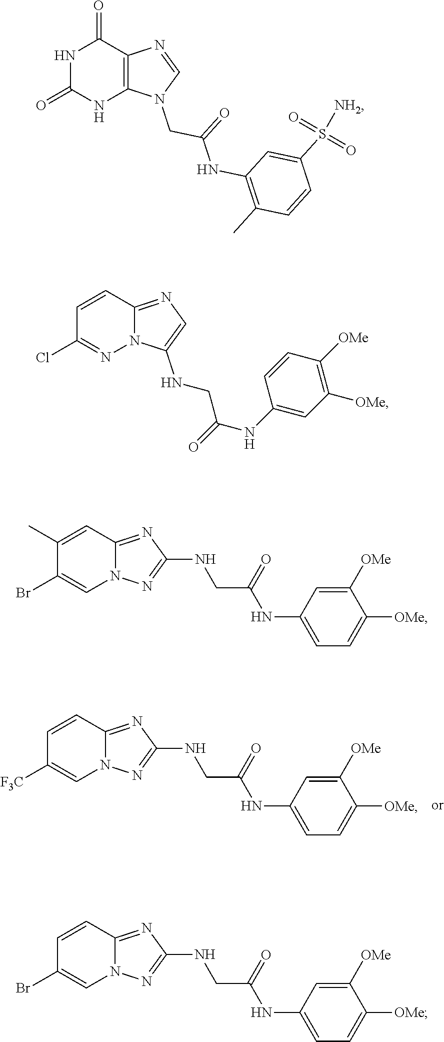 [SMILES: Cc1ccc(S(N)(=O)=O)cc1NC(=O)Cn1cnc2c(=O)[nH]c(=O)[nH]c21 ; COc1ccc(NC(=O)CNc2cnc3ccc(Cl)nn23)cc1OC ; COc1ccc(NC(=O)CNc2nc3cc(C)c(Br)cn3n2)cc1OC ; COc1ccc(NC(=O)CNc2nc3ccc(C(F)(F)F)cn3n2)cc1OC ; "or" ; COc1ccc(NC(=O)CNc2nc3ccc(Br)cn3n2)cc1OC]

or a pharmaceutically acceptable salt, tautomer, solvate, or hydrate thereof.

In other embodiments, the present disclosure provides compounds of Formula (Y) having one of the following structures:

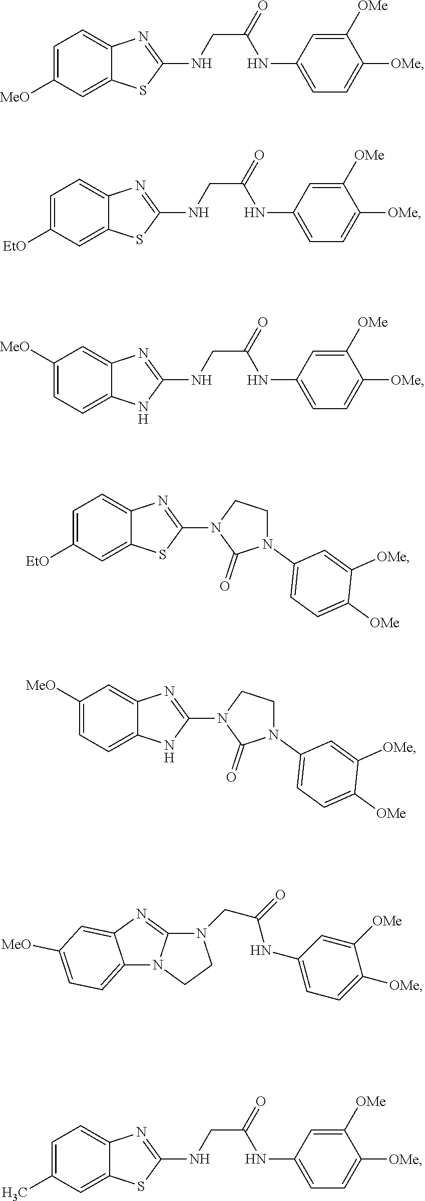

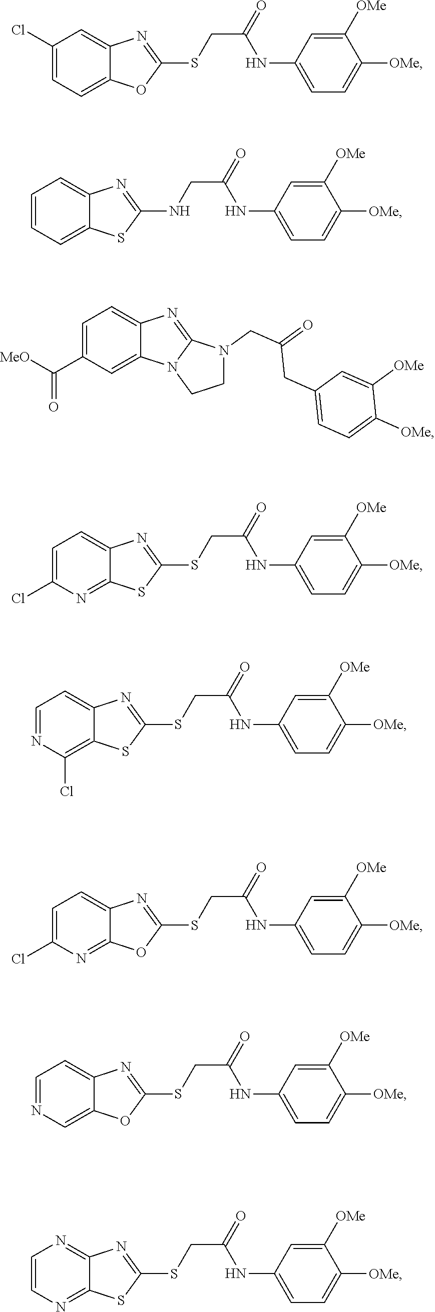

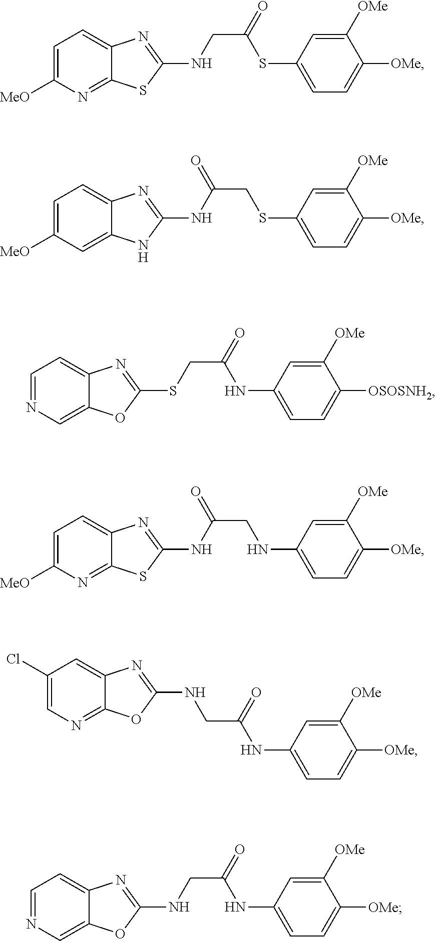

or a pharmaceutically acceptable salt, tautomer, solvate, or hydrate thereof.

In other embodiments, the present disclosure provides compounds of Formula (Y) having one of the following structures:

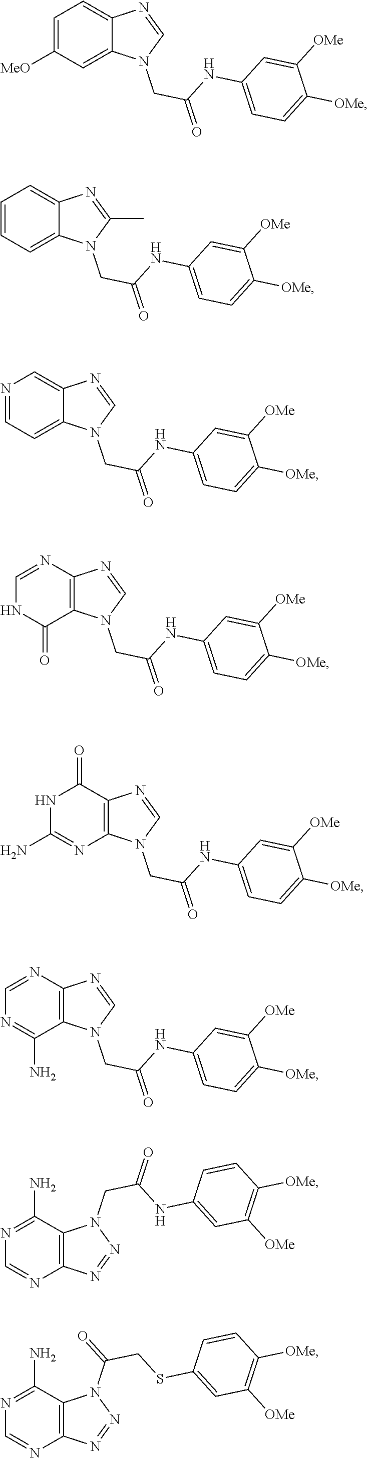

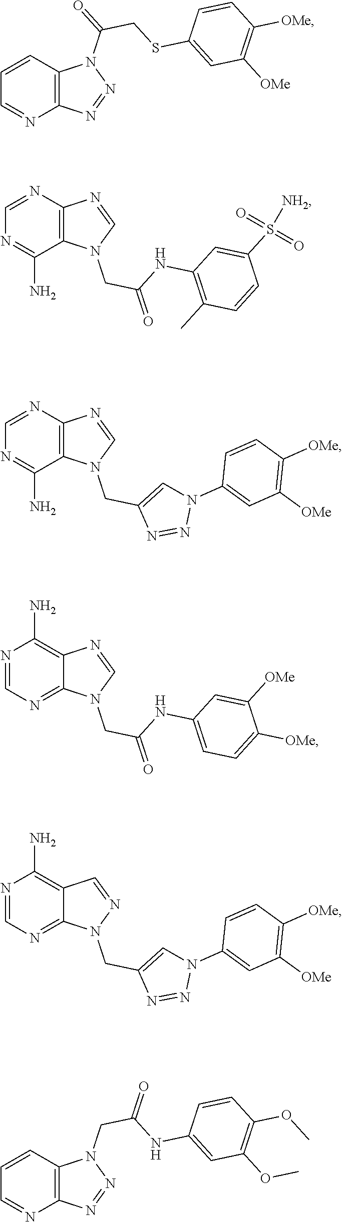 [SMILES: COc1ccc(SCC(=O)n2nnc3ncccc32)cc1OC ; Cc1ccc(S(N)(=O)=O)cc1NC(=O)Cn1cnc2ncnc(N)c21 ; COc1ccc(-n2cc(Cn3cnc4ncnc(N)c43)nn2)cc1OC ; COc1ccc(NC(=O)Cn2cnc3c(N)ncnc32)cc1OC ; COc1ccc(-n2cc(Cn3ncc4c(N)ncnc43)nn2)cc1OC ; COc1ccc(NC(=O)Cn2nnc3ncccc32)cc1OC]

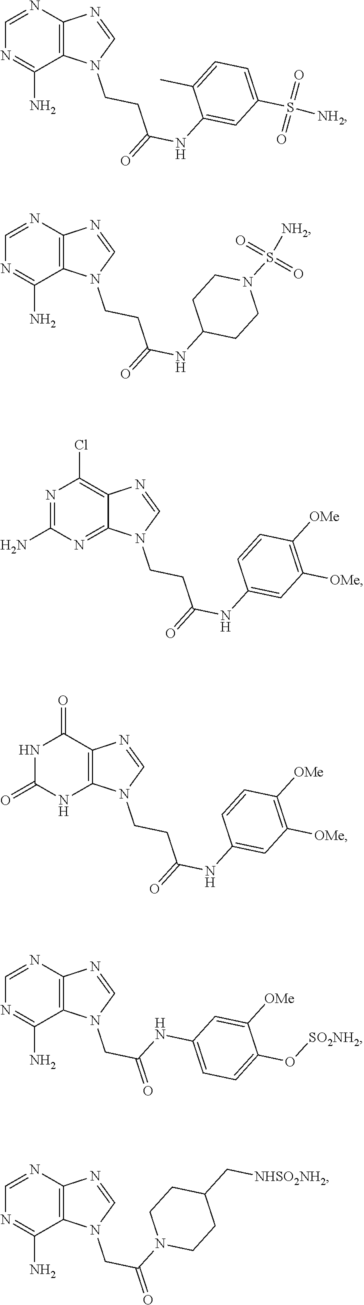

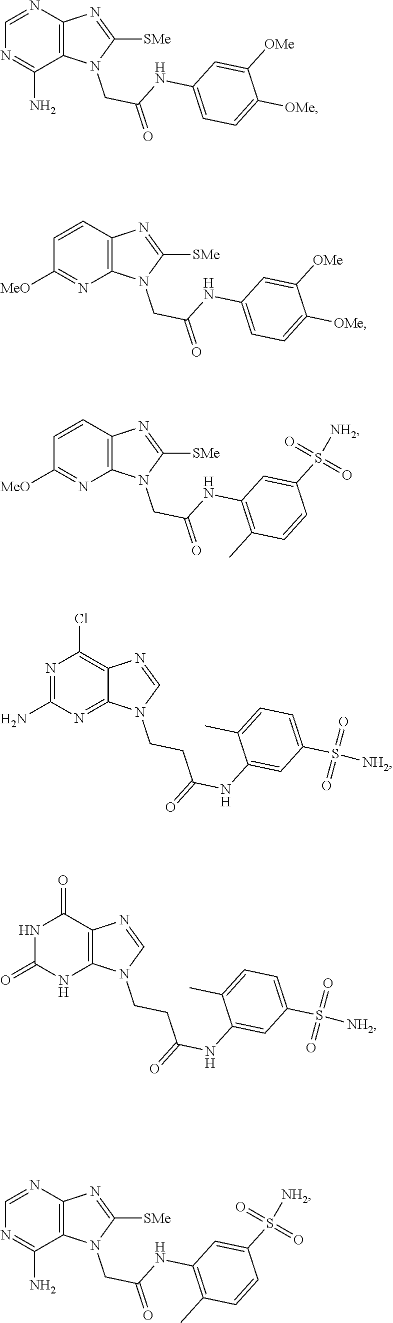 [SMILES: COc1ccc(NC(=O)Cn2c(SC)nc3ncnc(N)c32)cc1OC ; COc1ccc2nc(SC)n(CC(=O)Nc3ccc(OC)c(OC)c3)c2n1 ; COc1ccc2nc(SC)n(CC(=O)Nc3cc(S(N)(=O)=O)ccc3C)c2n1 ; Cc1ccc(S(N)(=O)=O)cc1NC(=O)CCn1cnc2c(Cl)nc(N)nc21 ; Cc1ccc(S(N)(=O)=O)cc1NC(=O)CCn1cnc2c(=O)[nH]c(=O)[nH]c21 ; CSc1nc2ncnc(N)c2n1CC(=O)Nc1cc(S(N)(=O)=O)ccc1C]

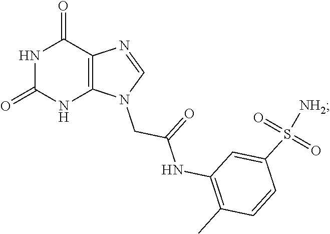 [SMILES: Cc1ccc(S(N)(=O)=O)cc1NC(=O)Cn1cnc2c(=O)[nH]c(=O)[nH]c21]

or a pharmaceutically acceptable salt, tautomer, solvate, or hydrate thereof.

In yet another embodiment, the present disclosure provides a compound of Formula (Y) having one of the following structures:

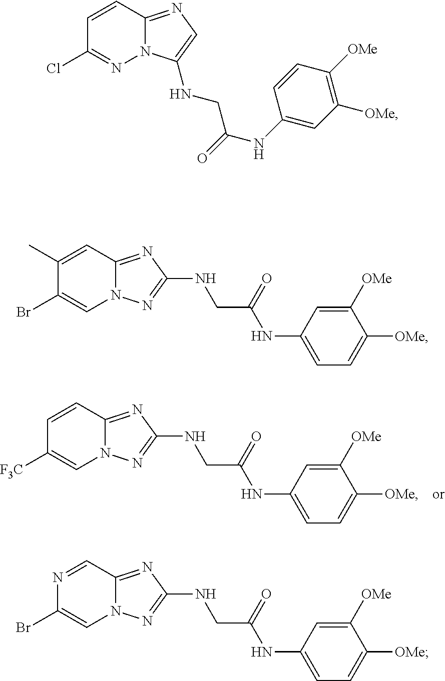 [SMILES: COc1ccc(NC(=O)CNc2cnc3ccc(Cl)nn23)cc1OC ; COc1ccc(NC(=O)CNc2nc3cc(C)c(Br)cn3n2)cc1OC ; COc1ccc(NC(=O)CNc2nc3ccc(C(F)(F)F)cn3n2)cc1OC ; COc1ccc(NC(=O)CNc2nc3cnc(Br)cn3n2)cc1OC]

or a pharmaceutically acceptable salt, tautomer, solvate, or hydrate thereof.

In various embodiments of the present disclosure, the compound of Formula (Y) is not a compound disclosed in the following publications:

(a) Chang, L., et al. J. Med. Chem. 2014, 57 (23), 10080-10100;

(b) Vankayalapati, H., et al. US20109/0031655.

In some embodiments of the present disclosure, the compound of Formula (Y) is a compound of Formula (YY) or a pharmaceutically acceptable salt, tautomer, solvate, or hydrate thereof:

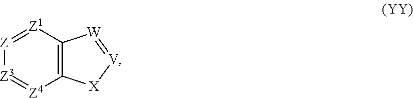

wherein V, W, X, Z¹, Z², Z³, and Z⁴ are as defined above for Formula (Y).

In some embodiments of the present disclosure, the compound of Formula (Y) is a compound of Formula (YYa) or a pharmaceutically acceptable salt, tautomer, solvate, or hydrate thereof:

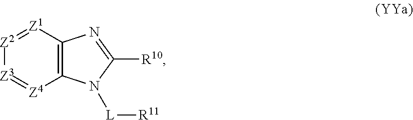

wherein:

R¹⁰ is H, alkyl, alkylenecarbocyclyl, carbocyclyl, —O-alkyl, —S-alkyl; and

L, R¹¹, Z¹, Z², Z³, and Z⁴ are as defined above for Formula (Y).

In some embodiments of Formulas (YYa), the alkyl is a C₁₋₅alkyl. In certain embodiments, the C₁₋₅alkyl is selected from the group consisting of methyl, ethyl, propyl, isopropyl, isoamyl, butyl, and isobutyl. In other embodiments, the C₁₋₅alkyl is selected from the group consisting of methyl, ethyl, and isopropyl.

In some embodiments of Formulas (YYa), the alkylene is an optionally substituted C₁₋₄alkylene. In some embodiments, the alkylene is an optionally substituted C₁₋₃alkylene. In some embodiments, the alkylene is an optionally substituted C₁₋₂alkylene. In some embodiments, the alkylene is an optionally substituted C₂₋₄alkylene. In some embodiments, the alkylene is an optionally substituted C₂₋₃alkylene. In some embodiments, the alkylene is an optionally substituted C₃₋₄alkylene. In some embodiments, when L comprises an alkylene, the alkylene is a C₁₋₄alkylene. In some embodiments, the alkylene is a C₁₋₃alkylene. In some embodiments, the alkylene is a C₁₋₂alkylene. In some embodiments, the alkylene is a C₂₋₄alkylene. In some embodiments, the alkylene is a C₂₋₃alkylene. In some embodiments, the alkylene is a C₃₋₄alkylene. In some embodiments, the alkylene is a methylene, an ethylene, a propylene, or a butylene, each of which is optionally substituted. In some embodiments, the alkylene is an ethylene, a propylene, or a butylene, each of which is optionally substituted. In some embodiments, the alkylene is an optionally substituted methylene. In some embodiments, the alkylene is an optionally substituted ethylene. In some embodiments, the alkylene is an optionally substituted propylene. In some embodiments, the alkylene is an optionally substituted butylene. In some embodiments, the alkylene is a methylene, an ethylene, a propylene, or a butylene. In some embodiments, the alkylene is a methylene. In some embodiments, the alkylene is an ethylene. In some embodiments, the alkylene is a propylene. In some embodiments, the alkylene is a butylene.

In some embodiments of Formulas (YYa), the carbocyclyl is a C₃₋₆carbocyclyl. In certain embodiments, the C₃₋₆carbocyclyl is cyclopropyl, cyclobutyl, cyclopentyl, or cyclohexyl.

In some embodiments of the present disclosure, the compound of Formula (Y) is a compound of Formula (YYb) or a pharmaceutically acceptable salt, tautomer, solvate, or hydrate thereof:

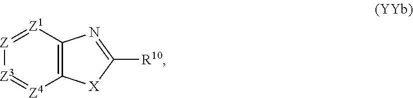

wherein:

X is O, S, or NR¹²;

R¹⁰ is —O-L-R¹¹, —S-L-R¹¹, —N(R¹²)-L-R¹¹, -L-R¹¹; and

R¹¹, R¹², L, Z¹, Z², Z³, and Z⁴ are as defined above for Formula (Y).

In some embodiments, the present disclosure provides a compound of Formula (Z) or a pharmaceutically acceptable salt, hydrate, or tautomer thereof:

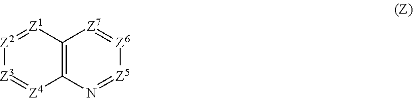

wherein:

Z¹, Z², Z³, Z⁴, Z⁵, Z⁶, and Z⁷ are each independently N or CR²², provided that
(a) one of Z¹, Z², Z³, Z⁴, Z⁵, Z⁶, or Z⁷ is -L-R¹¹—;
(b) no more than two of Z¹, Z², Z³, or Z⁴ are N; and
(c) one of Z⁶ or Z⁷ is N;

wherein:

L is a linker selected from —N(R¹⁹)—, -alkylene-(NR¹⁹)—,

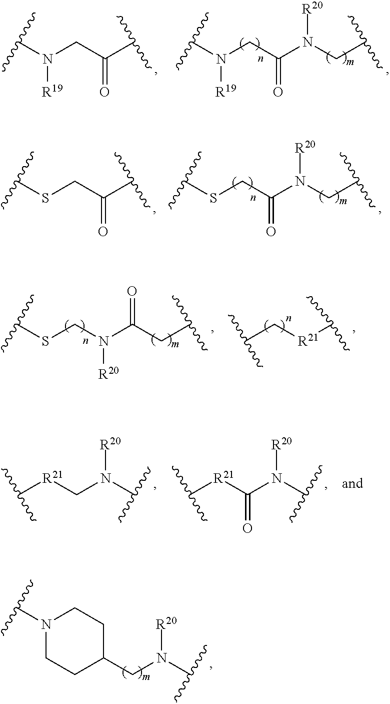

each of which is optionally substituted;

R¹⁸ is alkyl, carbocyclyl, heterocyclyl, aryl, or heteroaryl, each of which is optionally substituted;

R¹⁹ is H, alkyl, carbocyclyl, alkylenecarbocyclyl, or alkylenearyl;

R²⁰ is H, alkyl, alkylenecarbocyclyl, alkylenearyl;

R²¹ is carbocyclyl, heterocyclyl, or heteroaryl;

R²² is each independently halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN;

m is 0, 1, or 2; and

n is 1, 2, or 3.

In some embodiments of Formula (Z), each of Z¹, Z², Z³, and Z⁴ are independently N or CR²². In some embodiments, two of Z¹, Z², Z³, or Z⁴ are N. In some embodiments, one of Z¹, Z², Z³, or Z⁴ is N. In some embodiments, Z¹, Z², Z³, and Z⁴ are each CR²². In certain embodiments, Z¹ is N and Z², Z³, and Z⁴ are CR²². In other embodiments, Z² is N and Z¹, Z³, and Z⁴ are CR²². In yet other embodiments, Z³ is N and Z¹, Z², and Z⁴ are CR¹³. In still other embodiments, Z⁴ is N and Z¹, Z², and Z³ are CR²². In another embodiment, Z¹ and Z⁴ are each N, and Z² and Z³ are CR²². In yet another embodiment, Z¹ and Z³ are each N, and Z² and Z⁴ are CR²². In still another embodiment, Z² and Z⁴ are each N, and Z¹ and Z³ are CR²².

In some embodiments of Formula (Z), Z⁶ is N and one of Z¹, Z⁵, or Z⁷ is -L-R¹¹—. In some embodiments, Z⁶ is N and one of Z⁵ or Z⁷ is -L-R¹¹—. In other embodiments, Z⁶ is N, Z⁷ is CR²², and Z⁵ is -L-R¹¹—.

In some embodiments of Formula (Z), Z⁷ is N and one of Z¹, Z⁵, or Z⁶ is -L-R¹¹—. In some embodiments, Z⁷ is N and one of Z⁵ or Z⁶ is -L-R¹¹—. In other embodiments, Z⁷ is N, Z⁶ is CR²², and Z⁵ is -L-R¹¹—.

In some embodiments of Formula (Z), L is

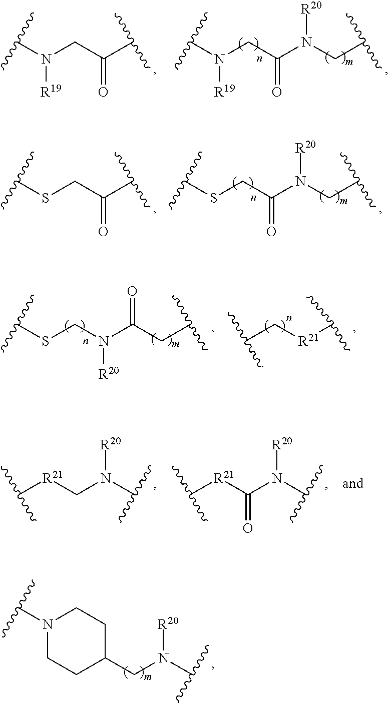

and each of which is optionally substituted. In some embodiments, L is

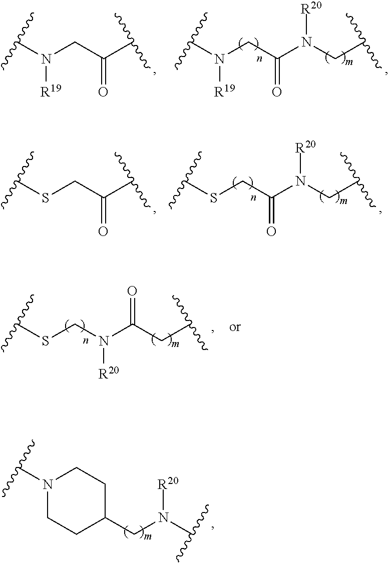

each of which is optionally substituted. In other embodiments, L is

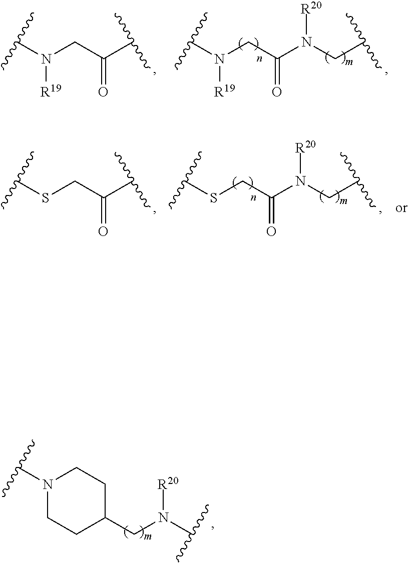

each of which is optionally substituted. In other embodiments, L is

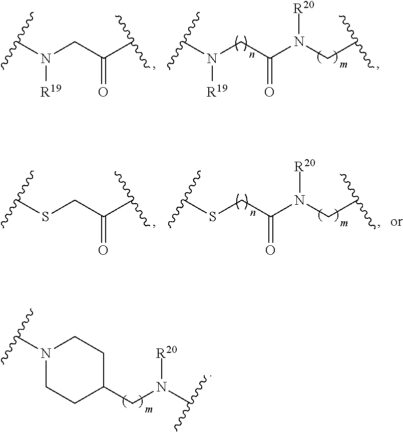

In some embodiments of Formula (Z), L is selected from the group consisting of

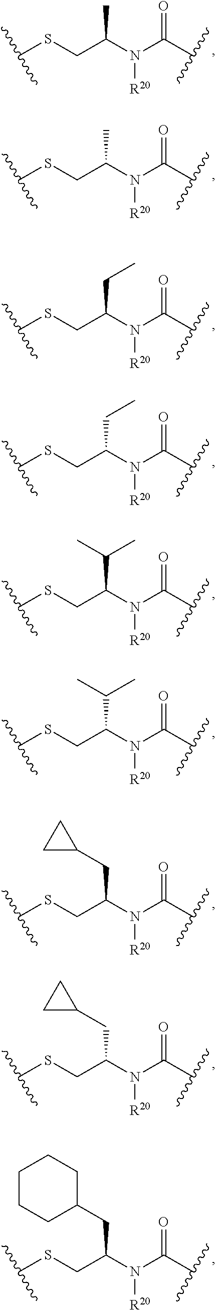

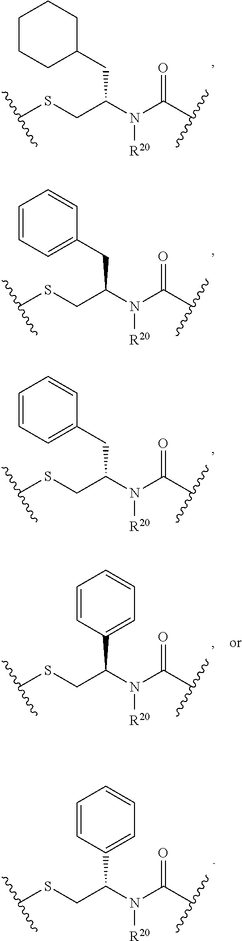

In some embodiments of Formula (Z), when L comprises an alkylene, the alkylene is an optionally substituted C₁₋₄alkylene. In some embodiments, the alkylene is an optionally substituted C₁₋₃alkylene. In some embodiments, the alkylene is an optionally substituted C₁₋₂alkylene. In some embodiments, the alkylene is an optionally substituted C₂₋₄alkylene. In some embodiments, the alkylene is an optionally substituted C₂₋₃alkylene. In some embodiments, the alkylene is an optionally substituted C₃₋₄alkylene. In some embodiments, when L comprises an alkylene, the alkylene is a C₁₋₄alkylene. In some embodiments, the alkylene is a C₁₋₃alkylene. In some embodiments, the alkylene is a C₁₋₂alkylene. In some embodiments, the alkylene is a C₂₋₄alkylene. In some embodiments, the alkylene is a C₂₋₃alkylene. In some embodiments, the alkylene is a C₃₋₄alkylene. In some embodiments, the alkylene is a methylene, an ethylene, a propylene, or a butylene, each of which is optionally substituted. In some embodiments, the alkylene is an ethylene, a propylene, or a butylene, each of which is optionally substituted. In some embodiments, the alkylene is an optionally substituted methylene. In some embodiments, the alkylene is an optionally substituted ethylene. In some embodiments, the alkylene is an optionally substituted propylene. In some embodiments, the alkylene is an optionally substituted butylene. In some embodiments, the alkylene is a methylene, an ethylene, a propylene, or a butylene. In some embodiments, the alkylene is a methylene. In some embodiments, the alkylene is an ethylene. In some embodiments, the alkylene is a propylene. In some embodiments, the alkylene is a butylene.

In some embodiments of Formula (Z), the optional substituent is selected from the group consisting of oxo, halogen, C₁₋₅alkyl, C₃₋₆carbocyclyl, alkylenecarbocyclyl, aryl, heteroaryl, alkylenearyl, and alkyleneheteroaryl. In some embodiments, the optional substituent is selected from the group consisting of oxo, C₁₋₅alkyl, and C₃₋₆cycloalkyl. In some embodiments, the optional substituent is selected from the group consisting of oxo and C₁₋₅alkyl. In some embodiments, the optional substituent is oxo. In other embodiments, the optional substituent is C₁₋₅alkyl. In some embodiments, the C₁₋₅alkyl is methyl, ethyl, propyl or isopropyl. In some embodiments, the C₁₋₅alkyl is methyl, ethyl, or isopropyl. In other embodiments, the C₁₋₅alkyl is methyl. In some embodiments, the C₃₋₆cycloalkyl is cyclopropyl or cyclohexyl. In some embodiments, the aryl is phenyl. In some embodiments, the alkylenecarbocyclyl is methylenecyclopropyl or methylenecyclohexyl. In some embodiments, the alkylenearyl is methylenephenyl.

In some embodiments of Formula (Z), R¹¹ is alkyl, heterocyclyl, aryl, or heteroaryl, each of which is optionally substituted.

In some embodiments of Formula (Z), R¹¹ is alkyl. In some embodiments, the alkyl is a C₁₋₅alkyl. In certain embodiments, the C₁₋₅alkyl is selected from the group consisting of methyl, ethyl, propyl, isopropyl, isoamyl, butyl, and isobutyl. In other embodiments, the C₁₋₅alkyl is selected from the group consisting of methyl, ethyl, and isopropyl.

In some embodiments, R¹¹ is aryl or heteroaryl, each of which is optionally substituted. In some embodiments, R¹¹ is optionally substituted aryl. In some embodiments, the optionally substituted aryl is a 6- to 12-membered aryl. In some embodiments, the aryl is an optionally substituted 6- to 12-membered aryl. In some embodiments, the aryl is an optionally substituted phenyl. In some embodiments of Formula (Z), the optionally substituted phenyl is selected from the group consisting the optionally substituted phenyl is selected from the group consisting of

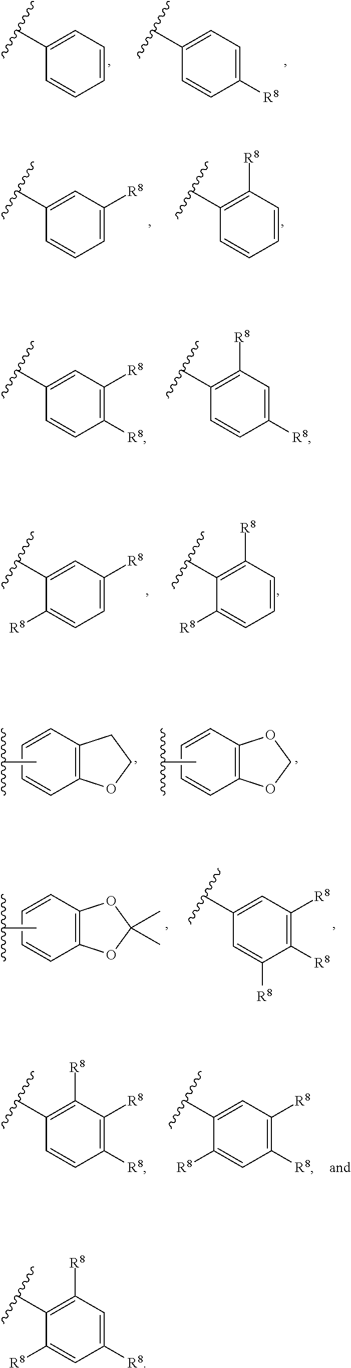

In some embodiments, R⁸ is an optionally substituted heteroaryl. In some embodiments, the optionally substituted heteroaryl is a 5- to 12-membered heteroaryl with 1, 2, or 3 heteroatoms selected from the group consisting of N, O, and S. In some embodiments, the heteroaryl is an optionally substituted 5- or 6-membered heteroaryl having 1, 2, or 3 heteroatoms selected from S, O, and N. In some embodiments, the 5- or 6-membered heteroaryl with 1, 2, or 3 heteroatoms selected from the group consisting of N, O, and S is oxazolyl, thiazolyl, triazolyl, oxadiazolyl, thiadiazolyl, imidazolyl, isoxazolyl, pyrazolyl, pyridinyl, pyrimidinyl, or pyrazinyl. In some embodiments, the optionally substituted heteroaryl is selected from the group consisting of

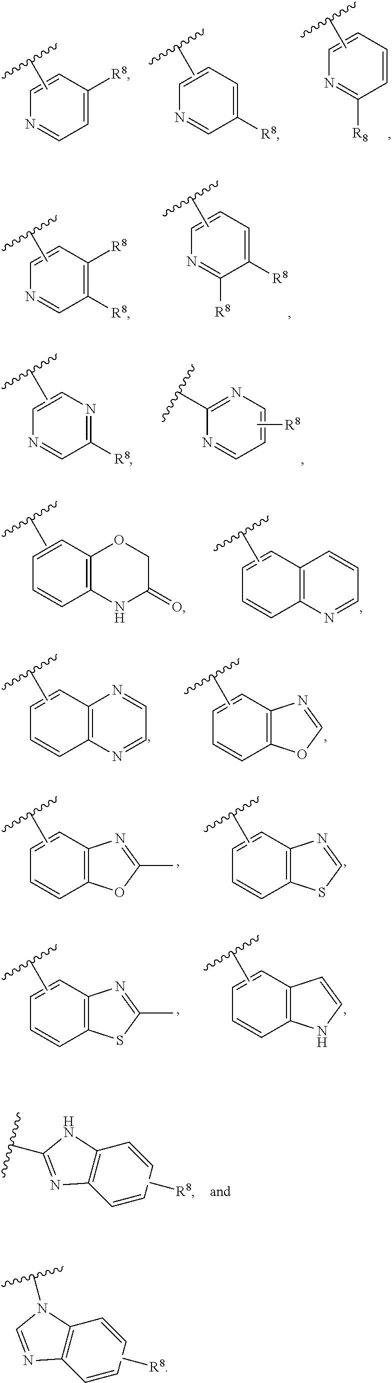

In some embodiments, the heteroaryl is an optionally substituted pyridinyl. In certain embodiments, the optionally substituted pyridinyl is selected from the group consisting of

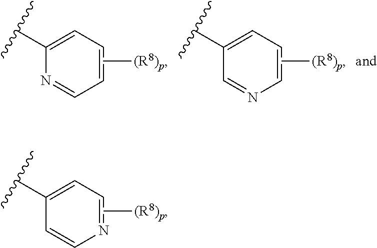

wherein p is 0, 1, or 2.

In some embodiments, the aryl or heteroaryl is optionally substituted with one or more H, halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN. In some embodiments, the aryl or heteroaryl is optionally substituted with one or more H, halogen, —C₁₋₅alkyl, CF₃, —OH, —O(C₁₋₅alkyl), —OCF₃, —OSO₂Me, —COOH, —C(O)OMe, or —SO₂Me.

In some embodiments, the heterocyclyl is an optionally substituted 3- to 12-membered heterocycle having 1, 2, or 3 heteroatoms selected from the group consisting of N, O, and S. In some embodiments, the heterocyclyl is an optionally substituted 3- to 6-membered heterocyclyl having 1 or 2 nitrogen atoms. In some embodiments, the 3- to 6-membered heterocyclyl is azetidinyl, pyrrolidinyl, piperidinyl, piperazinyl, morpholinyl, or thiomorpholinyl. In some embodiments, the 3- to 6-membered heterocyclyl is an optionally substituted pyrrolidinyl, piperidinyl, or piperazinyl. In other embodiments, the 3- to 6-membered heterocyclyl is an optionally substituted piperidinyl.

In some embodiments of Formula (Z), R¹⁹ is H or alkyl. In some embodiments, the alkyl is a C₁₋₅alkyl. In certain embodiments, the C₁₋₅alkyl is selected from the group consisting of methyl, ethyl, propyl, isopropyl, isoamyl, butyl, and isobutyl. In other embodiments, the C₁₋₅alkyl is selected from the group consisting of methyl, ethyl, and isopropyl. In some embodiments, R¹⁹ is H.

In some embodiments of Formula (Z), R²⁰ is H, —C₁₋₅alkyl, —C₃₋₆carbocyclyl, —CH₂-aryl, or —CH₂—(C₃₋₆carbocyclyl). In some embodiments, R²⁰ is H, Me, or —CH₂Ph. In other embodiments, R²⁰ is H.

In some embodiments of Formula (Z), R²¹ is heterocyclyl or heteroaryl.

In some embodiments of Formula (Z), the heteroaryl is an optionally substituted 5- or 6-membered heteroaryl having 1, 2, or 3 heteroatoms selected from S, O, and N. In certain embodiments, heteroaryl is selected from the group consisting of

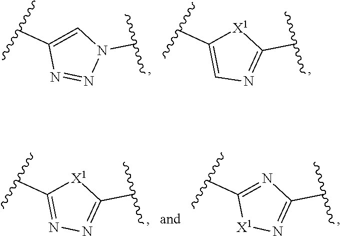

wherein: X¹ is NR¹⁶, S, or O; and R¹⁶ is H or alkyl.

In some embodiments of Formula (Z), the heterocyclyl is an optionally substituted 3-to 12-membered heterocyclyl having 1, 2, or 3 heteroatoms selected from S, O, and N. In some embodiments, the heterocyclyl is an optionally substituted 5- or 6-membered heterocyclyl 1 having up to 2 nitrogen atoms. In certain embodiments, the heterocyclyl is selected from the

group consisting of

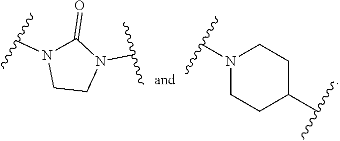 [SMILES: *N1CCN(*)C1=O ; *C1CCN(*)CC1]

In some embodiments of Formula (Z), m is 0 or 1. In some embodiments, m is 1 or 2. In some embodiments, m is 0 or 2. In some embodiments, m is 0. In some embodiments, m is 1. In some embodiments, m is 2.

In some embodiments of Formula (Z), n is 1 or 2. In some embodiments, n is 2 or 3. In some embodiments, n is 1 or 3. In some embodiments, n is 1. In some embodiments, n is 2. In some embodiments, n is 3.

In some embodiments of Formula (Z), m is 0 and n is 1. In other embodiments, m is 1 and n is 1. In still other embodiments, m is 0 and n is 2. In yet another embodiment, m is 2 and n is 1.

In some embodiments, the present disclosure provides a compound of Formula (Z) having one of the following structures:

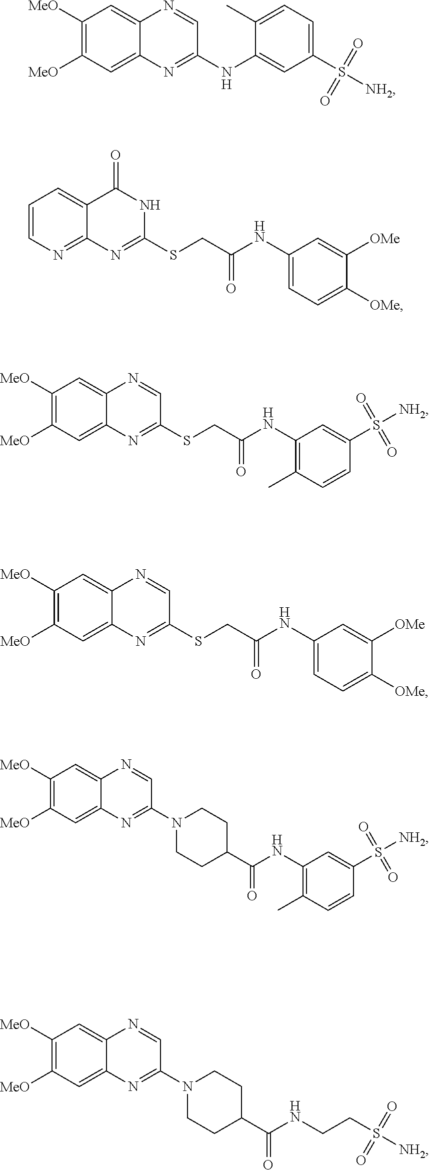 [SMILES: COc1cc2ncc(Nc3cc(S(N)(=O)=O)ccc3C)nc2cc1OC ; COc1ccc(NC(=O)CSc2nc3ncccc3c(=O)[nH]2)cc1OC ; COc1cc2ncc(SCC(=O)Nc3cc(S(N)(=O)=O)ccc3C)nc2cc1OC ; COc1ccc(NC(=O)CSc2cnc3cc(OC)c(OC)cc3n2)cc1OC ; COc1cc2ncc(N3CCC(C(=O)Nc4cc(S(N)(=O)=O)ccc4C)CC3)nc2cc1OC ; COc1cc2ncc(N3CCC(C(=O)NCCS(N)(=O)=O)CC3)nc2cc1OC]

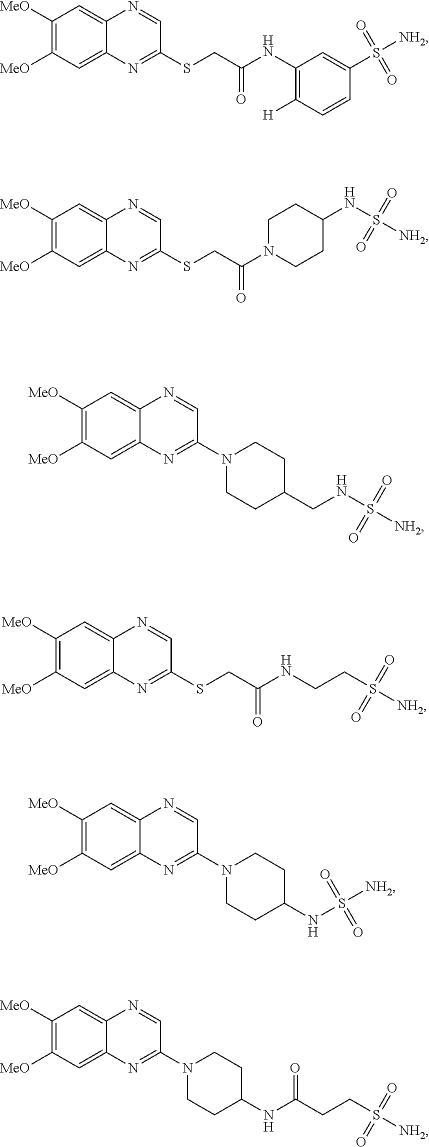 [SMILES: COc1cc2ncc(SCC(=O)Nc3cccc(S(N)(=O)=O)c3)nc2cc1OC ; COc1cc2ncc(SCC(=O)N3CCC(NS(N)(=O)=O)CC3)nc2cc1OC ; COc1cc2ncc(N3CCC(CNS(N)(=O)=O)CC3)nc2cc1OC ; COc1cc2ncc(SCC(=O)NCCS(N)(=O)=O)nc2cc1OC ; COc1cc2ncc(N3CCC(NS(N)(=O)=O)CC3)nc2cc1OC ; COc1cc2ncc(N3CCC(NC(=O)CCS(N)(=O)=O)CC3)nc2cc1OC]

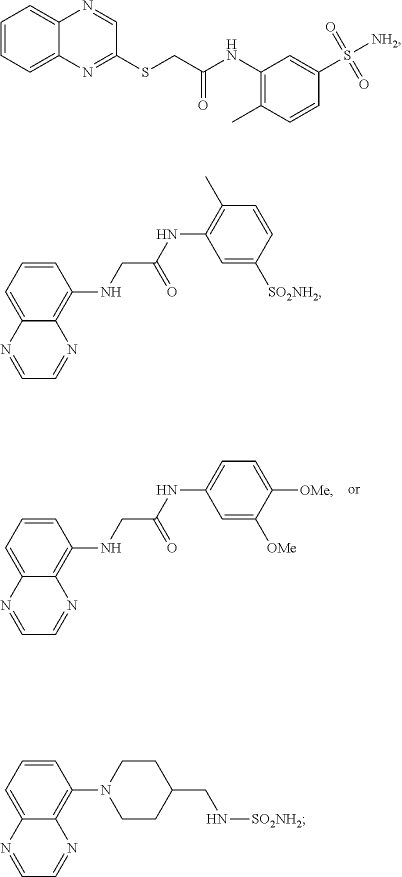 [SMILES: Cc1ccc(S(N)(=O)=O)cc1NC(=O)CSc1cnc2ccccc2n1 ; Cc1ccc(S(N)(=O)=O)cc1NC(=O)CNc1cccc2nccnc12 ; COc1ccc(NC(=O)CNc2cccc3nccnc23)cc1OC ; "or" ; NS(=O)(=O)NCC1CCN(c2cccc3nccnc23)CC1]

or a pharmaceutically acceptable salt, tautomer, hydrate, or solvate thereof.

In some embodiments of the present disclosure, the compound of Formula (Z) is a compound of Formula (ZZ):

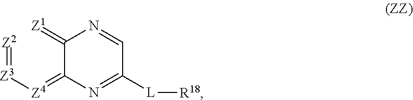

wherein:

L, R¹⁸, Z¹, Z², Z³, and Z⁴ are as defined above for Formula (Z).

In some embodiments of the present disclosure, the compound of Formula (Z) is a compound of Formula (ZZa):

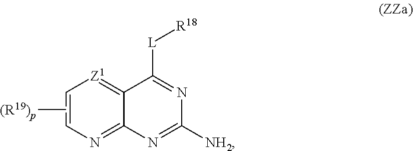

wherein:

L, R¹⁸, and Z¹ are as defined above for Formula (Z); and

p is 0, 1, or 2.

In various embodiments, the compound of Formula (Z), Formula (ZZ), and Formula (ZZa) excludes the compounds disclosed in WO 2019/051269.

The compounds described herein for Formula (X), Formula (XX), Formula (XXa), Formula (XXb), Formula (XXc), Formula (XXd), Formula (XXe), Formula (XXf), Formula (XXd1), Formula (XXf1), Formula (Y), Formula (YY), Formula (YYa), Formula (YYb), Formula (Z), Formula (ZZ), and Formula (ZZa) are meant to include all racemic mixtures, all individual enantiomers or combinations thereof, as well as all diastereomers or combinations thereof when two or more stereocenters are present, regardless of whether or not they are specifically depicted herein.

### Methods of Treatment

The present disclosure provides compounds and compositions that are useful in treating cancers and other conditions associated with ENPP1 dysfunction. Accordingly, in some embodiments, the compounds disclosed herein are inhibitors of ENPP1. In some embodiments, the compound of the present disclosure is cell permeable inhibitors of ENPP1.

In some embodiments, the present methods are useful in treating disorders of uncontrolled cellular proliferation in a subject in need thereof comprising administering to the subject a therapeutic amount of a compound disclosed herein (e.g., compounds of Formula (A1), Formula (A2), Formula (X), Formula (XX), Formula (XXa), Formula (XXb), Formula (XXc), Formula (XXd), Formula (XXe), Formula (XXf), Formula (XXd1), Formula (XXf1), Formula (Y), Formula (YY), Formula (YYa), Formula (YYb), Formula (Z), Formula (ZZ), and Formula (ZZa) a pharmaceutically acceptable salt, solvate, hydrate, or stereoisomer thereof, or a composition thereof. In some embodiments, the disorder of uncontrolled cellular proliferation is a cancer or a tumor. In some embodiments, the disorder or uncontrolled cellular proliferation is associated with an ENPP1 dysfunction, e.g., a dysfunction caused by a mutation to ENPP1.

In some embodiments, the present disclosure also provides a method for decreasing ENPP1 activity in a subject, the method comprising the step of administering to the subject an effective amount of a compound or composition disclosed herein. In some embodiments, the compound of the present disclosure is cell permeable.

In some embodiments, the present disclosure also provides a method for inhibiting ENPP1 activity in a subject by administering to the subject an effective amount of a compound or composition disclosed herein. In some embodiments, ENPP1 activity is inhibited by about 10%, about 20%, about 30%, about 40%, about 50%, about 60%, about 70%, about 80%, about 90%, or about 100%, including all ranges and values therebetween.

In some embodiments, the present disclosure provides method of treating cancer in a subject in need thereof comprising administering to the subject a therapeutic amount of a compound disclosed herein, a pharmaceutically acceptable salt, solvate, hydrate, or stereoisomer thereof, or a composition thereof. In some embodiments, the cancer is a solid tumor. In some embodiments, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioblastomas, melanoma and various head and neck tumors. In some embodiments, the solid tumor is breast cancer, lung cancer, or glioblastoma.

In some embodiments of the present disclosure, the cancer is a hematologic malignancy. In some embodiments, the hematologic malignancy is a leukemia, a lymphoma, or a myeloma. In some embodiments, the hematologic malignancy is a B-cell malignancy. In certain embodiments, the hematologic malignancy is multiple myeloma.

In some embodiments of the present disclosure, the cancer is a relapsed or refractory cancer. In some embodiments of the present disclosure, the cancer is a metastatic cancer.

In some embodiments, the present disclosure provides a method of treating a bacterial infection in a subject in need thereof comprising administering to the subject a therapeutic amount of a compound disclosed herein, a pharmaceutically acceptable salt, solvate, hydrate, or stereoisomer thereof, or a composition thereof. In some embodiments, the bacterial infection is a gram-positive infection. In other embodiments, the bacterial infection is a gram-negative infection. In some embodiments, the gram-positive infection is an infection caused by S. aureus (e.g., methicillin-susceptible or methicillin-resistant) or E. faecium. In other embodiments, the gram-negative infection is an infection caused by K. pneumoniae, P. aeruginosa, E. cloacae, or A. baumannii. In some embodiments, the bacterial infection is multidrug-resistant. In some embodiments, the bacterial infection is caused by M. tuberculosis. Accordingly, in various embodiments, the compounds and compositions of the present disclosure are effective in treating tuberculosis.

In some embodiments, the present disclosure provides a method of treating a viral infection in a subject in need thereof comprising administering to the subject a therapeutic amount of a compound disclosed herein, a pharmaceutically acceptable salt, solvate, hydrate, or stereoisomer thereof, or a composition thereof. In some embodiments, the viral infection is due to a DNA virus. In some embodiments, the viral infection is a due to a herpesvirus. In certain embodiments, the herpesvirus is selected from herpes simplex viruses 1 (HSV-1), herpes simplex viruses 2 (HSV-2), varicella-zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and Kaposi's sarcoma-associated herpesvirus (KSHV). In a specific embodiment, the herpesvirus is herpes simplex viruses 1 (HSV-1). In some embodiments of the present disclosure, the viral infection is a due to a retrovirus. In some embodiments, the retrovirus is human immunodeficiency virus (HIV). In some embodiments, the viral infection is a due to a hepatitis virus. In certain embodiments, the hepatitis virus is hepatitis B virus (HBV) or hepatitis D virus (HDV). In certain other embodiments, the viral infection is due to vaccinia virus (VACV), adenovirus, or human papillomaviruses (HPV). In some embodiments of the present disclosure, the viral infection is due to a RNA virus. In certain embodiments, the viral infection is due to dengue fever virus, yellow fever virus, ebola virus, Marburg virus, Venezuelan encephalitis virus, or zika virus.

### Compound Formulation

In some embodiments, the present disclosure provides pharmaceutical compositions comprisingan effective amount of a compound of Formula (A1), Formula (A2), Formula (X), Formula (XX), Formula (XXa), Formula (XXb), Formula (XXc), Formula (XXd), Formula (XXe), Formula (XXf), Formula (XXd1), Formula (XXf1), Formula (Y), Formula (YYa), Formula (YYb), Formula (Z), Formula (ZZ), or Formula (ZZa), or a pharmaceutically acceptable salt, tautomer, solvate, or hydrate thereof. The pharmaceutical compositions provided herein can comprise one or more pharmaceutically acceptable carriers or excipients.

In various embodiments, the pharmaceutical compositions of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The effective amount of a compound of the present disclosure, including pharmaceutically acceptable salts, esters, prodrugs, hydrates, solvates and isomers thereof, or pharmaceutical compositions thereof may be determined by one skilled in the art based on known methods.

In one embodiment, a pharmaceutical composition or a pharmaceutical formulation comprises a compound of the present disclosure, or a pharmaceutically acceptable salt thereof, and a pharmaceutically acceptable carrier, diluent, and/or excipient. Pharmaceutically acceptable carriers, diluents or excipients include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M, for example 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, for example sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets for example contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

The pharmaceutical composition of the present invention may be prepared into any type of formulation and drug delivery system by using any of the conventional methods well-known in the art. The inventive pharmaceutical composition may be formulated into injectable formulations, which may be administered by routes including intrathecal, intraventricular, intravenous, intraperitoneal, intranasal, intraocular, intramuscular, subcutaneous or intraosseous. Also, it may also be administered orally, or parenterally through the rectum, the intestines or the mucous membrane in the nasal cavity (see Gennaro, A. R., ed. (1995) Remington's Pharmaceutical Sciences). In particular embodiments, the composition is administered topically, instead of enterally. For instance, the composition may be injected, or delivered via a targeted drug delivery system such as a reservoir formulation or a sustained release formulation.

The pharmaceutical formulation of the present invention may be prepared by any well-known methods in the art, such as mixing, dissolving, granulating, dragee-making, levigating, emulsifying, encapsulating, entrapping, or lyophilizing processes. As mentioned above, the compositions of the present invention may include one or more physiologically acceptable carriers such as excipients and adjuvants that facilitate processing of active molecules into preparations for pharmaceutical use.

Proper formulation is dependent upon the route of administration chosen. For injection, for example, the composition may be formulated in an aqueous solution, such as in physiologically compatible buffers like as Hank's solution, Ringer's solution, or physiological saline buffer. For transmucosal or nasal administration, penetrants appropriate to the barrier to be permeated are used in the formulation. Such penetrants are generally known in the art. In a one embodiment of the present invention, the inventive compound may be prepared in an oral formulation. For oral administration, the compounds can be formulated readily by combining the active compounds with pharmaceutically acceptable carriers known in the art. Such carriers enable the disclosed compound to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use may be obtained as solid excipients, optionally grinding a resulting mixture, and processing the mixture of granules, after adding suitable adjuvants, if desired, to obtain tablets or dragee cores. Suitable excipients may be, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Also, wetting agents, such as sodium dodecyl sulfate and the like, may be added.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds doses.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All cited documents are herein incorporated by reference in their entirety for all purposes.

### EXAMPLES

Various derivatives of the above described formulas can be prepared from the appropriate starting materials and intermediates using the general methods described herein. Representative synthetic schemes are provided as follows.

Compound Synthesis

### Example 1: Compound 3

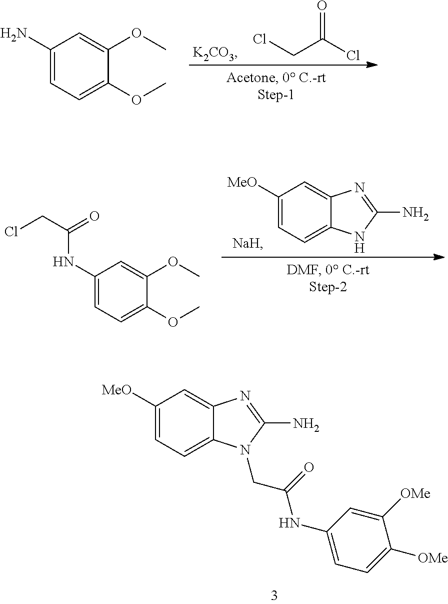

### Step 1: 2-chloro-N-(3,4-dimethoxyphenyl)acetamide

Procedure: To a stirred solution of 3,4-dimethoxyaniline (5 g, 32.64 mmol) in acetone (50 mL) was added potassium carbonate (9 g, 65.28 mmol) at 0° C. After 30 minute chloroacetyl chloride (3.5 mL, 48.96 mmol) was added in to it. The reaction mixture was allowed to stir at room temperature. Progress of the reaction was monitored by TLC. After completion of the reaction, the solvent was evaporated to dryness. The crude residue was suspended in water and extracted with ethyl acetate. The organic layer was dried using anhydrous sodium sulfate and concentrated to give crude residue. The purification was done by flash chromatography using hexane:ethylacetate as the eluent system. Yield: 4 g (54%)

### Step 2: N-(3,4-dimethoxyphenyl)-2-((5-methoxy-1H-benzo[d]imidazol-2-yl)amino)acetamide

Procedure: To a stirred solution of sodium hydride (24.5 mg, 0.61 mmol) in dimethylformamide (2 mL) was added 5-methoxy-1H-benzo[d]imidazol-2-amine (0.1 g, 0.61 mmol) at 0° C. After 30 minute 2-chloro-N-(3,4-dimethoxyphenyl)acetamide (0.154 g, 0.67 mmol) was added in to it. The reaction mixture was allowed to stir at room temperature. Progress of the reaction was monitored by TLC. After completion of the reaction, the reaction mixture was quenched with cold water and extracted with ethyl acetate. The organic layer was dried using anhydrous sodium sulfate and concentrated to give crude residue. The purification was done by flash chromatography using dichloromethane:methanol as the eluent system. Yield: 20 mg (9.15%)

### Example 2: Compound 10

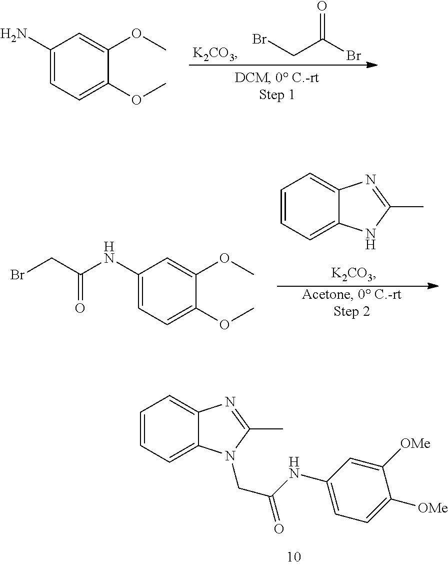

### Step 1: 2-bromo-N-(3,4-dimethoxyphenyl)acetamide

Procedure: To a stirred solution of 3,4-dimethoxyaniline (1 g, 6.52 mmol) in dichloromethane (10 mL) was added potassium carbonate (1.35 g, 9.78 mmol) at 0° C. After 30 minute bromoacetyl bromide (0.8 mL, 8.48 mmol) was added in to it. The reaction mixture was allowed to stir at room temperature. Progress of the reaction was monitored by TLC. After completion of the reaction, the reaction mixture was washed with water and brine. The organic layer was dried using anhydrous sodium sulfate and concentrated to give crude residue. The purification was done by flash chromatography using hexane:ethylacetate as the eluent system. Yield: 1.7 g (98%)

### Step 2: N-(3,4-dimethoxyphenyl)-2-(2-methyl-1H-benzo[d]imidazol-1-yl)acetamide

Procedure: To a stirred solution of 2-methyl-1H-benzo[d]imidazole (0.1 g, 0.75 mmol) in acetone (5 mL) was added potassium carbonate (0.15 g, 1.13 mmol) at 0° C. After 30 minute 2-bromo-N-(3,4-dimethoxyphenyl)acetamide (0.23 g, 0.83 mmol) was added in to it. The reaction mixture was allowed to stir at room temperature. Progress of the reaction was monitored by TLC. After completion of the reaction, the solvent was evaporated to dryness. The crude residue was suspended in water and extracted with ethyl acetate. The organic layer was dried using anhydrous sodium sulfate and concentrated to give crude residue. The purification was done by flash chromatography using dichloromethane:methanol as the eluent system. Yield: 0.4 g (16.25%)

### Example 3: Compound 37

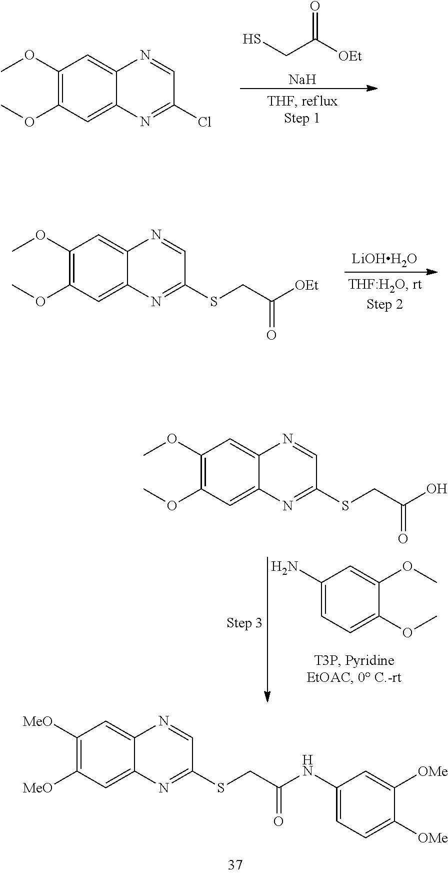

### Step 1: Ethyl 2-((6,7-dimethoxyquinoxalin-2-yl)thio)acetate

Procedure: To a stirred solution of sodium hydride (0.04 g, 1.59 mmol) in tetrahydrofuran (3 mL) was added ethyl 2-mercaptoacetate (0.18 mL, 1.46 mmol) at 0° C. The reaction mixture was allowed to reflux for 30 minute. 2-chloro-6,7-dimethoxyquinoxaline (0.3 g, 1.33 mmol) was added in to it. The reaction mixture was allowed to stir at reflux temperature. Progress of the reaction was monitored by TLC. After completion of the reaction, the reaction mixture was evaporated to dryness. The crude residue was purified by flash chromatography using hexane:ethylacetate as the eluent system. Yield: 340 mg (82.7%)

### Step 2: 2-((6,7-dimethoxyquinoxalin-2-yl)thio)acetic acid

Procedure: To a stirred solution of Ethyl 2-((6,7-dimethoxyquinoxalin-2-yl)thio)acetate (0.34 g, 1.10 mmol) in THF:H₂O (1:1, 5 mL) was added lithium hydroxide monohydrate (0.07 g, 1.65 mmol) at rt. Progress of the reaction was monitored by TLC. After completion of the reaction, the reaction mixture was evaporated to dryness. The crude residue was dissolved in water and neutralized with 2N HCl. After neutralization, it was extracted with ethylacetate. The organic layer was dried using anhydrous sodium sulfate and concentrated to give pure compound. Yield: 0.25 g (83%)

### Step 3: N-(3,4-dimethoxyphenyl)-2-((6,7-dimethoxyquinoxalin-2-yl)thio)acetamide

Procedure: To a stirred solution of 2-((6,7-dimethoxyquinoxalin-2-yl)thio)acetic acid (0.05 g, 0.17 mmol) in ethylacetate (5 mL) was added 3,4-dimethoxyaniline (0.025 g, 0.16 mmol) and pyridine (0.043 mL, 0.53 mmol) at 0° C. After 30 minute T3P (0.22 mL, 50% in ethylacetata, 0.71 mmol) was added in to it. Progress of the reaction was monitored by TLC. After completion of the reaction, the reaction mixture was washed with water and brine. The organic layer was dried using anhydrous sodium sulfate and concentrated to give crude residue. The purification was done by flash chromatography using dichloromethane: methanol as the eluent system. Yield: 0.20 g (27%)

### Example 4: Compound 46

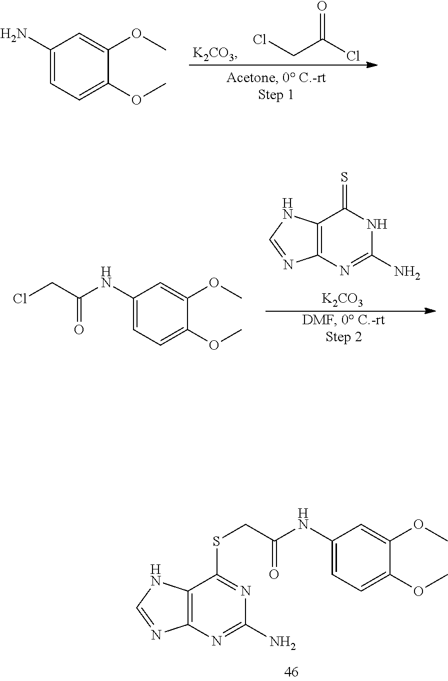

### Step 1: 2-chloro-N-(3,4-dimethoxyphenyl)acetamide

Procedure: To a stirred solution of 3,4-dimethoxyaniline (5 g, 32.64 mmol) in acetone (50 mL) was added potassium carbonate (9 g, 65.28 mmol) at 0° C. After 30 minute chloroacetyl chloride (3.5 mL, 48.96 mmol) was added in to it. The reaction mixture was allowed to stir at room temperature. Progress of the reaction was monitored by TLC. After completion of the reaction, the solvent was evaporated to dryness. The crude residue was suspended in water and extracted with ethyl acetate. The organic layer was dried using anhydrous sodium sulfate and concentrated to give crude residue. The purification was done by flash chromatography using hexane:ethylacetate as the eluent system. Yield: 4 g (54%)

### Step 2: 2-((2-amino-7H-purin-6-yl)thio)-N-(3,4-dimethoxyphenyl)acetamide

Procedure: To a stirred solution of 2-amino-1,7-dihydro-6H-purine-6-thione (0.1 g, 0.59 mmol) in dimethylformamide (3 mL) was added potassium carbonate (0.25 g, 1.79 mmol) at 0° C. After 30 minute 2-chloro-N-(3,4-dimethoxyphenyl)acetamide (0.15 g, 0.65 mmol) was added in to it. The reaction mixture was allowed to stir at room temperature. Progress of the reaction was monitored by TLC. After completion of the reaction, the solvent was evaporated to dryness. The crude residue was suspended in water and extracted with ethyl acetate. The organic layer was dried using anhydrous sodium sulfate and concentrated to give crude residue. The purification was done by flash chromatography using hexane:ethylacetate as the eluent system. Yield: 0.6 g (27.9%)

### Example 5: Compound 148

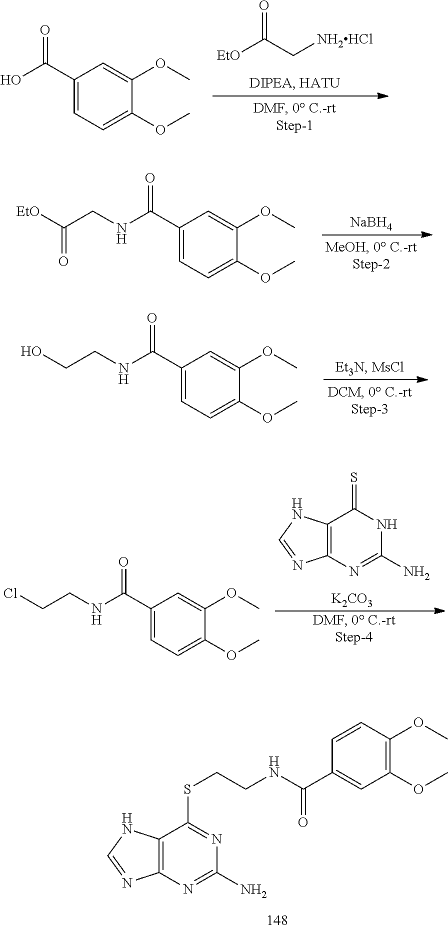

### Step 1: Ethyl (3,4-dimethoxybenzoyl)glycinate

Procedure: To a stirred solution of 3,4-dimethoxybenzoic acid (1 g, 5.48 mmol) and glycine ethyl ester hydrochloride (0.76 g, 5.48 mmol) in dimethylformamide (10 mL) was added diisopropylethylamine (3.35 mL, 19.2 mmol) at 0° C. After 30 minute HATU (3.1 g, 8.23 mmol) was added in to it. The reaction mixture was allowed to stir at room temperature. Progress of the reaction was monitored by TLC. After completion of the reaction, cold water was added in to it. The reaction mixture was extracted with ethyl acetate. The organic layer was dried using anhydrous sodium sulfate and concentrated to give crude residue. The purification was done by flash chromatography using hexane:ethylacetate as the eluent system. Yield: 1.34 g (91%)

### Step 2: N-(2-hydroxyethyl)-3,4-dimethoxybenzamide

Procedure: To a stirred solution of ethyl (3,4-dimethoxybenzoyl)glycinate (1.32 g, 4.93 mmol) in methanol (20 mL) was added sodium borohydride in fractions (0.93 g, 24.6 mmol) at 0° C. Progress of the reaction was monitored by TLC. After completion of the reaction, the reaction mixture was evaporated to dryness. The residue was dissolved in ethyl acetate and washed with water. The organic layer was dried using anhydrous sodium sulfate and concentrated to give crude residue. The purification was done by flash chromatography using hexane:ethylacetate as the eluent system. Yield: 0.61 g (54.4%)

### Step 3: N-(2-chloroethyl)-3,4-dimethoxybenzamide

Procedure: To a stirred solution of N-(2-hydroxyethyl)-3,4-dimethoxybenzamide (0.6 g, 2.68 mmol) in dichloromethane (10 mL) was added triethylamine (0.76 mL, 5.37 mmol) at 0° C. After 30 minute mesyl chloride (0.42 mL, 5.37 mmol) was added in to it. The reaction mixture was allowed to stir at room temperature. Progress of the reaction was monitored by TLC. After completion of the reaction, the reaction mixture was washed with water and brine. The organic layer was dried using anhydrous sodium sulfate and concentrated to give crude residue. The purification was done by flash chromatography using hexane:ethylacetate as the eluent system. Yield: 0.27 g (40.6%)

### Step 4: N-(2-((2-amino-7H-purin-6-yl)thio)ethyl)-3,4-dimethoxybenzamide

Procedure: To a stirred solution of 2-amino-1,7-dihydro-6H-purine-6-thione (0.1 g, 0.59 mmol) in dimethylformamide (3 mL) was added potassium carbonate (0.165 g, 1.19 mmol) at 0° C. After 30 minute N-(2-chloroethyl)-3,4-dimethoxybenzamide (0.159 g, 0.657 mmol) was added in to it. The reaction mixture was allowed to stir at room temperature. Progress of the reaction was monitored by TLC. After completion of the reaction, the solvent was evaporated to dryness. The crude residue was purified by flash chromatography using dichloromethane: methanol as the eluent system. Yield: 0.045 g (20.1%).

TABLE 1
Characterization data of synthesized compounds.

| Compd. | | |
|---|---|---|
| No. | Structure | Characterization Data |
| 3 | 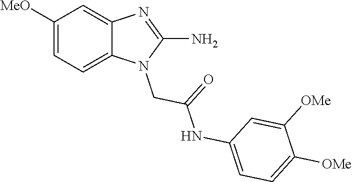 [SMILES: COc1ccc2c(c1)nc(N)n2CC(=O)Nc1ccc(OC)c(OC)c1] | White solid: Yield: 10%; ¹H NMR (400 MHz, DMSO-d₆): δ 10.18 (d, J = 3.2 Hz, 1H), 7.34 (dd, J = 6.0, 2.4 Hz, 1H), 7.08-7.04 (m, 1H), 6.96-6.74 (m, 3H), 6.61-6.50 (m, 3H), 4.78 (s, 2H), 3.70 (s, 9H): HPLC-Purity: 91.98%; LC- MS (m/z): 357.20 [M + H]+, calcd. for C₁₈H₂₀N₄O₄ m/z = 356.15. |
| 9 | 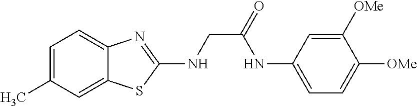 [SMILES: COc1ccc(NC(=O)CNc2nc3ccc(C)cc3s2)cc1OC] | White solid; Yield: 42%; ¹H NMR (400 MHz, DMSO-d₆): δ 9.94 (s, 1H), 9.57 (s, 1H), 7.34 (s, 1H), 7.19-7.16 (m, 2H), 7.03-7.01 (m, 2H), 6.87 (d, J = 8.8 Hz, 1H), 3.70 (s, 6H), 3.66 (s, 2H), 2.19 (s, 3H); HPLC-Purity: 98.8%; LC- MS (m/z): 358.15 [M − H]⁺, calcd. for C₁₈H₁₉N₃O₃S m/z = 357.11. |
| 10 | 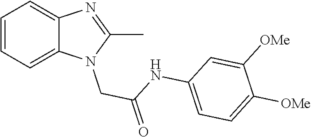 [SMILES: COc1ccc(NC(=O)Cn2c(C)nc3ccccc32)cc1OC] | White solid; Yield 16%; ¹H NMR (400 MHz, DMSO-d₆): δ 10.36 (s, 1H), 7.53 (dd, J = 7.2, 1.6 Hz, 1H), 7.44 (dd, J = 6.4, 2.0 Hz, 1H), 7.32 (d, J = 2.4 Hz, 1H) 7.19-7.04 (m, 3H), 6.90 (d, J = 8.8 Hz, 1H), 5.04 (s, 2H), 3.70 (s, 3H), 3.69 (s, 3H); LC-MS (m/z): 326.15[M + H]⁺, calcd. for C₁₈H₁₉N₃O₃ m/z = 325.14. |
| 11 | 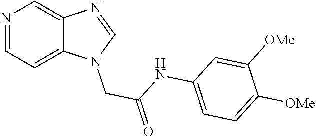 [SMILES: COc1ccc(NC(=O)Cn2cnc3cnccc32)cc1OC] | White solid; Yield 8% ; ¹H NMR (400 MHz, DMSO-d₆): δ 10.36 (s, 1H), 8.95 (s, 1H), 8.44 (s, 1 ), 8.35 (d, J = 5.6 Hz, 1H), 7.69 (d, J = 6.0 Hz, 1H), 7.32 (d, J = 4.0 Hz, 1H), 7.08 (dd, J = 8.0, 1.6 Hz, 1H), 6.91 (d, J = 8.0 Hz, 1H), 5.27 (s, 2H), 3.71 (s, 6H); LC-MS (m/z): 313.20 [M + H]⁺, calcd. for C₁₆H₁₆N₄O₃ = 312.12. |
| 18 | 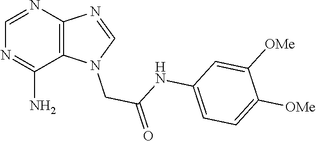 [SMILES: COc1ccc(NC(=O)Cn2cnc3ncnc(N)c32)cc1OC] | White solid; Yield 32% ; ¹H NMR (400 MHz, DMSO-d₆): δ 10.33 (s, 1H), 8.11 (s, 2H), 7.30 (d, J = 2.4 Hz, 1H), 7.23 (s, 2H), 7.05 (dd, J = 8.8, 2.4 Hz, 1H), 6.90 (d, J = 8.8 Hz, 1H), 5.02 (s, 2H), 3.71 (s, 6H); HPLC-Purity; 98.65%; LC-MS (m/z): 328.33 [M + H]⁺, calcd. for C₁₅H₁₆N₆O₃ m/z = 328.13. |
| 19 | 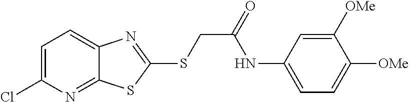 [SMILES: COc1ccc(NC(=O)CSc2nc3ccc(Cl)nc3s2)cc1OC] | White solid; Yield 10%; ¹H NMR (400 MHz, DMSO-d₆): δ 10.32 (s, 1H), 8.25 (d, J = 8.5 Hz, 1H), 7.62 (d, J = 8.5 Hz, 1H), 7.28 (d, J = 2.20 Hz, 1H), 7.08 (dd, J = 8.6, 2.3 Hz, 1H), 6.90 (d, J = 8.8 Hz, 1H), 4.40 (s, 2H) 3.71 (s, 6H); LC-MS (m/z): 396.20 [M + H]⁺, calcd. for C₁₆H₁₄ClN₃O₃S₂ m/z = 395.02. |
| 20 | 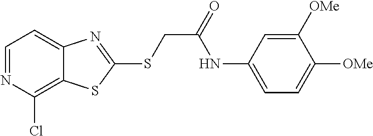 [SMILES: COc1ccc(NC(=O)CSc2nc3ccnc(Cl)c3s2)cc1OC] | White solid; Yield 30% ; ¹H NMR (400 MHz, DMSO-d₆): δ 10.31 (s, 1H), 8.24 (d, J = 8.8 Hz, 1H), 7.62 (d, J = 8.4 Hz, 1H), 7.28 (d, J = 2.0 Hz, 1H), 7.08 (dd, J = 8.8, 1.6 Hz, 1H), 6.90 (d, J = 8.8 Hz, 1H), 4.40 (s, 2H), 3.71 (s, 6H); HPLC-Purity: 98.65%; LC-MS (m/z): 396.15 [M + H]⁺, calcd. for C₁₆H₁₄ClN₃O₃S₂ m/z = 395.02. |
| 22 | 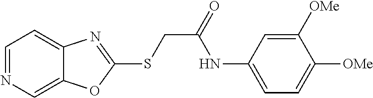 [SMILES: COc1ccc(NC(=O)CSc2nc3ccncc3o2)cc1OC] | White solid; Yield 38%; ¹H NMR (400 MHz, DMSO-d₆): δ 10.34 (s, 1H), 8.94 (s, 1H), 8.50 (d, J = 5.2 Hz, 1H), 7.70 (t, J = 5.2 Hz, 1H), 7.28 (d, J = 2.4 Hz, 1H), 7.08 (m, 1H), 6.91 (d, J = 6.4 Hz, 1H), 4.41 (s, 2H), 3.71 (s, 6H); LC-MS (m/z): 346.20 [M + H]⁺, calcd. for |
| | | C₁₆H₁₅N₃O₄S m/z = 345.08. |
| 23 | 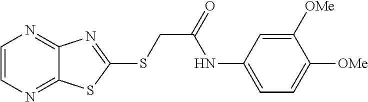 [SMILES: COc1ccc(NC(=O)CSc2nc3nccnc3s2)cc1OC] | White solid; Yield: 42%; ¹H NMR (400 MHz, DMSO-d₆): δ 10.36 (s, 1H), 8.67 (d, J = 2.4 Hz, 1H), 8.54 (d, J = 2.4 Hz, 1H), 7.29 (d, J = 2.0 Hz, 1H), 7.08 (dd, J = 8.8, 2.4 Hz, 1H), 6.90 (d, J = 8.4 Hz, 1H), 4.47 (s, 2H), 3.71 (s, 6H); HPLC-Purity: 97.8%; LC-MS (m/z): 363.20 [M + H]⁺, calcd. for C₁₅H₁₄N₄O₃S₂ |
| | | m/z = 362.05. |
| 25 | 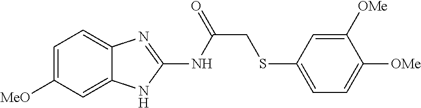 [SMILES: COc1ccc2nc(NC(=O)CSc3ccc(OC)c(OC)c3)[nH]c2c1] | White solid; Yield 38%; ¹H NMR (400 MHz, DMSO-d₆): δ 11.92 (s, 1H), 11.52 (s, 1H), 7.28 (dd, J = 8.0, 1.6 Hz, 1H), 7.16 (s, 1H), 7.05-6.98 (m, 2H), 6.88 (d, J = 8.4 Hz, 1H), 6.70 (d, J = 8.8 Hz, 1H), 3.83 (s, 2H), 3.74 (s, 9H); HPLC-Purity: 93.31%; LC-MS (m/z): 374.25 [M + H]⁺, calcd. for C₁₈H₁₉N₃O₄S |
| | | m/z = 373.11. |
| 30 | 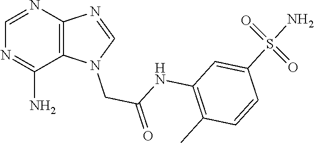 [SMILES: Cc1ccc(S(N)(=O)=O)cc1NC(=O)Cn1cnc2ncnc(N)c21] | White solid; Yield 27%; ¹H NMR (400 MHz, DMSO-d₆): δ 9.97 (s, 1H), 8.14 (s, 2H), 7.96 (d, J = 4.0 Hz, 1H), 7.53 (dd, J = 8.0, 2.0 Hz, 1H), 7.41 (d, J = 8.0 Hz, 1H), 7.31 (s, 2H), 7.23 (s, 1H), 5.15 (s, 2H), 2.50 (s, 3H); HPLC-Purity; 98.65%; LC-MS (m/z): 362.20 [M + H]⁺, calcd. for C₁₄H₁₅N₇O₃S m/z = 361.10. |
| 32 | 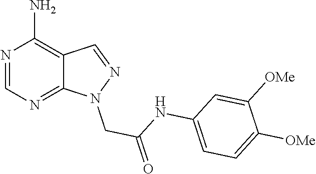 [SMILES: COc1ccc(NC(=O)Cn2ncc3c(N)ncnc32)cc1OC] | White solid; Yield 37% ; ¹H NMR (400 MHz, DMSO-d₆): δ 10.35 (s, 1H), 8.39 (s, 1H), 8.13 (s, 1H), 7.78 (s, 1H), 7.55 (s, 1H), 7.31 (d, J = 2.4 Hz, 1H), 7.08 (dd, J = 8.8, 2.4 Hz, 1H), 6.92 (d, J = 8.8 Hz, 1H), 5.23 (s, 2H), 3.71 (s, 6H); LC-MS (m/z): 329.15 [M + H]⁺, calcd. for C₁₅H₁₆N₆O₃ m/z = 328.13. |
| 33 | 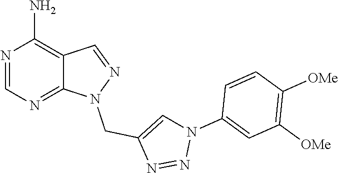 [SMILES: COc1ccc(-n2cc(Cn3ncc4c(N)ncnc43)nn2)cc1OC] | White solid; Yield 8%; ¹H NMR (400 MHz, DMSO-d₆): δ 9.04 (s, 1H), 8.67 (s, 1H), 8.22 (s, 1H), 8.11 (s, 2H), 7.42(m, 2H), 7.10 (d, J = 8.8 Hz, 1H), 5.62 (s, 2H), 3.71(m, 6H); LC-MS (m/z): 391.10 [M + K]⁺, calcd. for C₁₆H₁₆N₈O₂ m/z = 352.14. |
| 34 | 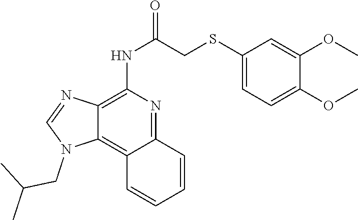 [SMILES: COc1ccc(SCC(=O)Nc2nc3ccccc3c3c2ncn3CC(C)C)cc1OC] | White solid; Yield 13%; ¹H NMR (400 MHz, DMSO-d₆): δ 10.34 (s, 1H), 8.40 (s, 1H), 8.25 (d, J = 7.83 Hz, 1H), 7.95 (d, J = 7.34 Hz, 1H), 7.68-7.60 (m, 2H), 7.09 (d, J = 1.9 Hz, 1H), 7.02 (dd, J = 8.3, 2.2 Hz, 1H), 6.87 (d, J = 8.3Hz, 1H), 4.50 (d, J = 7.3 Hz, 2H), 4.15 (s, 2H), 3.70 (d, J = 1.4 Hz, 6H), 2.32-2.04 (m, 1H), 0.93 (d, J = 6.6 Hz, 6H): LC-MS (m/z): 451.30 [M + H]⁺, calcd. for C₂₄H₂₆N₄O₃S m/z = 450.17. |
| 36 | 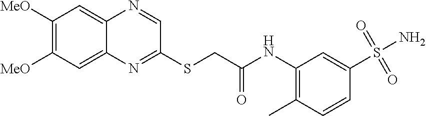 [SMILES: COc1cc2ncc(SCC(=O)Nc3cc(S(N)(=O)=O)ccc3C)nc2cc1OC] | White solid; Yield: 31%; ¹H NMR (400 MHz, DMSO-d₆): δ 9.87 (s, 1H), 8.69 (s, 1H), 7.93 (d, J = 2.0 Hz, 1H), 7.52 (dd, J = 8.0, 2.0 Hz, 1H), 7.40-7.39 (m, 2H), 7.31 (s, 2H), 7.27 (s, 1H), 4.28 (s, 2H), 3.94 (s, 6H), 2.24 (s, 3H); HPLC-Purity: 94.77%; LC-MS (m/z): 449.10 [M + H]⁺, calcd. for C₁₉H₂₀N₄O₅S₂ m/z = |
| | | 448.02. |
| 37 | 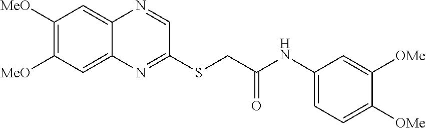 [SMILES: COc1ccc(NC(=O)CSc2cnc3cc(OC)c(OC)cc3n2)cc1OC] | White solid; Yield: 27%; ¹H NMR (400 MHz, DMSO-d₆): δ 10.22 (s, 1H), 8.67 (s, 1H), 7.37 (s, 1H), 7.31 (d, J = 2.4 Hz, 2H), 7.24 (s, 1H), 7.09 (dd, J = 8.4, 2.4 Hz, 1H), 6.88 (d, J = 8.8 Hz, 1H), 4.19 (s, 2H), 3.93 (s, 3H), 3.91 (s, 3H), 3.70 (s, 6H); HPLC-Purity: 96.92%; LC-MS (m/z): 416.10 [M + H]⁺, calcd. for C₂₀H₂₁N₃O₅S m/z = 415.12. |
| 40 | 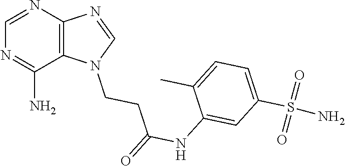 [SMILES: Cc1ccc(S(N)(=O)=O)cc1NC(=O)CCn1cnc2ncnc(N)c21] | White solid; Yield: 56%; ¹H NMR (400 MHz, DMSO-d₆): δ 9.56 (s, 1H), 8.16 (s, 1H), 8.06 (s, 1H), 7.88 (d, J = 1.2 Hz, 1H), 7.50 (dd, J = 8.0, 1.6 Hz, 1H), 7.36 (d, J = 8.0 Hz, 1H), 7.30 (s, 2H), 7.20 (s, 2H), 4.46 (t, J = 6.8 Hz, 2H), 3.01 (t, J = 6.8 Hz, 2H), 2.11 (s, 3H); LC-MS (m/z): 376.15 [M + H]⁺, calcd. for C₁₅H₁₇N₇O₃S m/z = 375.11. |
| 42 | 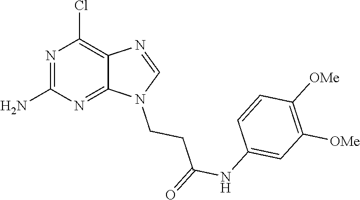 [SMILES: COc1ccc(NC(=O)CCn2cnc3c(Cl)nc(N)nc32)cc1OC] | White solid; Yield: 15%; ¹H NMR (400 MHz, DMSO-d₆): δ 9.86 (s, 1H), 8.04 (s, 1H), 7.20 (d, J = 2.4 Hz, 1H), 7.02 (dd, J = 8.8, 2.4 Hz, 1H), 6.95 (s, 2H), 6.85 (d, J = 8.8 Hz, 1H), 4.33 (t, J = 6.4 Hz, 2H), 3.69 (s, 6H), 2.85 (t, J = 6.4 Hz, 2H); HPLC-Purity: 93.45%; LC-MS (m/z): 377.15 [M + H]⁺, calcd. for C₁₆H₁₇ClN₆O₃ m/z = 376.11. |
| 45 | 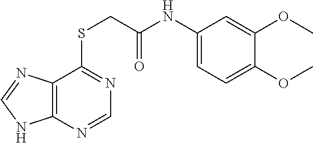 [SMILES: COc1ccc(NC(=O)CSc2ncnc3[nH]cnc23)cc1OC] | White solid; Yield: 85%; ¹H NMR (400 MHz, DMSO-d₆): δ 13.56 (s, 1H), 10.22 (s, 1H), 8.67 (s, 1H), 8.46 (s, 1H), 7.30 (d, J = 2.0 Hz, 1H), 7.07 (dd, J = 8.4, 2.0 Hz, 1H), 6.88 (d, J = 8.8 Hz, 1H), 4.30 (s, 2H), 3.70 (s, 6H); HPLC-Purity: 95.6%; LC-MS (m/z): 346.15 [M + H]⁺, calcd. for C₁₃H₁₀F₂N₆O₂S m/z = 345.09. |
| 46 | 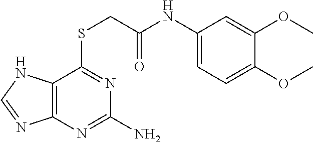 [SMILES: COc1ccc(NC(=O)CSc2nc(N)nc3nc[nH]c23)cc1OC] | White solid; Yield 28% ¹H NMR (400 MHz, DMSO-d₆): δ 12.60 (s, 1H), 10.06 (s, 1H), 7.94 (s, 1H), 7.29 (d, J = 2.2 Hz, 1H), 7.10 (dd, J = 8.6, 2.3 Hz, 1H), 6.88 (d, J = 8.8 Hz, 1H), 6.51 (s, 2H), 4.10 (s, 2H), 3.70 (s, 6H); HPLC-Purity; 96.68%; LC-MS (m/z): 361.15 [M + H]⁺, calcd. for C₁₅H₁₆N₆O₃S m/z = 360.10. |
| 52 | 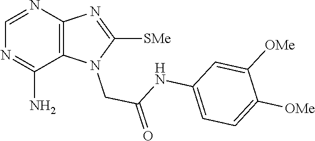 [SMILES: COc1ccc(NC(=O)Cn2c(SC)nc3ncnc(N)c32)cc1OC] | White solid; Yield 22%; ¹H NMR (400 MHz, DMSO-d₆): δ 10.36 (s, 1H), 8.06 (s, 1H), 7.29 (d, J = 2.4 Hz, 1H), 7.15 (s, 2H), 7.04 (dd, J = 8.5, 2.2 Hz, 1H), 6.91 (d, J = 8.8 Hz, 1H), 5.12 (s, 2H), 3.70 (s, 6H), 2.56 (s, 3H); LC- MS (m/z): 375.15 [M + H]⁺, calcd. For C₁₆H₁₈N₆O₃S m/z = 374.12. |
| 56 | 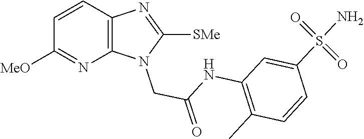 [SMILES: COc1ccc2nc(SC)n(CC(=O)Nc3cc(S(N)(=O)=O)ccc3C)c2n1] | White solid; Yield: 13%; ¹H NMR (400 MHz, DMSO-d₆): δ 10.04 (s, 1H), 7.93 (d, J = 1.6 Hz, 1H), 7.90 (d, J = 8.4 Hz, 1H), 7.54 (dd, J = 8.0, 2.0 Hz, 1H), 7.42 (d, J = 8.0 Hz, 1H), 7.32 (s, 2H), 6.66 (d, J = 8.4 Hz, 1H), 5.05 (s, 2H), 3.89 (s, 3H), 2.66 (s, 3H), 2.32 (s, 3H): HPLC-Purity; 96.32%; LC-MS (m/z): 422.10 [M + H]⁺, calcd. for C₁₇H₁₉N₅O₄S₂ m/z = 421.09. |
| 57 | 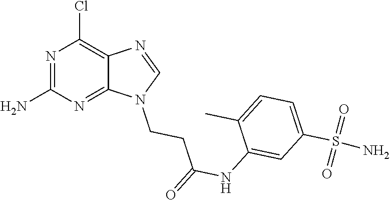 [SMILES: Cc1ccc(S(N)(=O)=O)cc1NC(=O)CCn1cnc2c(Cl)nc(N)nc21] | White solid; Yield: 28%; ¹H NMR (400 MHz, DMSO-d₆): δ 9.58 (s, 1H), 8.06 (s, 1H), 7.88 (s, 1H), 7.52-7.50 (d, J = 8.0 Hz, 1H), 7.37 (d, J = 8.0 Hz, 1H), 7.32 (s, 2H), 6.95 (s, 2H), 4.35 (t, J = 6.8 Hz, 2H), 2.98 (t, J = 6.8 Hz, 2H), 2.13 (s, 3H); LC-MS (m/z): 410.10 [M + H]⁺, calcd. for C₁₅H₁₆ClN₇O₃S m/z = 409.07. |
| 63 | 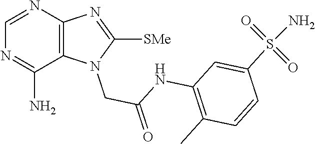 [SMILES: CSc1nc2ncnc(N)c2n1CC(=O)Nc1cc(S(N)(=O)=O)ccc1C] | White solid; Yield 26%; ¹H NMR (400 MHz, DMSO-d₆): δ 10.08 (s, 1H), 8.22 (s, 1H), 7.93 (s, 1H), 7.73 (s, 1H), 7.53 (d, J = 7.6 Hz, 1H), 7.42 (d, J = 7.6 Hz, 1H), 7.31 (s, 2H), 7.14 (s, 2H), 5.23 (s, 2H), 2.57 (s, 3H), 2.32 (s, 3H); LC-MS (m/z): 408.10 [M + H]⁺, calcd. for C₁₅H₁₇N₇O₃S₂ m/z = 407.08. |
| 66 | 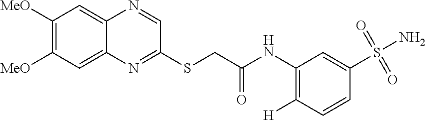 [SMILES: COc1cc2ncc(SCC(=O)Nc3cccc(S(N)(=O)=O)c3)nc2cc1OC] | White solid; Yield 38.70%; ¹H NMR (400 MHz, DMSO-d₆): δ 10.69 (s, 1H), 8.68 (s, 1H), 8.18 (s, 1H), 7.77 (s, 1H), 7.51 (d, J = 2.5 Hz, 2H), 7.37 (s, 3H), 7.19 (s, 1H), 4.24 (s, 2H), 3.93 (s, 3H), 3.88 (s, 3H); HPLC- Purity; 99.20%; LC-MS (m/z): 435.15 [M + H]⁺, calcd. for C₁₈H₁₈N₄O₅S₂ m/z = 434.07 |
| 69 | 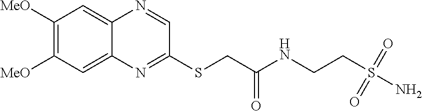 [SMILES: COc1cc2ncc(SCC(=O)NCCS(N)(=O)=O)nc2cc1OC] | White solid; Yield 72.63%; ¹H NMR (400 MHz, DMSO-d₆): δ 8.63 (s, 1H), 8.36 (s, 1H), 7.37 (s, 1H), 7.27 (s, 1H), 6.90 (s, 2H), 3.98 (s, 2H), 3.95 (s, 3H), 3.93 (s, 3H), 3.47 (t, J = 6.4 Hz, 2H), 3.12 (t, J = 6.8 Hz, 2H); LC-MS (m/z): 387.15 [M + H]⁺, calcd. for C₁₄H₁₈N₄O₅S₂ m/z = 386.07. |
| 70 | 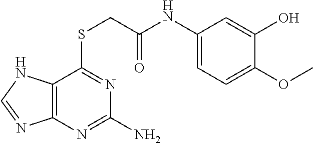 [SMILES: COc1ccc(NC(=O)CSc2nc(N)nc3nc[nH]c23)cc1O] | White solid; Yield 64%; ¹H NMR (400 MHz, DMSO-d₆): δ 12.60 (s, 1H), 9.93 (s, 1H), 9.04 (s, 1H), 7.94 (s, 1H), 7.16 (d, J = 1.9 Hz, 1H), 6.92 (dd, J = 8.6, 2.3 Hz, 1H), 6.82 (d, J = 8.8 Hz, 1H), 6.48 (s, 2H), 4.08 (s, 2H), 3.70 (s, 3H); LC-MS (m/z): 347.15 [M + H]⁺, calcd. for C₁₄H₁₄N₆O₃S m/z = 346.08. |
| 71 | 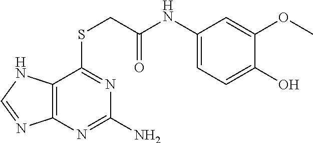 [SMILES: COc1cc(NC(=O)CSc2nc(N)nc3nc[nH]c23)ccc1O] | White solid; Yield 66%; ¹H NMR (400 MHz, DMSO-d₆): δ 12.60 (s, 1H), 9.97 (s, 1H), 8.75 (s, 1H), 7.93 (s, 1H), 7.26 (d, J = 1.2 Hz, 1H), 6.95 (dd, J = 8.8, 1.6 Hz, 1H), 6.69 (d, J = 8.8 Hz, 1H), 6.51 (s, 2H), 4.08 (s, 2H), 3.71 (s, 3H); HPLC-Purity; 91.75%; LC-MS (m/z): 347.15 [M + H]⁺, calcd. for C₁₄H₁₄N₆O₃S m/z = 346.08. |
| 72 | 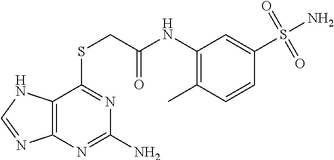 [SMILES: Cc1ccc(S(N)(=O)=O)cc1NC(=O)CSc1nc(N)nc2nc[nH]c12] | White solid; Yield 15% ; ¹H NMR (400 MHz, DMSO-d₆): δ ppm 12.62 (s, 1H), 9.66 (s, 1H), 8.02 (s, 1H), 7.95 (s, 1H), 7.52 (d, J = 8.4 Hz, 1H), 7.37 (d, J = 8.4 Hz, 1H), 7.32 (s, 3H), 6.51 (s, 2H), 4.19 (s, 2H), 2.17 (s, 3H); LC- MS (m/z): 394.15 [M + H]⁺, calcd. for C₁₄H₁₅N₇O₃S₂ m/z = 393.07. |
| 83 | 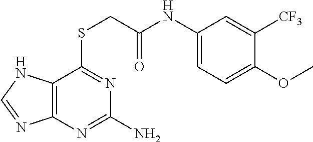 [SMILES: COc1ccc(NC(=O)CSc2nc(N)nc3nc[nH]c23)cc1C(F)(F)F] | White solid; Yield: 76%; ¹H NMR (400 MHz, DMSO-d₆): δ 12.59 (s, 1H), 10.30 (s, 1H), 7.93 (s, 2H), 7.77 (dd, J = 11.6 2.8 Hz, 1H), 7.24 (d, J = 12.0 Hz, 1H), 6.47 (s, 2H), 4.12 (s, 2H), 3.84 (s, 3H); LC-MS (m/z): 399.10 [M + H]⁺, calcd. for C₁₅H₁₃F₃N₆O2_{S} m/z = 398.08. |
| 84 | 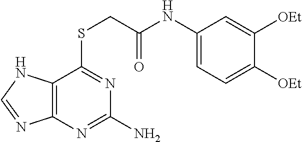 [SMILES: CCOc1ccc(NC(=O)CSc2nc(N)nc3nc[nH]c23)cc1OCC] | White solid; Yield: 39% ; ¹H NMR (400 MHz, DMSO-d₆): δ 12.60 (s, 1H), 10.03 (s, 1H), 7.94 (s, 1H), 7.27 (s, 1H), 7.06 (dd, J = 8.4, 1.6 Hz, 1H), 6.86 (d, J = 8.8 Hz, 1H), 6.51 (s, 2H), 4.08 (s, 2H), 3.97-3.92 (m, 4H), 1.33- 1.26 (m, 6H): LC-MS (m/z): 389.20 [M + H]⁺, calcd. for C₁₅H₁₃F₃N₆O₂S m/z = 388.13. |
| 85 | 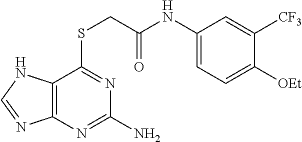 [SMILES: CCOc1ccc(NC(=O)CSc2nc(N)nc3nc[nH]c23)cc1C(F)(F)F] | White solid; Yield: 25%; ¹H NMR (400 MHz, DMSO-d₆): δ 12.60 (s, 1H), 10.29 (s, 1H), 7.93 (s, 2H), 7.75 (dd, J = 9.2, 1.2 Hz, 1H), 7.22 (d, J = 9.2 Hz, 1H), 6.47 (s, 2H), 4.14- 4.08 (m, 4H), 1.30 (t, J = 6.8 Hz, 3H); LC- MS (m/z): 413.15 [M + H]⁺, calcd. for C₁₇H₂₀N₆O₃S m/z = 412.09. |
| 86 | 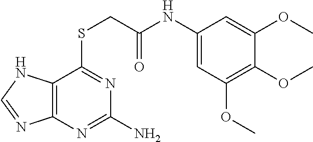 [SMILES: COc1cc(NC(=O)CSc2nc(N)nc3nc[nH]c23)cc(OC)c1OC] | White solid; Yield 15%; ¹H NMR (400 MHz, DMSO-d₆): δ 12.61 (s, 1H), 10.16 (s, 1H), 7.94 (s, 1H), 6.99 (s, 2H), 6.52 (s, 2H), 4.10 (s, 2H), 3.72 (s, 6H), 3.60 (s, 3H); HPLC- Purity: 96.50%; LC-MS (m/z): 391.15 [M + H]⁺, calcd. for C₁₆H₁₅F₃N₆O₂S m/z = 390.11. |
| 87 | 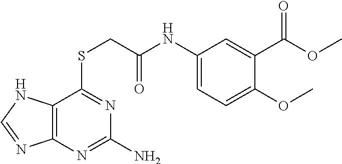 [SMILES: COC(=O)c1cc(NC(=O)CSc2nc(N)nc3nc[nH]c23)ccc1OC] | White solid; Yield: 42%; ¹H NMR (400 MHz, DMSO-d₆): δ 12.59 (s, 1H), 10.20 (s, 1H), 7.95-7.93 (m, 2H), 7.70 (dd, J = 8.8, 2.4 Hz, 1H), 7.11 (d, J = 8.9 Hz, 1H), 6.46 (s, 2H), 4.11 (s, 2H), 3.77 (s, 6H): HPLC-Purity: 95.01%; LC-MS (m/z): 389.2 [M + H]⁺, calcd. for C₁₆H₁₆N₆O₄S m/z = 388.10. |
| 88 | 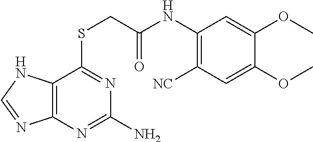 [SMILES: COc1cc(C#N)c(NC(=O)CSc2nc(N)nc3nc[nH]c23)cc1OC] | White solid; Yield: 13%; ¹H NMR (400 MHz, DMSO-d₆): δ 12.59 (s, 1H), 10.24 (s, 1H), 7.94 (s, 1H), 7.32 (s, 1H), 7.18 (s, 1H), 6.41 (s, 2H), 4.17 (s, 2H), 3.78 (s, 6H): LC-MS (m/z): 386.2 [M + H]⁺, calcd. for C₁₆H₁₆N₆O₄S m/z = 385.10. |
| 89 | 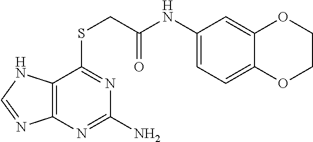 [SMILES: Nc1nc(SCC(=O)Nc2ccc3c(c2)OCCO3)c2[nH]cnc2n1] | White solid; Yield: 88%; ¹H NMR (400 MHz, DMSO-d₆): δ 12.60 (s, 1H), 10.01 (s, 1H), 7.93 (s, 1H), 7.19 (s, 1H), 6.97(dd, J = 8.8, 2.4 Hz 1H), 6.77 (d, J = 8.4 Hz, 1H), 6.48 (s, 2H), 4.19 (m, 4H), 4.07 (s, 2H); LC-MS (m/z): 359.10 [M + H]⁺, calcd. for C₁₅H₁₄N₆O₃S m/z = 358.08. |
| 101 | 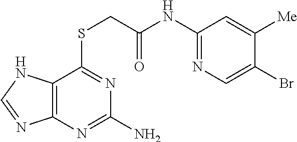 [SMILES: Cc1cc(NC(=O)CSc2nc(N)nc3nc[nH]c23)ncc1Br] | White solid; Yield 15%: ¹H NMR (400 MHz, DMSO-d₆): δ 12.60 (s, 1H), 10.82 (s, 1H), 8.40 (s, 1H), 8.05 (s, 1H), 7.94 (s, 1H), 6.35 (s, 2H), 4.24 (s, 2H), 2.34 (s, 3H); LC-MS (m/z): 394.00 [M + H]⁺, calcd. for C₁₃H₁₂BrN₇OS m/z = 393.00. |
| 103 | 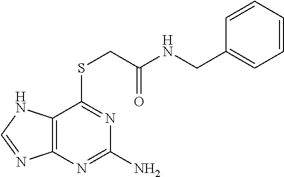 [SMILES: Nc1nc(SCC(=O)NCc2ccccc2)c2[nH]cnc2n1] | White solid; Yield 54%; ¹H NMR (400 MHz, DMSO-d₆): δ 8.78 (s, 1H), 8.37 (s, 1H), 7.48 (d, J = 7.2 Hz, 1H), 7.49-7.37 (m, 2H), 7.35- 7.23 (m, 5H), 4.31 (d, J = 6.0 Hz, 2H), 4.13 (s, 2H); LC-MS (m/z): 315.1 [M + H]⁺, calcd. for C₁₄H₁₄N₆OS m/z = 314.09. |
| 108 | 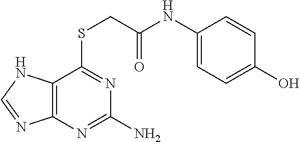 [SMILES: Nc1nc(SCC(=O)Nc2ccc(O)cc2)c2[nH]cnc2n1] | White solid; Yield 66%; ¹H NMR (400 MHz, DMSO-d₆): δ 12.60 (s, 1H), 9.93 (s, 1H), 9.21 (s, 1H), 7.94 (s, 1H), 7.36 (d, J = 8.8 Hz, 2H), 6.68 (d, J = 8.8 Hz, 2H), 6.49 (s, 2H), 4.08 (s, 2H); LC-MS (m/z): 317.1 [M + H]⁺, calcd. for C₁₃H₁₂N₆O₂S m/z = 316.07. |
| 110 | 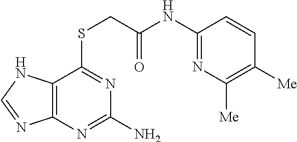 [SMILES: Cc1ccc(NC(=O)CSc2nc(N)nc3nc[nH]c23)nc1C] | White solid; Yield- 61%; ¹H NMR (400 MHz, DMSO-d₆): δ 12.59 (s, 1H), 10.06 (s, 1H), 7.93 (s, 1H), 7.73 (d, J = 10.8 Hz, 1H), 7.49 (d, J = 11.2 Hz, 1H), 6.38 (s, 2H), 4.20 (s, 2H), 2.35 (s, 3H), 2.19 (s, 3H); LC-MS (m/z): 330.05 [M + H]⁺, calcd. for C₁₄H₁₅N₇OS m/z = 329.11. |
| 117 | 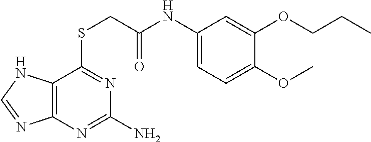 [SMILES: CCCOc1cc(NC(=O)CSc2nc(N)nc3nc[nH]c23)ccc1OC] | White solid; Yield 28%; ¹H NMR (400 MHz, DMSO-d₆): δ 12.60 (s, 1H), 10.04 (s, 1H), 7.94 (s, 1H), 7.30 (d, J = 3.2 Hz, 1H), 7.08 (dd, J = 14.4, 2.8 Hz, 1H), 6.88 (d, J = 11.6 Hz, 1H), 6.50 (s, 2H), 4.10 (s, 2H), 3.85 (t, J = 8.8 Hz, 2H), 3.71 (s, 3H), 1.72 (q, J = 18.8, 9.2 Hz, 2H), 0.97 (t, J = 9.6 Hz, 3H); HPLC-Purity: 99.4%; LC-MS (m/z): 389.2 [M + H]⁺, calcd. for C₁₇H₂₀N₆O₃S m/z = |
| | | 388.13. |
| 125 | 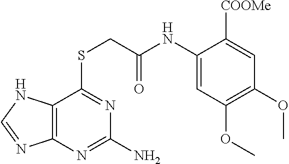 [SMILES: COC(=O)c1cc(OC)c(OC)cc1NC(=O)CSc1nc(N)nc2nc[nH]c12] | White solid; Yield 42%; ¹H NMR (400 MHz, DMSO-d₆): δ 12.60 (s, 1H), 11.17 (s, 1H), 8.19 (s, 1H), 7.94 (s, 1H), 7.39 (s, 1H), 6.33 (s, 2H), 4.21 (s, 2H), 3.80 (s, 6H), 3.76 (s, 3H); LC-MS (m/z): 419.2 [M + H]⁺, calcd. for C₁₇H₁₈N₆O₅S m/z = 418.11. |
| 126 | 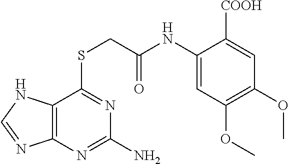 [SMILES: COc1cc(NC(=O)CSc2nc(N)nc3nc[nH]c23)c(C(=O)O)cc1OC] | White solid; Yield 10%; ¹H NMR (400 MHz, DMSO-d₆): δ 13.52 (s, 1H), 11.68 (s, 1H), 8.31 (s, 1H), 8.04 (s, 1H), 7.41 (s, 1H), 6.44 (s, 2H), 4.21(s, 2H), 3.80 (s, 3H), 3.74 (s, 3H); LC-MS (m/z): 405.2 [M + H]⁺, calcd. for C₁₆H₁₆N₆O₅S m/z = 404.09. |
| 127 | 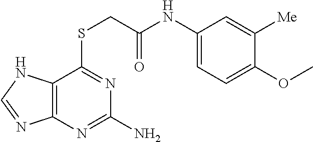 [SMILES: COc1ccc(NC(=O)CSc2nc(N)nc3nc[nH]c23)cc1C] | White solid; Yield: 49%; ¹H NMR (400 MHz, DMSO-d₆): δ 12.58 (s, 1H), 9.96 (s, 1H), 7.93 (s, 1H), 7.36 (d, J = 11.6 Hz, 2H), 6.86 (d, J = 11.6 Hz, 1H), 6.47 (s, 2H), 4.09 (s, 2H), 3.73 (s, 3H), 2.10 (s, 3H); LC-MS (m/z): 345.1 [M + H]⁺, calcd. for C₁₅H₁₆N₆O₂S m/z = 344.11. |
| 128 | 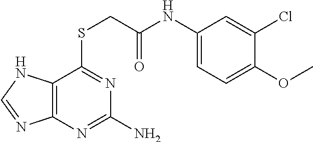 [SMILES: COc1ccc(NC(=O)CSc2nc(N)nc3nc[nH]c23)cc1Cl] | White solid; Yield 45%; ¹H NMR (400 MHz, DMSO-d₆): δ 12.62 (s, 1H), 10.19 (s, 1H), 7.94 (s, 1H), 7.74 (d, J = 2.4 Hz, 1H), 7.45 (dd, J = 8.8, 2.4 Hz, 1H), 7.11 (d, J = 8.8 Hz, 1H), 6.48 (s, 2H), 4.11 (s, 2H), 3.81 (s, 3H); LC-MS (m/z): 365.05 [M + H]⁺, calcd. for C₁₃H₁₀F₂N₆O₂S m/z = 364.05. |
| 130 | 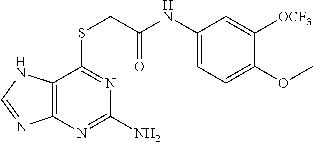 | White solid; Yield: 33%; ¹H NMR (400 MHz, DMSO-d₆): δ 12.58 (s, 1H), 10.03 (s, 1H), 7.93 (s, 1H), 7.27 (s, 1H), 7.09 (dd, J = 11.6, 2.4 Hz, 1H), 6.87 (d, J = 11.6 Hz, 1H), 6.49 (s, 2H), 4.09 (s, 2H), 3.98-3.91 (m, 2H), 3.70 (s, 3H), 1.31 (t, J = 8.8 Hz, 3H); LC-MS (m/z): 375.2 [M + H]⁺, calcd. for C₁₆H₁₅F₃N₆O₂S m/z = 374.12 |
| 135 | 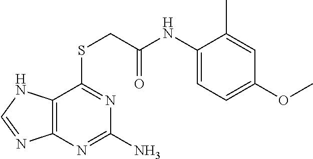 [SMILES: COc1ccc(NC(=O)CSc2nc(N)nc3nc[nH]c23)c(C)c1] | White solid; Yield 33%; ¹H NMR (400 MHz, DMSO-d₆): δ 12.60 (s, 1H), 9.36 (s, 1H), 7.94 (s, 1H), 7.25 (d, J = 12.0 Hz, 1H), 6.74 (t, J = 9.2 Hz, 2H), 6.48 (s, 2H), 4.10 (s, 2H), 3.70 (s, 3H), 2.06 (s, 3H); LC-MS (m/z): 345.05 [M + H] calcd. for C₁₅H₁₆N₆O₂S m/z = 344.11. |
| 136 | 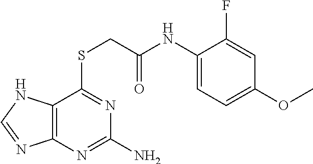 [SMILES: COc1ccc(NC(=O)CSc2nc(N)nc3nc[nH]c23)c(F)c1] | White solid; Yield: 75%; ¹H NMR (400 MHz, DMSO-d₆): δ 12.61 (s, 1H), 9.84 (s, 1H), 7.94 (s, 1H), 7.64 (t, J = 12.0 Hz, 1H), 6.87 (dd, J = 16.8, 3.6 Hz, 1H), 6.76-6.72 (dd, J = 12 Hz, 2.4 Hz, 1H), 6.43 (s, 2H), 4.14 (s, 2H), 3.73 (s, 3H); LC-MS (m/z): 349.05 [M − H]⁺, calcd. for C₁₄H₁₃FN₆O₂S m/z = 348.08. |
| 137 | 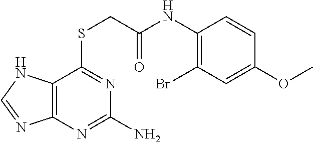 [SMILES: COc1ccc(NC(=O)CSc2nc(N)nc3nc[nH]c23)c(Br)c1] | White solid; Yield 40%; ¹H NMR (400 MHz, DMSO-d₆): δ 12.61 (s, 1H), 9.56 (s, 1H), 7.95 (s, 1H), 7.51 (d, J = 8.8 Hz, 1H), 7.20 (d, J = 2.4 Hz, 1H), 6.95 (dd, J = 9.2, 2.8 Hz, 1H) 6.45 (s, 2H), 4.16 (s, 2H), 3.75 (s, 3H); LC- MS (m/z): 408.95 [M + H]⁺, calcd. for C₁₂H₁₂N₈O₂S m/z = 408.00. |
| 138 | 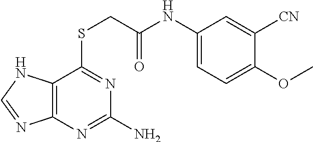 [SMILES: COc1ccc(NC(=O)CSc2nc(N)nc3nc[nH]c23)cc1C#N] | White solid; Yield 44% ¹H NMR (400 MHz, DMSO-d₆): δ 12.60 (s, 1H), 10.33 (s, 1H), 7.94-7.91 (m, 2H), 7.81-7.78 (m, 1H), 7.23 (d, J = 9.2 Hz 1H), 6.47 (s, 2H), 4.12 (s, 2H), 3.87 (s, 3H); LC-MS (m/z): 356.05 [M + H], calcd. for C₁₅H₁₃N₇O₂S m/z = 355.09. |
| 143 | 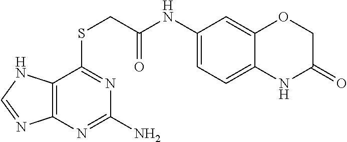 [SMILES: Nc1nc(SCC(=O)Nc2ccc3c(c2)OCC(=O)N3)c2[nH]cnc2n1] | White solid; Yield 9%; ¹H NMR (400 MHz, DMSO-d₆): δ 12.60 (s, 1H), 10.73 (s, 1H), 10.15 (s, 1H), 7.93 (s, 1H) 7.39 (d, J = 2.4 Hz, 1H), 7.00 (dd, J = 8.4, 2.4 Hz, 1H), 6.88 (d, J = 8.8 Hz, 1H), 6.44 (s, 2H), 4.50 (s, 2H), 4.17 (s, 2H); LC-MS (m/z): 372.20 [M + H]⁺, calcd. for C₁₅H₁₃N₇O₃S m/z = 371.08. |
| 146 | 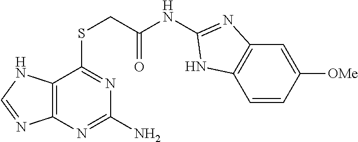 [SMILES: COc1ccc2[nH]c(NC(=O)CSc3nc(N)nc4nc[nH]c34)nc2c1] | White solid; Yield 50%; ¹H NMR (400 MHz, DMSO-d₆): δ 12.59 (s, 1H), 11.91 (s, 1H), 11.71 (S, 1H), 7.94 (s, 1H), 7.30 (d, J = 8.4 Hz, 1H), 6.98 (s, 1H), 6.70 (s, 1H), 6.35 (s, 2H), 4.28 (s, 2H), 3.74 (s, 3H); LC-MS (m/z): 371.2 [M + H]⁺, calcd. for C₁₅H₁₃N₇O₃S m/z = 370.10. |
| 147 | 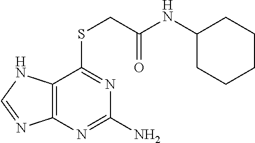 [SMILES: Nc1nc(SCC(=O)NC2CCCCC2)c2[nH]cnc2n1] | White solid; Yield: 55%; ¹H NMR (400 MHz, DMSO-d₆): δ 12.56 (s, 1H), 7.94-7.91 (d, J = 10.8 Hz, 2H), 6.39 (s, 2H), 3.88 (s, 2H), 1.70- 1.50 (m, 5H), 1.33-1.22 (m, 6H); LC-MS (m/z): 305.2 [M − H]⁺, calcd. for C₁₄H₁₅N₇OS m/z = 306.13. |
| 148 | 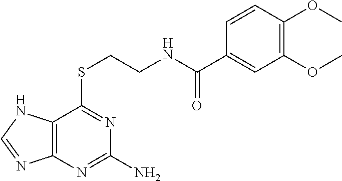 [SMILES: COc1ccc(C(=O)NCCSc2nc(N)nc3nc[nH]c23)cc1OC] | White solid; Yield: 21%; ¹H NMR (400 MHz, DMSO-d₆): δ 12.52 (s, 1H), 8.54 (t, J = 5.3 Hz, 1H), 7.89 (s, 1H), 7.46 (dd, J = 8.4, 2.0 Hz, 1H), 7.42 (d, J = 2.0 Hz, 1H), 7.00 (d, J = 8.4 Hz, 1H), 6.35 (s, 2H), 3.79 (s, 6H), 3.78- 3.56 (m, 2H), 3.45 (t, J = 6.4 Hz, 2H); HPLC- Purity: 95.6%; LC-MS (m/z): 375.10 [M + H]⁺, calcd. for C₁₆H₁₈N₆O₃S m/z = 374.12. |
| 151 | 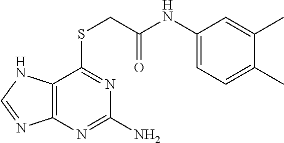 [SMILES: Cc1ccc(NC(=O)CSc2nc(N)nc3nc[nH]c23)cc1C] | White solid; Yield 67%; ¹H NMR (400 MHz, DMSO-d₆): δ 12.60 (s, 1H), 10.03 (s, 1H), 7.94 (s, 1H), 7.36 (s, 1H), 7.29 (d, J = 8.4 Hz, 1H), 7.04 (d, J = 8.4 Hz, 1H), 6.48 (s, 2H), 4.11 (s, 2H), 2.16 (s, 3H), 2.14 (s, 3H); LC-MS (m/z): 329.05 [M + H]⁺, calcd. for C₁₅H₁₆N₆OS m/z = 328.11. |
| 163 | 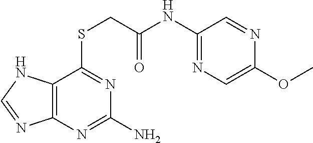 [SMILES: COc1cnc(NC(=O)CSc2nc(N)nc3nc[nH]c23)cn1] | White solid; Yield 5%; ¹H NMR (400 MHz, DMSO-d₆): δ 12.60 (s, 1H), 10.80 (s, 1H), 8.80 (s, 1H), 8.12 (d, J = 1.6 Hz, 1H), 7.94 (s, 1H), 6.37 (s, 2H), 4.25 (s, 2H), 3.88 (s, 3H); LC-MS (m/z): 333.10 [M + H]⁺, calcd. for C₁₂H₁₂N₈O₂S m/z = 332.08. |
| 167 | 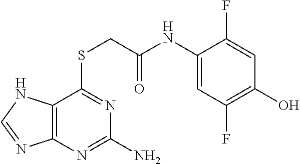 [SMILES: Nc1nc(SCC(=O)Nc2cc(F)c(O)cc2F)c2[nH]cnc2n1] | White solid; Yield 23%; ¹H NMR (400 MHz, DMSO-d₆): δ 12.62 (s, 1H), 10.19 (s, 1H), 9.93 (s, 1H), 7.94 (s, 1H), 7.65-7.60 (m, 1H), 6.83-6.78 (m, 1H), 6.45 (s, 2H), 4.11 (s, 2H); LC-MS (m/z): 353.10 [M + H]⁺, calcd. for C₁₃H₁₀F₂N₆O₂S m/z = 352.06. |
| 168 | 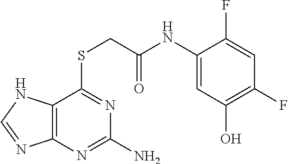 [SMILES: Nc1nc(SCC(=O)Nc2cc(O)c(F)cc2F)c2[nH]cnc2n1] | White solid; Yield: 85%; ¹H NMR (400 MHz, DMSO-d₆): δ 12.63 (s, 1H), 9.95 (s, 1H), 9.88 (s, 1H), 7.97 (s, 1H), 7.58 (t, J = 8.4 Hz, 1H), 7.23 (t, J = 11.2 Hz, 1H), 6.45 (s, 2H), 4.12 (s, 2H); LC-MS (m/z): 353.10 [M + H], calcd. for C₁₃H₁₀F₂N₆O₂S m/z = 352.06. |
| 176 | 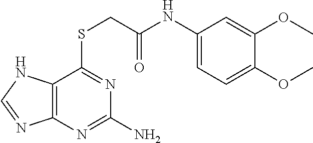 | White solid; Yield: 51%; ¹H NMR (400 MHz, DMSO-d₆): δ 12.52 (s, 1H), 7.89 (s, 1H), 6.72 (d, J = 8.4 Hz, 1H), 6.32 (s, 2H), 6.31 (d, J = 2.8 Hz, 1H), 6.13 (dd, J = 8.8, 2.8 Hz, 1H), 5.49 (s, 1H), 3.67 (s, 3H), 3.61 (s, 3H), 3.42 (t, J = 6.4 Hz, 2H), 1.23 (d, J = 8.4 Hz, 1H); LC-MS (m/z): 347.10 [M + H]⁺, calcd. for C₁₅H₁₈N₆O₂S m/z = 346.12. |
| 205 | 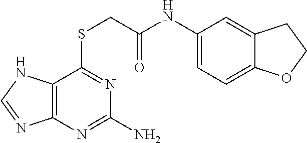 [SMILES: Nc1nc(SCC(=O)Nc2ccc3c(c2)CCO3)c2[nH]cnc2n1] | White solid; Yield 34%; ¹H NMR (400 MHz, DMSO-d₆): δ 12.59 (s, 1H), 9.97 (s, 1H), 7.93 (s, 1H), 7.49 (d, J = 1.6 Hz, 1H), 7.22 (dd, J = 8.4, 2 Hz, 1H), 6.67 (d, J = 8.8 Hz, 1H), 6.47 (s, 2H), 4.48 (t, J = 8.4 Hz, 2H), 4.11 (t, J = 6.0 Hz, 2H), 3.15 (t, J = 8.4 Hz, 2H); LC-MS (m/z): 343.10 [M + H]⁺, calcd. for C₁₃H₁₀F₂N₆O₂S m/z = 342.09. |

Biological Evaluation

### Example 6: Colorimetric NPP Assay Using p-Nitrophenyl-5-TMP as a Substrate

Ectonucleotide Pyrophosphatase (ENPP1) belongs to the ecto-nucleotide pyrophosphatase/phosphodiesterase (ENPP) family. ENPP1 is a type II transmembrane glycoprotein that hydrolyzes nucleotides and nucleotide derivatives with the formation of nucleotide-5′-monophosphates. The ENPP1 inhibitors were synthesized and assessed for ENPP1 inhibition potency through a colorimetric assay using Thymidine 5′-monophosphate p-nitrophenyl ester (5′-TMP-pNP) as a substrate. ENPP1 hydrolyzes 5′-TMP-pNP to form a chromogenic product p-nitrophenolate. The amount of p-nitrophenolate formed is directly proportional to the ENPP1 enzyme activity and was measured using its absorbance at 405 nm.

The enzyme inhibition assays were performed in a clear 96-well microplate. The reaction mixture contains different concentrations of ENPP1 inhibitor and 20 ng human ENPP1 in 1 mM CaCl₂, 200 μM ZnCl₂, 50 mM Tris, pH 9.0. This reaction mixture was pre-incubated for 10 minutes at 37° C. and absorbance was measured at 405 nm as a pre-read using microplate reader. The reaction was then initiated by the addition of 5′-TMP-pNP substrate at a final concentration of 400 μM and kept for incubation for 20 min at 37° C. Thereafter, the enzymatic reaction was terminated by the addition of 20 μL of 1.0 N NaOH. The amount of released p-nitrophenolate was measured at 405 nm (as post-read). Appropriate control for test sample and respective blank controls were taken to eliminate background absorbance. The incubation and operation conditions remain the same as described above. Percentage inhibition was determined for different concentrations of the test inhibitor by comparing the absorbance of inhibitor versus blank.

The IC50 values were determined by plotting the percent inhibition versus inhibitor concentration curves using a three-parameter non-linear regression curve fit in GraphPad Prism® software. Ki values were derived from the IC50 values using the Cheng-Prusoff equation:

Ki=IC50/(1+[TMP-pNP]/Km),
where routinely [TMP-pNP]=400 μM Michaelis-Menten constant (Km) value for 5′-TMP-pN is 222 μM

### Example 7: Capillary Electrophoresis-Based NPP Assay with ATP as a Substrate

ENPP1 is a eukaryotic protein with broad substrate specificity, being capable of hydrolyzing nucleotides, for example, ATP to AMP. Here, a Capillary Electrophoresis based method was used to determine the IC50 values for ENPP1 Inhibitors for ENNP1 protein.

The inhibitors synthesized were assessed for ENPP1 inhibitory potency against the natural substrate ATP. The enzyme inhibition assays were performed in 10 mM 2-(N-cyclohexylamino)-ethanesulfonic acid (CHES) buffer (pH 10.0) including 1 mM MgCl₂, 2 mM CaCl₂), and 400 μM of ATP, with different inhibitor concentrations. The reaction mixture was incubated with 20 ng human NPP1 at 37° C. for 30 min in a final volume of 100 μL, and the reactions were stopped by heating at 90° C. for 3 min. Finally, the reaction mixtures were directly measured by capillary electrophoresis (CE). The CE instrumentation and operating conditions were as follows: P/ACE MDQ capillary electrophoresis system (Beckman Instruments, Fullerton, Calif., USA) with a DAD detection system, polyacrylamide-coated capillaries of 40 cm effective length×50 m (id) obtained from CS Chromatographie GmbH (Langerwehe, Germany), 50 mM phosphate buffer (pH 6.5) as running buffer, electrokinetic injection (−6 kV, 60 s), separation voltage of −15 kV. The amounts of AMP produced were measured at 260 nm. Data collection and peak area analysis were performed by 32 Karat software obtained from Beckman Coulter (Fullerton, Calif., USA).

The IC50 values of test compounds were calculated by plotting data in the program Prism 5.0, and the Ki values were calculated from the IC50 values with the Cheng-Prusoff equation (Tables 2-4).

Ki=IC50/(1+[ATP]/Km),
where routinely [ATP]=400 μM and Michaelis-Menten constant (Km) value for ATP is 8.17 μM.

TABLE 2
In vitro data for evaluated compounds.
Compounds with ENPP1 Inhibition (Kᵢ) ≤ 100 nM

| | |
|---|---|
| 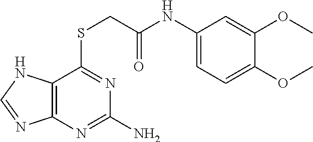 [SMILES: COc1ccc(NC(=O)CSc2nc(N)nc3nc[nH]c23)cc1OC] | (46) |
| 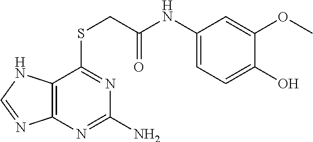 [SMILES: COc1cc(NC(=O)CSc2nc(N)nc3nc[nH]c23)ccc1O] | (71) |
| 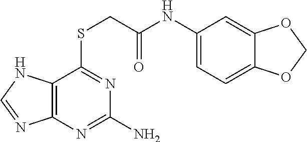 [SMILES: Nc1nc(SCC(=O)Nc2ccc3c(c2)OCO3)c2[nH]cnc2n1] | (78) |
| 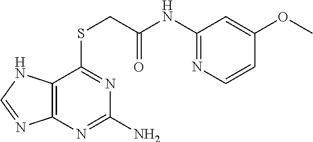 [SMILES: COc1ccnc(NC(=O)CSc2nc(N)nc3nc[nH]c23)c1] | (109) |
| 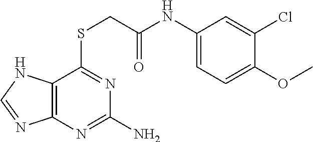 [SMILES: COc1ccc(NC(=O)CSc2nc(N)nc3nc[nH]c23)cc1Cl] | (128) |
| 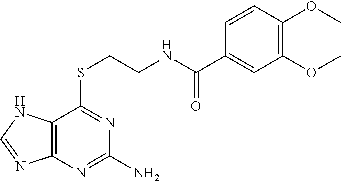 [SMILES: COc1ccc(C(=O)NCCSc2nc(N)nc3nc[nH]c23)cc1OC] | (148) |
| 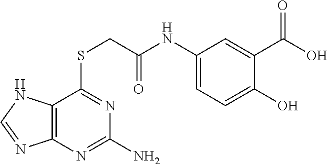 [SMILES: Nc1nc(SCC(=O)Nc2ccc(O)c(C(=O)O)c2)c2[nH]cnc2n1] | (155) |
| 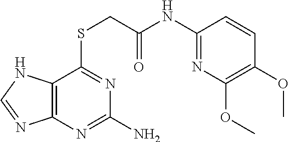 [SMILES: COc1ccc(NC(=O)CSc2nc(N)nc3nc[nH]c23)nc1OC] | (160) |
| 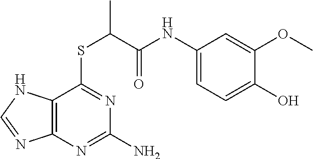 [SMILES: COc1cc(NC(=O)C(C)Sc2nc(N)nc3nc[nH]c23)ccc1O] | (173) |
| 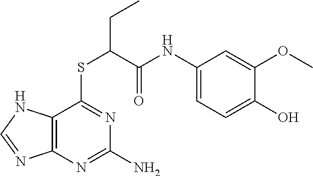 [SMILES: CCC(Sc1nc(N)nc2nc[nH]c12)C(=O)Nc1ccc(O)c(OC)c1] | (174) |
| 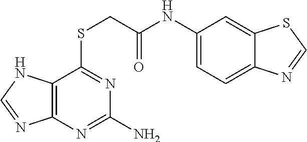 [SMILES: Nc1nc(SCC(=O)Nc2ccc3ncsc3c2)c2[nH]cnc2n1] | (181) |
| 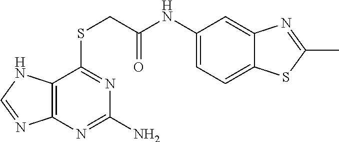 [SMILES: Cc1nc2cc(NC(=O)CSc3nc(N)nc4nc[nH]c34)ccc2s1] | (182) |
| 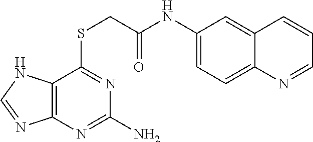 [SMILES: Nc1nc(SCC(=O)Nc2ccc3ncccc3c2)c2[nH]cnc2n1] | (184) |
| 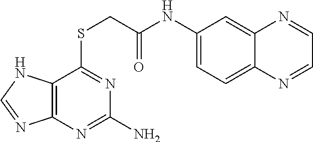 [SMILES: Nc1nc(SCC(=O)Nc2ccc3nccnc3c2)c2[nH]cnc2n1] | (185) |
| 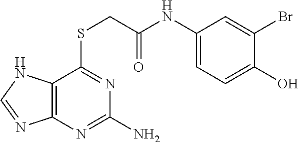 [SMILES: Nc1nc(SCC(=O)Nc2ccc(O)c(Br)c2)c2[nH]cnc2n1] | (194) |
| 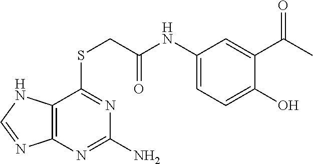 [SMILES: CC(=O)c1cc(NC(=O)CSc2nc(N)nc3nc[nH]c23)ccc1O] | (199) |
| 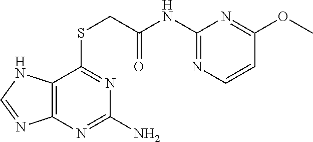 [SMILES: COc1ccnc(NC(=O)CSc2nc(N)nc3nc[nH]c23)n1] | (201) |
| 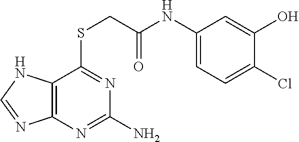 [SMILES: Nc1nc(SCC(=O)Nc2ccc(Cl)c(O)c2)c2[nH]cnc2n1] | (207) |
| 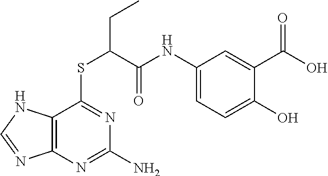 [SMILES: CCC(Sc1nc(N)nc2nc[nH]c12)C(=O)Nc1ccc(O)c(C(=O)O)c1] | (249) |
| 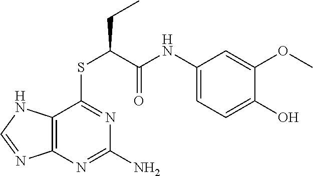 [SMILES: CC[C@H](Sc1nc(N)nc2nc[nH]c12)C(=O)Nc1ccc(O)c(OC)c1] | (260) |
| 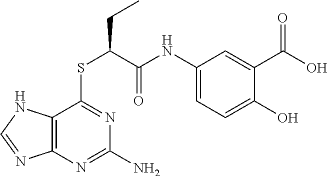 [SMILES: CC[C@H](Sc1nc(N)nc2nc[nH]c12)C(=O)Nc1ccc(O)c(C(=O)O)c1] | (267) |
| 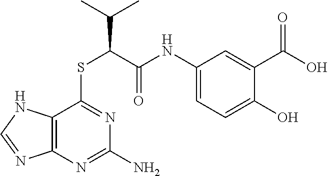 [SMILES: CC(C)[C@H](Sc1nc(N)nc2nc[nH]c12)C(=O)Nc1ccc(O)c(C(=O)O)c1] | (269) |
| 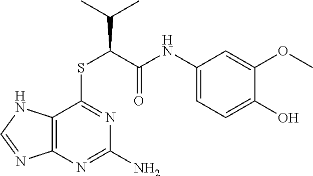 [SMILES: COc1cc(NC(=O)[C@@H](Sc2nc(N)nc3nc[nH]c23)C(C)C)ccc1O] | (270) |
| 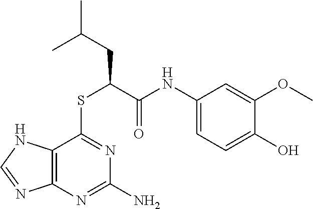 [SMILES: COc1cc(NC(=O)[C@H](CC(C)C)Sc2nc(N)nc3nc[nH]c23)ccc1O] | (272) |
| 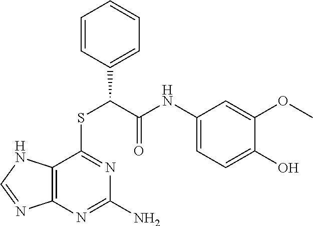 [SMILES: COc1cc(NC(=O)[C@H](Sc2nc(N)nc3nc[nH]c23)c2ccccc2)ccc1O] | (329) |
| 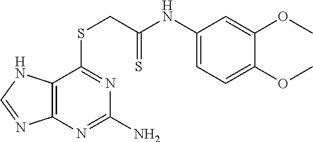 [SMILES: COc1ccc(NC(=S)CSc2nc(N)nc3nc[nH]c23)cc1OC] | (355) |
| 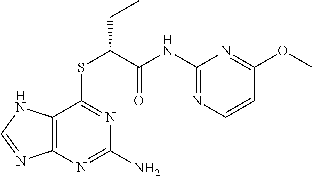 [SMILES: CC[C@@H](Sc1nc(N)nc2nc[nH]c12)C(=O)Nc1nccc(OC)n1] | (389) |
| 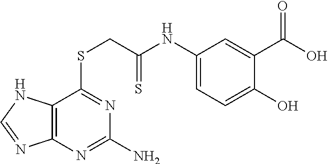 [SMILES: Nc1nc(SCC(=S)Nc2ccc(O)c(C(=O)O)c2)c2[nH]cnc2n1] | (408) |
| 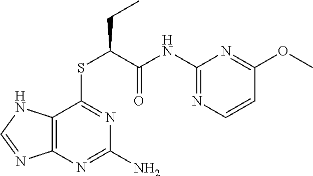 [SMILES: CC[C@H](Sc1nc(N)nc2nc[nH]c12)C(=O)Nc1nccc(OC)n1] | (435) |
| 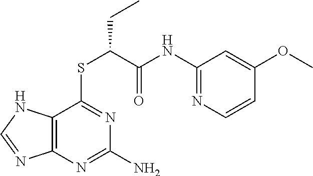 [SMILES: CC[C@@H](Sc1nc(N)nc2nc[nH]c12)C(=O)Nc1cc(OC)ccn1] | (471) |
| 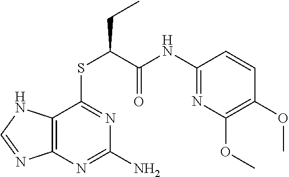 [SMILES: CC[C@H](Sc1nc(N)nc2nc[nH]c12)C(=O)Nc1ccc(OC)c(OC)n1] | (472) |
| 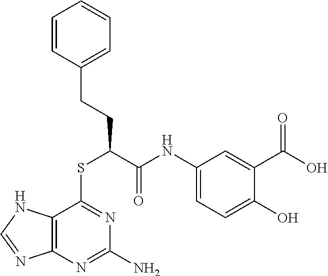 [SMILES: Nc1nc(S[C@@H](CCc2ccccc2)C(=O)Nc2ccc(O)c(C(=O)O)c2)c2[nH]cnc2n1] | (485) |
| 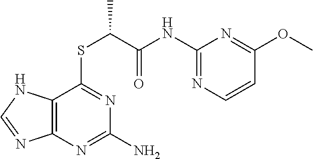 [SMILES: COc1ccnc(NC(=O)[C@@H](C)Sc2nc(N)nc3nc[nH]c23)n1] | (486) |
| 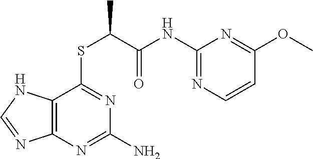 [SMILES: COc1ccnc(NC(=O)[C@H](C)Sc2nc(N)nc3nc[nH]c23)n1] | (487) |
| 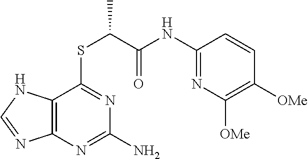 [SMILES: COc1ccc(NC(=O)[C@@H](C)Sc2nc(N)nc3nc[nH]c23)nc1OC] | (490) |
| 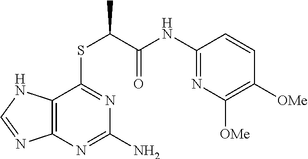 [SMILES: COc1ccc(NC(=O)[C@H](C)Sc2nc(N)nc3nc[nH]c23)nc1OC] | (491) |
| 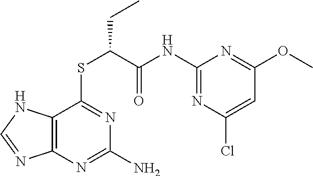 [SMILES: CC[C@@H](Sc1nc(N)nc2nc[nH]c12)C(=O)Nc1nc(Cl)cc(OC)n1] | (496) |
| 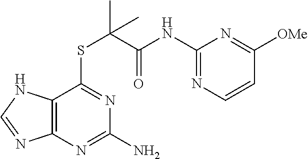 [SMILES: COc1ccnc(NC(=O)C(C)(C)Sc2nc(N)nc3nc[nH]c23)n1] | (504) |
| 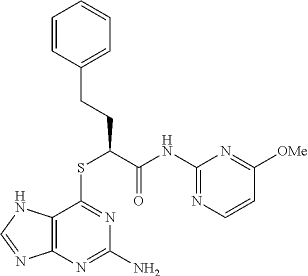 [SMILES: COc1ccnc(NC(=O)[C@H](CCc2ccccc2)Sc2nc(N)nc3nc[nH]c23)n1] | (506) |
| 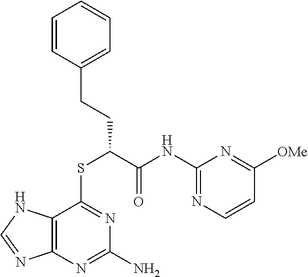 [SMILES: COc1ccnc(NC(=O)[C@@H](CCc2ccccc2)Sc2nc(N)nc3nc[nH]c23)n1] | (507) |
| 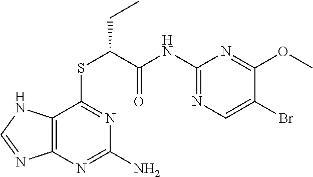 [SMILES: CC[C@@H](Sc1nc(N)nc2nc[nH]c12)C(=O)Nc1ncc(Br)c(OC)n1] | (508) |
| 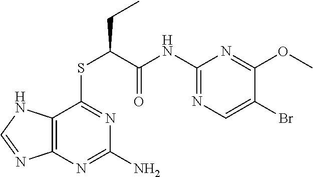 [SMILES: CC[C@H](Sc1nc(N)nc2nc[nH]c12)C(=O)Nc1ncc(Br)c(OC)n1] | (509) |
| 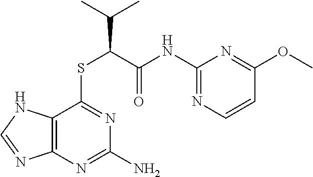 [SMILES: COc1ccnc(NC(=O)[C@@H](Sc2nc(N)nc3nc[nH]c23)C(C)C)n1] | (510) |
| 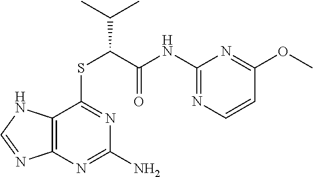 [SMILES: COc1ccnc(NC(=O)[C@H](Sc2nc(N)nc3nc[nH]c23)C(C)C)n1] | (511) |
| 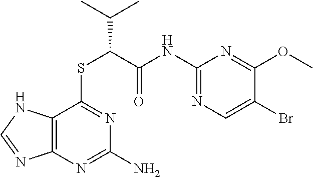 [SMILES: COc1nc(NC(=O)[C@H](Sc2nc(N)nc3nc[nH]c23)C(C)C)ncc1Br] | (514) |
| 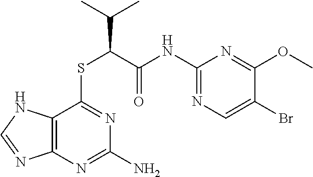 [SMILES: COc1nc(NC(=O)[C@@H](Sc2nc(N)nc3nc[nH]c23)C(C)C)ncc1Br] | (515) |
| 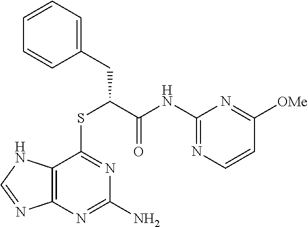 [SMILES: COc1ccnc(NC(=O)[C@@H](Cc2ccccc2)Sc2nc(N)nc3nc[nH]c23)n1] | (516) |
| 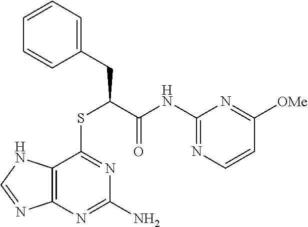 [SMILES: COc1ccnc(NC(=O)[C@H](Cc2ccccc2)Sc2nc(N)nc3nc[nH]c23)n1] | (517) |
| 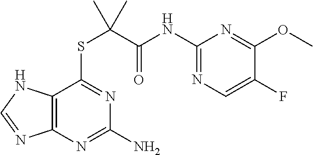 [SMILES: COc1nc(NC(=O)C(C)(C)Sc2nc(N)nc3nc[nH]c23)ncc1F] | (529) |
| 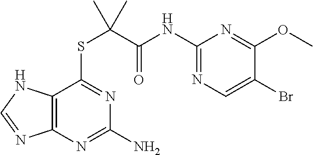 [SMILES: COc1nc(NC(=O)C(C)(C)Sc2nc(N)nc3nc[nH]c23)ncc1Br] | (530) |
| 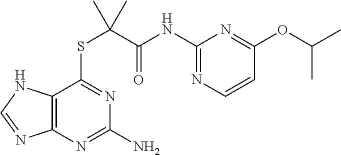 [SMILES: CC(C)Oc1ccnc(NC(=O)C(C)(C)Sc2nc(N)nc3nc[nH]c23)n1] | (534) |
| 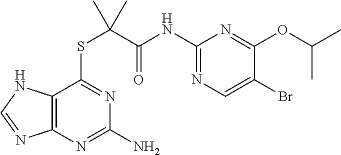 [SMILES: CC(C)Oc1nc(NC(=O)C(C)(C)Sc2nc(N)nc3nc[nH]c23)ncc1Br] | (543) |
| 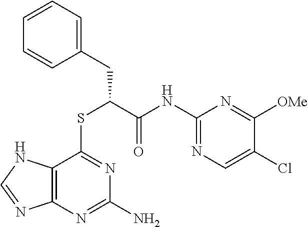 [SMILES: COc1nc(NC(=O)[C@@H](Cc2ccccc2)Sc2nc(N)nc3nc[nH]c23)ncc1Cl] | (551) |
| 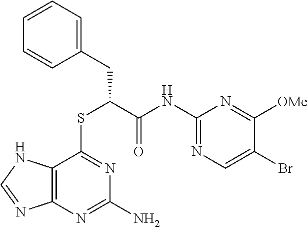 [SMILES: COc1nc(NC(=O)[C@@H](Cc2ccccc2)Sc2nc(N)nc3nc[nH]c23)ncc1Br] | (552) |
| 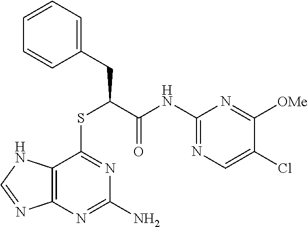 [SMILES: COc1nc(NC(=O)[C@H](Cc2ccccc2)Sc2nc(N)nc3nc[nH]c23)ncc1Cl] | (554) |
| 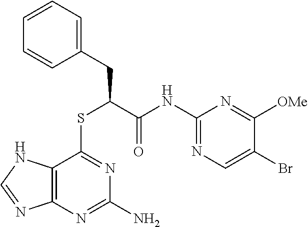 [SMILES: COc1nc(NC(=O)[C@H](Cc2ccccc2)Sc2nc(N)nc3nc[nH]c23)ncc1Br] | (555) |
| 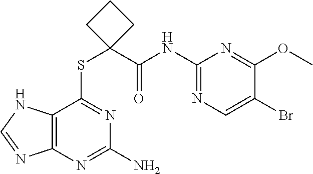 [SMILES: COc1nc(NC(=O)C2(Sc3nc(N)nc4nc[nH]c34)CCC2)ncc1Br] | (558) |
| 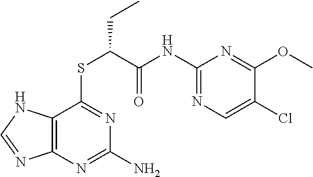 [SMILES: CC[C@@H](Sc1nc(N)nc2nc[nH]c12)C(=O)Nc1ncc(Cl)c(OC)n1] | (608) |
| 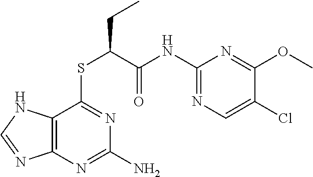 [SMILES: CC[C@H](Sc1nc(N)nc2nc[nH]c12)C(=O)Nc1ncc(Cl)c(OC)n1] | (609) |
| 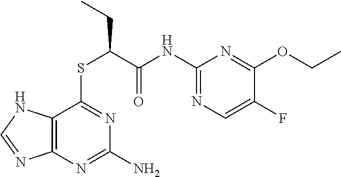 [SMILES: CCOc1nc(NC(=O)[C@H](CC)Sc2nc(N)nc3nc[nH]c23)ncc1F] | (673) |
| 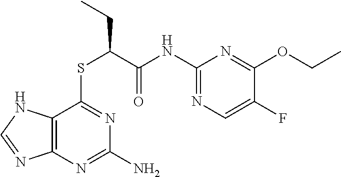 [SMILES: CCOc1nc(NC(=O)[C@H](CC)Sc2nc(N)nc3nc[nH]c23)ncc1F] | (676) |
| 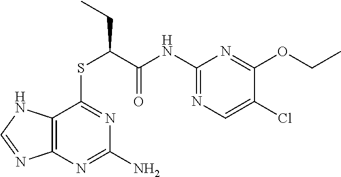 [SMILES: CCOc1nc(NC(=O)[C@H](CC)Sc2nc(N)nc3nc[nH]c23)ncc1Cl] | (677) |
| 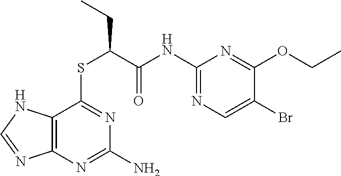 [SMILES: CCOc1nc(NC(=O)[C@H](CC)Sc2nc(N)nc3nc[nH]c23)ncc1Br] | (678) |
| 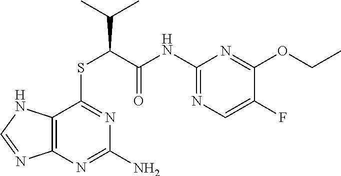 [SMILES: CCOc1nc(NC(=O)[C@@H](Sc2nc(N)nc3nc[nH]c23)C(C)C)ncc1F] | (679) |
| 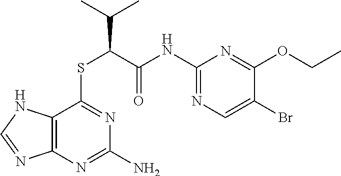 [SMILES: CCOc1nc(NC(=O)[C@@H](Sc2nc(N)nc3nc[nH]c23)C(C)C)ncc1Br] | (681) |
| 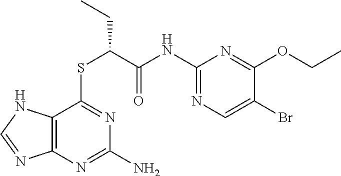 [SMILES: CCOc1nc(NC(=O)[C@@H](CC)Sc2nc(N)nc3nc[nH]c23)ncc1Br] | (712) |

TABLE 3
In vitro data for evaluated compounds.
Compounds with ENPP1 Inhibition (Kᵢ) between 100 nM and 1 μM

| | |
|---|---|
| 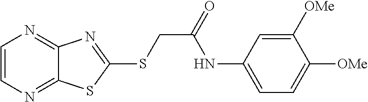 [SMILES: COc1ccc(NC(=O)CSc2nc3nccnc3s2)cc1OC] | (23) |
| 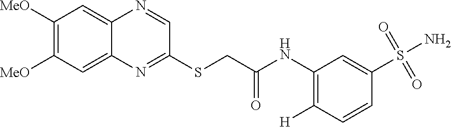 [SMILES: COc1cc2ncc(SCC(=O)Nc3cccc(S(N)(=O)=O)c3)nc2cc1OC] | (66) |
| 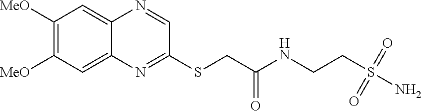 [SMILES: COc1cc2ncc(SCC(=O)NCCS(N)(=O)=O)nc2cc1OC] | (69) |
| 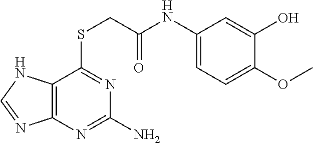 [SMILES: COc1ccc(NC(=O)CSc2nc(N)nc3nc[nH]c23)cc1O] | (70) |
| 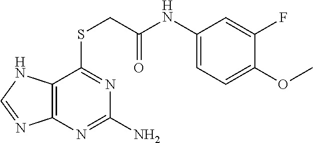 [SMILES: COc1ccc(NC(=O)CSc2nc(N)nc3nc[nH]c23)cc1F] | (81) |
| 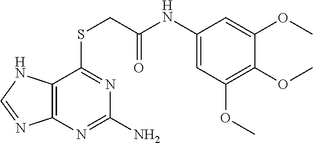 [SMILES: COc1cc(NC(=O)CSc2nc(N)nc3nc[nH]c23)cc(OC)c1OC] | (86) |
| 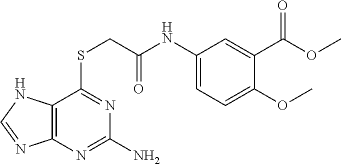 [SMILES: COC(=O)c1cc(NC(=O)CSc2nc(N)nc3nc[nH]c23)ccc1OC] | (87) |
| 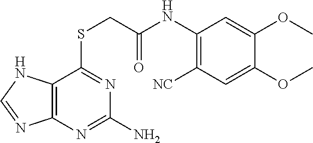 [SMILES: COc1cc(C#N)c(NC(=O)CSc2nc(N)nc3nc[nH]c23)cc1OC] | (88) |
| 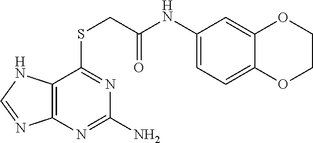 [SMILES: Nc1nc(SCC(=O)Nc2ccc3c(c2)OCCO3)c2[nH]cnc2n1] | (89) |
| 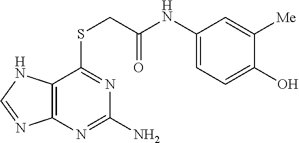 [SMILES: Cc1cc(NC(=O)CSc2nc(N)nc3nc[nH]c23)ccc1O] | (91) |
| 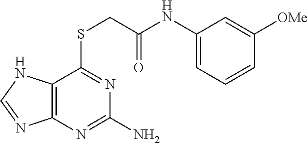 [SMILES: COc1cccc(NC(=O)CSc2nc(N)nc3nc[nH]c23)c1] | (106) |
| 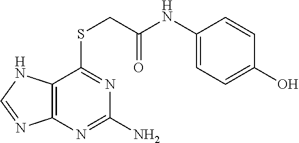 [SMILES: Nc1nc(SCC(=O)Nc2ccc(O)cc2)c2[nH]cnc2n1] | (108) |
| 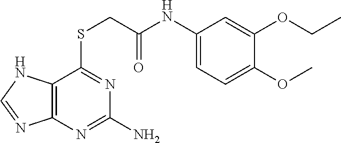 [SMILES: CCOc1cc(NC(=O)CSc2nc(N)nc3nc[nH]c23)ccc1OC] | (116) |
| 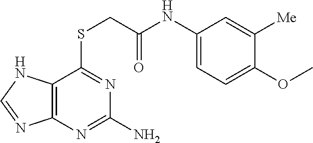 [SMILES: COc1ccc(NC(=O)CSc2nc(N)nc3nc[nH]c23)cc1C] | (127) |
| 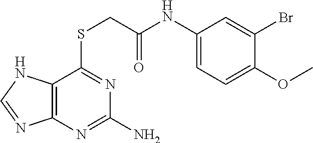 [SMILES: COc1ccc(NC(=O)CSc2nc(N)nc3nc[nH]c23)cc1Br] | (129) |
| 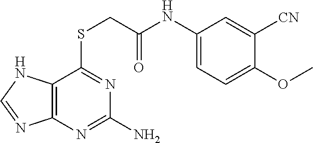 [SMILES: COc1ccc(NC(=O)CSc2nc(N)nc3nc[nH]c23)cc1C#N] | (138) |
| 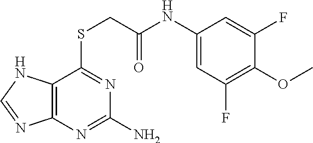 [SMILES: COc1c(F)cc(NC(=O)CSc2nc(N)nc3nc[nH]c23)cc1F] | (139) |
| 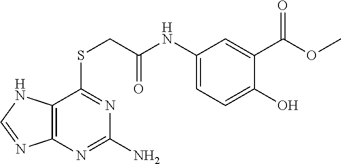 [SMILES: COC(=O)c1cc(NC(=O)CSc2nc(N)nc3nc[nH]c23)ccc1O] | (154) |
| 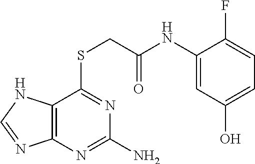 [SMILES: Nc1nc(SCC(=O)Nc2cc(O)ccc2F)c2[nH]cnc2n1] | (165) |
| 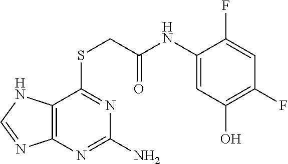 [SMILES: Nc1nc(SCC(=O)Nc2cc(O)c(F)cc2F)c2[nH]cnc2n1] | (168) |
| 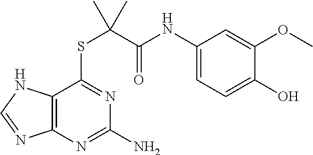 [SMILES: COc1cc(NC(=O)C(C)(C)Sc2nc(N)nc3nc[nH]c23)ccc1O] | (172) |
| 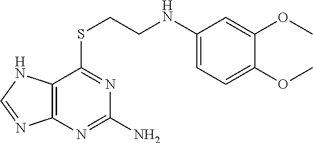 [SMILES: COc1ccc(NCCSc2nc(N)nc3nc[nH]c23)cc1OC] | (176) |
| 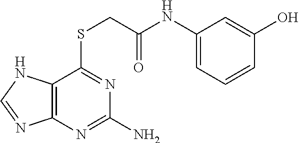 [SMILES: Nc1nc(SCC(=O)Nc2cccc(O)c2)c2[nH]cnc2n1] | (192) |
| 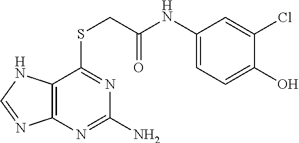 [SMILES: Nc1nc(SCC(=O)Nc2ccc(O)c(Cl)c2)c2[nH]cnc2n1] | (193) |
| 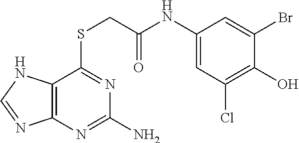 [SMILES: Nc1nc(SCC(=O)Nc2cc(Cl)c(O)c(Br)c2)c2[nH]cnc2n1] | (195) |
| 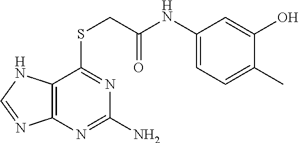 [SMILES: Cc1ccc(NC(=O)CSc2nc(N)nc3nc[nH]c23)cc1O] | (197) |
| 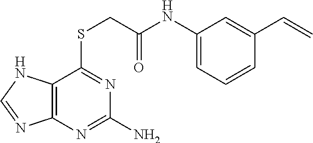 [SMILES: C=Cc1cccc(NC(=O)CSc2nc(N)nc3nc[nH]c23)c1] | (200) |
| 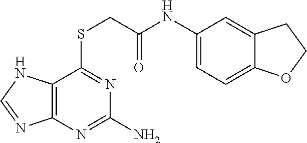 [SMILES: Nc1nc(SCC(=O)Nc2ccc3c(c2)CCO3)c2[nH]cnc2n1] | (205) |
| 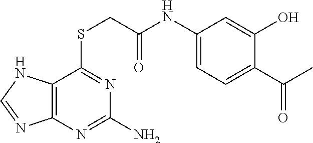 [SMILES: CC(=O)c1ccc(NC(=O)CSc2nc(N)nc3nc[nH]c23)cc1O] | (209) |
| 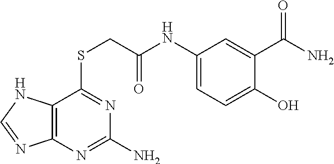 [SMILES: NC(=O)c1cc(NC(=O)CSc2nc(N)nc3nc[nH]c23)ccc1O] | (230) |
| 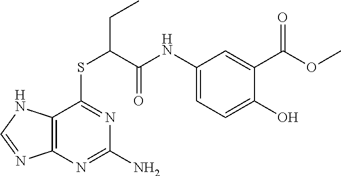 [SMILES: CCC(Sc1nc(N)nc2nc[nH]c12)C(=O)Nc1ccc(O)c(C(=O)OC)c1] | (258) |
| 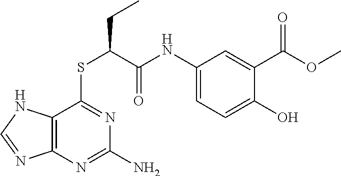 [SMILES: CC[C@H](Sc1nc(N)nc2nc[nH]c12)C(=O)Nc1ccc(O)c(C(=O)OC)c1] | (266) |
| 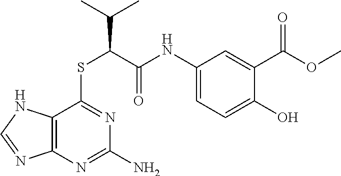 [SMILES: COC(=O)c1cc(NC(=O)[C@@H](Sc2nc(N)nc3nc[nH]c23)C(C)C)ccc1O] | (268) |
| 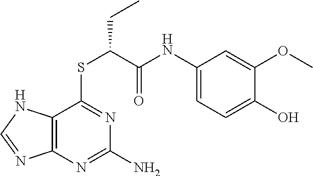 [SMILES: CC[C@@H](Sc1nc(N)nc2nc[nH]c12)C(=O)Nc1ccc(O)c(OC)c1] | (275) |
| 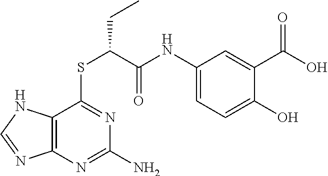 [SMILES: CC[C@@H](Sc1nc(N)nc2nc[nH]c12)C(=O)Nc1ccc(O)c(C(=O)O)c1] | (276) |
| 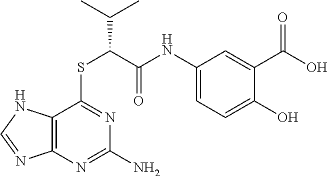 [SMILES: CC(C)[C@@H](Sc1nc(N)nc2nc[nH]c12)C(=O)Nc1ccc(O)c(C(=O)O)c1] | (278) |
| 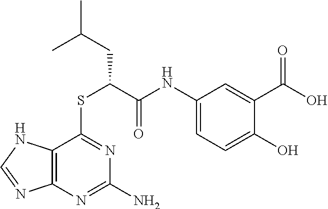 [SMILES: CC(C)C[C@@H](Sc1nc(N)nc2nc[nH]c12)C(=O)Nc1ccc(O)c(C(=O)O)c1] | (280) |
| 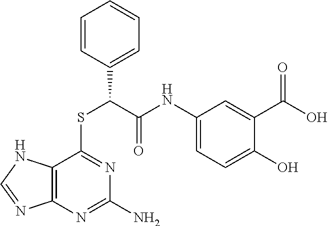 [SMILES: Nc1nc(S[C@@H](C(=O)Nc2ccc(O)c(C(=O)O)c2)c2ccccc2)c2[nH]cnc2n1] | (287) |
| 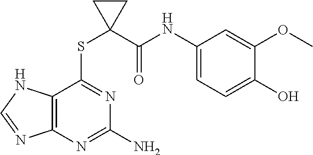 [SMILES: COc1cc(NC(=O)C2(Sc3nc(N)nc4nc[nH]c34)CC2)ccc1O] | (288) |
| 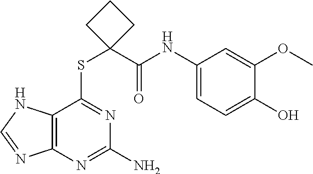 [SMILES: COc1cc(NC(=O)C2(Sc3nc(N)nc4nc[nH]c34)CCC2)ccc1O] | (290) |
| 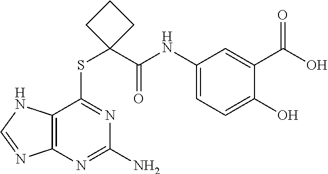 [SMILES: Nc1nc(SC2(C(=O)Nc3ccc(O)c(C(=O)O)c3)CCC2)c2[nH]cnc2n1] | (291) |
| 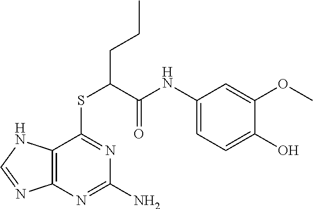 [SMILES: CCCC(Sc1nc(N)nc2nc[nH]c12)C(=O)Nc1ccc(O)c(OC)c1] | (299) |
| 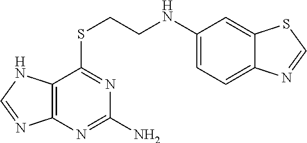 [SMILES: Nc1nc(SCCNc2ccc3ncsc3c2)c2[nH]cnc2n1] | (379) |
| 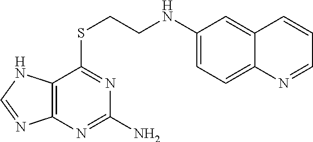 [SMILES: Nc1nc(SCCNc2ccc3ncccc3c2)c2[nH]cnc2n1] | (381) |
| 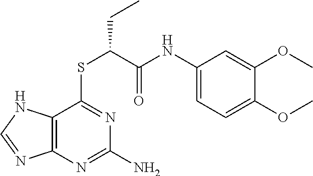 [SMILES: CC[C@@H](Sc1nc(N)nc2nc[nH]c12)C(=O)Nc1ccc(OC)c(OC)c1] | (383) |
| 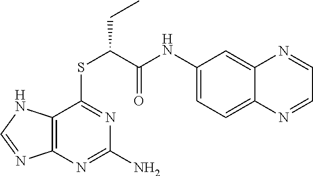 [SMILES: CC[C@@H](Sc1nc(N)nc2nc[nH]c12)C(=O)Nc1ccc2nccnc2c1] | (385) |
| 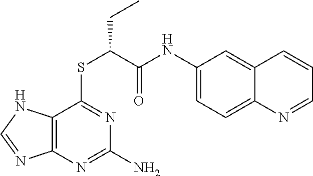 [SMILES: CC[C@@H](Sc1nc(N)nc2nc[nH]c12)C(=O)Nc1ccc2ncccc2c1] | (386) |
| 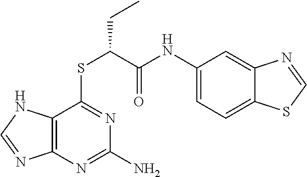 [SMILES: CC[C@@H](Sc1nc(N)nc2nc[nH]c12)C(=O)Nc1ccc2scnc2c1] | (413) |
| 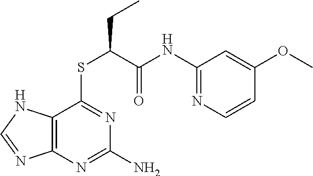 [SMILES: CC[C@H](Sc1nc(N)nc2nc[nH]c12)C(=O)Nc1cc(OC)ccn1] | (470) |
| 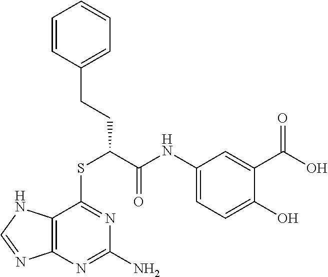 [SMILES: Nc1nc(S[C@H](CCc2ccccc2)C(=O)Nc2ccc(O)c(C(=O)O)c2)c2[nH]cnc2n1] | (481) |
| 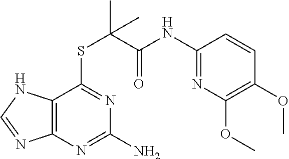 [SMILES: COc1ccc(NC(=O)C(C)(C)Sc2nc(N)nc3nc[nH]c23)nc1OC] | (503) |
| 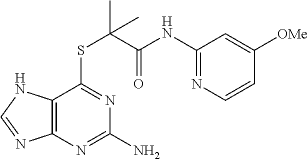 [SMILES: COc1ccnc(NC(=O)C(C)(C)Sc2nc(N)nc3nc[nH]c23)c1] | (505) |
| 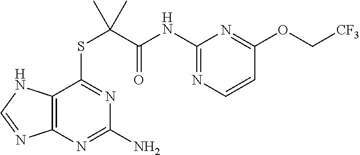 [SMILES: CC(C)(Sc1nc(N)nc2nc[nH]c12)C(=O)Nc1nccc(OCC(F)(F)F)n1] | (533) |

TABLE 4
In vitro data for evaluated compounds.
Compounds with ENPP1 Inhibition (Kᵢ) between ≥1 μM

| | |
|---|---|
| 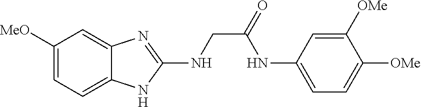 [SMILES: COc1ccc2[nH]c(NCC(=O)Nc3ccc(OC)c(OC)c3)nc2c1] | (3) |
| 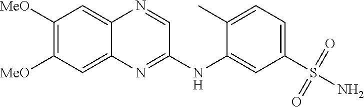 [SMILES: COc1cc2ncc(Nc3cc(S(N)(=O)=O)ccc3C)nc2cc1OC] | (8) |
| 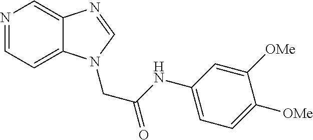 [SMILES: COc1ccc(NC(=O)Cn2cnc3cnccc32)cc1OC] | (11) |
| 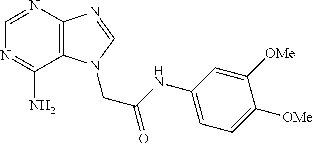 [SMILES: COc1ccc(NC(=O)Cn2cnc3ncnc(N)c32)cc1OC] | (18) |
| 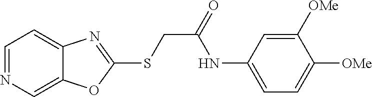 [SMILES: COc1ccc(NC(=O)CSc2nc3ccncc3o2)cc1OC] | (22) |
| 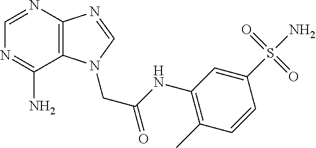 [SMILES: Cc1ccc(S(N)(=O)=O)cc1NC(=O)Cn1cnc2ncnc(N)c21] | (30) |
| 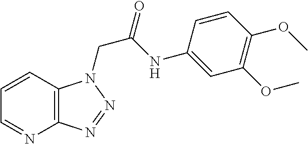 [SMILES: COc1ccc(NC(=O)Cn2nnc3ncccc32)cc1OC] | (35) |
| 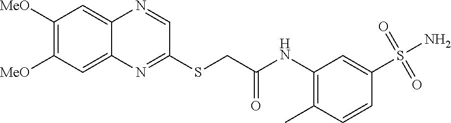 [SMILES: COc1cc2ncc(SCC(=O)Nc3cc(S(N)(=O)=O)ccc3C)nc2cc1OC] | (36) |
| 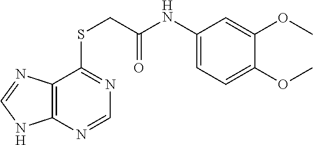 [SMILES: COc1ccc(NC(=O)CSc2ncnc3[nH]cnc23)cc1OC] | (45) |
| 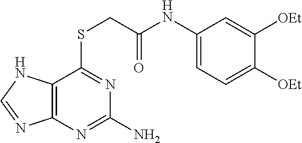 [SMILES: CCOc1ccc(NC(=O)CSc2nc(N)nc3nc[nH]c23)cc1OCC] | (84) |
| 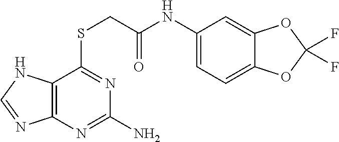 [SMILES: Nc1nc(SCC(=O)Nc2ccc3c(c2)OC(F)(F)O3)c2[nH]cnc2n1] | (90) |
| 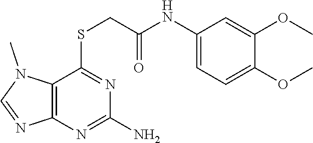 [SMILES: COc1ccc(NC(=O)CSc2nc(N)nc3ncn(C)c23)cc1OC] | (98) |
| 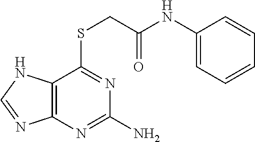 [SMILES: Nc1nc(SCC(=O)Nc2ccccc2)c2[nH]cnc2n1] | (102) |
| 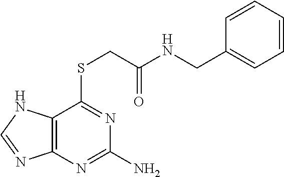 [SMILES: Nc1nc(SCC(=O)NCc2ccccc2)c2[nH]cnc2n1] | (103) |
| 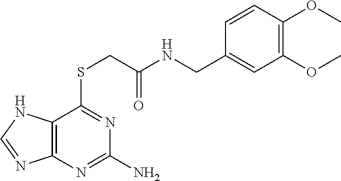 [SMILES: COc1ccc(CNC(=O)CSc2nc(N)nc3nc[nH]c23)cc1OC] | (105) |
| 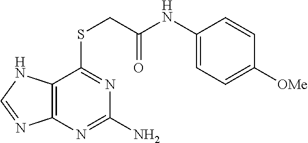 [SMILES: COc1ccc(NC(=O)CSc2nc(N)nc3nc[nH]c23)cc1] | (107) |
| 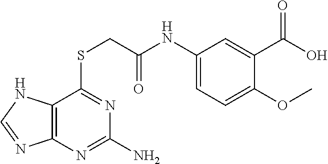 [SMILES: COc1ccc(NC(=O)CSc2nc(N)nc3nc[nH]c23)cc1C(=O)O] | (144) |
| 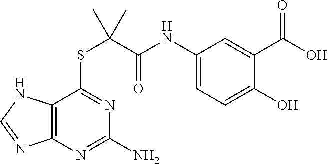 [SMILES: CC(C)(Sc1nc(N)nc2nc[nH]c12)C(=O)Nc1ccc(O)c(C(=O)O)c1] | (251) |

### Example 8: In Vivo Efficacy Evaluation of Compound 155, Compound 173, and Compound 174 in Combination with an Anti-PD-1 Antibody, CD279, in LLC1 Syngeneic Tumor Model

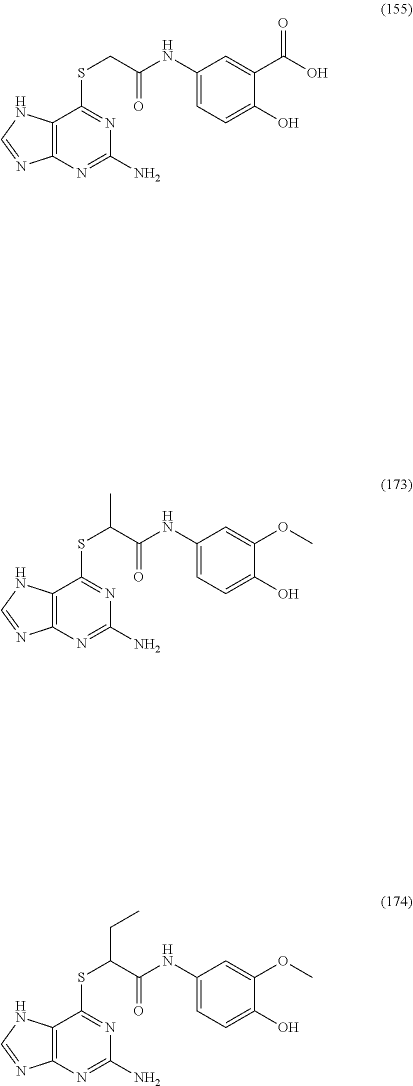 [SMILES: Nc1nc(SCC(=O)Nc2ccc(O)c(C(=O)O)c2)c2[nH]cnc2n1 ; COc1cc(NC(=O)C(C)Sc2nc(N)nc3nc[nH]c23)ccc1O ; CCC(Sc1nc(N)nc2nc[nH]c12)C(=O)Nc1ccc(O)c(OC)c1]

Study Overview

The anti-tumor efficacy of Compound 155, 173 and 174 was evaluated in combination with an anti-PD-1 antibody (check point inhibitor) in LLC1, a syngeneic tumor model. Compounds were administered orally at a dose of 100 mg/kg, once daily. IV doses of the compound were administered at 10 mg/kg dose, twice weekly or Q3D (Compound 155 at 2 mg/Kg IV). Anti-PD-1 antibody was administered at a dose of 200 μg/animal via IP route on days 1, 5 and 9 (Table 5).

Study Design

C57BL/6 mice (female, 7-8 weeks of age) were implanted subcutaneously with 0.2×10⁶ LLC1 cells (ATCC® CRL-1642^{T}m) to evaluate tumor growth in a murine Lewis lung carcinoma model. Tumor-bearing animals were randomized into different treatment groups of 8 animals based on tumor size criteria of about 50-60 mm³ and dosed according to the schedule in Table 5.

TABLE 5
Test groups and dosing schedule for murine Lewis lung
carcinoma model

| | Dose | | | |
|---|---|---|---|---|
| | Test | | Dose frequency | |
| | compound | Anti-PD-1 | Test | Anti- |
| Groups | (mg/kg) | (μg/animal) | Items | PD-1 |
| G1: Vehicle | — | — | — | — |
| control | | | | |
| G2: Anti-PD-1 | — | 200 | — | Q4D × 3 |
| (IP) | | | | |
| G3: 155 (PO) | 100 | — | QD × 14 | — |
| G4: 155 (PO) + | 100 | 200 | QD × 14 | Q4D × 3 |
| Anti-PD-1 (IP) | | | | |
| G5: 155 (IV) | 2 | — | TW × 14 | — |
| G6: 155 (IV) + | 2 | 200 | TW × 14 | Q4D × 3 |
| Anti-PD-1 (IP) | | | | |
| G7: 173 (PO) | 100 | — | QD × 14 | — |
| G8: 173 (PO) + | 100 | 200 | QD × 14 | Q4D × 3 |
| Anti-PD-1 (IP) | | | | |
| G9: 173 (IV) | 10 | — | TW × 14 | — |
| G10: 173 (IV) + | 10 | 200 | TW × 14 | Q4D × 3 |
| Anti-PD-1 (IP) | | | | |
| G11: 174 (PO) | 100 | — | QD × 14 | — |
| G12: 174 (PO) + | 100 | 200 | QD × 14 | Q4D × 3 |
| Anti-PD-1 (IP) | | | | |
| G13: 174 (IV) | 10 | — | TW × 14 | — |
| G14: 174 (IV) + | 10 | 200 | TW × 14 | Q4D × 3 |
| Anti-PD-1 (IP) | | | | |

Note
QD: Once daily. Q4D: Every 4 days once and TW: Twice weekly (Q3D)
Oral formulation: 0.4% Tween 80, 2% Glycerol and 97.6% of 15% (w/v) HPβCD
IV formulation: 5% (v/v) DMA, 15% (v/v) Solutol, 30% (v/v) of 60% (w/v) HPβCD and 50% (v/v) sodium carbonate buffer pH 9.2
Anti-PD-1: Dilution Buffer (pH 7.0) (BioXcell, West Lebanon, NH)

Dosing protocol: The frequency for oral administration was once daily and IV administration was twice weekly for two weeks; anti-PD-1 antibody was administered via IP route on Days 1, 5 and 9.

Tumor measurement: Tumor growth was measured thrice weekly using a digital Vernier caliper. Tumor volume was calculated as: Tumor Volume (TV)=[Length (L)×Width (W)2]/2 (where length is the largest diameter and width is the smallest diameter of the tumor)

Terminal Endpoints: Animals were subjected to blood sampling after 2 weeks of treatment for analysis of cytokine levels (IFN-0 and IP-10) in serum. At the end of the study, tumor samples were harvested from 4/8 animals from each of the treatment groups for analysis of TILs by flow cytometry.

Efficacy Evaluation: Tumor growth inhibition (TGI) was calculated as:

$$\% TGI = \frac{\{ ( TV {Control}_{Final} - TV {Control}_{Initial} ) - ( TV {Treated}_{Final} - TV {Treated}_{Initial} ) \} \times 100}{( TV {Control}_{Final} - TV {control}_{Initial} )}$$

Data Analysis: Statistical analysis of the data was performed by One-way ANOVA followed by Dunnett's test using GraphPad Prism (version 5.03).

Results

Anti-Tumor Efficacy of Compound 155

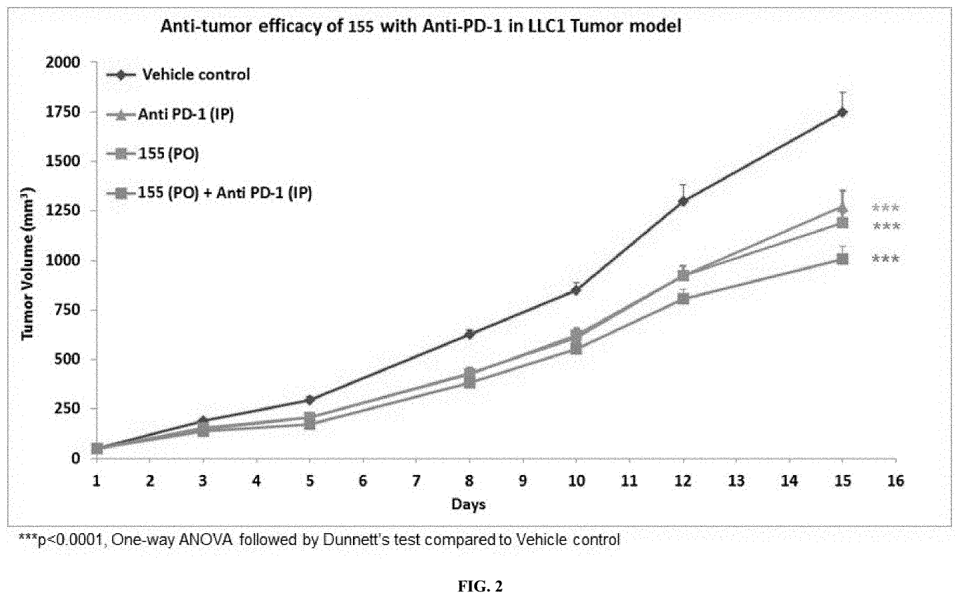

PO Dosing: Once daily oral doses of Compound 155 for 2 weeks resulted in 33% TGI compared to the control group, which was statistically significant (p<0.0001). Combination of Compound 155 (PO) with anti-PD-1 antibody resulted in a significant (p<0.0001) tumor growth inhibition (TGI) of 44% (FIG. 2).

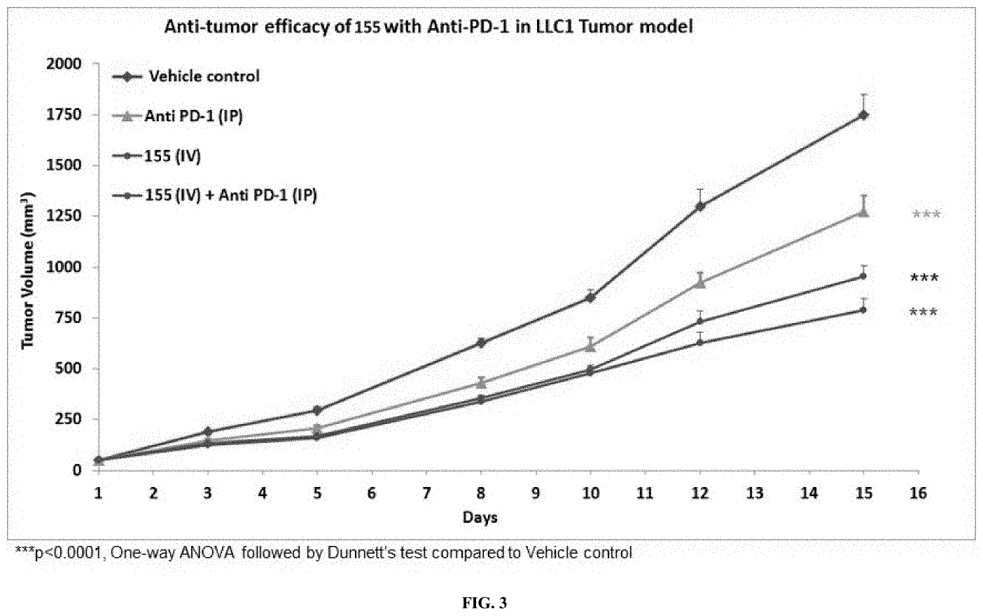

IV Dosing: Twice weekly IV administration of Compound 155 resulted in a significant (p<0.0001) tumor growth Inhibition (TGI) of 47%. Combination of Compound 155 (IV) with anti-PD-1 antibody resulted in a significant (p<0.0001) tumor growth inhibition (TGI) of 57% (FIG. 3).

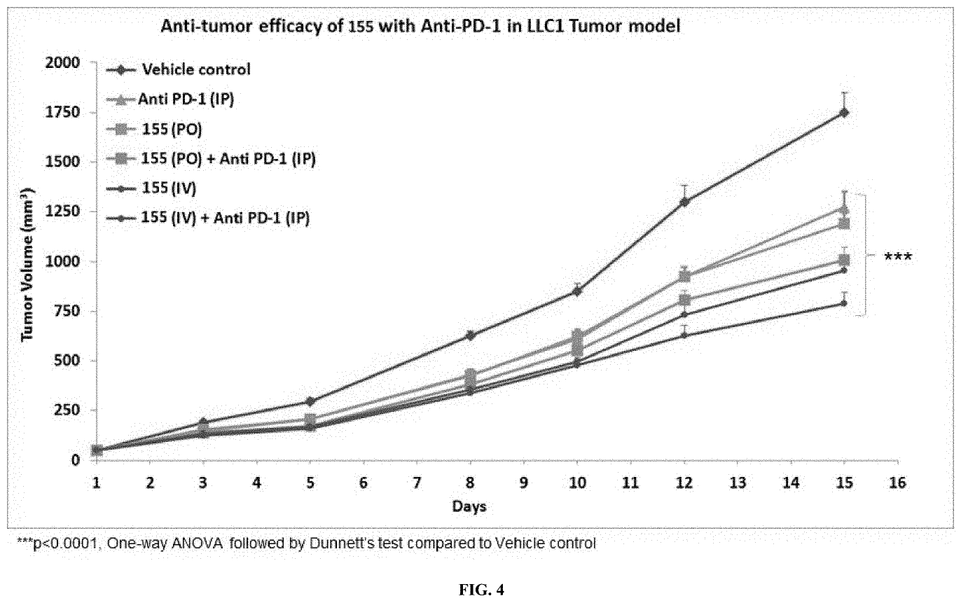

As shown in FIG. 4, PO and IV dosing of Compound 155 alone or in combination with anti-PD-1 antibody reduced tumor volume in the murine Lewis lung carcinoma model, with IV dosing of Compound 155 in combination with anti-PD-1 antibody providing the greatest reduction.

Anti-Tumor Efficacy of Compound 173

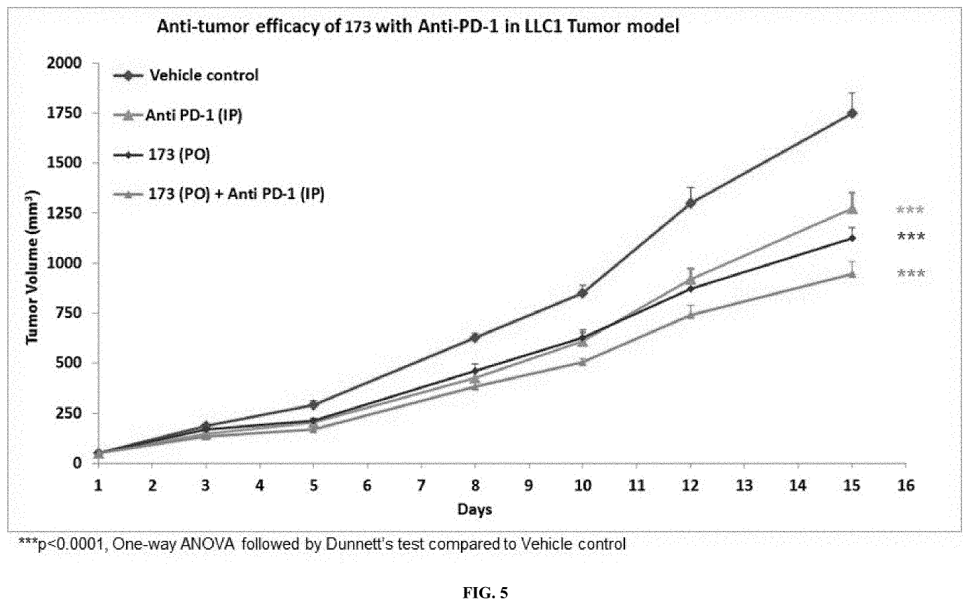

PO Dosing: Once daily oral administration of Compound 173 for 2 weeks resulted in 37% TGI compared to control group, which was statistically significant (p<0.0001). Combination of Compound 173 (PO) with anti-PD-1 antibody resulted in a significant (p<0.0001) tumor growth inhibition (TGI) of 47% (FIG. 5).

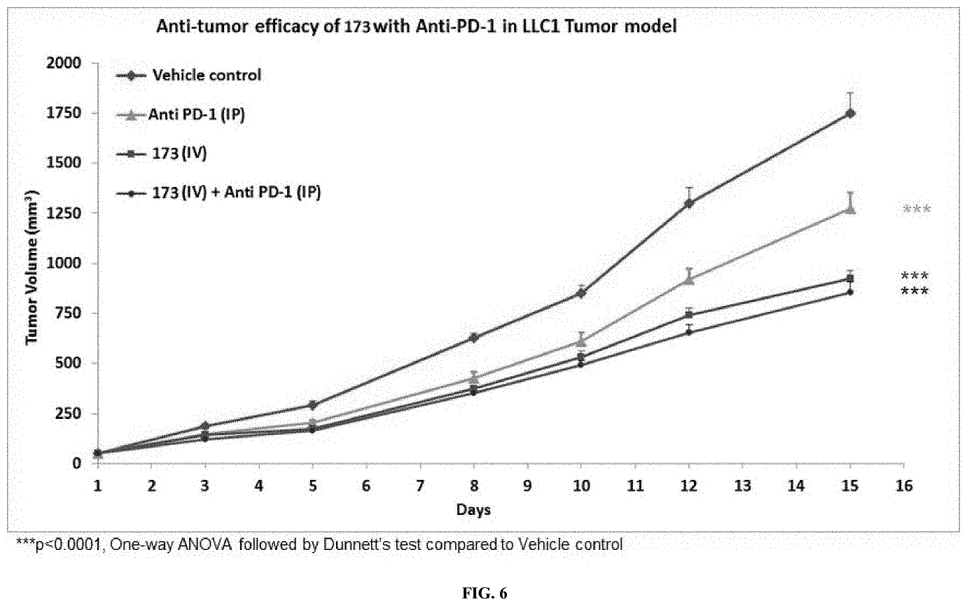

IV Dosing: Twice weekly IV administration of Compound 173 resulted in a significant (p<0.0001) tumor growth Inhibition (TGI) of 48%. Combination of Compound 173 (IV) with anti-PD-1 antibody resulted in a significant (p<0.0001) tumor growth inhibition (TGI) of 53% (FIG. 6).

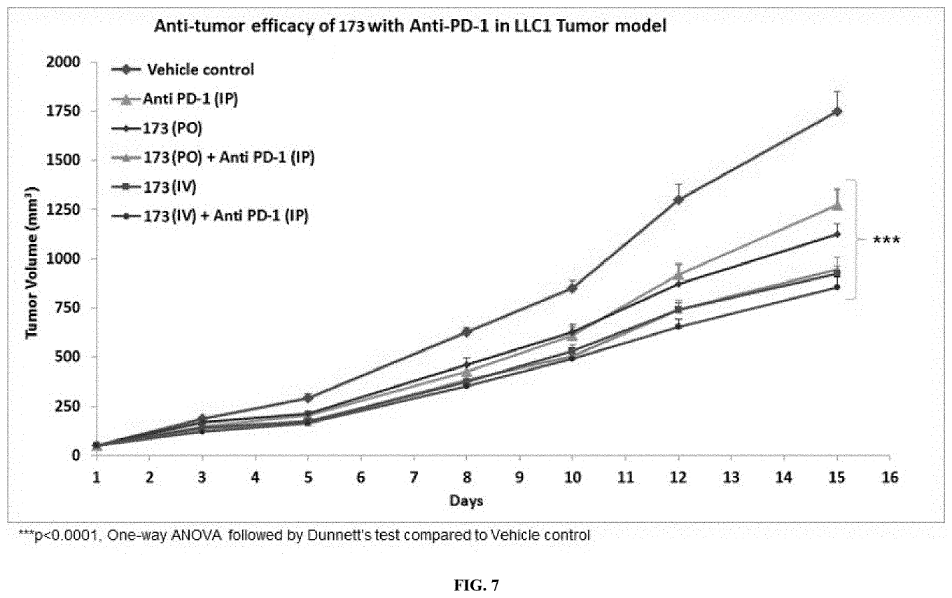

As shown in FIG. 7, PO and IV dosing of Compound 173 alone or in combination with anti-PD-1 antibody reduced tumor volume in the murine Lewis lung carcinoma model, with IV dosing of Compound 173 in combination with anti-PD-1 antibody providing the greatest reduction.

Anti-Tumor Efficacy of Compound 174

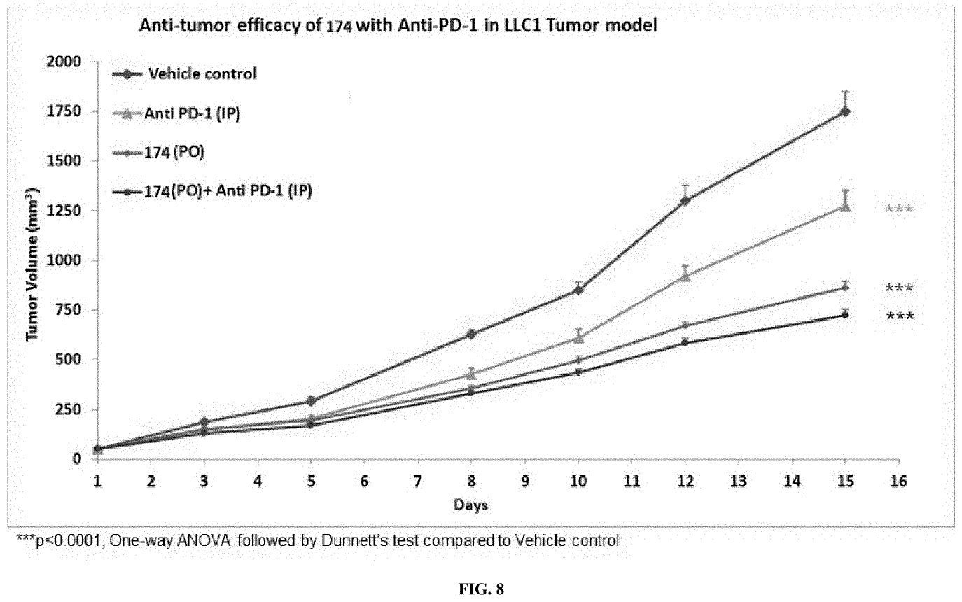

PO Dosing: Once daily oral administration of Compound 174 for 2 weeks resulted in 52% TGI compared to control group, which was statistically significant (p<0.0001). Combination of Compound 174 (PO) with anti-PD-1 antibody resulted in a significant (p<0.0001) tumor growth inhibition (TGI) of 60% (FIG. 8).

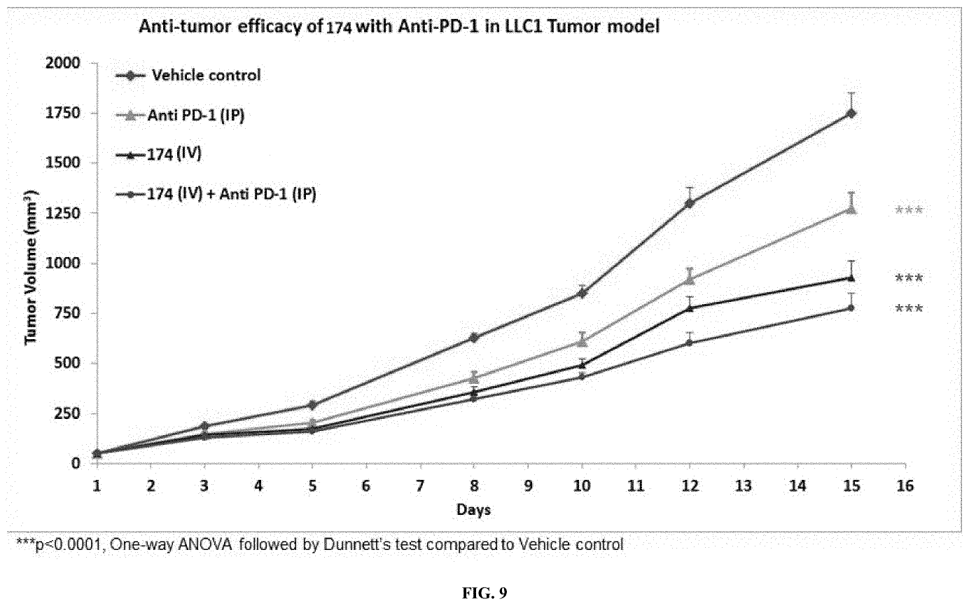

IV Dosing: Twice weekly IV administration of Compound 174 resulted in a significant (p<0.0001) tumor growth Inhibition (TGI) of 48%. Combination of Compound 174 (IV) with anti-PD-1 antibody resulted in a significant (p<0.0001) tumor growth inhibition (TGI) of 57% (FIG. 9).

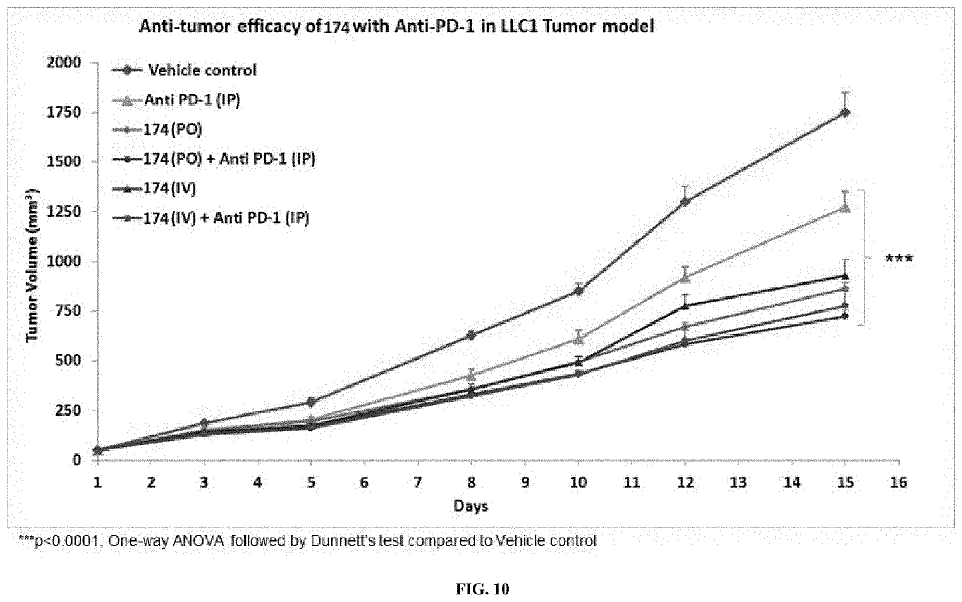

As shown in FIG. 10, PO and IV dosing of Compound 173 alone or in combination with anti-PD-1 antibody reduced tumor volume in the murine Lewis lung carcinoma model, with IV dosing of Compound 174 in combination with anti-PD-1 antibody providing the greatest reduction.

TABLE 6
Efficacy of Compound 155, 173, and 174 in a murine Lewis lung carcinoma model.

| | Dose | | Tumor volume (mm³), | | | |
|---|---|---|---|---|---|---|
| | Test compound | Anti-PD-1 | Mean ± SE | | | % TGI |
| Group | (mg/kg) | (μg/animal) | Day 1 | Day 15 | | (vs Control) |
| G1: Vehicle control | — | — | 50 ± 1.6 | 1750 ± | 99.3 | — |
| G2: Anti-PD-1 (IP) | — | 200 | 50 ± 1.6 | 1273 ± | 78.1*** | 28 |
| G3: 155 (PO) | 100 | — | 50 ± 1.4 | 1191 ± | 52.8*** | 33 |
| G4: 155 (PO) + Anti-PD-1 (IP) | 100 | 200 | 50 ± 1.4 | 1008 ± | 65.1*** | 44 |
| G5: 155 (IV) | 2 | — | 51 ± 2.0 | 956 ± | 52.4*** | 47 |
| G6: 155 (IV) + Anti-PD-1 (IP) | 2 | 200 | 50 ± 1.4 | 787 ± | 5 [symbol] .4*** | 57 |
| G7: 173 (PO) | 100 | — | 50 ± 1.4 | 1126 ± | 53.3*** | 37 |
| G8: 173 (PO) + Anti-PD-1 (IP) | 100 | 200 | 50 ± 1.4 | 945 ± | 62.1*** | 47 |
| G9: 173 (IV) | 10 | — | 50 ± 1.3 | 926 ± | 39.6*** | 48 |
| G10: 173 (IV) + Anti-PD-1 (IP) | 10 | 200 | 50 ± 1.4 | 856 ± | 53.1*** | 53 |
| G11: 174 (PO) | 100 | — | 50 ± 1.3 | 8 [symbol] ± | 31.0*** | 52 |
| G12: 174 (PO) + Anti-PD-1 (IP) | 100 | 200 | 50 ± 1.2 | 724 ± | 31.1*** | 60 |
| G13: 174 (IV) | 10 | — | 50 ± 1.1 | 930 ± | 80.7*** | 48 |
| G14: 174 (IV) + Anti-PD-1 (IP) | 10 | 200 | 50 ± 1.2 | 778 ± | 72.4*** | 57 |

***p < 0.0001. One-way ANOVA followed by Dunnell's test compared to Vehicle control [symbol]
indicates data missing or illegible when filed

The invention is further described by the following numbered embodiments:

1. A compound of Formula (X) or a pharmaceutically acceptable salt, hydrate, or tautomer thereof:

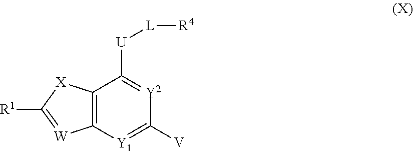

wherein:

L is a linker selected from alkylene, alkenylene, optionally substituted alkylene-S—, optionally substituted alkylene-O—, optionally substituted -alkylene-(NR⁵)—, optionally substituted

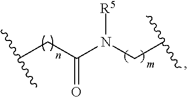

optionally substituted

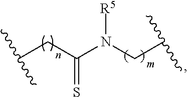

optionally substituted

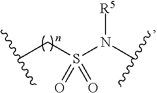

optionally substituted

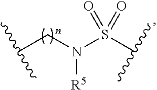

optionally substituted

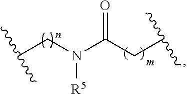

optionally substituted

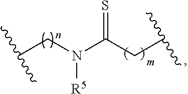

optionally substituted

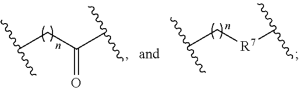

U is S or NH;

V is OH, NR²N³ or V and Y¹ taken together with the atoms to which they are attached form an optionally substituted phenyl or pyridinyl ring;

W is CH or N;

X is O, S, NR⁶, —CH═CH—, or —CH═N—;

Y¹ and Y² are each independently CH or N;

R¹ is H, OH, O-alkyl, alkyl or carbocyclyl;

R² and R³ are each independently H, alkyl, alkylenearyl, or —C(O)alkyl;

R⁴ is carbocyclyl, heterocyclyl, aryl, or heteroaryl, each of which is optionally substituted;

R⁵ is H, alkyl, —C(O)alkyl, carbocyclyl, alkylenecarbocyclyl, or alkylenearyl;

R⁶ is H, alkyl, carbocyclyl, alkylenecarbocyclyl, alkylenearyl, —C(O)alkyl, or —C(O)Oalkylenearyl;

R⁷ is carbocyclyl, heterocyclyl, or heteroaryl;

m is 0, 1, or 2; and

n is 1, 2, or 3.

2. The compound of embodiment 1, wherein L is a

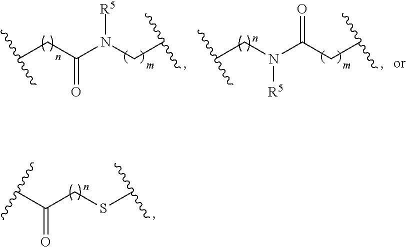

each of which is optionally substituted.
2a. The compound of embodiment 2, wherein the optionally substituted

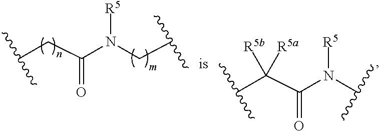

wherein:

R⁵ is H, alkyl, —C(O)alkyl, carbocyclyl, alkylenecarbocyclyl, or alkylenearyl; and

R^{5a} and R^{5b} are each independently selected from the group consisting of H, halogen, C₁₋₅alkyl, C₃₋₆carbocyclyl, alkylene-C₃₋₆carbocyclyl, aryl, alkylenearyl, or NH₂; wherein two —C₁₋₅alkyl taken together with the carbon atom to which they are attached form a C₃₋₆carbocyclyl.

2b. The compound of embodiment 2a, wherein

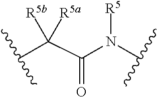

is selected from the group consisting of:

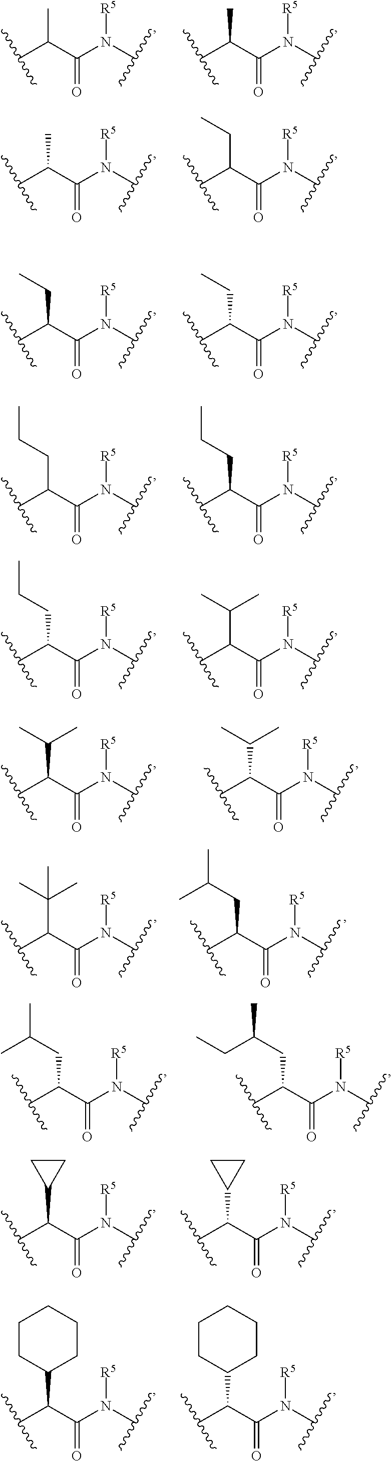

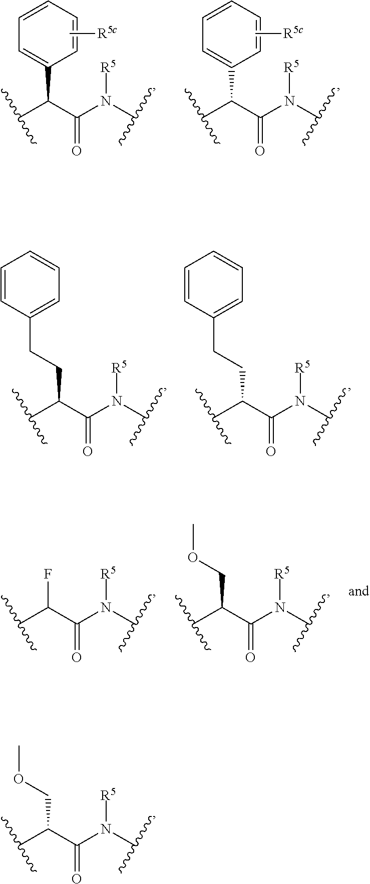

wherein:

R⁵ is H, Me, or —C(O)alkyl; and

R^{5c} is halogen, alkyl, haloalkyl, hydroxy, or alkoxy.

2c. The compound of embodiment 2a, wherein

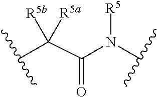

is selected from the group consisting of:

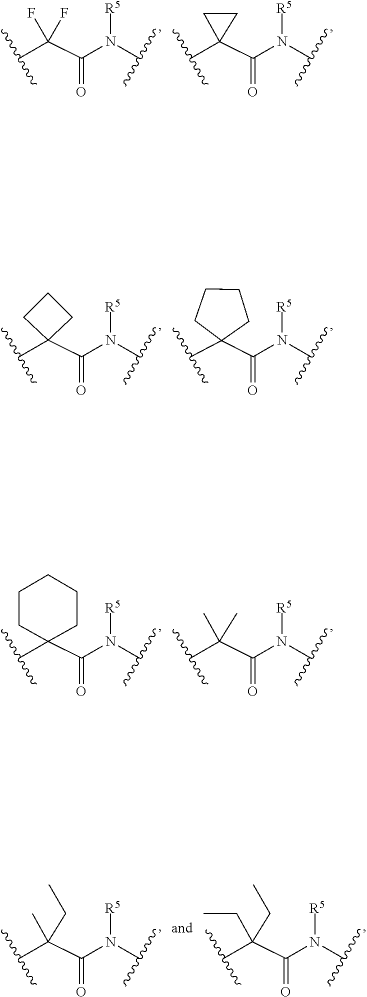

wherein R⁵ is H, Me, or —C(O)alkyl.
3. The compound of any one of embodiments, 1-2c, wherein U is S.
4. The compound of any one of embodiments 1-3, wherein V is NR²R³.
5. The compound of any one of embodiments 1-4, wherein W is N.
6. The compound of any one of embodiments 1-5, wherein X is NR⁶.
7. The compound of any one of embodiments 1-6, wherein Y¹ and Y² are both N.
8. The compound of any one of embodiments 1-7, wherein R¹ is H, OH, or C₁₋₅alkyl.
9. The compound of any one of embodiments 1-8, wherein R² and R³ are independently H, —C₁₋₅alkyl, —CH₂Ph, or —C(O)(C₁₋₅alkyl).
10. The compound of any one of embodiments 1-9, wherein R⁴ is aryl or heteroaryl, each of which is optionally substituted.
10a. The compound of any one of claims 1-10, wherein R⁴ is selected from the group consisting of:

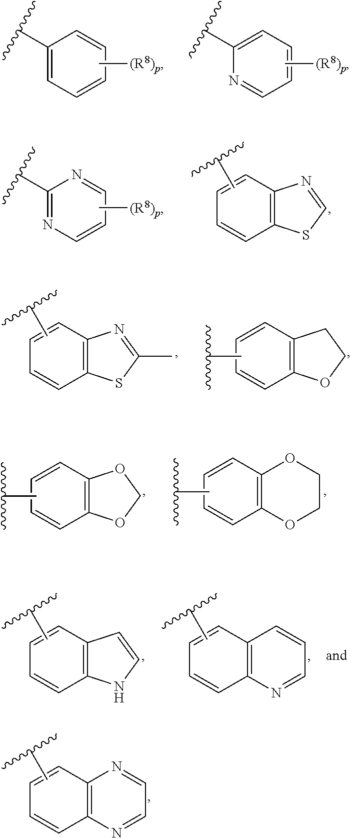

wherein:

each R⁸ is independently halogen, C₁₋₅ alkyl, —OH, —OC₁₋₅alkyl, —COOH, or —CO₂C₁₋₅alkyl; and

p is an integer from 0-3.

10b. The compound of any one of claims 1-10, wherein R⁴ is selected from the group consisting of:

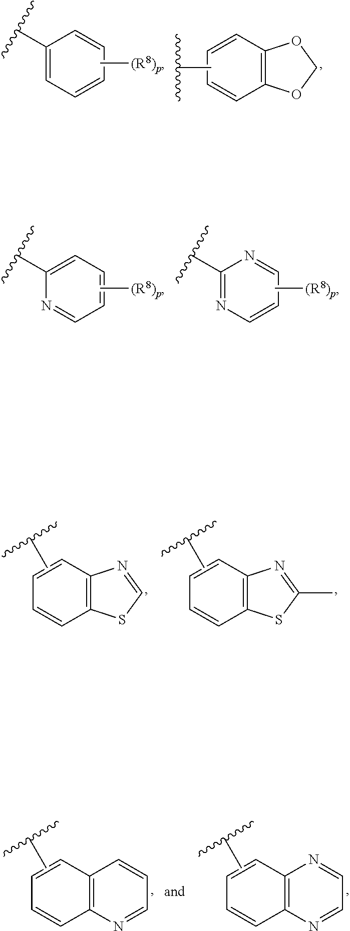

wherein:

each R⁸ is independently halogen, C₁₋₅ alkyl, —OH, —OC₁₋₅alkyl, —COOH, or —CO₂C₁₋₅alkyl; and

p is an integer from 0-3.

11. The compound of any one of embodiments 1-10, wherein R⁵ is H, —C₁₋₅alkyl, —C₃₋₆-carbocyclyl, —CH₂-aryl, or —CH₂—(C₃₋₆carbocyclyl).
12. The compound of any one of embodiments 1-11, wherein R⁶ is H, —C₁₋₅alkyl, —CH₂aryl, or —CH₂—(C₃₋₆carbocyclyl).
13. The compound of any one of embodiments 1-12, wherein R⁷ is a C₃₋₆carbocyclyl, a 3- to 6-membered heterocyclyl, or a 5- to 6-membered heteroaryl.
14. The compound of any one of embodiments 1-13, wherein m is 0 or 1.
15. The compound of any one of embodiments 1-14, wherein n is 1 or 2.
16. The compound of embodiment 1, having one of the following structures:

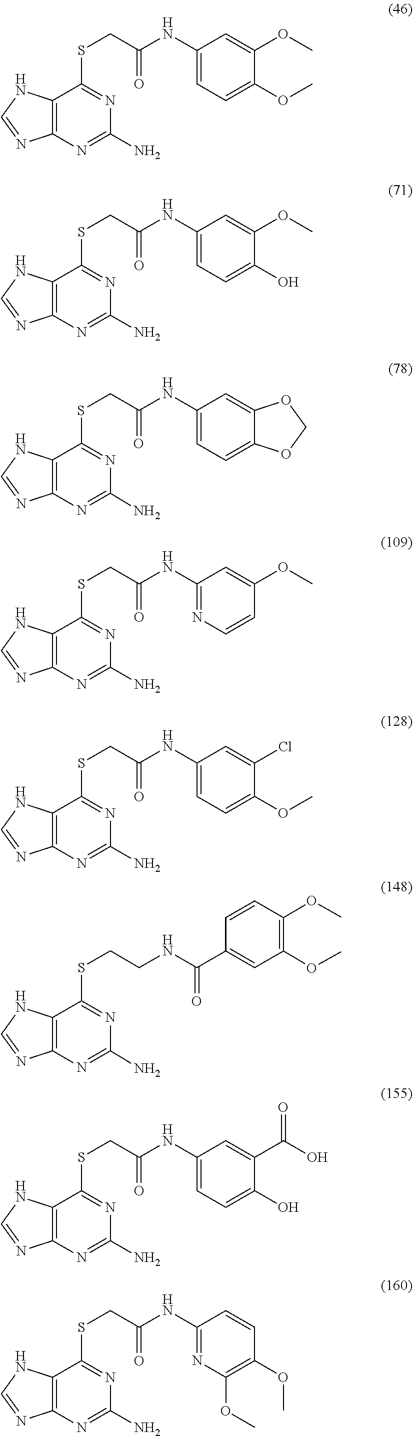

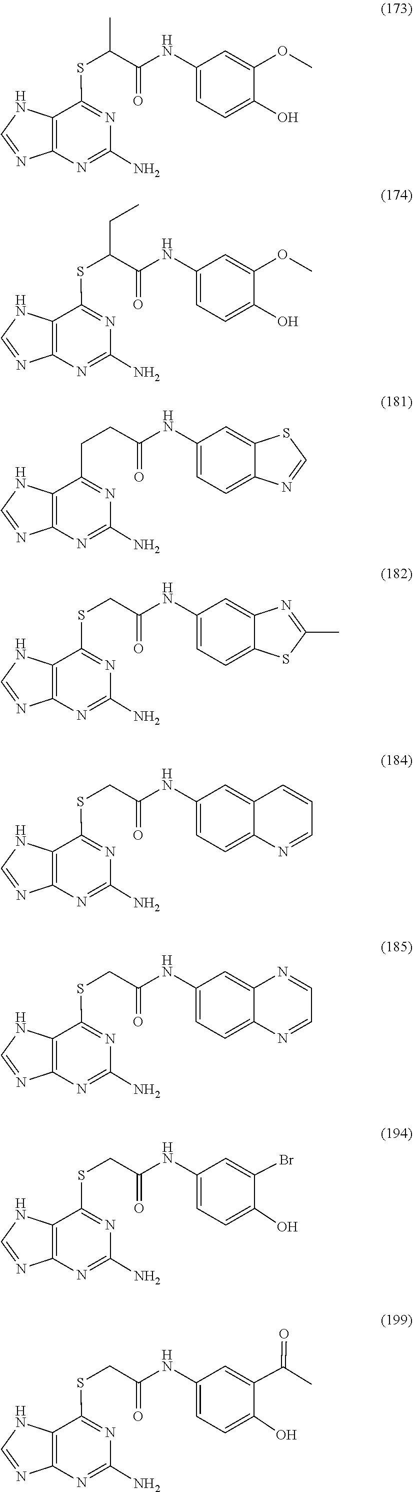

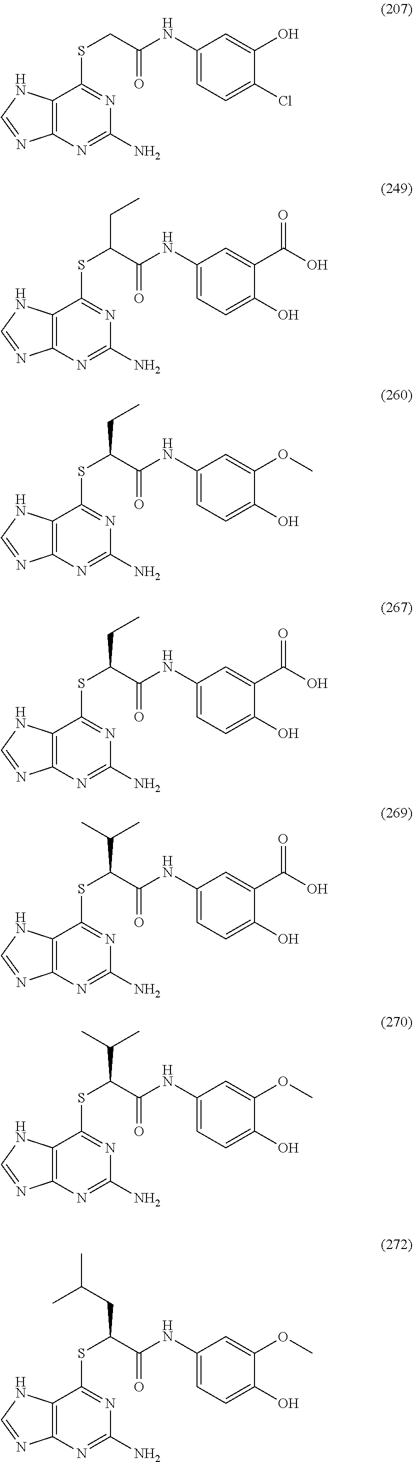

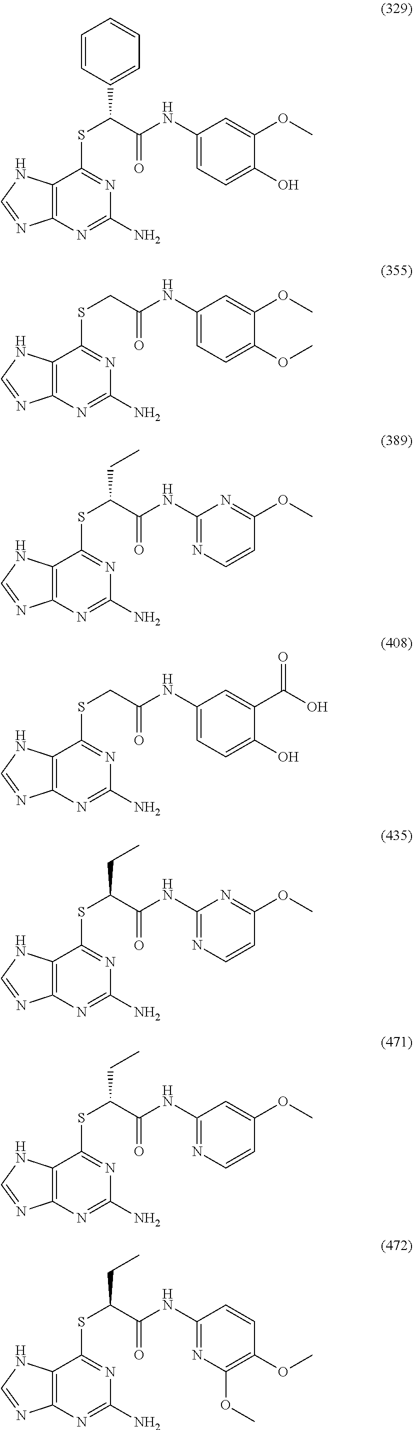

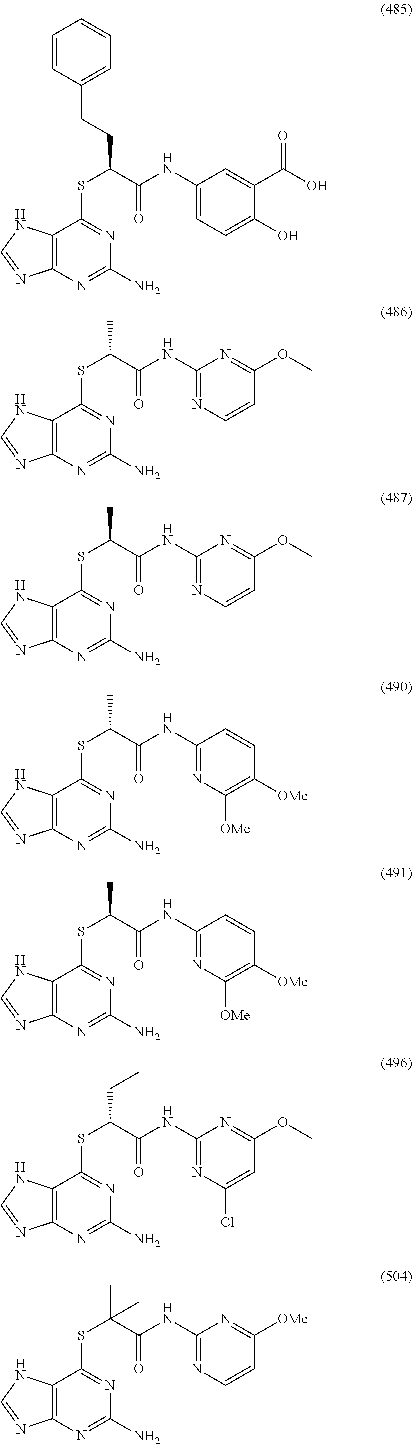

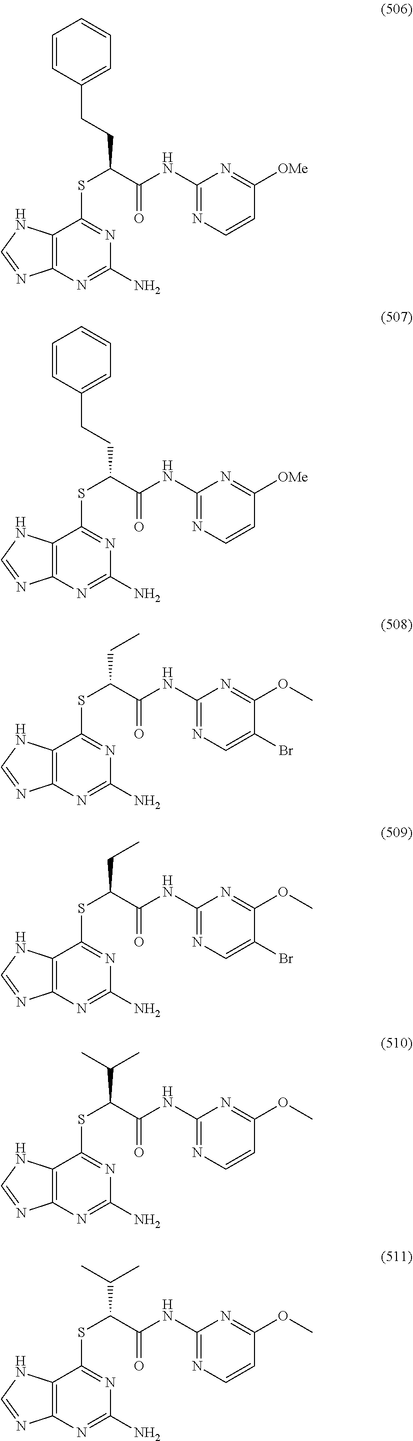

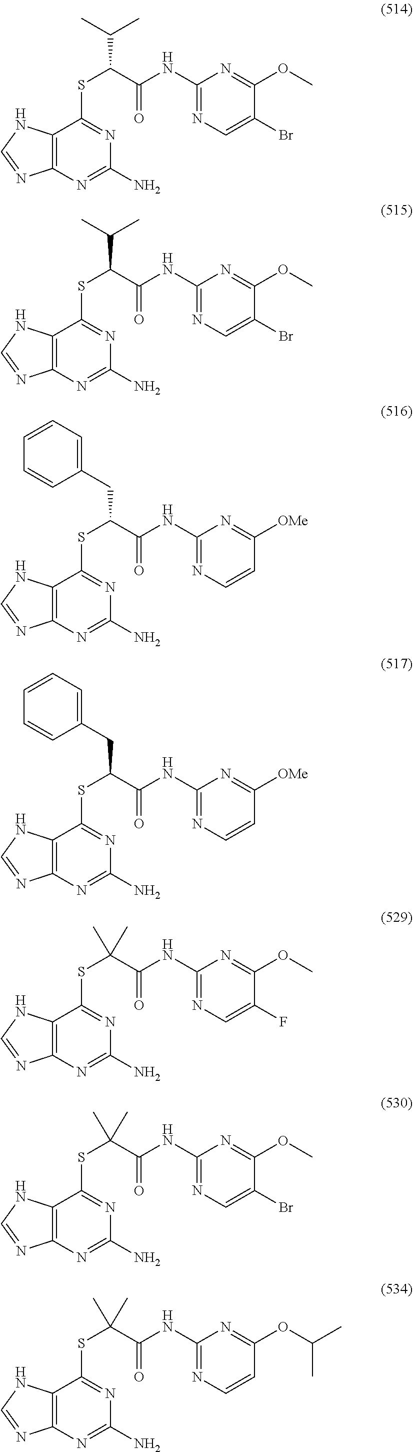

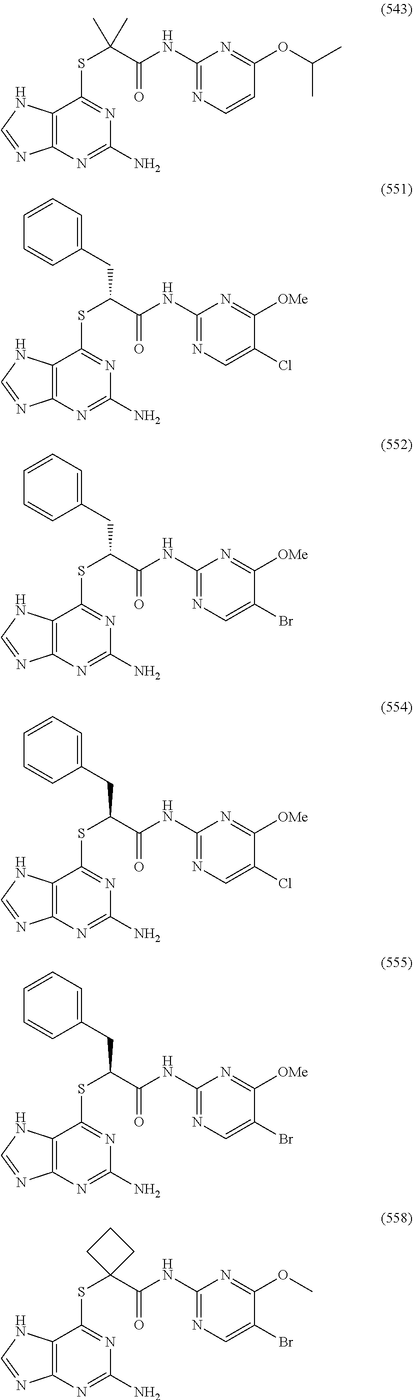

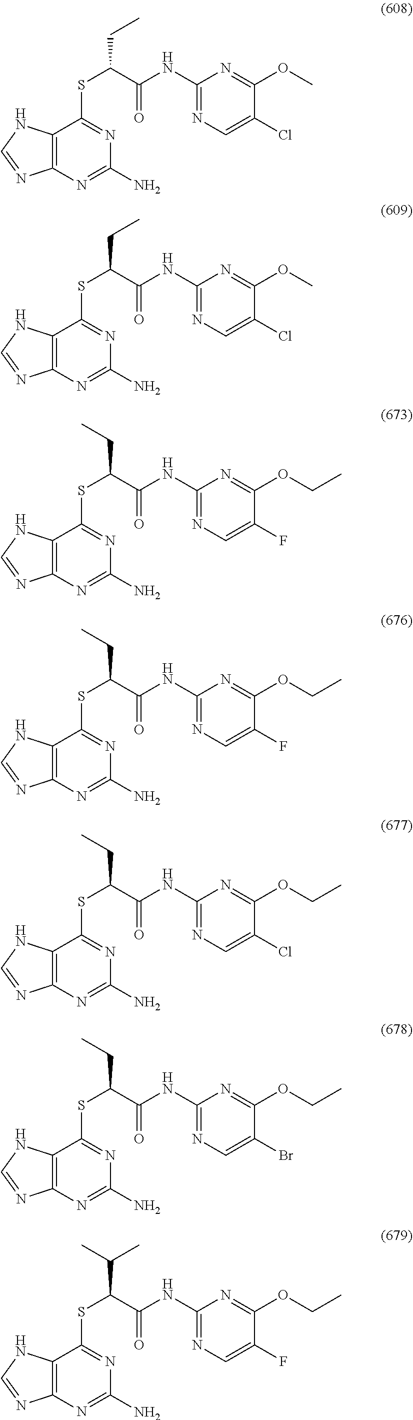

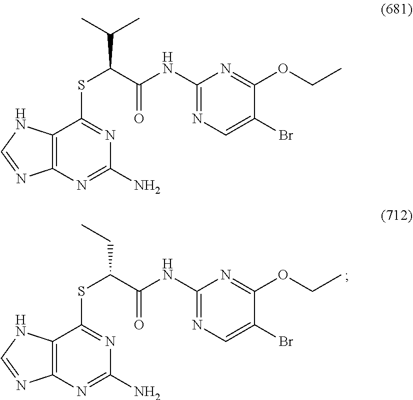

or a pharmaceutically acceptable salt, tautomer, hydrate or solvate thereof.
17. A compound of Formula (Y) or a pharmaceutically acceptable salt, hydrate, or tautomer thereof:

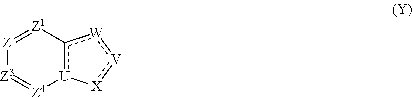

wherein:

U is C or N;
wherein
when U is C, Y is

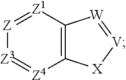

when U is N, Y is

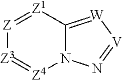

V is N or CR¹⁰;

W is CH or N;

X is S, O, N-L-R¹¹, or NR¹²;

L is selected from alkylene, alkenylene, optionally substituted -alkylene-(NR¹²)—, optionally substituted

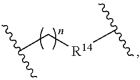

optionally substituted

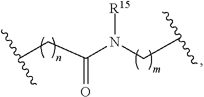

optionally substituted

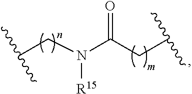

optionally substituted

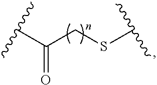

optionally substituted

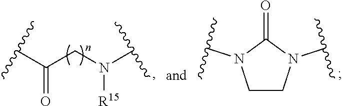

R¹⁰ is H, alkyl, —O-alkyl, —S-alkyl, carbocyclyl, alkylenecarbocyclyl, —O-L-R¹¹, —S-L-R¹¹, —N(R¹²)-L-R¹¹, -L-R¹¹;

R¹¹ is alkyl, carbocyclyl, heterocyclyl, aryl, or heteroaryl, each of which is optionally substituted; and

R¹² is each independently H, alkyl, alkylenecarbocyclyl, or carbocyclyl, wherein two R¹² groups taken together with the carbon atom to which they are attached can form a heterocyclyl;

R¹⁴ is carbocyclyl, heterocyclyl, or heteroaryl;

R¹⁵ is H, alkyl, carbocyclyl, alkylenecarbocyclyl, or alkylenearyl;
wherein:
   when X is N-L-R¹¹, V is N or CR¹⁰, wherein R¹⁰ is H, alkyl, —O-alkyl, —S-alkyl, carbocyclyl, or alkylenecarbocyclyl;
   when X is S, O, NR¹²; V is CR¹⁰, wherein R¹⁰ is —O-L-R¹¹, —S-L-R¹¹, —N(R¹²)-L-R¹¹, or -L-R¹¹; or
   when U is N, V is CR¹⁰, wherein R¹⁰ is —O-L-R¹¹, —S-L-R¹¹, —N(R¹²)-L-R¹¹, or -L-R¹¹;

Z¹, Z², Z³, and Z⁴ are each independently CR¹³ or N;

R¹³ is H, halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN, wherein two R¹³ taken together with the atoms to which they are attached can form carbocyclyl, heterocyclyl, or heteroaryl, each of which is optionally substituted;

m is 0, 1, or 2; and

n is 1, 2, or 3.

18. The compound of embodiment 17, wherein U is C.
19. The compound of embodiment 17 or 18, wherein W is N.
20. The compound of embodiment 17-19, wherein when X is S, O, or NH, V is CR¹⁰, wherein R¹⁰ is —O-L-R¹¹, —S-L-R¹¹, —N(R¹²)-L-R¹¹, or -L-R¹¹.
21. The compound of any one of embodiments 17-20, wherein X is N-L-R¹¹ and V is CR¹⁰, wherein R¹⁰ is H, alkyl, —O-alkyl, or —S-alkyl.
22. The compound of any one of embodiments 17-21, wherein L is -alkylene-(NR¹²)—,

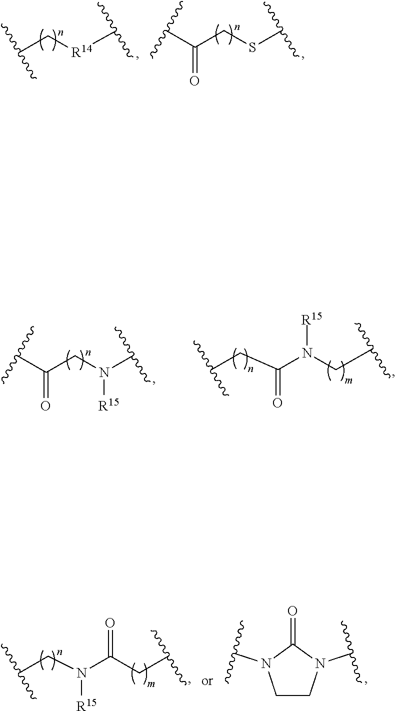

each of which is optionally substituted.
23. The compound of any one of embodiments 17-22, wherein R¹¹ is heterocyclyl, aryl, or heteroaryl, each of which is optionally substituted.
24. The compound of any one of embodiments 17-23, wherein R¹² is each independently H or C₁₋₅alkyl.
25. The compound of any one of embodiments 17-24, wherein R¹⁴ is heterocyclyl or heteroaryl.
26. The compound of any one of embodiments 17-25, wherein R¹⁵ is H or alkyl.
27. The compound of any one of embodiments 17-26, wherein each of Z¹, Z², Z³, and Z⁴ is CR¹³.
28. The compound of any one of embodiments 17-26, wherein at least one of Z¹, Z², Z³, and Z⁴ is N.
29. The compound of any one of embodiments 17-28, wherein m is 0 or 1.
30. The compound of any one of embodiments 17-29, wherein n is 1 or 2.
31. The compound of embodiment 17, having one of the following structures:

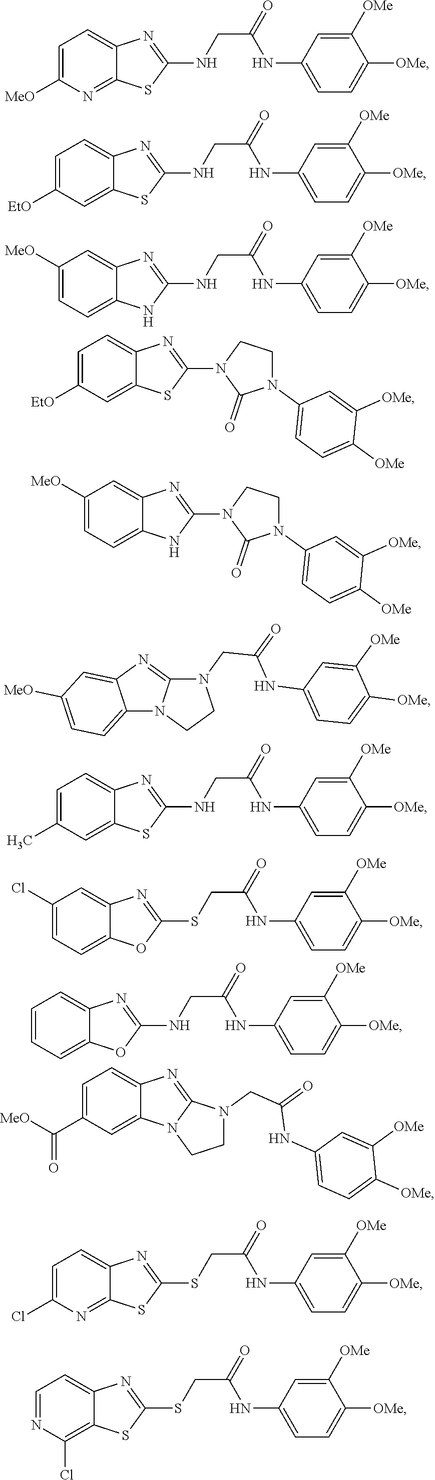

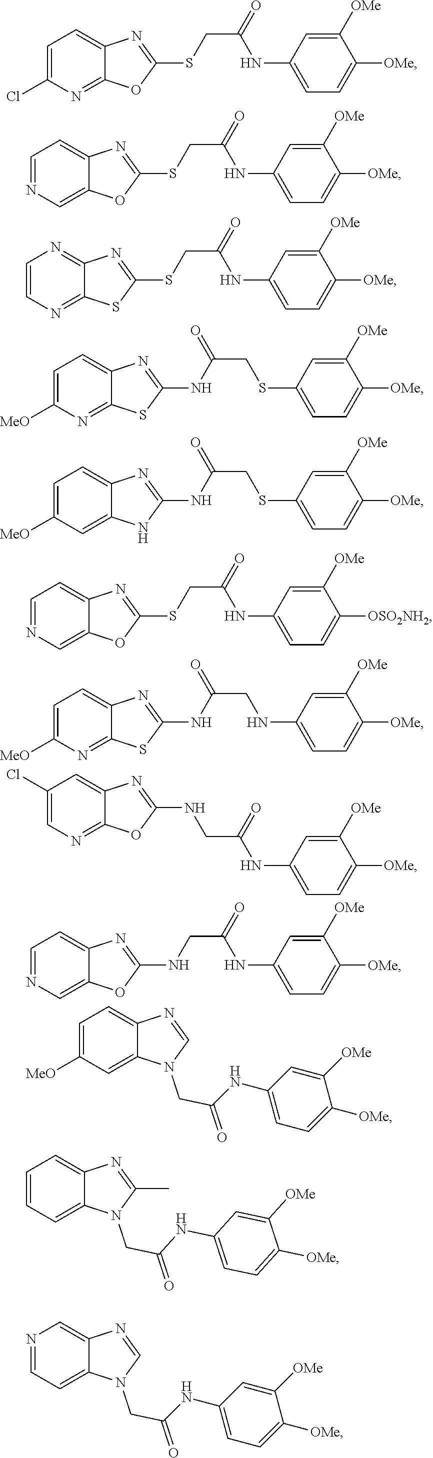

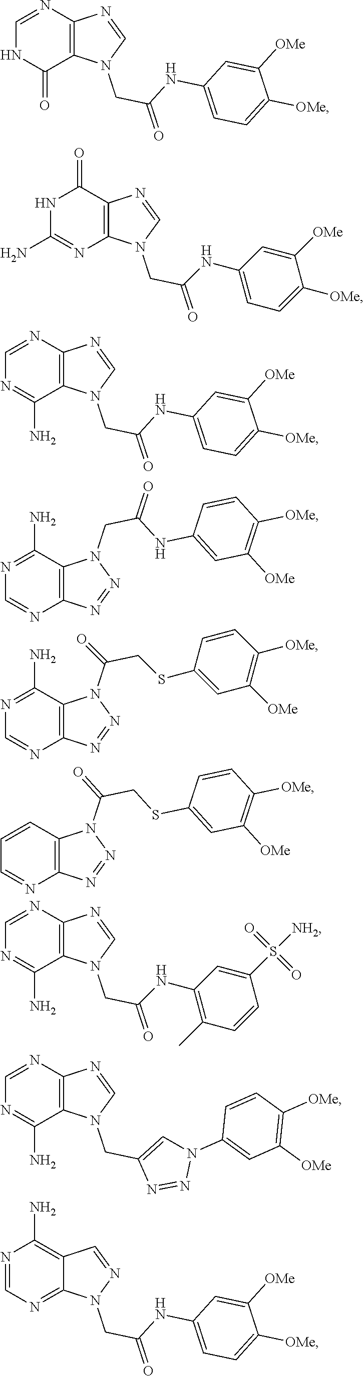

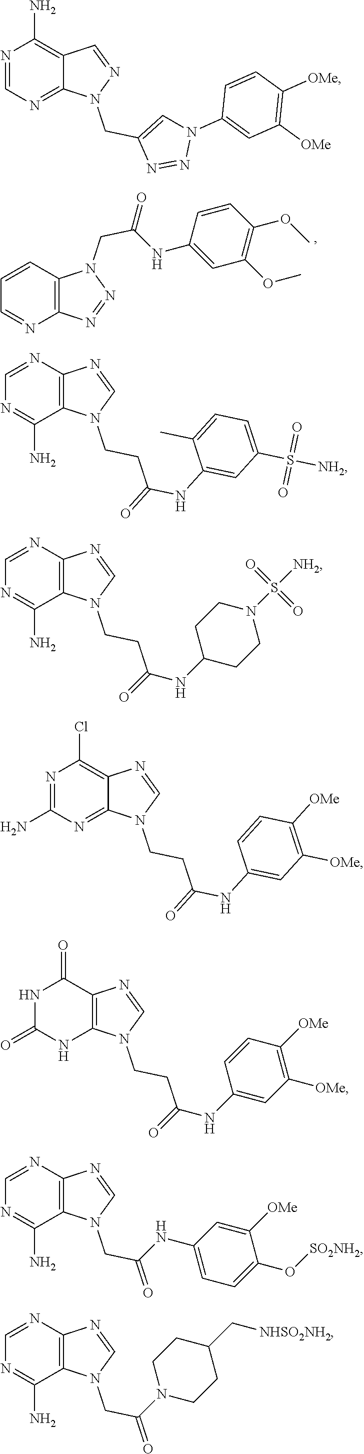

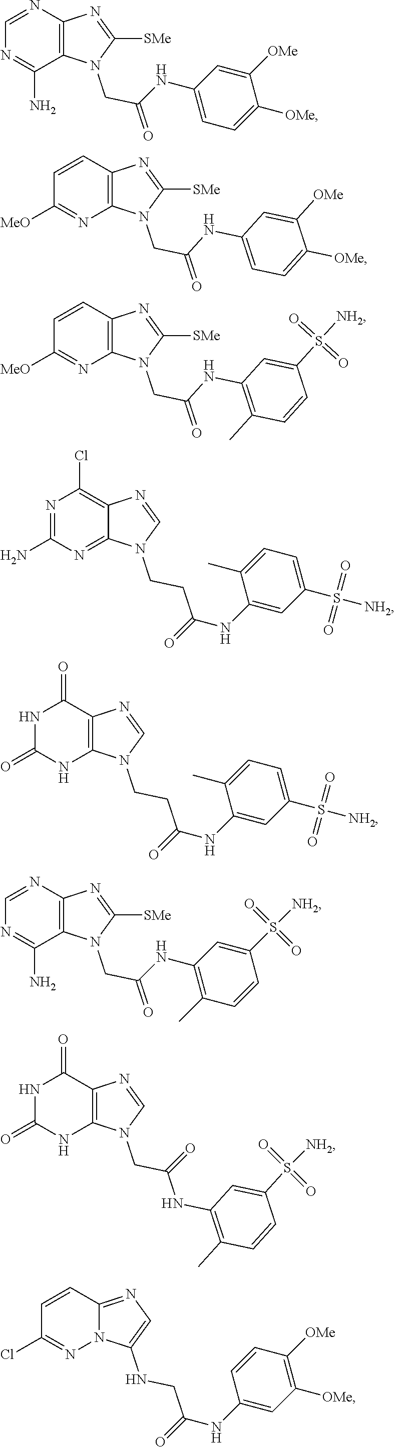

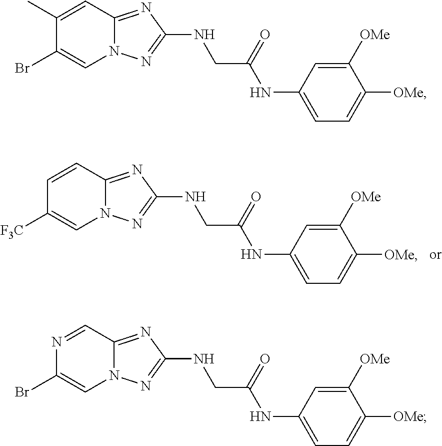 [SMILES: COc1ccc(NC(=O)CNc2nc3cc(C)c(Br)cn3n2)cc1OC ; COc1ccc(NC(=O)CNc2nc3ccc(C(F)(F)F)cn3n2)cc1OC ; COc1ccc(NC(=O)CNc2nc3cnc(Br)cn3n2)cc1OC]

or a pharmaceutically acceptable salt, tautomer, solvate, or hydrate thereof.
32. A compound of Formula (Z) or a pharmaceutically acceptable salt, hydrate, or tautomer thereof:

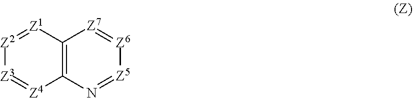

wherein:

Z¹, Z², Z³, Z⁴, Z⁵, Z⁶, and Z⁷ are each independently N or CR²², provided that
(a) one of Z¹, Z², Z³, Z⁴, Z⁵, Z⁶, or Z⁷ is -L-R¹⁸—;
(b) no more than two of Z¹, Z², Z³, or Z⁴ are N; and
(c) one of Z⁶ or Z⁷ is N;

wherein:

L is a linker selected from —N(R¹⁹)—, -alkylene-(NR¹⁹)—,

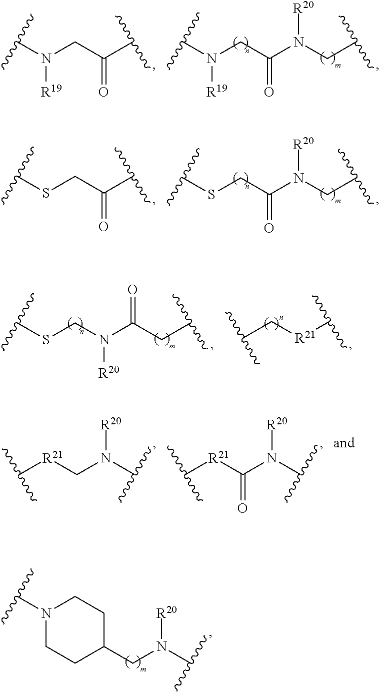

each of which is optionally substituted;

R¹⁸ is alkyl, carbocyclyl, heterocyclyl, aryl, or heteroaryl, each of which is optionally substituted;

R¹⁹ is H, alkyl, carbocyclyl, alkylenecarbocyclyl, or alkylenearyl;

R²⁰ is H, alkyl, alkylenecarbocyclyl, alkylenearyl;

R²¹ is carbocyclyl, heterocyclyl, or heteroaryl;

R²² is each independently halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN;

m is 0, 1, or 2; and

n is 1, 2, or 3.

33. The compound of embodiment 32, wherein Z¹, Z², Z³, and Z⁴ are each independently N or CR²².
34. The compound of embodiment 32 or 33, wherein Z⁶ is N and one of Z¹, Z⁵, or Z⁷ is -L-R¹⁸—.
35. The compound of embodiment 32 or 33, wherein Z⁷ is N and one of Z¹, Z⁵, or Z⁶ is -L-R¹⁸—.
36. The compound of any one of embodiments 32-35, wherein L is

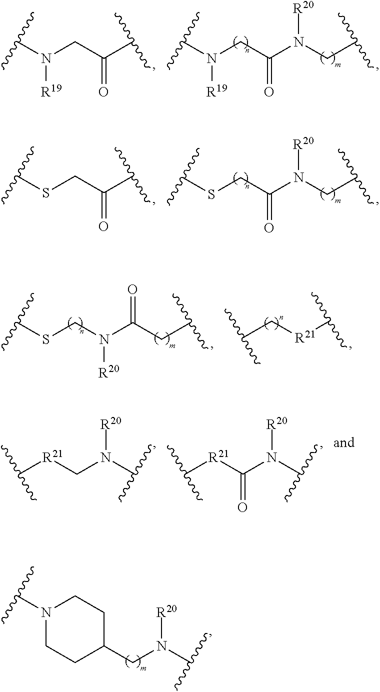

each of which is optionally substituted.
37. The compound of any one of embodiments 32-36, wherein R¹¹ is alkyl, heterocyclyl, aryl, or heteroaryl, each of which is optionally substituted.
38. The compound of any one of embodiments 32-37, wherein R¹⁹ is H or alkyl.
39. The compound of any one of embodiments 32-38, wherein R²⁰ is H, —C₁₋₅alkyl, —C₃₋₆carbocyclyl, —CH₂-aryl, or —CH₂—(C₃₋₆carbocyclyl).
40. The compound of any one of embodiments 32-39, wherein R²¹ is heterocyclyl or heteroaryl.
41. The compound of any one of embodiments 32-40, wherein m is 0 or 1.
42. The compound of any one of embodiments 32-41, wherein n is 1 or 2.
43. The compound of embodiment 32, having one of the following structures:

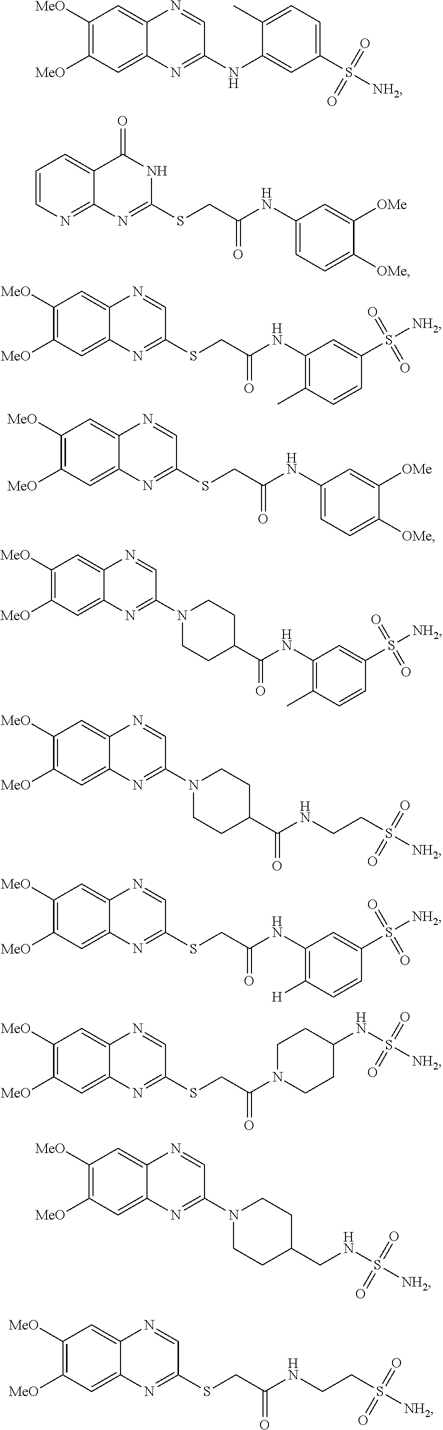

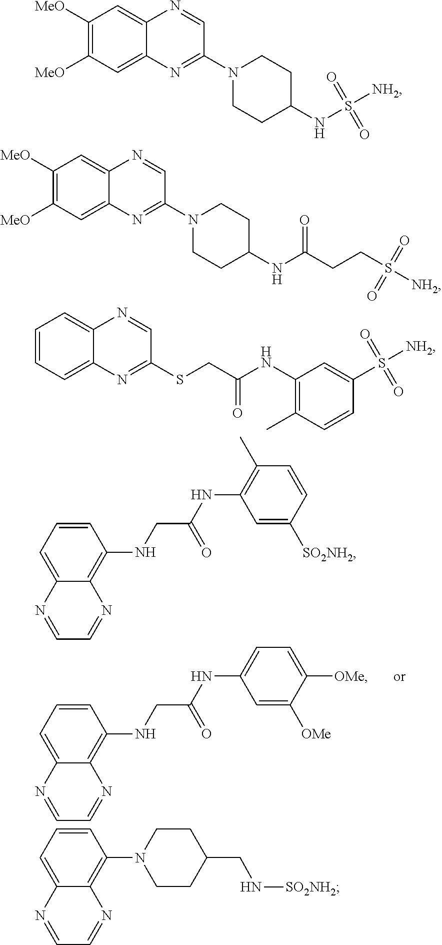

or a pharmaceutically acceptable salt, tautomer, hydrate, or solvate thereof.
44. The compound of embodiment 43, having one of the following structures:

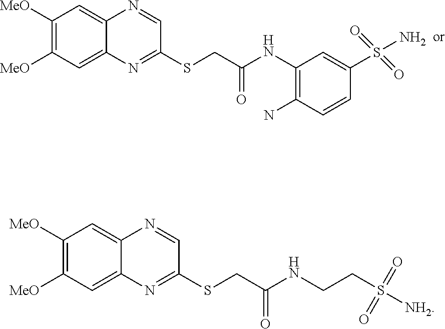 [SMILES: COc1cc2ncc(SCC(=O)Nc3cc(S(N)(=O)=O)ccc3N)nc2cc1OC ; COc1cc2ncc(SCC(=O)NCCS(N)(=O)=O)nc2cc1OC]

We claim:

1. A compound of Formula (X) or a pharmaceutically acceptable salt, hydrate, or tautomer thereof: 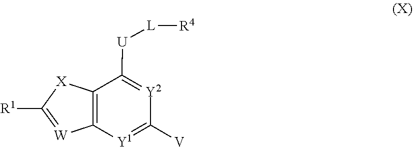
wherein:
L is a linker selected from alkylene, alkenylene, optionally substituted alkylene-S—, optionally substituted alkylene-O—, optionally substituted -alkylene-(NR⁵)—, optionally substituted 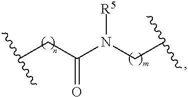
optionally substituted 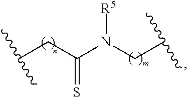
optionally substituted 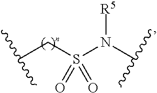
optionally substituted 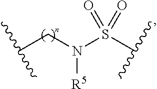
optionally substituted 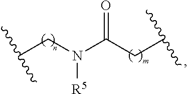
optionally substituted 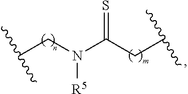
optionally substituted 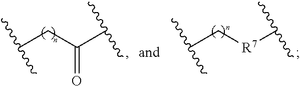
U is S or NH;
V is OH, NR²N³ or V and Y¹ taken together with the atoms to which they are attached form an optionally substituted phenyl or pyridinyl ring;
W is CH or N;
X is O, S, NR⁶, —CH═CH—, or —CH═N—;
Y¹ and Y² are each independently CH or N;
R¹ is H, OH, O-alkyl, alkyl or carbocyclyl;
R² and R³ are each independently H, alkyl, alkylenearyl, or —C(O)alkyl;
R⁴ is carbocyclyl, heterocyclyl, aryl, or heteroaryl, each of which is optionally substituted;
R⁵ is H, alkyl, —C(O)alkyl, carbocyclyl, alkylenecarbocyclyl, or alkylenearyl;
R⁶ is H, alkyl, carbocyclyl, alkylenecarbocyclyl, alkylenearyl, —C(O)alkyl, or —C(O)Oalkylenearyl;
R⁷ is carbocyclyl, heterocyclyl, or heteroaryl;
m is 0, 1, or 2; and
n is 1, 2, or 3.

2. The compound of claim 1, wherein L is a 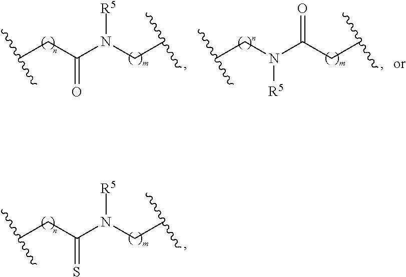
each of which is optionally substituted.

3. The compound of claim 2, wherein the optionally substituted 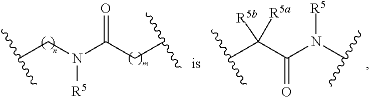
wherein:
R⁵ is H, alkyl, —C(O)alkyl, carbocyclyl, alkylenecarbocyclyl, or alkylenearyl; and
R^{5a} and R^{5b} are each independently selected from the group consisting of H, halogen, C₁₋₅alkyl, C₃₋₆carbocyclyl, alkylene-C₃₋₆carbocyclyl, aryl, alkylenearyl, or NH₂; wherein two —C₁₋₅alkyl taken together with the carbon atom to which they are attached form a C₃₋₆carbocyclyl.

4. The compound of claim 3, wherein 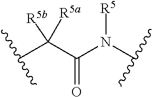
is selected from the group consisting of: 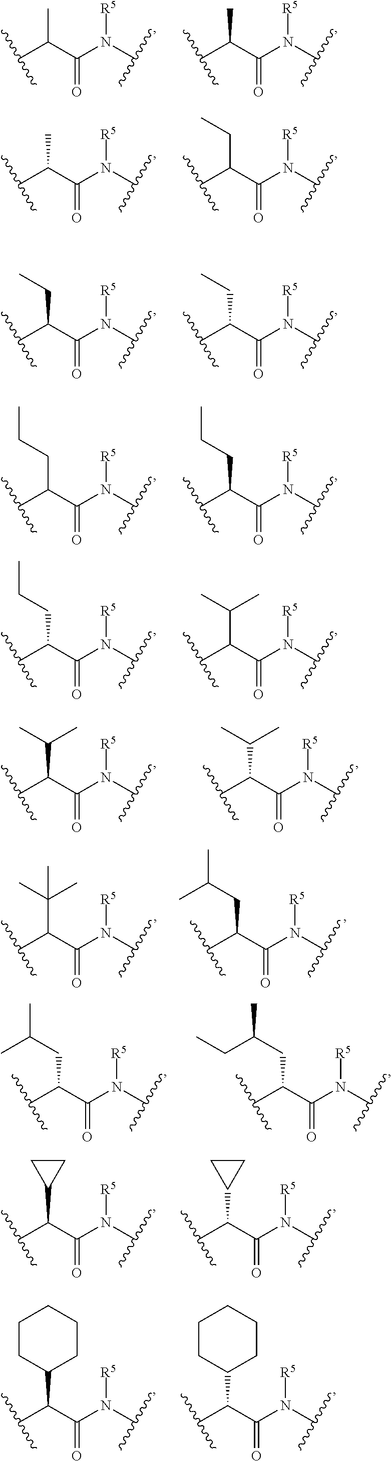 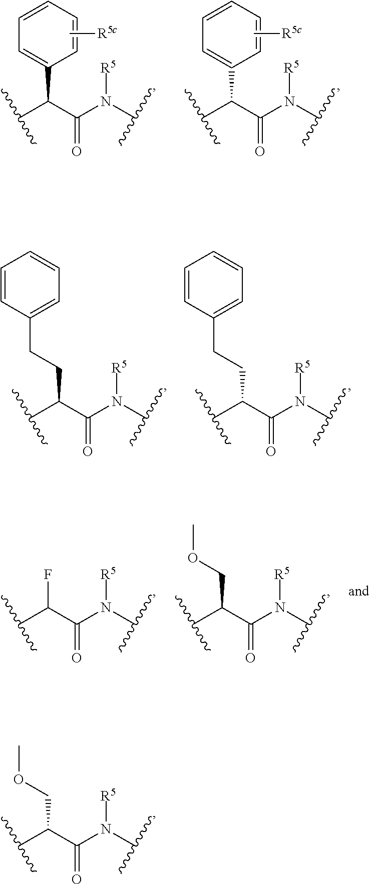
wherein:
R⁵ is H, Me, or —C(O)alkyl; and
R^{5c} is halogen, alkyl, haloalkyl, hydroxy, or alkoxy.

5. The compound of claim 3, wherein 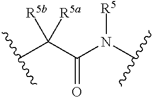
is selected from the group consisting of: 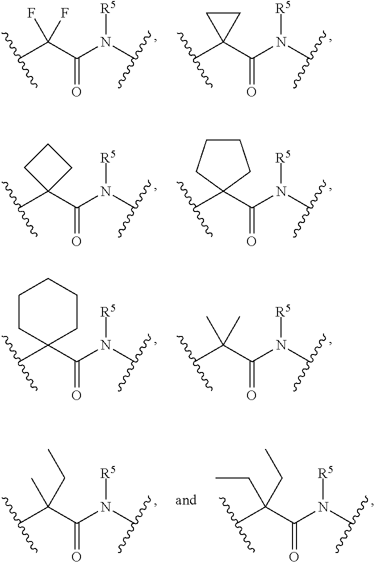
wherein R⁵ is H, Me, or —C(O)alkyl.

6. The compound of any one of claims 1-5, wherein U is S.

7. The compound of any one of claims 1-6, wherein V is NR²R³.

8. The compound of any one of claims 1-7, wherein W is N.

9. The compound of any one of claims 1-8, wherein X is NR⁶.

10. The compound of any one of claims 1-9, wherein Y¹ and Y² are both N.

11. The compound of any one of claims 1-10, wherein R¹ is H, OH, or C₁₋₅alkyl.

12. The compound of any one of claims 1-11, wherein R² and R³ are independently H, —C₁₋₅alkyl, —CH₂Ph, or —C(O)(C₁₋₅alkyl).

13. The compound of any one of claims 1-12, wherein R⁴ is aryl or heteroaryl, each of which is optionally substituted.

14. The compound of any one of claims 1-13, wherein R⁴ is selected from the group consisting of: 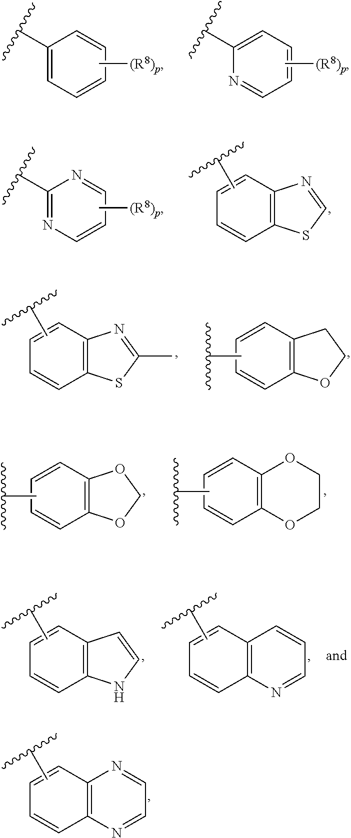
wherein:
each R⁸ is independently halogen, C₁₋₅ alkyl, —C₁₋₅ alkenyl, —OH, —OC₁₋₅alkyl, —COOH, or —CO₂C₁₋₅alkyl; and
p is an integer from 0-3.

15. The compound of any one of claims 1-14, wherein R⁴ is selected from the group consisting of: 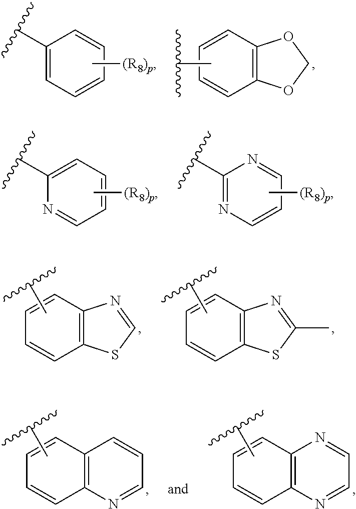
wherein:
each R⁸ is independently halogen, C₁₋₅ alkyl, —OH, —OC₁₋₅alkyl, —COOH, or —CO₂C₁₋₅alkyl; and
p is an integer from 0-3.

16. The compound of any one of claims 1-15, wherein R⁵ is H, —C₁₋₅alkyl, —C(O)C₁₋₅alkyl, —C₃₋₆carbocyclyl, —CH₂-aryl, or —CH₂—(C₃₋₆carbocyclyl).

17. The compound of any one of claims 1-16, wherein R⁶ is H, —C₁₋₅alkyl, —CH₂aryl, or —CH₂—(C₃₋₆carbocyclyl).

18. The compound of any one of claims 1-17, wherein R⁷ is a C₃₋₆carbocyclyl, a 3- to 6-membered heterocyclyl, or a 5- to 6-membered heteroaryl.

19. The compound of any one of claims 1-18, wherein m is 0 or 1.

20. The compound of any one of claims 1-19, wherein n is 1 or 2.

21. The compound of claim 1, having one of the following structures: 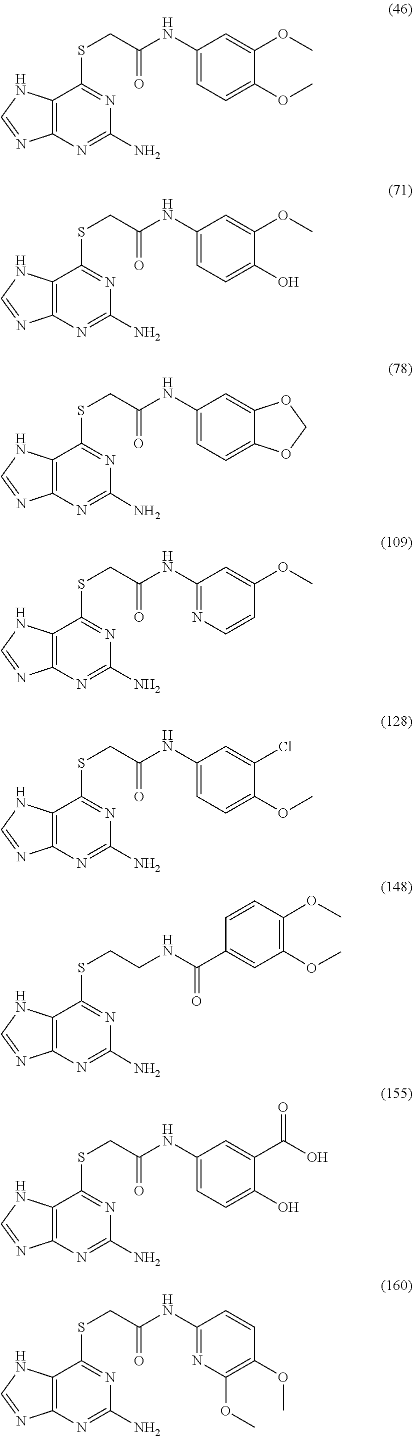 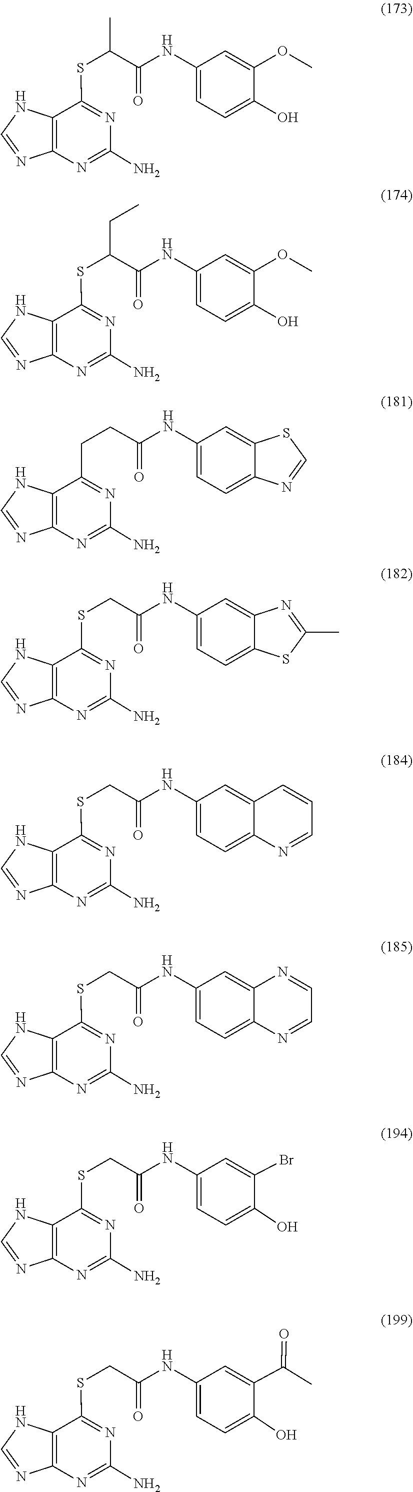 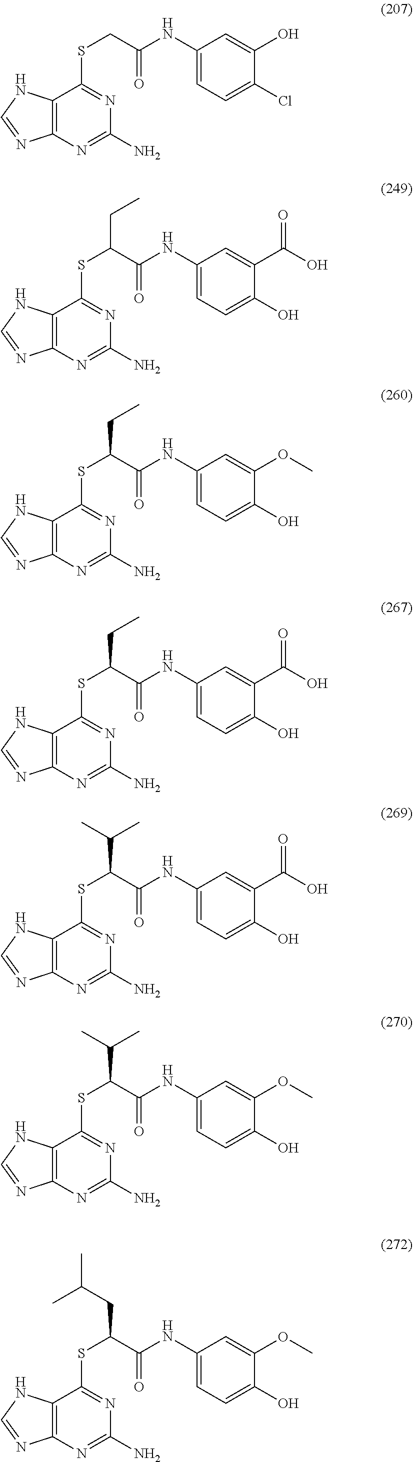 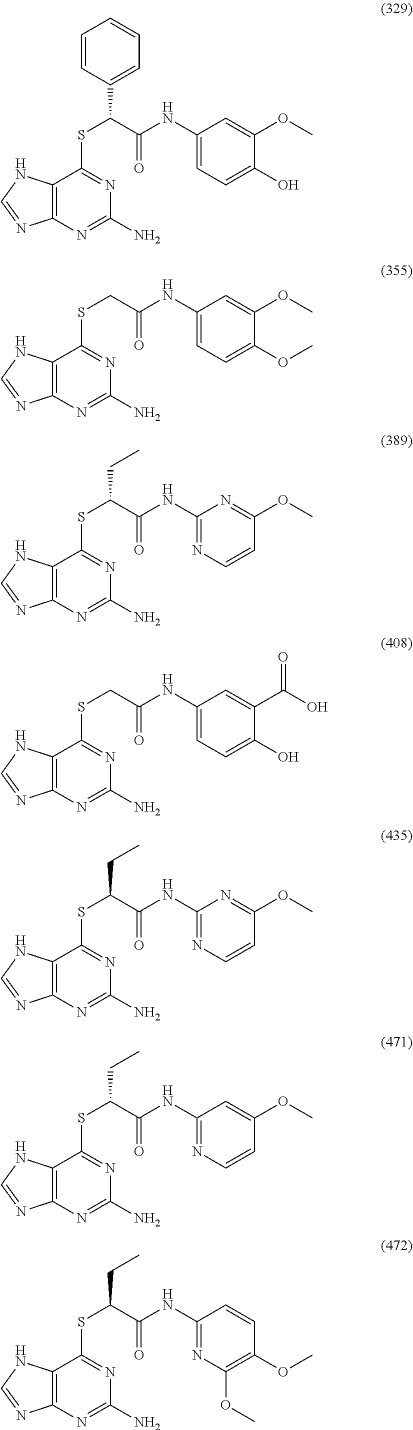 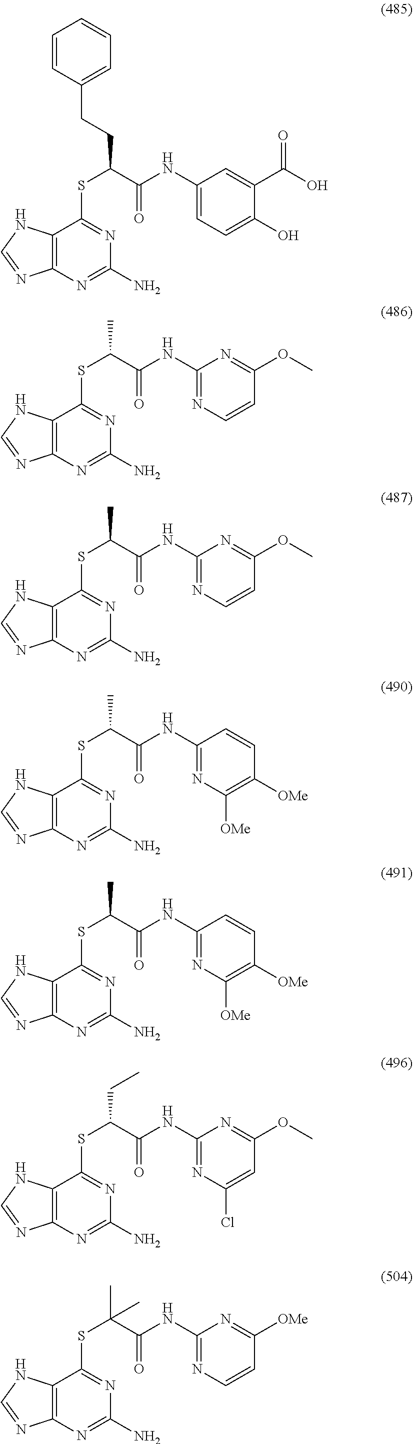 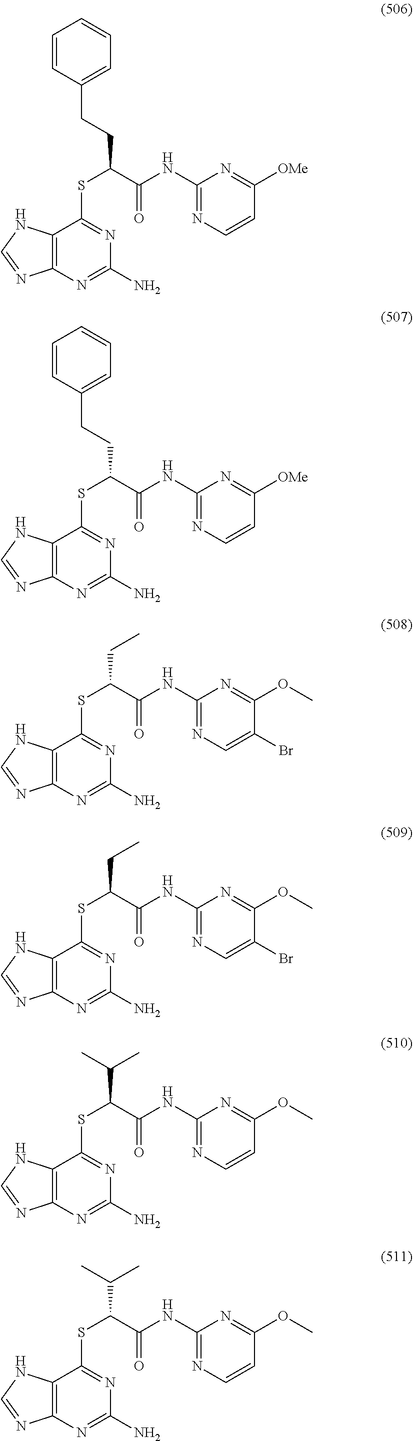 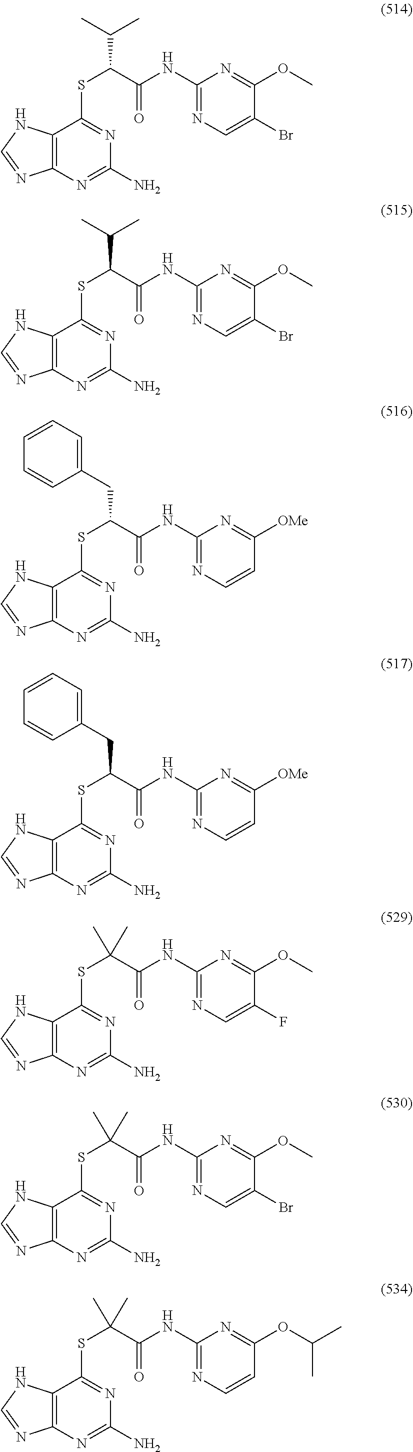 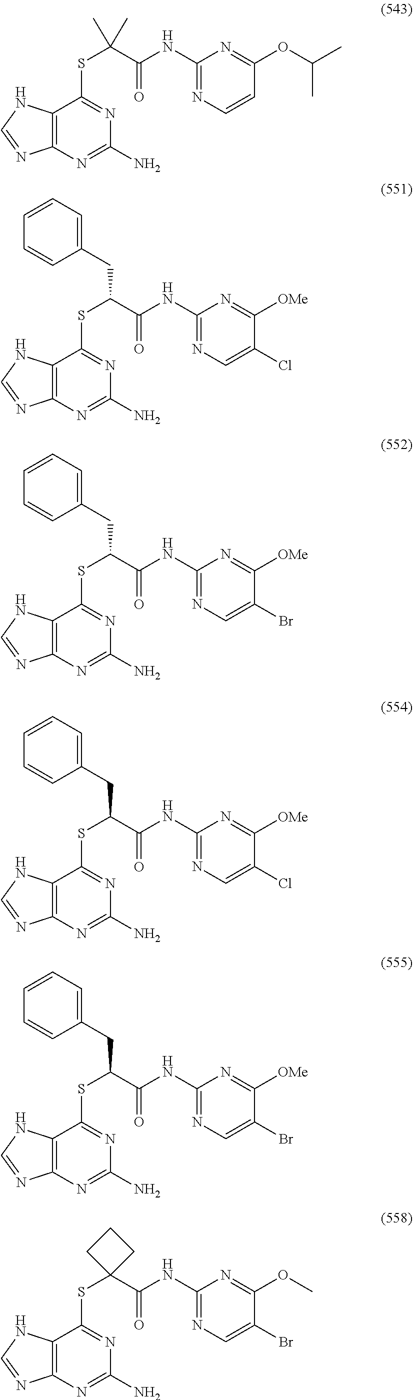 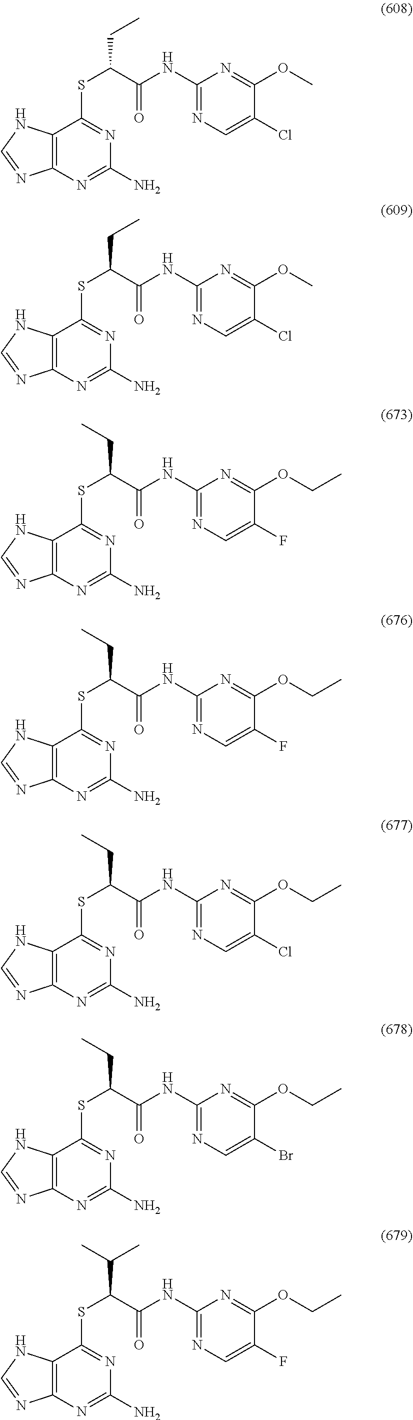 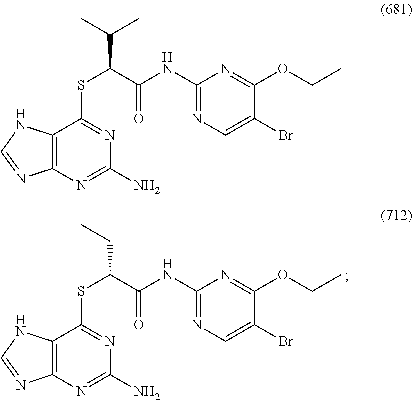
or a pharmaceutically acceptable salt, tautomer, hydrate or solvate thereof.

22. A compound of Formula (Y) or a pharmaceutically acceptable salt, hydrate, or tautomer thereof: 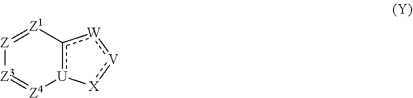
wherein:
U is C or N;
wherein
when U is C, Y is 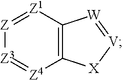
or
when U is N, Y is 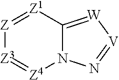
V is N or CR¹⁰;
W is CH or N;
X is S, O, N-L-R¹¹, or NR¹²;
L is selected from alkylene, alkenylene, optionally substituted -alkylene-(NR¹²)—, optionally substituted 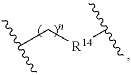
optionally substituted 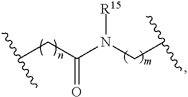
optionally substituted 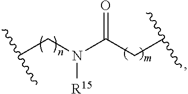
optionally substituted 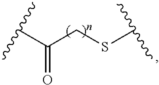
optionally substituted 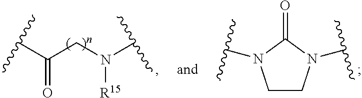
R¹⁰ is H, alkyl, —O-alkyl, —S-alkyl, carbocyclyl, alkylenecarbocyclyl, —O-L-R¹¹, —S-L-R¹¹, —N(R¹²)-L-R¹¹, -L-R¹¹;
R¹¹ is alkyl, carbocyclyl, heterocyclyl, aryl, or heteroaryl, each of which is optionally substituted; and
R¹² is each independently H, alkyl, alkylenecarbocyclyl, or carbocyclyl, wherein two R¹² groups taken together with the carbon atom to which they are attached can form a heterocyclyl;
R¹⁴ is carbocyclyl, heterocyclyl, or heteroaryl;
R¹⁵ is H, alkyl, carbocyclyl, alkylenecarbocyclyl, or alkylenearyl;
wherein:
when X is N-L-R¹¹, V is N or CR¹⁰, wherein R¹⁰ is H, alkyl, —O-alkyl, —S-alkyl, carbocyclyl, or alkylenecarbocyclyl;
when X is S, O, NR¹²; V is CR¹⁰, wherein R¹⁰ is —O-L-R¹¹, —S-L-R¹¹, —N(R¹²)-L-R¹¹, or -L-R¹¹; or
when U is N, V is CR¹⁰, wherein R¹⁰ is —O-L-R¹¹, —S-L-R¹¹, —N(R¹²)-L-R¹, or -L-R¹¹;
Z¹, Z², Z³, and Z⁴ are each independently CR¹³ or N;
R¹³ is H, halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN, wherein two R¹³ taken together with the atoms to which they are attached can form carbocyclyl, heterocyclyl, or heteroaryl, each of which is optionally substituted;
m is 0, 1, or 2; and
n is 1, 2, or 3.

23. The compound of claim 22, wherein U is C.

24. The compound of claim 22 or 23, wherein W is N.

25. The compound of claim 22-24, wherein when X is S, O, or NH, V is CR¹⁰, wherein R¹⁰ is —O-L-R¹¹, —S-L-R¹¹, —N(R¹²)-L-R¹¹, or -L-R¹¹.

26. The compound of any one of claims 22-25, wherein X is N-L-R¹¹ and V is CR¹⁰, wherein R¹⁰ is H, alkyl, —O-alkyl, or —S-alkyl.

27. The compound of any one of claims 22-26, wherein L is -alkylene-(NR¹²)—, 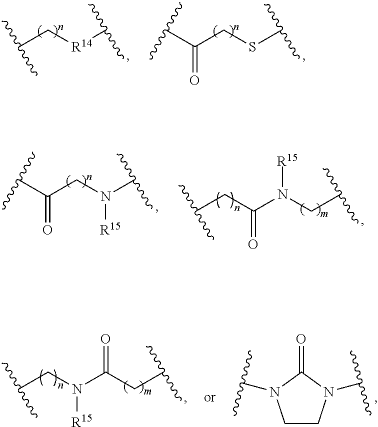
each of which is optionally substituted.

28. The compound of any one of claims 22-27, wherein R¹¹ is heterocyclyl, aryl, or heteroaryl, each of which is optionally substituted.

29. The compound of any one of claims 22-28, wherein R¹² is each independently H or C₁₋₅alkyl.

30. The compound of any one of claims 22-29, wherein R¹⁴ is heterocyclyl or heteroaryl.

31. The compound of any one of claims 22-30, wherein R¹⁵ is H or alkyl.

32. The compound of any one of claims 22-31, wherein each of Z¹, Z², Z³, and Z⁴ is CR¹³.

33. The compound of any one of claims 22-31, wherein at least one of Z¹, Z², Z³, and Z⁴ is N.

34. The compound of any one of claims 22-33, wherein m is 0 or 1.

35. The compound of any one of claims 22-34, wherein n is 1 or 2.

36. The compound of claim 22, having one of the following structures: 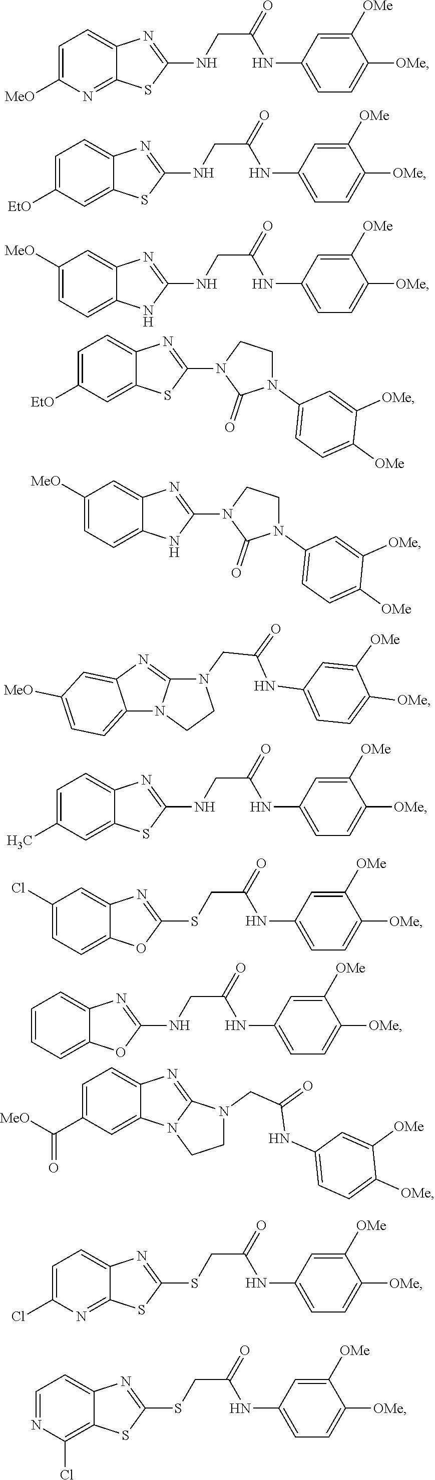 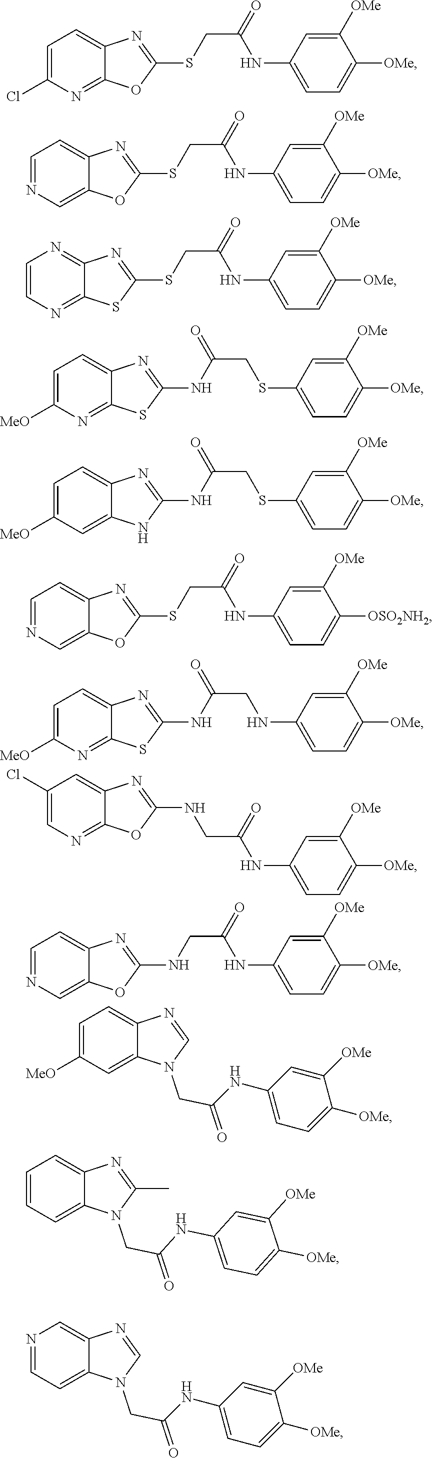 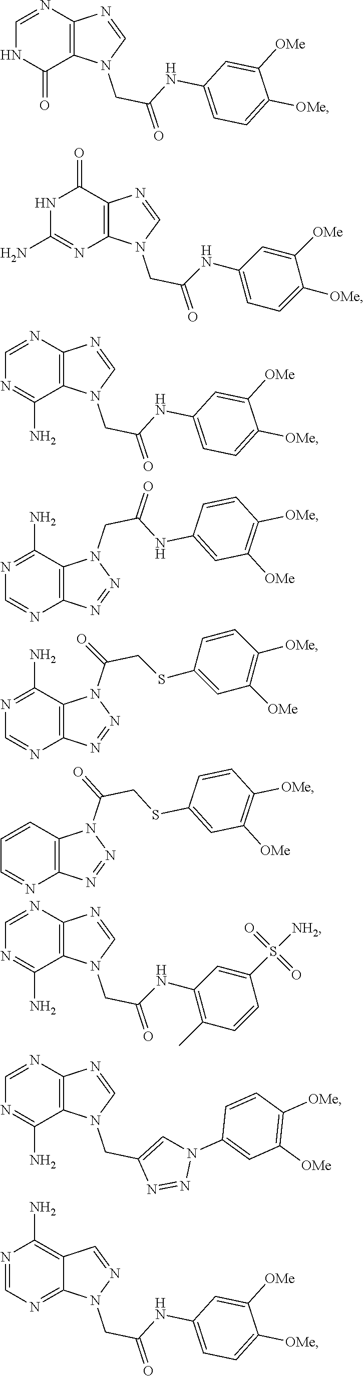 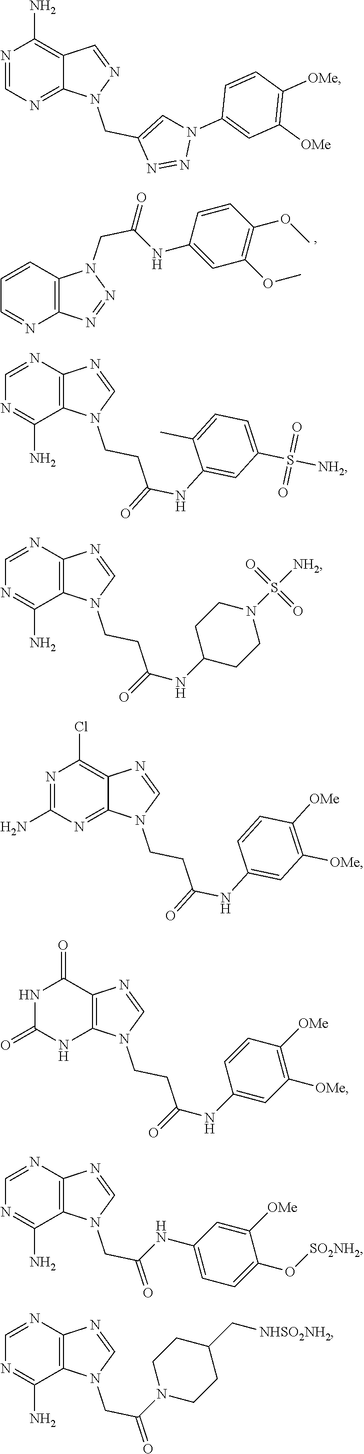 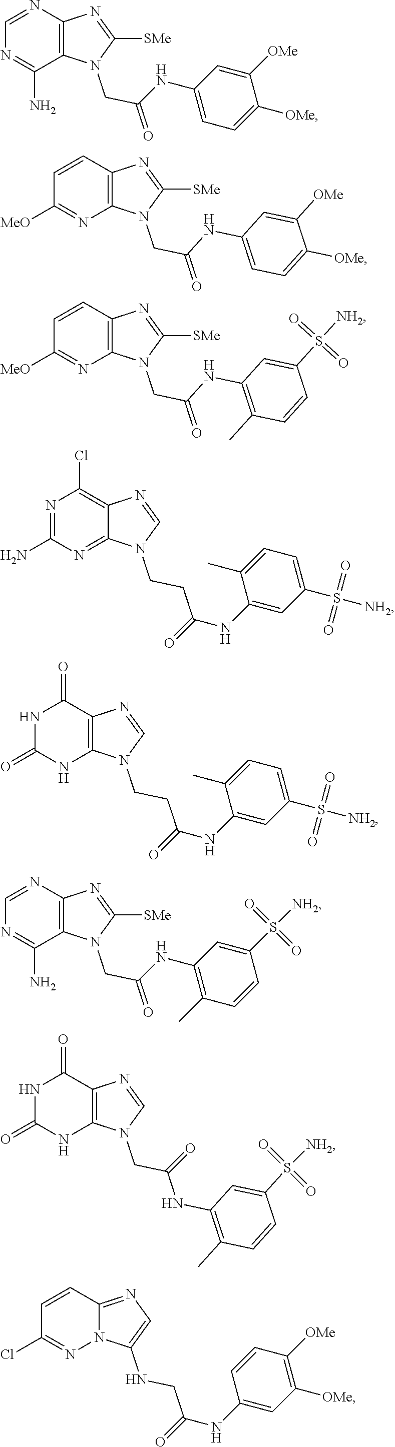 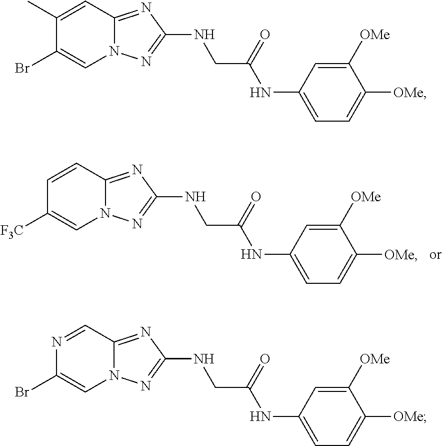 [SMILES: COc1ccc(NC(=O)CNc2nc3cc(C)c(Br)cn3n2)cc1OC ; COc1ccc(NC(=O)CNc2nc3ccc(C(F)(F)F)cn3n2)cc1OC ; COc1ccc(NC(=O)CNc2nc3cnc(Br)cn3n2)cc1OC]
or a pharmaceutically acceptable salt, tautomer, solvate, or hydrate thereof.

37. A compound of Formula (Z) or a pharmaceutically acceptable salt, hydrate, or tautomer thereof: 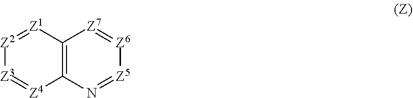
wherein:
Z¹, Z², Z³, Z⁴, Z⁵, Z⁶, and Z⁷ are each independently N or CR²², provided that
(a) one of Z¹, Z², Z³, Z⁴, Z⁵, Z⁶, or Z⁷ is -L-R⁸—;
(b) no more than two of Z¹, Z², Z³, or Z⁴ are N; and
(c) one of Z⁶ or Z⁷ is N;
wherein:
L is a linker selected from —N(R¹⁹)—, -alkylene-(NR¹⁹)—, 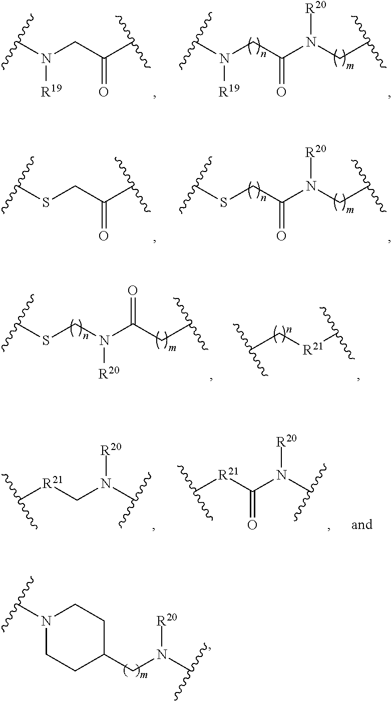
each of which is optionally substituted;
R¹⁸ is alkyl, carbocyclyl, heterocyclyl, aryl, or heteroaryl, each of which is optionally substituted;
R¹⁹ is H, alkyl, carbocyclyl, alkylenecarbocyclyl, or alkylenearyl;
R²⁰ is H, alkyl, alkylenecarbocyclyl, alkylenearyl;
R²¹ is carbocyclyl, heterocyclyl, or heteroaryl;
R²² is each independently halogen, alkyl, alkene, alkyne, haloalkyl, carbocyclyl, OH, O-alkyl, O-haloalkyl, O-carbocyclyl, OSO₂-alkyl, OSO₂-aryl, —C(O)alkyl, —C(O)Oalkyl, —C(O)Oalkylenearyl, —C(O)Oaryl, —SO₂NH₂, —SO₂NHalkyl, —SO₂NH(alkyl)₂, —NH₂, —NHalkyl, —N(alkyl)₂, —N(H)SO₂alkyl, —N(H)SO₂aryl, or —CN;
m is 0, 1, or 2; and
n is 1, 2, or 3.

38. The compound of claim 37, wherein Z¹, Z², Z³, and Z⁴ are each independently N or CR²².

39. The compound of claim 37or 38, wherein Z⁶ is N and one of Z¹, Z⁵, or Z⁷ is -L-R¹⁸—.

40. The compound of claim 37or 38, wherein Z⁷ is N and one of Z¹, Z⁵, or Z⁶ is -L-R¹¹—.

41. The compound of any one of claims 37-40, wherein L is 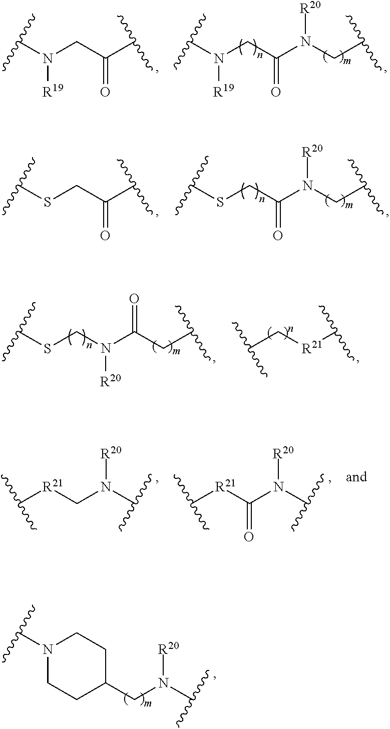
each of which is optionally substituted.

42. The compound of any one of claims 37-41, wherein R¹¹ is alkyl, heterocyclyl, aryl, or heteroaryl, each of which is optionally substituted.

43. The compound of any one of claims 37-42, wherein R¹⁹ is H or alkyl.

44. The compound of any one of claims 37-43, wherein R²⁰ is H, —C₁₋₅alkyl, —C₃₋₆carbocyclyl, —CH₂-aryl, or —CH₂—(C₃₋₆carbocyclyl).

45. The compound of any one of claims 37-44, wherein R²¹ is heterocyclyl or heteroaryl.

46. The compound of any one of claims 37-45, wherein m is 0 or 1.

47. The compound of any one of claims 37-46, wherein n is 1 or 2.

48. The compound of claim 37, having one of the following structures: 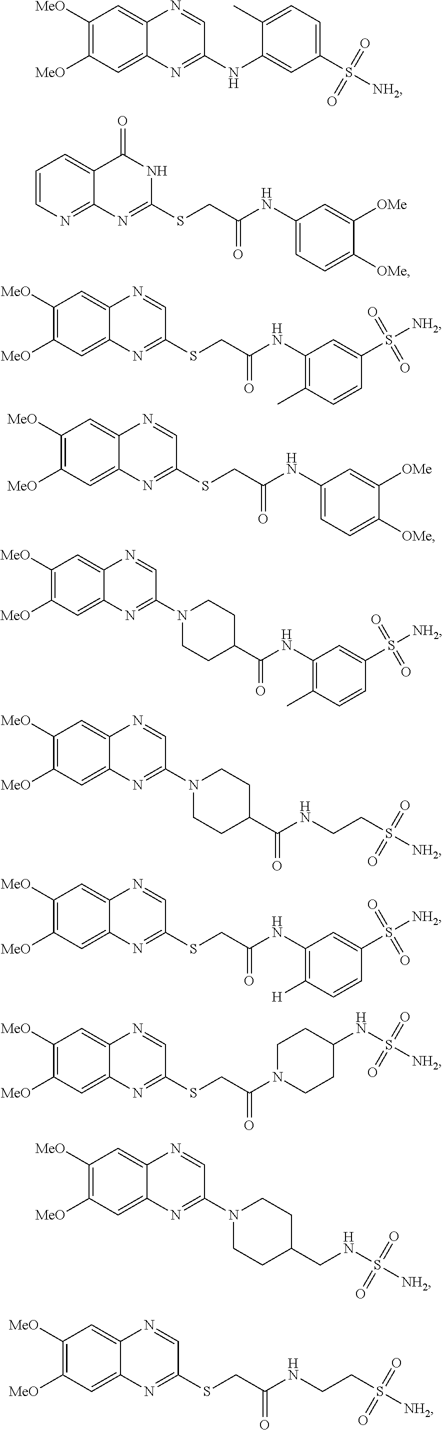 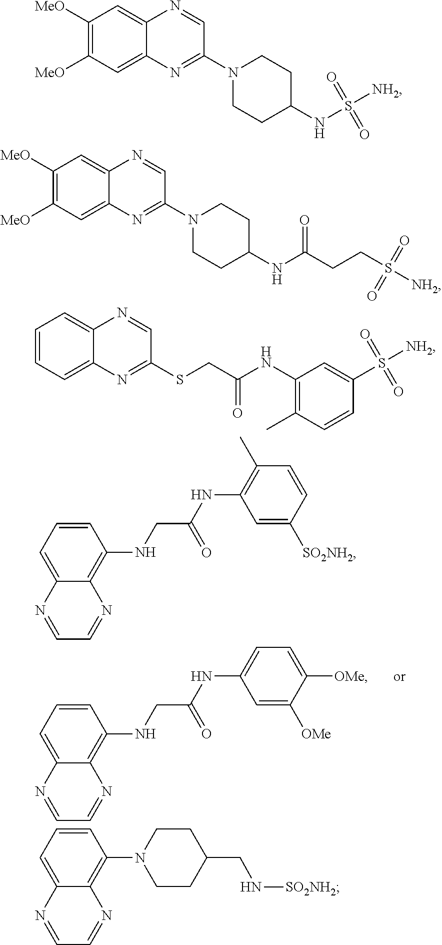
or a pharmaceutically acceptable salt, tautomer, hydrate, or solvate thereof one of the following structures.

49. The compound of embodiment 48, having one of the following structures: 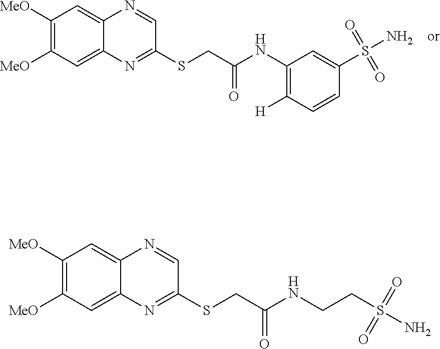 [SMILES: COc1cc2ncc(SCC(=O)Nc3cccc(S(N)(=O)=O)c3)nc2cc1OC ; COc1cc2ncc(SCC(=O)NCCS(N)(=O)=O)nc2cc1OC]
